SEQUENCE LISTING (98 sequences)

SEQ ID NO: 1 - protein, 1337 aa, Artificial Sequence
  synthetic construct; RGX-Dys1
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 2 - protein, 1191 aa, Artificial Sequence
  synthetic construct; RGX-Dys3
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 3 - protein, 240 aa, Artificial Sequence
  synthetic construct; microdystrophin segment
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLP

SEQ ID NO: 4 - protein, 12 aa, Artificial Sequence
  synthetic construct; microdystrophin segment
QQVSIEAIQEVE

SEQ ID NO: 5 - protein, 75 aa, Artificial Sequence
  synthetic construct; microdystrophin segment
MLPRPPKVTKEEHFQLHHQMHYSQQITVSLAQGYERTSSPKPRFKSYAYTQAAYVTTSDPTRSPFPSQHLEAPED

SEQ ID NO: 6 - protein, 9 aa, Artificial Sequence
  synthetic construct; microdystrophin segment
KSFGSSLME

SEQ ID NO: 7 - protein, 111 aa, Artificial Sequence
  synthetic construct; microdystrophin segment
SEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHR

SEQ ID NO: 8 - protein, 109 aa, Artificial Sequence
  synthetic construct; microdystrophin segment
VLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQD

SEQ ID NO: 9 - protein, 2 aa, Artificial Sequence
  synthetic construct; microdystrophin segment
IL

SEQ ID NO: 10 - protein, 109 aa, Artificial Sequence
  synthetic construct; microdystrophin segment
LKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQ

SEQ ID NO: 11 - protein, 47 aa, Artificial Sequence
  synthetic construct; microdystrophin segment
QPDLAPGLTTIGASPTQTVTLVTQPVVTKETAISKLEMPSSLMLEVP

SEQ ID NO: 12 - protein, 3 aa, Artificial Sequence
  synthetic construct; microdystrophin segment
TLE

SEQ ID NO: 13 - protein, 106 aa, Artificial Sequence
  synthetic construct; microdystrophin segment
RLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHE

SEQ ID NO: 14 - protein, 72 aa, Artificial Sequence
  synthetic construct; microdystrophin segment
AHRDFGPASQHFLSTSVQGPWERAISPNKVPYYINHETQTTCWDHPKMTELYQSLADLNNVRFSAYRTAMKL

SEQ ID NO: 15 - protein, 248 aa, Artificial Sequence
  synthetic construct; microdystrophin segment
RRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYC

SEQ ID NO: 16 - protein, 194 aa, Artificial Sequence
  synthetic construct; microdystrophin segment
TPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 17 - protein, 4 aa, Artificial Sequence
  synthetic construct; microdystrophin segment
ETLE

SEQ ID NO: 18 - protein, 2 aa, Artificial Sequence
  synthetic construct; microdystrophin segment
LE

SEQ ID NO: 19 - DNA, 6 bp, Artificial Sequence
  synthetic construct; RGX-Dys segment
agtgtg

SEQ ID NO: 20 - DNA, 4017 bp, Artificial Sequence
  synthetic construct; RGX-Dys1
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatga

SEQ ID NO: 21 - DNA, 3573 bp, Artificial Sequence
  synthetic construct; RGX-Dys3
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacc

SEQ ID NO: 22 - DNA, 720 bp, Artificial Sequence
  synthetic construct; microdystrophin segment
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcct

SEQ ID NO: 23 - DNA, 36 bp, Artificial Sequence
  synthetic construct; microdystrophin segment
caacaagtgagcattgaagccatccaggaagtggaa

SEQ ID NO: 24 - DNA, 225 bp, Artificial Sequence
  synthetic construct; microdystrophin segment
atgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagac

SEQ ID NO: 25 - DNA, 27 bp, Artificial Sequence
  synthetic construct; microdystrophin segment
aagtcatttggcagttcattgatggag

SEQ ID NO: 26 - DNA, 333 bp, Artificial Sequence
  synthetic construct; microdystrophin segment
agtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacataga

SEQ ID NO: 27 - DNA, 327 bp, Artificial Sequence
  synthetic construct; microdystrophin segment
gttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagac

SEQ ID NO: 28 - DNA, 6 bp, Artificial Sequence
  synthetic construct; microdystrophin segment
atcctt

SEQ ID NO: 29 - DNA, 327 bp, Artificial Sequence
  synthetic construct; microdystrophin segment
ctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacag

SEQ ID NO: 30 - DNA, 141 bp, Artificial Sequence
  synthetic construct; microdystrophin segment
cagcctgacctagctcctggactgaccactattggagcctctcctactcagactgttactctggtgacacaacctgtggttactaaggaaactgccatctccaaactagaaatgccatcttccttgatgttggaggtacct

SEQ ID NO: 31 - DNA, 9 bp, Artificial Sequence
  synthetic construct; microdystrophin segment
acccttgaa

SEQ ID NO: 32 - DNA, 315 bp, Artificial Sequence
  synthetic construct; microdystrophin segment
agactccaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaa

SEQ ID NO: 33 - DNA, 216 bp, Artificial Sequence
  synthetic construct; microdystrophin segment
gcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactc

SEQ ID NO: 34 - DNA, 744 bp, Artificial Sequence
  synthetic construct; microdystrophin segment
cgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgc

SEQ ID NO: 35 - DNA, 582 bp, Artificial Sequence
  synthetic construct; microdystrophin segment
actccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgg

SEQ ID NO: 36 - DNA, 12 bp, Artificial Sequence
  synthetic construct; microdystrophin segment
gagacccttgaa

SEQ ID NO: 37 - DNA, 6 bp, Artificial Sequence
  synthetic construct; microdystrophin segment
cttgaa

SEQ ID NO: 38 - DNA, 12 bp, Artificial Sequence
  synthetic construct; microdystrophin segment
cagacactggaa

SEQ ID NO: 39 - DNA, 328 bp, Artificial Sequence
  synthetic construct; spc5-12 promoter
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 40 - DNA, 174 bp, Artificial Sequence
  synthetic construct; promoter
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaag

SEQ ID NO: 41 - DNA, 82 bp, Artificial Sequence
  synthetic construct; intron
gtgagtatctcagggatccagacatggggatatgggaggtgcctctgatcccagggctcactgtgggtctctctgttcacag

SEQ ID NO: 42 - DNA, 50 bp, Artificial Sequence
  synthetic construct; polyA
aggcctaataaagagctcagatgcatcgatcagagtgtgttggttttttg

SEQ ID NO: 43 - DNA, 25 bp, Artificial Sequence
  synthetic construct; primer
tgactcgagaggcctaataaagagc

SEQ ID NO: 44 - DNA, 22 bp, Artificial Sequence
  synthetic construct; primer
ccttggagactgtggagaggtg

SEQ ID NO: 45 - protein, 738 aa, Artificial Sequence
  synthetic construct; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 46 - DNA, 12 bp, Artificial Sequence
  synthetic construct; microdystrophin segment
caaacccttgaa

SEQ ID NO: 47 - DNA, 162 bp, Artificial Sequence
  synthetic construct; microdystrophin segment
gccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgc

SEQ ID NO: 48 - protein, 738 aa, Artificial Sequence
  synthetic construct; capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 49 - protein, 736 aa, Artificial Sequence
  synthetic construct; capsid
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 737 aa, Artificial Sequence
  synthetic construct; capsid
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - DNA, 865 bp, Artificial Sequence
  synthetic construct; promoter
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 52 - DNA, 1725 bp, Artificial Sequence
  synthetic construct; promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 53 - DNA, 4734 bp, Artificial Sequence
  synthetic construct; promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatgactcgagaggcctaataaagagctcagatgcatcgatcagagtgtgttggttttttgtgtgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 212 bp, Artificial Sequence
  synthetic construct; promoter
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 55 - DNA, 4364 bp, Artificial Sequence
  synthetic construct; RGX-Dys3 cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaaggtgagtatctcagggatccagacatggggatatgggaggtgcctctgatcccagggctcactgtgggtctctctgttcacaggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacctgatgagtcgacaggcctaataaagagctcagatgcatcgatcagagtgtgttggttttttgtgtggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - protein, 251 aa, Artificial Sequence
  synthetic construct; promoter
  misc (1)..(4): Xaa can be any amino acid
ATGGAGGCGGTACTATGTAGATGAGAATTCAGGAGCAAACTGGGAAAAGCAACTGCTTCCAAATATTTGTGATTTTTACAGTGTAGTTTTGGAAAAACTCTTAGCCTACCAATTCTTCTAAGTGTTTTAAAATGTGGGAGCCAGTACACATGAAGTTATAGAGTGTTTTAATGAGGCTTAAATATTTACCGTAACTATGAAATGCTACGCATATCATGCTGTTCAGGCTCCGTGGCCACGCAACTCATACT

SEQ ID NO: 57 - DNA, 720 bp, Artificial Sequence
  synthetic construct; RGX-Dys segment
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccc

SEQ ID NO: 58 - DNA, 36 bp, Artificial Sequence
  synthetic construct; RGX-Dys segment
cagcaagtgtccattgaggccattcaagaggttgag

SEQ ID NO: 59 - DNA, 225 bp, Artificial Sequence
  synthetic construct; RGX-Dys segment
atgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggac

SEQ ID NO: 60 - DNA, 27 bp, Artificial Sequence
  synthetic construct; RGX-Dys segment
aagagctttggcagcagcctgatggaa

SEQ ID NO: 61 - DNA, 333 bp, Artificial Sequence
  synthetic construct; RGX-Dys segment
tctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacaga

SEQ ID NO: 62 - DNA, 327 bp, Artificial Sequence
  synthetic construct; RGX-Dys segment
gtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggac

SEQ ID NO: 63 - DNA, 6 bp, Artificial Sequence
  synthetic construct; RGX-Dys segment
attctg

SEQ ID NO: 64 - DNA, 327 bp, Artificial Sequence
  synthetic construct; RGX-Dys segment
ctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccag

SEQ ID NO: 65 - DNA, 141 bp, Artificial Sequence
  synthetic construct; RGX-Dys segment
caacctgatcttgcccctggcctgaccacaattggagcctctccaacacagactgtgaccctggttacccagccagtggtcaccaaagagacagccatcagcaaactggaaatgcccagctctctgatgctggaagtcccc

SEQ ID NO: 66 - DNA, 9 bp, Artificial Sequence
  synthetic construct; RGX-Dys segment
acactggaa

SEQ ID NO: 67 - DNA, 318 bp, Artificial Sequence
  synthetic construct; RGX-Dys segment
aggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgag

SEQ ID NO: 68 - DNA, 216 bp, Artificial Sequence
  synthetic construct; RGX-Dys segment
gcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctc

SEQ ID NO: 69 - DNA, 744 bp, Artificial Sequence
  synthetic construct; RGX-Dys segment
agaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgc

SEQ ID NO: 70 - DNA, 588 bp, Artificial Sequence
  synthetic construct; RGX-Dys segment
accccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatga

SEQ ID NO: 71 - DNA, 12 bp, Artificial Sequence
  synthetic construct; RGX-Dys segment
  misc (3)..(3): n can be A or G
  misc_feature (3)..(3): n is a, c, g, or t
ganacactggaa

SEQ ID NO: 72 - DNA, 6 bp, Artificial Sequence
  synthetic construct; RGX-Dys segment
ctggaa

SEQ ID NO: 73 - DNA, 129 bp, Artificial Sequence
  synthetic construct; ITR
cgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 74 - DNA, 130 bp, Artificial Sequence
  synthetic construct; ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 75 - DNA, 133 bp, Artificial Sequence
  synthetic construct; human desmin
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 76 - DNA, 97 bp, Artificial Sequence
  synthetic construct; MHCK7
gtaagtttagtctttttgtcttttatttcaggtcccggatccggtggtggtgcaaatcaaagaactgctcctcagtggatgttgcctttacttctag

SEQ ID NO: 77 - protein, 738 aa, Artificial Sequence
  synthetic construct; AAV8 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 78 - protein, 736 aa, Artificial Sequence
  synthetic construct; AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 79 - protein, 1283 aa, Artificial Sequence
  synthetic construct; RGX-Dys5
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 80 - DNA, 426 bp, Artificial Sequence
  synthetic construct; RGX-Dys segment
accccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatga

SEQ ID NO: 81 - DNA, 3855 bp, Artificial Sequence
  synthetic construct; RGX-Dys5
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatga

SEQ ID NO: 82 - DNA, 4560 bp, Artificial Sequence
  synthetic construct; RGX-Dys5 cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatgagtcgacaggcctaataaagagctcagatgcatcgatcagagtgtgttggttttttgtgtggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 83 - protein, 140 aa, Artificial Sequence
  synthetic construct; RGX-Dys segment
TPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 84 - protein, 35 aa, Artificial Sequence
  synthetic construct; RGX-Dys segment
MENSNGSYLNDSISPNESIDDEHLLIQHYCQSLNQ

SEQ ID NO: 85 - protein, 738 aa, Artificial Sequence
  synthetic construct; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 86 - DNA, 714 bp, Artificial Sequence
  synthetic construct; promoter
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 87 - protein, 7 aa, Artificial Sequence
  synthetic construct; capsid protein
LGETTRP

SEQ ID NO: 88 - protein, 9 aa, Artificial Sequence
  synthetic construct; capsid protein
LALGETTRP

SEQ ID NO: 89 - DNA, 240 bp, Artificial Sequence
  Synthetic Construct; human desmin promoter
ctgcagacatgcttgctgcctgccctggcgtgccctggcgaggcttgccgtcacaggacccccgctggctgactcaggggcgcaggctcttgcgggggagctggcctcccgcccccacggccacgggccctttcctggcaggacagcgggatcttgcagctgtcaggggaggggatgacgggggactgatgtcaggaggggatacaaatagtgccgaacaaggaccggattagatctacc

SEQ ID NO: 90 - protein, 54 aa, Artificial Sequence
  Synthetic Construct; Microdystrophin segment (CR Short)
AKHQAKCNICKECPIIGFRYRSLKHFNYDICQSCFFSGRVAKGHKMHYPMVEYC

SEQ ID NO: 91 - protein, 48 aa, Artificial Sequence
  Synthetic Construct; dystrophin portion
TPTTSGEDVRDFAKVLKNKFRTKRYFAKHPRMGYLPVQTVLEGDNMET

SEQ ID NO: 92 - DNA, 335 bp, Artificial Sequence
  Synthetic Construct; Spc5v1 Promoter
agaggccgtccgccctcggcaccatcctcacgacacccaaatatggcgacgggtgaggaatggtggggagttatttttagagcggtgaggaaggtgggcaggcagcaggtgttggcgctccatatttggcgggagttatttttagagcggaggaatggtggacacccaaatatggcgacggttcctcacccgtcgctaaaaataactccgtgtccgccctcggccggggccgcattcctgggggccgggcggtgctcccgcccgcctcgataaaaggctccggggccggcggcggcccacgagctacccggaggagcgggaggcgccaagcggaa

SEQ ID NO: 93 - DNA, 328 bp, Artificial Sequence
  Synthetic Construct; Spc5v2 Promoter
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctcggccggggccgcattcctgggggccgggcggtgctcccgcccgcctcgataaaaggctccggggccggcggcggcccacgagctacccggaggagcgggaggcgccaagc

SEQ ID NO: 94 - DNA, 792 bp, Artificial Sequence
  Synthetic Construct; promoter
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 95 - protein, 3685 aa, Artificial Sequence
  Synthetic Construct; Human Dystrophin Fragment
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 96 - DNA, 162 bp, Artificial Sequence
  Synthetic Construct; Microdystrophin segment (CR Short)
gccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgc

SEQ ID NO: 97 - DNA, 4741 bp, Artificial Sequence
  Synthetic Construct; encodes RGX-DYS1; full genome SPc5-12v1 to polyA including intervening sequences and flanking ITR sequences
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaagaggccgtccgccctcggcaccatcctcacgacacccaaatatggcgacgggtgaggaatggtggggagttatttttagagcggtgaggaaggtgggcaggcagcaggtgttggcgctccatatttggcgggagttatttttagagcggaggaatggtggacacccaaatatggcgacggttcctcacccgtcgctaaaaataactccgtgtccgccctcggccggggccgcattcctgggggccgggcggtgctcccgcccgcctcgataaaaggctccggggccggcggcggcccacgagctacccggaggagcgggaggcgccaagcggaaggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatgactcgagaggcctaataaagagctcagatgcatcgatcagagtgtgttggttttttgtgtgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 98 - DNA, 4734 bp, Artificial Sequence
  Artifical Construct; encodes RGX-DYS1; full genome SPc5-12v2 to polyA including intervening sequences and flanking ITR sequences
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatgactcgagaggcctaataaagagctcagatgcatcgatcagagtgtgttggttttttgtgtgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2024/0358857 A1
(43) Pub. Date: Oct. 31, 2024

(54) TREATMENT OF DUCHENNE MUSCULAR DYSTROPHY AND COMBINATIONS THEREOF

(71) Applicant: REGENXBIO INC., Rockville, MD (US)

(72) Inventors: Olivier Danos, Princeton, NJ (US); SunJung Kim, Fairfax, VA (US); Nicolas Buss, Rockville, MD (US); Michele Fiscella, Bethesda, MD (US); Ye Liu, Rockville, MD (US); Chunping Qiao, Chapel Hill, NC (US)

(21) Appl. No.: 18/559,078

(22) PCT Filed: May 11, 2022
(86) PCT No.: PCT/US2022/028832

§ 371 Date: Nov. 6, 2023

Related U.S. Application Data
(60) Provisional application No. 63/187,349, filed on May 11, 2021.

(51) Int. Cl. A61K 48/00; A61K 38/17; A61K 45/06; C12N 15/86

(52) U.S. Cl. CPC A61K 48/0058; A61K 38/1709; A61K 45/06; C12N 15/86; C12N 2750/14143

(57) ABSTRACT

Provided are methods of treating or ameliorating the symptoms of dystrophinopathies, such as Duchenne muscular dystrophy and Becker muscular dystrophy by administration of therapeutically effective doses of recombinant adeno-associated viruses (rAAV) containing a transgene encoding a microdystrophin in combination with a second therapeutic which is effective to treat or ameliorate the symptoms of the dystrophinopathy.

### 1. FIELD OF THE INVENTION

The present invention relates to treatment of dystrophinopathies by administration of doses of gene therapy vectors, such as AAV gene therapy vectors in which the transgene encodes a microdystrophin in combination with a second therapeutic for treating dystrophinopathies.

### 2. BACKGROUND

A group of neuromuscular diseases called dystrophinopathies are caused by mutations in the DMD gene. Each dystrophinopathy has a distinct phenotype, with all patients suffering from muscle weakness and ultimately cardiomyopathy with ranging severity. Duchenne muscular dystrophy (DMD) is a severe, X-linked, progressive neuromuscular disease affecting approximately one in 3,600 to 9,200 live male births. The disorder is caused by frameshift mutations in the dystrophin gene abolishing the expression of the dystrophin protein. Due to the lack of the dystrophin protein, skeletal muscle, and ultimately heart and respiratory muscles (e.g., intercostal muscles and diaphragm), degenerate causing premature death. Progressive weakness and muscle atrophy begin in childhood. Affected individuals experience breathing difficulties, respiratory infections, and swallowing problems. Almost all DMD patients will develop cardiomyopathy. Pneumonia compounded by cardiac involvement is the most frequent cause of death, which frequently occurs before the third decade.

Becker muscular dystrophy (BMD) has less severe symptoms than DMD, but still leads to premature death. Compared to DMD, BMD is characterized by later-onset skeletal muscle weakness. Whereas DMD patients are wheelchair dependent before age 13, those with BMD lose ambulation and require a wheelchair after age 16. BMD patients also exhibit preservation of neck flexor muscle strength, unlike their counterparts with DMD. Despite milder skeletal muscle involvement, heart failure from DMD-associated dilated cardiomyopathy (DCM) is a common cause of morbidity and the most common cause of death in BMD, which occurs on average in the mid-40s.

Dystrophin is a cytoplasmic protein encoded by the DMD gene, and functions to link cytoskeletal actin filaments to membrane proteins. Normally, the dystrophin protein, located primarily in skeletal and cardiac muscles, with smaller amounts expressed in the brain, acts as a shock absorber during muscle fiber contraction by linking the actin of the contractile apparatus to the layer of connective tissue that surrounds each muscle fiber. In muscle, dystrophin is localized at the cytoplasmic face of the sarcolemma membrane.

The DMD gene is the largest known human gene. The most common mutations that cause DMD or BMD are large deletion mutations of one or more exons (60-70%), but duplication mutations (5-10%), and single nucleotide variants (including small deletions or insertions, single-base changes, and splice site changes accounting for approximately 25-35% of pathogenic variants in males with DMD and about 10-20% of males with BMD), can also cause pathogenic dystrophin variants. In DMD, mutations often lead to a frame shift resulting in a premature stop codon and a truncated, non-functional or unstable protein. Nonsense point mutations can also result in premature termination codons with the same result. While mutations causing DMD can affect any exon, exons 2-20 and 45-55 are common hotspots for large deletion and duplication mutations. In-frame deletions result in the less severe Becker muscular dystrophy (BMD), in which patients express a truncated, partially functional dystrophin.

Full-length dystrophin is a large (427 kDa) protein comprising a number of subdomains that contribute to its function. These subdomains include, in order from the amino-terminus toward the carboxy-terminus, the N-terminal actin-binding domain, a central so-called “rod” domain, a cysteine-rich domain and lastly a carboxy-terminal domain or region. The rod domain is comprised of 4 proline-rich hinge domains (abbreviated H), and 24 spectrin-like repeats (abbreviated R) in the following order: a first hinge domain (H1), 3 spectrin-like repeats (R1, R2, R3), a second hinge domain (H2), 16 more spectrin-like repeats (R4, R5, R6, R7, R8, R9, R10, R11, R12, R13, R14, R15, R16, R17, R18, R19), a third hinge domain (H3), 5 more spectrin-like repeats (R20, R21, R22, R23, R24), and a fourth hinge domain (H4) (including the WW domain). Following the rod domain are the cysteine-rich domain, and the COOH (C)-terminal (CT) domain.

With advances in use of adeno-associated virus (AAV) mediated gene therapy to potentially treat a variety of rare diseases, there has been hope and interest that AAV could be used to treat DMD, BMD and less severe dystrophinopathies. Due to limits on payload size of AAV vectors, attention has focused on creating micro- or mini-dystrophins, smaller versions of dystrophin that eliminate non-essential subdomains while maintaining at least some function of the full-length protein. AAV-mediated microdystrophin gene therapy in mdx mice, an animal model for DMD, was reported as exhibiting efficient expression in muscle and improved muscle function (See, e.g., Wang et al., J. Orthop. Res. 27:421 (2009)).

Thus, there exists a need in the art for methods of administering AAV vectors encoding microdystrophins in combination with other therapeutics for treatment or amelioration of symptoms of dystrophinopathies, including DMD or BMD, and minimizing immune responses to the therapeutic protein.

### 3. SUMMARY OF THE INVENTION

Provided are methods of treating or ameliorating the symptoms of dystrophinopathies by administration of rAAV vector particles containing nucleic acid constructs encoding microdystrophins, such as those constructs in FIG. 2, in combination with a second therapeutic.

Embodiments described herein are methods of treating dystrophinopathy in a subject comprising administering to the subject a first therapeutic and a second therapeutic which is different from said first therapeutic, wherein the first therapeutic is a microdystrophin pharmaceutical composition comprising a therapeutically effective amount of a recombinant adeno-associated vector (rAAV) particle and a pharmaceutically acceptable carrier, wherein the rAAV particle comprises a transgene that encodes a microdystrophin protein a microdystrophin protein having from amino-terminus to the carboxy terminus: ABD-H1-R1-R2-R3-H3-R24-H4-CR-CT wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, CR is the cysteine-rich region of dystrophin or at least a portion thereof which binds β-dystroglycan, and CT is at least a portion of a C-terminal region of dystrophin, where the portion comprises a α1-syntrophin binding site and/or an α-dystrobrevin binding site. In certain embodiments, the CT domain comprises or consists of the proximal 194 amino acids of the C-terminus of dystrophin (the amino acid sequence of SEQ ID NO: 16) or at least the proximal portion of the C-terminus encoding human dystrophin amino acid residues 3361-3554 of SEQ ID NO:95 (UniProtKB-P11532) or at least the proximal portion of the C-terminus encoded by exons 70 to 74 and the first 36 amino acids of the amino acid sequence encoded by the nucleotide sequence of exon 75. Alternatively, the CT domain is truncated and comprises an α1-syntrophin binding site but not an α-dystrobrevin binding site, such as, the amino acid sequence of SEQ ID NO: 83. The constructs include regulatory sequences, such as muscle-specific promoter sequences, including the Spc5-12 promoter (SEQ ID NO:39), or alternatively, a truncated SPc5-12 promoter (SEQ ID NO:40) or a SPc5-12 promoter variant, mutant or transcriptionally active portion thereof (such as, modified Spc5-12 promoters Spc5v1 (SEQ ID NO:92) or Spc5v2 (SEQ ID NO:93)), and polyadenylation signal sequences, such as, the small polyA signal sequence (SEQ ID NO:42). Specific constructs include RGX-DYS1 and RGX-DYS5 (see FIG. 2) having nucleotide sequences of SEQ ID NO:20 and SEQ ID NO:81, respectively, and are flanked by AAV2 ITR sequences, where the entire construct has the nucleotide sequence of SEQ ID NO:53 and 82, respectively. The rAAV particles containing the constructs are, in embodiments, AAV8. For example, the rAAV particle or gene therapy vector is AAV8-RGX-DYS1 (recombinant AAV8 comprising a polynucleotide with the nucleotide sequence of SEQ ID NO:53).

In certain embodiments, the second therapeutic is a mutation suppression therapy, an exon skipping therapy, a steroid therapy, an immunosuppressive/anti-inflammatory therapy, or a therapy that treats one or more symptoms of the dystrophinopathy.

In certain embodiments, a mutation suppression therapy can be ataluren or gentamycin.

In certain embodiments, an exon skipping therapy can be any one of the exon skipping therapies that results in skipping of one or more of exons 2, 43, 44, 45, 50, 51, 52, 53, 55 of the human dystrophin gene to express a form of dystrophin protein. For example, an exon skipping therapy can skip exon 45, such as casimersen, SRP-5045, or DS-5141B. In some embodiments, an exon skipping therapy can skip exon 50, such as SRP-5050. In some embodiments, an exon skipping therapy can skip exon 51, such as eteplirsen or SRP-5051. In some embodiments, an exon skipping therapy can skip exon 53, such as golodirsen, SRP-5053, viltolarsen. In some embodiments, an exon skipping therapy can skip exon 52, such as SRP-5052. In some embodiments, an exon skipping therapy can skip exon 44, such as SRP-5044 or NS-089/NCNP-02. In some embodiments, an exon skipping therapy can skip exon 2, such as scAAV9.U7.ACCA.

In certain embodiments, a steroid therapy can be prednisone, deflazacort, Vamorolone, or Spironolactone, or a combination thereof.

In certain embodiments, the therapy that treats one or more symptoms of the dystrophinopathy can be a therapy that improves muscle mass and/or strength such as spironolactone, Follistatin, SERCA2a, EDG-5506, tamoxifen, Givinostat, ASP0367.

In certain embodiments, the therapy that treats one or more symptoms of the dystrophinopathy can be a therapy that improves a cardiac condition such as ifetroban, bisoprolol fumarate, eplerenone, or a combination thereof.

In certain embodiments, the therapy that treats one or more symptoms of the dystrophinopathy can be a therapy that treats a respiratory symptom such as idebenone.

In certain embodiments, the therapy that treats one or more symptoms of the dystrophinopathy can be a therapy that provides orthopedic management, endocrinologic management, gastrointestinal management, urologic management, or a combination thereof.

In certain embodiments, the therapeutically effective amount of an rAAV particle is administered intravenously or intramuscularly at a dose of 5×10¹³ to 1×10¹⁵ genome copies/kg, including 1×10¹⁴ genome copies/kg, 2×10¹⁴ genome copies/kg or 3×10¹⁴ genome copies/kg. In certain embodiments, the therapeutically effective amount of an rAAV particle is administered intravenously or intramuscularly at a dose of 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ genome copies/kg. In certain embodiments, the therapeutically effective amount of an rAAV particle is administered intravenously at a dose 1×10¹⁴ genome copies/kg. In other embodiments, the therapeutically effective amount of an rAAV particle is administered intravenously at a dose 2×10¹⁴ genome copies/kg. In still other embodiments, the therapeutically effective amount of an rAAV particle is administered intravenously at a dose 3×10¹⁴ genome copies/kg.

In certain embodiments, the therapeutically effective amount of an rAAV particle containing a transgene encoding a microdystrophin of the disclosure is administered intravenously or intramuscularly at a dose of 5×10¹³ to 1×10¹⁵ genome copies/kg, including 1×10¹⁴ genome copies/kg, 2×10¹⁴ genome copies/kg or 3×10¹⁴ genome copies/kg. In certain embodiments, the therapeutically effective amount of an rAAV particle is administered intravenously or intramuscularly at a dose of 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ genome copies/kg.

In certain embodiments, the pharmaceutically acceptable carrier comprises a modified Dulbecco's phosphate buffered saline (DPBS) with sucrose buffer comprising 0.2 g/L potassium chloride, 0.2 g/L potassium phosphate monobasic, 1.2 g/L sodium phosphate dibasic anhydrous, 5.8 g/L sodium chloride, 40 g/L sucrose, and 0.01 g/L poloxamer 188, pH 7.4.

Also provided are pharmaceutical compositions comprising the recombinant vectors encoding the microdystrophins provided herein, including with a pharmaceutically acceptable excipient and methods of treatment for any dystrophinopathy, such as for Duchenne muscular dystrophy (DMD) and Becker muscular dystrophy (BMD), X-linked dilated cardiomyopathy, as well as DMD or BMD female carriers, by administration of the gene therapy vectors described herein to a subject in need thereof, including administration intravenously at dosages of 5×10¹³ to 1×10¹⁵ genome copies/kg, including 1×10¹⁴ genome copies/kg, 2×10¹⁴ genome copies/kg or 3×10¹⁴ genome copies/kg genome copies/kg in combination with a second therapeutic for the dystrophinopathy. Provided are methods of treating, ameliorating the symptoms of or managing a dystrophinopathy, such as Duchenne muscular dystrophy (DMD) and Becker muscular dystrophy (BMD), X-linked dilated cardiomyopathy by administration of an rAAV containing a transgene or gene cassette described herein, by administration to a subject in need thereof such that the microdystrophin is delivered to the muscle (including skeletal muscle, cardiac muscle, and/or smooth muscle). In particular embodiments, the rAAV is administered systemically.

Also provided are methods of decreasing inflammation and/or muscle degeneration in a muscle of a subject in need thereof comprising administering one or more of the disclosed pharmaceutical compositions.

The present inventions are illustrated by way of examples infra describing the construction and making of microdystrophin vectors and in vitro and in vivo assays demonstrating effectiveness.

### 4. BRIEF DESCRIPTION OF THE FIGURES

FIGS. 1A-B. Illustration of the sarcolemma showing interaction among (A) the RGX-DYS1 microdystrophin (which has a C Terminal domain containing dystrobrevin and α1-syntrophin binding sites (as well as β1-syntrophin binding sites), and (B) the wild-type dystrophin protein and the dystrophin-associated protein complex (DAPC) with the actin cytoskeleton. It is envisioned that RGX-DYS1 having at least dystrobrevin and α1-syntrophin binding sites, will partly recruit and anchor nNOS to the sarcolemma through α1-syntrophin. Syn: Syntrophin; Dbr: Dystrobrevin; CR: Cysteine rich domain; nNOS: Neuronal nitric oxide synthase; DG: Dystroglycan; H: hinge; R: spectral-like repeat; SG: Sarcoglycan

FIG. 2. illustrates vector gene expression cassettes, otherwise referred to as microdystrophin constructs or transgenes, for use in a cis plasmid for gene therapy that result in AAV recombinant genomes. DNA length for each component and complete transgene are listed for each construct. SPc5-12: synthetic muscle-specific promoter; CT1.5: truncated/minimal CT domain containing 140 amino acids of the CT domain (SEQ ID NO:83) including an α1-syntrophin binding site but not a dystrobrevin binding site; VH4: human immunoglobin heavy chain variable region intron; ABD: actin binding domain; H: hinge; R: rod; CR: cysteine rich domain; CT: C-terminal domain; smPA: small polyA; ABD: Actin Binding Domain 1 (ABD1).

FIGS. 3A-3D: Western blot against dystrophin extracted from AAV-microdystrophin vector-injected gastrocnemius muscle tissues. Lanes 1 through 4=protein samples from AAV8-RGX-DYS1-injected mdx mice, Lanes 5 through 8=protein samples from AAV8-RGX-DYS5 injected mdx mice, and Lanes 9 through 12=protein samples from AAV8-RGX-DYS3 injected mdx mice. α1-actin serves as the loading control in each lane. Mdx (Lane 13) indicated an un-injected mdx mice. For dystrophin blot, mouse anti-dystrophin monoclonal antibody was used (1:100 dilution). For anti-alpha1-actin blot, polyclonal antibody was used at a dilution factor of 1:10,000, and the secondary (anti-rabbit) antibody was used at 1:20,000. Quantification of microdystrophin bands on western blot analysis of protein samples from A (B), AAV-μ-Dys vector copy numbers in the gastrocnemius muscle by ddPCR (C), and quantification of microdystrophin protein bands normalized by AAV-μ-Dys vector copy numbers (D). * p<0.05; ** P<0.01; ***P<0001.

FIGS. 4A-4B: mRNA expression of microdystrophin and wild-type (WT) dystrophin in skeletal muscles (gastroenemius). Total RNA was extracted from the skeletal muscles and cDNA synthesized. The copy numbers of microdystrophin, WT-dystrophin, and endogenous control Glyceraldehyde 3-phosphate dehydrogenase (GAPDH) mRNA were measured using digital PCR (Naica Crystal Digital PCR system, Stilla technologies). A. Relative micro- or WT-dystrophin mRNA expression normalized by GAPDH. The ratio of WT-dystrophin to GAPDH in B6-WT skeletal muscle was considered as 1. B. Relative micro- or WT-dystrophin mRNA expression in a single cell. Micro- or WT-dystrophin mRNA expression copy numbers were normalized by GAPDH and genome copy numbers per cell.

FIG. 5. Gastrocnemius muscle extracted from mdx mice, tissue sections prepared and immunofluorescently (IF) stained against dystrophin and dystrophin associated protein complexes including dystrobrevin, β-dystroglycan, and syntrophin. Mice were treated as described: B16 (untreated wild-type mice); RGX-DYS1 (mouse ID 3553, and mouse ID 3588); RGX-DYS3 (mouse ID 5, and mouse ID 7); and RGX-DYS5 (mouse ID 9, and mouse ID 11). Objective lens: 40×.

FIGS. 6A-6E: Alpha-syntrophin expression in skeletal muscles. A. Gastrocnemius muscle extracted from mdx mice, tissue sections prepared and immunofluorescently (IF) stained against syntrophin-alpha 1. Mice were treated as described: B16 (untreated wild-type mice); RGX-DYS1 (mouse ID 3553, and mouse ID 3588); RGX-DYS3 (mouse ID 5, and mouse ID 7); and RGX-DYS5 (mouse ID 9, and mouse ID 11). Objective lens: 40×. B. Western blot against syntrophin from muscle tissue lysate. C. Quantification of western blot bands. *, p<0.05; ***, p<0.0001. D. Western blot against syntrophin from total muscle membrane protein. E. Quantification of western blot bands.

FIGS. 7A-7C: nNOS expression in skeletal muscles. A. Immunofluorescent staining against nNOS. B. Western blot against nNOS. C. Quantification of western blot bands.

FIGS. 8A-C: Transduction of satellite cells and amelioration of cell regeneration by AAV vector encoding microdystrophin gene. A. Percentage of AAV-DMD transduced satellite cells. B. Total satellite cell counting in RNAscope® images. C. Pax7 mRNA expression in skeletal muscles from different groups revealed by ddPCR. The primers and probe against microdystrophin were the same as previously described. The ratio of pax7 to GAPDH in B6-WT skeletal muscle was considered as 1. **, p<0.01; ***, p<0.001; ****, p<0.0001 as compared to the untreated mdx mice.

FIG. 9 shows a comparison of biodistribution and transgene expression of AAV8 and AAV9 in NHP. AAV8 and AAV9 packaging CAG-GFP cassette with a unique barcode were produced individually and pooled together with other capsids in approximately equal concentration to generate a library of 118 barcoded AAVs. This library (PAVE118) was administered intravenously to three cynomolgus macaques at a dose of 1.77e13 GC/kg. DNA and RNA isolated from various NHP tissues at 3 weeks post dosing were subjected to NGS analysis for relative abundance. There was no significant difference between DNA and RNA levels from AAV8 and AAV9 capsid in skeletal muscle, cardiac muscle, and liver of NHP.

FIGS. 10A-10D: Grip Strength and In Vitro Force of the EDL Muscle. AAV8-RGX-DYS1 administration improved muscle functions in mdx mice. (A and B) Grip strength at Week 5 was measured. (A) maximal force, and (B) the normalized forelimb value that was calculated by each mouse's body weight. (C and D). In vitro force of the EDL muscle was conducted at Week 6. (C) absolute forelimb and (D) specific force of the EDL muscle that was by normalizing the maximal force produced by the cross-sectional area of the muscle. The wild-type (WT) data was from the age-matched HCD at the testing facility. *** p<0.001. vs wild-type HCD data; *** p<0.001. vs vehicle control mdx; student's t-test was used. Data are presented as mean±SEM.

FIGS. 11A-11K: Muscle Pathology in mdx Vehicle Control Mice and mdx Mice Administered AAV8-RGX-DYS1. AAV8-RGX-DYS1 administration attenuated skeletal muscle inflammation, degeneration, and regeneration in mdx mice. (A-C) Inflammation was assessed based on H&E staining. Yellow dashed lines represent the area of inflammatory foci within the tissue. Percent inflammation in the TA (B) and diaphragm (C) was measured. (D-F) Regenerating fibers in the TA and diaphragm were examined by anti-eMHC staining, a marker of regeneration; red dashed lines represent the area of degenerating area. The positive fibers were counted on the TA (E) and diaphragm (F) and were normalized over the total area of that section in mm² (fibers/mm²). (G-I) Degenerating fibers in the TA and diaphragm were examined by anti-IgM staining; red dashed lines represent the area of degenerating area. The positive fibers were counted on the TA (H) and diaphragm (I) and normalized over the total area of that section in mm² (fibers/mm²). (J and K) Percent central nucleation (%) was performed on five random fields of the TA (J) and diaphragm (K) tissue sections. In each field, central nucleated fibers and total fibers were counted and a percentage of centrally nucleated fibers (CNFs) was calculated. All representative images of the TA and diaphragm at 20× with zoomed area (Bar=200 μm). Age-matched BL10 wild-type control for the TA muscle (n=3) was stained from the test facility tissue bank. For the diaphragm, the aged-matched BL10 wild-type HCD was used. *** p<0.001.vs wild-type HCD data. * p<0.05, *** p<0.001.vs vehicle control mdx; student's t-test was used. Data are presented as mean±SEM.

FIGS. 12A-12B: RGX-DYS1 Vector DNA Biodistribution in mdx Mice at a Dose of 2×10¹⁴ GC/kg. Tissues collected from the AAV8-RGX-DYS1-administered group (n=12) and liver from the vehicle control group (n=11) were analyzed by ddPCR. All tissues collected from mice in the AAV8-RGX-DYS1-administered group were 2-4 log above the estimated lower limit of quantification (LLOQ) (˜0.08 GC/dg) and liver samples from mice in the vehicle control group were approximately at LLOQ. The vector genome copy data was presented in both GC/dg (A) and GC/μg DNA (B). EDL: Extensor Digitorum Longus muscle; TA: Tibialis Anterior. Data are presented as mean±SEM.

FIG. 13: RGX-DYS1 Microdystrophin Levels in Diaphragm, Gastrocnemius and TA Muscles from AAV8-RGX-DYS1-Administered mdx Mice, as Measured by Western Blot. Bars show mean percent dystrophin+S.D., based on the standard curve made of a mixture of BL10 wild type mouse and German Shorthaired Pointer Muscular Dystrophy (GSHPMD) dog muscle lysates, n=10 per tissue.

FIGS. 14A-14C: RGX-DYS1 Microdystrophin Transgene Expression by Immunofluorescence. A. Immunofluorescence for microdystrophin/dystrophin was performed in TA and diaphragm 6 weeks after vehicle or AAV8-RGX-DYS1 administration. AAV8-RGX-DYS1 administration results in 96% fibers and 89.1% fibers localized to the membrane with microdystrophin in the TA (B) and diaphragm (C), respectively. All representative images of TA and diaphragm at 20× with zoomed area (Bar=200 μm). Age-matched BL10 wild-type control for TA muscle (n=3) was stained from the test facility tissue bank. For diaphragm, the aged-matched BL10 wild-type HCD from the testing facility was used. *** p<0.001 vs. wild type; * p<0.05, *** p<0.001. vs vehicle control mdx; student's t-test was used. Data are presented as mean±SEM.

FIG. 15: Dystrophin-Associated Protein Complex (DAPC) by Immunofluorescence. DAPC proteins: α1-syntrophin, dystrobrevin, nNOS-1, and β-dystroglycan with dystrophin were measured in TA tissues by immunofluorescence. AAV8-RGX-DYS1 administration restored syntrophin and dystrobrevin expression that were localized with RGX-DYS1 microdystrophin-positive fibers; β-dystroglycan expression was partially restored. nNOS expression was detectable in AAV8-RGX-DYS1-administered mdx mice and higher than the vehicle control mdx mice, but not as robust as wild type. All representative images of TA at 20× with zoomed area (Bar=100 μm). In each group, stars indicate the same fibers. Age-matched BL10 wild-type control for TA muscle (n=5) was stained from the testing facility tissue bank.

FIG. 16: Gait Overall Score from Fine Motor Kinematic Gait Analysis. Automated gait analysis was conducted 6 weeks and 12 weeks after AAV8-RGX-DYS1 administration (n=8-10 per group) to mdx mice. In overall gait score, a clear mdx mice model effect was observed at 6 weeks post dosing and even greater at 12 weeks post dosing. At Week 12, overall gait scores were significantly improved in mdx mice administered AAV8-RGX-DYS1 at doses of 1×10¹⁴, 3×10¹⁴, and 5×10¹⁴ GC/kg. Data are presented as mean±SEM. Statistical significances: * p<0.05, *** p<0.001, vs. wild type vehicle (RM two-way ANOVA, Sidak's post hoc); * p<0.05, ** p<0.01, vs. mdx vehicle (Mixed effects model ANOVA, Dunnett's post hoc).

FIGS. 17A-17E: T2-Magnetic Resonance Imaging. T2 weighted MRI was conducted 6 and 12 weeks after vehicle or AAV8-RGX-DYS1 administration to mdx mice (n=8-10 per group). (A) The representative images are presented. Hyperintense lesions are indicated by yellow arrows (6 week) and red arrows (12 weeks). (B) Gastrocnemius muscle volumes (mm3) (C) Gastrocnemius muscle hyperintensity percentages (%), obtained using automated threshold analysis (D) T2-relaxation time (milliseconds, ms) in the gastrocnemius muscle lesions and non-lesions (E) were measured. All data were obtained from both legs combined. Data are presented as mean±SEM. Statistical significances: * p<0.05, *** p<0.001, vs. wild type vehicle (RM two-way ANOVA, Sidak's post hoc); ** p<0.01, *** p<0.001, **** p<0.0001 vs. mdx vehicle control (Mixed effects model ANOVA, Dunnett's post hoc).

FIG. 18: Grip Strength (Normalized to Body Weight) at Week 6, 9, and 12. Grip strength was conducted 6, 9, and 12 weeks after vehicle or AAV8-RGX-DYS1 administration (n=8-10 per group) was administered at indicated doses. A clear mdx mouse model effect was not observed 6- and 9-weeks post dosing. At 12 weeks post dosing, a difference between wild-type and mdx mice was noted but no statistical significance. At week 12, grip strength was significantly improved in mdx mice administered AAV8-RGX-DYS1 at doses of 3×10¹⁴ and 5×10¹⁴ GC/kg when compared to the vehicle control mdx mice. Data are presented as mean±SEM. Statistical significances: * p<0.05, *** p<0.001, vs. mdx vehicle control (Mixed effects model ANOVA, Dunnett's post hoc).

FIG. 19: Creatine Kinase Concentration at Week 7 and 12. CK analysis from serum was conducted at 7 and 12 weeks after vehicle or AAV8-RGX-DYS1 administration to mdx mice (n=8-10 per group). Data are presented as mean±SEM. Statistical significances: **** p<0.0001, vs. wild-type (RM two-way ANOVA, Sidak's post hoc); * p<0.05, **** p<0.0001 vs. mdx vehicle control (Mixed effects model ANOVA, Dunnett's post hoc).

FIG. 20: Vector DNA Biodistribution in Liver and Muscle Tissues of BL10 Wild-Type and mdx Mice (Vehicle- or AAV8-RGX-DYS1-Administered). Bars show mean value+S.D., with n=5 for each tissue per group. Tissues collected from BL10 wild-type mice and mdx vehicle control mice showed vector DNA level at either below quantitation limit (BQL=50 copies/μg DNA) or limit of detection (LOD=11.96 copies/μg DNA).

FIGS. 21A and 21B: RGX-DYS1 Microdystrophin/Dystrophin Protein Expression in Gastrocnemius, Diaphragm, and Heart. (A). Bars show mean percent dystrophin+S.E, based on the standard curve made of a mixture of BL10 mouse and GSHPMD dog muscle lysates. BL10 wild type (n=10); mdx vehicle control (n=9); AAV8-RGX-DYS1 mdx (n=8-10 per dose group). **** p<0.0001 vs. wild type; * p<0.05, **** p<0.0001, vs. vehicle control mdx mice (B) Representative Western Blot of RGX-DYS1 microdystrophin/dystrophin protein in the diaphragm. Solid box: expected molecular location of wild-type dystrophin; dotted box: expected molecular location of RGX-DYS1 microdystrophin images analysis from serum was conducted.

### 5. DETAILED DESCRIPTION

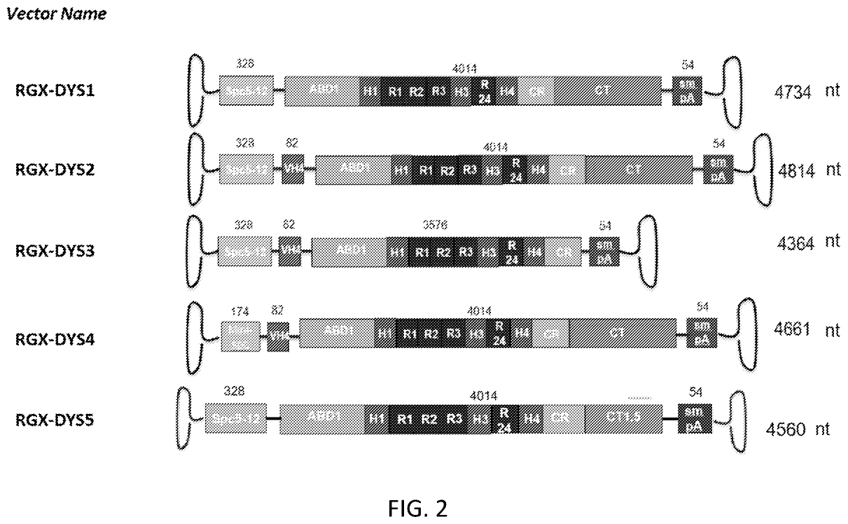

Provided are methods of administering gene therapy vectors, particularly AAV vectors, comprising genomes with transgenes encoding microdystrophin proteins for treatment of dystrophinopathies in combination with a second therapeutic effective for treating or ameliorating the symptoms of dystrophinopathies, including but not limited to Duchenne muscular dystrophy (DMD), Becker muscular dystrophy (BMD), and X-linked dilated cardiomyopathy. The microdystrophin proteins encoded by the transgene consists of dystrophin domains arranged from amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site, and may comprise or consist of at least the proximal portion of the C-terminus encoding human dystrophin amino acid residues 3361-3354 of SEQ ID NO:95 (UniProt-KB-P111532), or the amino acid sequence of SEQ ID NO:16, or alternatively, the CT is a truncated CT comprising or consisting of SEQ ID NO:83, and the microdystrophins may have amino acid sequences of SEQ ID NO:1 (RGX-DYS1) or SEQ ID NO:79 (RGX-DYS5). The transgenes further comprise regulatory sequences, including, for example, a muscle specific promoter, such as SPc5-12 (SEQ ID NO:39) and a polyadenylation signal, such as the small polyA signal (SEQ ID NO:42). Exemplary constructs are depicted, for example, in FIG. 2 and may have nucleotide sequences of (SEQ ID NO:20 for RGX-DYS1 and SEQ ID NO:81 for RGX-DYS5) and, in the constructs, are between ITR sequences (including, AAV2 ITR sequences). The gene therapy vectors may be AAV8 or AAV9 serotype vectors.

Based upon pharmacology studies conducted in mdx mice (Examples 6, 7 and 8 infra, therapeutically effective single doses for peripheral (including intravenous) administration of the rAAVs containing the transgenes described herein (including RGX-DYS1 and RGX-DYS5) are between 5×10¹³ GC/kg to 1×10¹⁵ GC/kg and include dosages within that range, including 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ GC/kg. Administration of such therapeutically effective dosages of the rAAVs comprising transgenes described herein results in amelioration of one or more indicators of dystrophinopathy disease, such as, reduction in creatine kinase activity, reduction in muscle volume, muscle lesions, improvement in gait or ambulatory score (such as NSAA score) or other measure of strength or mobility within 12 weeks, 26 weeks, 52 weeks or longer from the administration. The rAAVs are administered in combination with a second therapeutic effective to treat or ameliorate one or more symptoms of the dystrophinopathy.

Accordingly, provided and described herein are methods of administering an rAAV, including an rAAV8, comprising a genome comprising a transgene encoding a microdystrophin, including the RGX-DYS1 and RGX-DYS5 constructs in combination with a second therapeutic that is effective to treat or ameliorate one or more symptoms of the dystrophinopathy, to a subject, including a human subject, in need thereof, wherein the administration is intravenous or other peripheral administration at a dosage of between 5×10¹³ GC/kg to 1×10¹⁵ GC/kg and include dosages within that range, including 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ GC/kg. Also provided are pharmaceutical compositions formulated for peripheral, including, intravenous, administration of the microdystrophin-encoding rAAV described herein.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein having a modified sequence and/or a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

Amino acid residues as disclosed herein can be modified by conservative substitutions to maintain, or substantially maintain, overall polypeptide structure and/or function. As used herein, “conservative amino acid substitution” indicates that: hydrophobic amino acids (i.e., Ala, Cys, Gly, Pro, Met, Val, lie, and Leu) can be substituted with other hydrophobic amino acids; hydrophobic amino acids with bulky side chains (i.e., Phe, Tyr, and Trp) can be substituted with other hydrophobic amino acids with bulky side chains; amino acids with positively charged side chains (i.e., Arg, His, and Lys) can be substituted with other amino acids with positively charged side chains; amino acids with negatively charged side chains (i.e., Asp and Glu) can be substituted with other amino acids with negatively charged side chains; and amino acids with polar uncharged side chains (i.e., Ser, Thr, Asn, and Gln) can be substituted with other amino acids with polar uncharged side chains.

The terms “subject”, “host”, and “patient” are used interchangeably. A subject may be a mammal such as a non-primate (e.g., cows, pigs, horses, cats, dogs, rats etc.) or a primate (e.g., monkey and human), and includes a human.

The term “therapeutically functional microdystrophin” means that the microdystrophin exhibits therapeutic efficacy in one or more of the assays for therapeutic utility described in Section 5.4 herein or in assessment of methods of treatment described in Section 5.5 herein.

The terms “therapeutic agent” refers to any agent which can be used in treating, managing, or ameliorating symptoms associated with a disease or disorder, where the disease or disorder is associated with a function to be provided by a transgene. A “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The term “prophylactic agent” refers to any agent which can be used in the prevention, reducing the likelihood of, delay, or slowing down of the progression of a disease or disorder, where the disease or disorder is associated with a function to be provided by a transgene. A “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent, reduce the likelihood of, or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The term “CpG islands” means those distinctive regions of the genome that contain the dinucleotide CpG (e.g. C (cytosine) base followed immediately by a G (guanine) base (a CpG)) at high frequency, thus the G+C content of CpG islands is significantly higher than that of non-island DNA. CpG islands can be identified by analysis of nucleotide length, nucleotide composition, and frequency of CpG dinucleotides. CpG island content in any particular nucleotide sequence or genome may be measured using the following criteria: island size greater than 100, GC Percent greater than 50.0%, and ratio greater than 0.6 of observed number of CG dinucleotides to the expected number on the basis of the number of Gs and Cs in the segment (Obs/Exp greater than 0.6).

Obs/Exp CpG=Number of CpG*N/(Number of C*Number of G)
where N=length of sequence.

Various software tools are available for such calculations, such as world-wide-web.urogene.org/cgi-bin/methprimer/methprimer.cgi, world-wide-web.cpgislands.usc.edu/, world-wide-web.ebi.ac.uk/Tools/emboss/cpgplot/index.html and world-wide-web.bioinformatics.org/sms2/cpg_islands.html. (See also Gardiner-Garden and Frommer, J Mol Biol. 1987 Jul. 20; 196(2):261-82; Li L C and Dahiya R. MethPrimer: designing primers for methylation PCRs. Bioinformatics. 2002 November; 18(11):1427-31.). In one embodiment the algorithm to identify CpG islands is found at www.urogene.org/cgi-bin/methprimer/methprimer.cgi.

### 5.2. Microdystrophin Transgenes

### 5.2.1 Microdystrophins Encoded by the Transgenes

Encoded by the transgenes provided herein for the methods of the invention are microdystrophins that consist of dystrophin domains arranged amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is a hinge 4 region of dystrophin, CR is a cysteine-rich region of dystrophin and CT is the C terminal domain (and comprises at least the portion of the CT domain containing the al-syntrophin binding site, including SEQ ID NO:84). Table 1 below has the amino acid sequences for these components, in particular from the full length human DMD protein (UniProtDB-11532, which is incorporated by reference herein) and they are encoded by the nucleotide sequences in Tables 3 and 4 (including the wild type and codon optimized sequences).

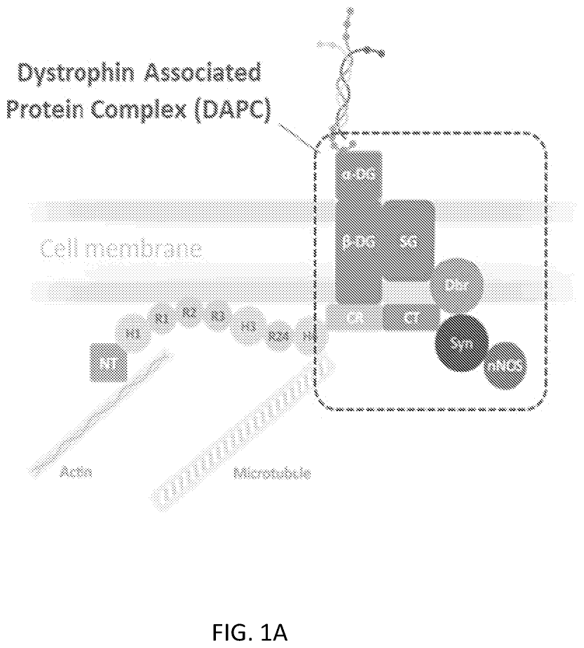

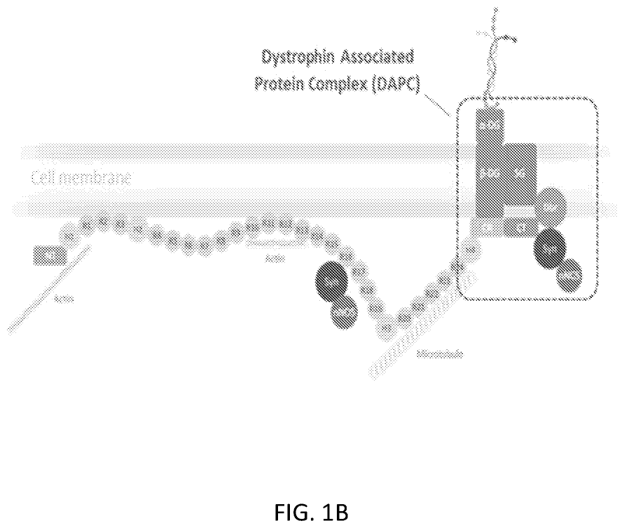

To overcome the packaging limitation that is typical of AAV vectors, many of the microdystrophin genes developed for clinical use are lacking the CT domain. Several researchers have indicated that the DAPC does not even require the C-terminal domain in order to assemble or that the C-terminus is non-essential [Crawford, et al., J Cell Biol, 2000, 150(6):1399-1409; and Ramos, J. N, et al. Molecular Therapy 2019, 27(3):1-13]. However, overexpression of a microdystrophin gene containing helix 1 of the coiled-coil motif of the CT domain in skeletal muscle of mdx mice increased the recruitment α1-syntrophin and α-dystrobrevin, which are members of DAP complex, serving as modular adaptors for signaling proteins recruited to the sarcolemma membrane [Koo, T., et al., Delivery of AAV2/9-microdystrophin genes incorporating helix 1 of the coiled-coil motif in the C-terminal domain of dystrophin improves muscle pathology and restores the level of α1-syntrophin and α-dystrobrevin in skeletal muscles of mdx mice. Hum Gene Ther, 2011. 22(11): p. 1379-88]. Overexpression of the longer version of microdystrophin also improved the muscle resistance to lengthening contraction-induced muscle damage in the mdx mice as compared with the shorter version [Koo, T., et al. 2011, supra]. The CT domain does play a role in the formation of the Dystrophin Associated Protein Complex (DAPC) (see FIG. 1B).

The CT domain of dystrophin contains two polypeptide stretches that are predicted to form α-helical coiled coils similar to those in the rod domain (see H1 indicated by single underlining and H2 indicated by double underlining in SEQ ID 16 in Table 1 below). Each coiled coil has a conserved repeating heptad (a,b,c,d,e,f,g)ₙ similar to those found in leucine zippers where leucine predominates at the “d” position. This domain has been named the CC (coiled coil) domain. The CC region of dystrophin forms the binding site for dystrobrevin and may modulate the interaction between α1-syntrophin and other dystrophin-associated proteins.

Both syntrophin isoforms, α1-syntrophin and β1-syntrophin are thought to interact directly with dystrophin through more than one binding site in dystrophin exons 73 and 74 (Yang et al, JBC 270(10):4975-8 (1995)). α1- and β1-syntrophin bind separately to the dystrophin C-terminal domain, and the binding site for al-syntrophin reportedly resides at least within the amino acid residues 3447 to 3481, while that for β1-syntrophin has been reported to reside within the amino acid residues 3495 to 3535 (as numbered in the DMD protein of UniProtDB-11532 (SEQ ID NO:95), see also Table 1, SEQ ID NO:16, italic). Alpha1-(α1-) syntrophin and alpha-syntrophin are used interchangeably throughout.

Microdystrophin constructs disclosed herein were found to bind to and recruit nNOS, as well as alpha-syntrophin, alpha-dystrobrevin and beta-dystroglycan. Binding to nNOS, in the context of a microdystrophin construct including a C-terminal domain of dystrophin binding to nNOS, means that the microdystrophin construct expressed in muscle tissue was determined by immunostaining with appropriate antibodies to identify each of alpha-syntrophin, alpha-dystrobrevin, and nNOS in or near the sarcolemma in a section of the transduced muscle tissue. See Examples 4 and 5, infra. In certain embodiments, the microdystrophin protein has a C-terminal domain that “increases binding” to α1-syntrophin, β-syntrophin and/or dystrobrevin compared to a comparable microdystrophin that does not contain the C-terminal domain (but has the same amino acid sequence otherwise, that is a “reference microdystrophin protein”), meaning that the DAPC is stabilized or anchored to the sarcolemma, to a greater extent than a reference microdystrophin that does not have the C-terminal domain (but has the same amino acid sequence otherwise as the microdystrophin), as determined by greater levels of one or more DAPC components in the muscle membrane by immunostaining of muscle sections or western blot analysis of muscle tissue lysates or muscle membrane preparations for one of more DAPC components, including α1-syntrophin, β-syntrophin, α-dystrobrevin, β-dystroglycan or nNOS in mdx mouse muscle treated with the microdystrophin having the C-terminal domain, as compared to the mdx mouse muscle treated with the reference microdystrophin protein (having the same sequence and dystrophin components except not having the C-terminal domain) (see Examples 4 and 5 in Sections 6.4 and 6.5, infra).

In some embodiments, the microdystrophin construct including a C-terminal domain of dystrophin comprises an α1-syntrophin binding site and/or a dystrobrevin binding site in the C-terminal domain. In some embodiments, the C-terminal domain comprising an α1-syntrophin binding site is a truncated C-terminal domain. The α1-syntrophin binding site functions in part to recruit and anchor nNOS to the sarcolemma through α1-syntrophin (See FIGS. 1A and 1B).

The embodiments described herein can comprise all or a portion of the CT domain comprising the Helix 1 of the coiled-coil motif. The C Terminal sequence may be defined by the coding sequence of the exons of the DMD gene, in particular exons 70 to 74, and a portion of exon 75 (in particular, the nucleotide sequence encoding the first 36 amino acids of the amino acid sequence encoded by exon 75, or by the sequence of the human DMD protein, for example, the sequence of UniProtKB-P11532 (SEQ ID NO:95) (the CT is amino acids 3361 to 3554 of the UniProtKB-P11532 sequence), or comprising or consisting of binding sites for dystrobrevin and/or α1-syntrophin (indicated in Table 1, SEQ ID NO:16). In certain embodiments, the CT domain consists of or comprises the 194 C-terminal amino acids of the DMD protein, for example, residues 3361 to 3554 of the amino acid sequence of UniProtKB-P11532 (SEQ ID NO:95), the amino acids encoded by exons 70 to 74, and the nucleotide sequence encoding the first 36 nucleotides of the nucleotide sequence of exon 75 of the DMD gene, or the amino acid sequence of SEQ ID NO:16 (see Table 1). For example, RGX-DYS1 has the 194 amino acid CT sequence of SEQ ID NO: 16. In other embodiments, the amino acid sequence of the C-terminal domain is truncated and comprises at least the binding sites for dystrobrevin and/or α1-syntrophin. In certain embodiments, the truncated C-terminal domain comprises the amino acid sequence MENSNGSYLNDSISPNESIDDEHLLIQHYCQSLNQ (α1-syntrophin binding site) (SEQ ID NO:84). In certain embodiments, the truncated C-terminal domain comprises an α1-syntrophin binding site, wherein the binding site has amino acid sequence MENSNGSYLNDSISPNESIDDEHLLIQHYCQSLNQ (SEQ ID NO:84). In particular embodiments, the CT domain sequence has the amino acid sequence of SEQ ID NO:83 or amino acids 3361 to 3500 of the UniProtKB-P11532 human DMD sequence. For example, RGX-DYS5 has a CT domain having the amino acid sequence of SEQ ID NO:83. In alternative embodiments, the microdystrophin lacks a CT domain, and may have the domains arranged as follows: ABD1-L1-H1-L2-R1-R2-L3-R3-H3-L4-R24-H4-CR, for example RGX-DYS3 (FIG. 2; SEQ ID NO:2).

The NH₂ terminus and a region in the rod domain of dystrophin bind directly to but do not cross-link cytoskeletal actin. The rod domain of wild type dystrophin is composed of 24 repeating units that are similar to the triple helical repeats of spectrin. This repeating unit accounts for the majority of the dystrophin protein and is thought to give the molecule a flexible rod-like structure similar to 0-spectrin. These α-helical coiled-coil repeats are interrupted by four proline-rich hinge regions. At the end of the 24th repeat is the fourth hinge region that is immediately followed by the WW domain [Blake, D. et al, Function and Genetics of Dystrophin and Dystrophin-Related Proteins in Muscle. Physiol. Rev. 82: 291-329, 2002]. Microdystrophins disclosed herein do not include R4 to R23, and only include 3 of the 4 hinge regions or portions thereof. In some embodiments, no new amino acid residues or linkers are introduced into the microdystrophin.

In some embodiments, microdystrophin comprises H3 (e.g., SEQ ID NOS: 1, 2, or 79). In embodiments, H3 can be a full endogenous H3 domain from N-terminal to C-terminal, e.g., SEQ ID NO:11. Stated another way, some microdystrophin embodiments do not contain a fragment of the H3 domain but contain the entire H3 domain. In some embodiments, the C-terminal amino acid of the R3 domain is coupled directly (or covalently bonded to) the N-terminal amino acid of the H3 domain. In some embodiments, the C-terminal amino acid of the R3 domain coupled to the N-terminal amino acid of the H3 domain is Q. In some embodiments, the 5′ amino acid of the H3 domain coupled to the R3 domain is Q.

Without being bound by any one theory, a full hinge domain may be appropriate in any microdystrophin construct in order to convey full activity upon the derived microdystrophin protein. Hinge segments of dystrophin have been recognized as being proline-rich in nature and may therefore confer flexibility to the protein product (Koenig and Kunkel, 265(6):4560-4566, 1990). Any deletion of a portion of the hinge, especially removal of one or more proline residues, may reduce its flexibility and therefore reduce its efficacy by hindering its interaction with other proteins in the DAP complex.

Microdystrophins disclosed herein comprise the wild-type dystrophin H4 sequence (which contains the WW domain) to and including the CR domain (which contains the ZZ domain, represented by a single underline (UniProtKB-P11532 aa 3307-3354) in SEQ ID NO:15). The WW domain is a protein-binding module found in several signaling and regulatory molecules. The WW domain binds to proline-rich substrates in an analogous manner to the src homology-3 (SH3) domain. This region mediates the interaction between β-dystroglycan and dystrophin, since the cytoplasmic domain of β-dystroglycan is proline rich. The WW domain is in the Hinge 4 (H4 region). The CR domain contains two EF-hand motifs that are similar to those in α-actinin and that could bind intracellular Ca²⁺. The ZZ domain contains a number of conserved cysteine residues that are predicted to form the coordination sites for divalent metal cations such as Zn²⁺. The ZZ domain is similar to many types of zinc finger and is found both in nuclear and cytoplasmic proteins. The ZZ domain of dystrophin binds to calmodulin in a Ca²⁺-dependent manner. Thus, the ZZ domain may represent a functional calmodulin-binding site and may have implications for calmodulin binding to other dystrophin-related proteins.

Microdystrophin embodiments can further comprise linkers (L1, L2, L3, L4, L4.1 and/or L4.2) or portions thereof connected the domains as shown as follows: ABD1-L1-H1-L2-R1-R2-L3-R3-H3-L4-R24-H4-CR-CT (e.g., SEQ ID NO: 1, 79, or 91) or ABD1-L1-H1-L2-R1-R2-L3-R3-H3-L4-R24-H4-CR (e.g., SEQ ID NO: 2) L1 can be an endogenous linker L1 (e.g., SEQ ID NO:4) that can couple ABD1 to H1. L2 can be an endogenous linker L2 (e.g., SEQ ID NO:6) that can couple H1 to R1. L3 can be an endogenous linker L3 (e.g., SEQ ID NO:9) that can couple R2 to R3.

L4 can also be an endogenous linker that can couple H3 and R24. In some embodiments, L4 is 3 amino acids, e.g. TLE (SEQ ID NO:12) that precede R24 in the native dystrophin sequence. In other embodiments, L4 can be the 4 amino acids that precede R24 in the native dystrophin sequence (SEQ ID NO:17) or the 2 amino acids that precede R24 (SEQ ID NO:18). In other embodiments, there is no linker, L4 or otherwise, in between H3 and R24. On the 5′ end of H3, as mentioned above, no linker is present, but rather R3 is directly coupled to H3, or alternatively H2.

The above described components of microdystrophin other domains not specifically described can have the amino acid sequences as provided in Table 1 below. The amino acid sequences for the domains provided herein correspond to the dystrophin isoform of UniProtKB-P11532 (DMD_HUMAN) (SEQ ID NO:95), which is herein incorporated by reference. Other embodiments can comprise the domains from naturally-occurring functional dystrophin isoforms known in the art, such as UniProtKB-A0A075B6G3 (A0A075B6G3_HUMAN), (incorporated by reference herein) wherein, for example, R24 has an R substituted for the Q at amino acid 3 of SEQ ID NO:13.

Additional embodiments are disclosed in International Application PCT/US2020/062484, filed Nov. 27, 2020, which is hereby incorporated by reference in its entirety.

TABLE 1
segment amino acid sequences

| | SEQ | |
|---|---|---|
| Structure | ID | Sequence |
| ABD1 | 3 | MLWWEEVEDCYEREDVQKKTFTKWVNAQFSKFGKQHIENLFSDL |
| | | QDGRRLLDLLEGLTGQKLPKEKGSTRVHALNNVNKALRVLQNNN |
| | | VDLVNIGSTDIVDGNHKLTLGLIWNIILHWQVKNVMKNIMAGLQ |
| | | QTNSEKILLSWVRQSTRNYPQVNVINFTTSWSDGLALNALIHSH |
| | | RPDLFDWNSVVCQQSATQRLEHAFNIARYQLGIEKLLDPEDVDT |
| | | TYPDKKSILMYITSLFQVLP |
| L1 | 4 | QQVSIEAIQEVE |
| H1 | 5 | MLPRPPKVTKEEHFQLHHQMHYSQQITVSLAQGYERTSSPKPRF |
| | | KSYAYTQAAYVTTSDPTRSPFPSQHLEAPED |
| L2 | 6 | KSFGSSLME |
| R1 | 7 | SEVNLDRYQTALEEVLSWLLSAEDTLQAQGEISNDVEVVKDQFH |
| | | THEGYMMDLTAHQGRVGNILQLGSKLIGTGKLSEDEETEVQEQM |
| | | NLLNSRWECLRVASMEKQSNLHR |
| R2 | 8 | VLMDLQNQKLKELNDWLTKTEERTRKMEEEPLGPDLEDLKRQVQ |
| | | QHKVLQEDLEQEQVRVNSLTHMVVVVDESSGDHATAALEEQLKV |
| | | LGDRWANICRWTEDRWVLLQD |
| L3 | 9 | IL |
| R3 | 10 | LKWQRLTEEQCLFSAWLSEKEDAVNKIHTTGFKDQNEMLSSLQK |
| | | LAVLKADLEKKKQSMGKLYSLKQDLLSTLKNKSVTQKTEAWLDN |
| | | FARCWDNLVQKLEKSTAQISQ |
| H3 | 11 | QPDLAPGLTTIGASPTQTVTLVTQPVVTKETAISKLEMPSSLML |
| | | EVP |
| L4 | 12 | TLE |
| R24 | 13 | RLQELQEATDELDLKLRQAEVIKGSWQPVGDLLIDSLQDHLEKV |
| | | KALRGEIAPLKENVSHVNDLARQLTTLGIQLSPYNLSTLEDLNT |
| | | RWKLLQVAVEDRVRQLHE |
| H4 | 14 | AHRDFGPASQHELSTSVQGPWERAISPNKVPYYINHETQTTCWD |
| | | HPKMTELYQSLADLNNVRFSAYRTAMKL |
| | | WW domain is represented by a single underline (UniProtKB- |
| | | P11532 aa 3055-3088) |
| Cysteine-rich | 15 | RRLQKALCLDLLSLSAACDALDQHNLKQNDQPMDILQIINCLTT |
| domain (CR) | | IYDRLEQEHNNLVNVPLCVDMCLNWLLNVYDTGRIGRIRVLSFK |
| | | TGIISLCKAHLEDKYRYLFKQVASSTGFCDQRRLGLLLHDSIQI |
| | | PRQLGEVASFGGSNIEPSVRSCFQFANNKPEIEAALFLDWMRLE |
| | | PQSMVWLPVLHRVAAAETAKHQAKCNICKECPIIGFRYRSLKHF |
| | | NYDICQSCFFSGRVAKGHKMHYPMVEYC |
| | | ZZ domain is represented by a single underline (UniProtKB- |
| | | P11532 aa 3307-3354) |
| CR short | 90 | AKHQAKCNICKECPIIGFRYRSLKHFNYDICQSCFFSGRVAKGH |
| | | KMHYPMVEYC |
| C-terminal | 16 | TPTTSGEDVRDFAKVLKNKFRTKRYFAKHPRMGYLPVQTVLEGD |
| Domain (CT) | | NMETPVTLINFWPVDSAPASSPQLSHDDTHSRIEHYASRLAEME |
| | | NSNGSYLNDSISPNESIDDEHLLIQHYCQSLNQDSPLSQPRSPA |
| | | QILISLESEERGELERILADLEEENRNLQAEYDRLKQQHEHKGL |
| | | SPLPSPPEMMPTSPQSPR |
| | | Coiled-coil motif H1 is represented by a single underline; motif |
| | | H2 is represented by a double underline; dystrobrevin-binding side |
| | | is in italics. |
| Minimal/ | 83 | TPTTSGEDVRDFAKVLKNKFRTKRYFAKHPRMGYLPVQTVLEGD |
| truncated C- | | NMETPVTLINFWPVDSAPASSPQLSHDDTHSRIEHYASRLAEME |
| terminal | | NSNGSYLNDSISPNESIDDEHLLIQHYCQSLNQDSPLSQPRSPA |
| Domain | | QILISLES |
| (CT1.5) | | α1-syntrophin-binding site is in italics. |
| L4 | 17 | ETLE |
| L4 | 18 | LE |
| Minimal alpha- | 84 | MENSNGSYLNDSISPNESIDDEHLLIQHYCQSLNQ |
| syntrophin | | |
| binding site | | |
| Human | 95 | MLWWEEVEDC YEREDVQKKT FTKWVNAQFS KFGKQHIENL |
| dystrophin | | FSDLQDGRRL LDLLEGLTGQ KLPKEKGSTR VHALNNVNKA |
| (UniProtKB- | | LRVLQNNNVD LVNIGSTDIV DGNHKLTLGL IWNIILHWQV |
| P11532) | | KNVMKNIMAG LQQTNSEKIL LSWVRQSTRN YPQVNVINFT |
| | | TSWSDGLALN ALIHSHRPDL FDWNSVVCQQ SATQRLEHAF |
| | | NIARYQLGIE KLLDPEDVDT TYPDKKSILM YITSLFQVLP |
| | | QQVSIEAIQE VEMLPRPPKV TKEEHFQLHH QMHYSQQITV |
| | | SLAQGYERTS SPKPRFKSYA YTQAAYVTTS DPTRSPFPSQ |
| | | HLEAPEDKSF GSSLMESEVN LDRYQTALEE VLSWLLSAED |
| | | TLQAQGEISN DVEVVKDQFH THEGYMMDLT AHQGRVGNIL |
| | | QLGSKLIGTG KLSEDEETEV QEQMNLLNSR WECLRVASME |
| | | KQSNLHRVLM DLQNQKLKEL NDWLIKTEER TRKMEEEPLG |
| | | PDLEDLKRQV QQHKVLQEDL EQEQVRVNSL THMVVVVDES |
| | | SGDHATAALE EQLKVLGDRW ANICRWTEDR WVLLQDILLK |
| | | WQRLTEEQCL FSAWLSEKED AVNKIHTTGF KDQNEMLSSL |
| | | QKLAVLKADL EKKKQSMGKL YSLKQDLLST LKNKSVTQKT |
| | | EAWLDNFARC WDNLVQKLEK STAQISQAVT TTQPSLTQTT |
| | | VMETVTTVTT REQILVKHAQ EELPPPPPQK KRQITVDSEI |
| | | RKRLDVDITE LHSWITRSEA VLQSPEFAIF RKEGNFSDLK |
| | | EKVNAIEREK AEKFRKLQDA SRSAQALVEQ MVNEGVNADS |
| | | IKQASEQLNS RWIEFCQLLS ERLNWLEYQN NIIAFYNQLQ |
| | | QLEQMTTTAE NWLKIQPTTP SEPTAIKSQL KICKDEVNRL |
| | | SDLQPQIERL KIQSIALKEK GQGPMFLDAD FVAFTNHFKQ |
| | | VFSDVQAREK ELQTIFDTLP PMRYQETMSA IRTWVQQSET |
| | | KLSIPQLSVT DYEIMEQRLG ELQALQSSLQ EQQSGLYYLS |
| | | TTVKEMSKKA PSEISRKYQS EFEEIEGRWK KLSSQLVEHC |
| | | QKLEEQMNKL RKIQNHIQTL KKWMAEVDVF LKEEWPALGD |
| | | SEILKKQLKQ CRLLVSDIQT IQPSLNSVNE GGQKIKNEAE |
| | | PEFASRLETE LKELNTQWDH MCQQVYARKE ALKGGLEKTV |
| | | SLQKDLSEMH EWMTQAEEEY LERDFEYKTP DELQKAVEEM |
| | | KRAKEEAQQK EAKVKLLTES VNSVIAQAPP VAQEALKKEL |
| | | ETLTTNYQWL CTRLNGKCKT LEEVWACWHE LLSYLEKANK |
| | | WLNEVEFKLK TTENIPGGAE EISEVLDSLE NLMRHSEDNP |
| | | NQIRILAQTL TDGGVMDELI NEELETFNSR WRELHEEAVR |
| | | RQKLLEQSIQ SAQETEKSLH LIQESLTFID KQLAAYIADK |
| | | VDAAQMPQEA QKIQSDLTSH EISLEEMKKH NQGKEAAQRV |
| | | LSQIDVAQKK LQDVSMKERL FQKPANFEQR LQESKMILDE |
| | | VKMHLPALET KSVEQEVVQS QLNHCVNLYK SLSEVKSEVE |
| | | MVIKTGRQIV QKKQTENPKE LDERVTALKL HYNELGAKVT |
| | | ERKQQLEKCL KLSRKMRKEM NVLTEWLAAT DMELTKRSAV |
| | | EGMPSNLDSE VAWGKATQKE IEKQKVHLKS ITEVGEALKT |
| | | VLGKKETLVE DKLSLLNSNW IAVTSRAEEW LNLLLEYQKH |
| | | METFDQNVDH ITKWIIQADT LLDESEKKKP QQKEDVLKRL |
| | | KAELNDIRPK VDSTRDQAAN LMANRGDHCR KLVEPQISEL |
| | | NHRFAAISHR IKTGKASIPL KELEQFNSDI QKLLEPLEAE |
| | | IQQGVNLKEE DFNKDMNEDN EGTVKELLQR GDNLQQRITD |
| | | ERKREEIKIK QQLLQTKHNA LKDLRSQRRK KALEISHQWY |
| | | QYKRQADDLL KCLDDIEKKL ASLPEPRDER KIKEIDRELQ |
| | | KKKEELNAVR RQAEGLSEDG AAMAVEPTQI QLSKRWREIE |
| | | SKFAQFRRLN FAQIHTVREE TMMVMTEDMP LEISYVPSTY |
| | | LTEITHVSQA LLEVEQLLNA PDLCAKDFED LFKQEESLKN |
| | | IKDSLQQSSG RIDIIHSKKT AALQSATPVE RVKLQEALSQ |
| | | LDFQWEKVNK MYKDRQGRFD RSVEKWRRFH YDIKIFNQWL |
| | | TEAEQFLRKT QIPENWEHAK YKWYLKELQD GIGQRQTVVR |
| | | TLNATGEEII QQSSKTDASI LQEKLGSLNL RWQEVCKQLS |
| | | DRKKRLEEQK NILSEFQRDL NEFVLWLEEA DNIASIPLEP |
| | | GKEQQLKEKL EQVKLLVEEL PLRQGILKQL NETGGPVLVS |
| | | APISPEEQDK LENKLKQTNL QWIKVSRALP EKQGETEAQI |
| | | KDLGQLEKKL EDLEEQLNHL LLWLSPIRNQ LEIYNQPNQE |
| | | GPFDVKETEI AVQAKQPDVE EILSKGQHLY KEKPATQPVK |
| | | RKLEDLSSEW KAVNRLLQEL RAKQPDLAPG LTTIGASPTQ |
| | | TVTLVTQPVV TKETAISKLE MPSSLMLEVP ALADFNRAWT |
| | | ELTDWLSLLD QVIKSQRVMV GDLEDINEMI IKQKATMQDL |
| | | EQRRPQLEEL ITAAQNLKNK TSNQEARTII TDRIERIQNQ |
| | | WDEVQEHLQN RRQQLNEMLK DSTQWLEAKE EAEQVLGQAR |
| | | AKLESWKEGP YTVDAIQKKI TETKQLAKDL RQWQTNVDVA |
| | | NDLALKLLRD YSADDTRKVH MITENINASW RSIHKRVSER |
| | | EAALEETHRL LQQFPLDLEK FLAWLTEAET TANVLQDATR |
| | | KERLLEDSKG VKELMKQWQD LQGEIEAHTD VYHNLDENSQ |
| | | KILRSLEGSD DAVLLQRRLD NMNFKWSELR KKSLNIRSHL |
| | | EASSDQWKRL HLSLQELLVW LQLKDDELSR QAPIGGDEPA |
| | | VQKQNDVHRA FKRELKTKEP VIMSTLETVR IFLTEQPLEG |
| | | LEKLYQEPRE LPPEERAQNV TRLLRKQAEE VNTEWEKLNL |
| | | HSADWQRKID ETLERLQELQ EATDELDLKL RQAEVIKGSW |
| | | QPVGDLLIDS LQDHLEKVKA LRGEIAPLKE NVSHVNDLAR |
| | | QLTTLGIQLS PYNLSTLEDL NTRWKLLQVA VEDRVRQLHE |
| | | AHRDFGPASQ HFLSTSVQGP WERAISPNKV PYYINHETQT |
| | | TCWDHPKMTE LYQSLADLNN VRFSAYRTAM KLRRLQKALC |
| | | LDLLSLSAAC DALDQHNLKQ NDQPMDILQI INCLITIYDR |
| | | LEQEHNNLVN VPLCVDMCLN WLLNVYDTGR TGRIRVLSFK |
| | | TGIISLCKAH LEDKYRYLFK QVASSTGFCD QRRLGLLLHD |
| | | SIQIPRQLGE VASFGGSNIE PSVRSCFQFA NNKPEIEAAL |
| | | FLDWMRLEPQ SMVWLPVLHR VAAAETAKHQ AKCNICKECP |
| | | IIGFRYRSLK HFNYDICQSC FFSGRVAKGH KMHYPMVEYC |
| | | TPTTSGEDVR DFAKVLKNKF RTKRYFAKHP RMGYLPVQTV |
| | | LEGDNMETPV TLINFWPVDS APASSPQLSH DDTHSRIEHY |
| | | ASRLAEMENS NGSYLNDSIS PNESIDDEHL LIQHYCQSLN |
| | | QDSPLSQPRS PAQILISLES EERGELERIL ADLEEENRNL |
| | | QAEYDRLKQQ HEHKGLSPLP SPPEMMPTSP QSPRDAELIA |
| | | EAKLLRQHKG RLEARMQILE DHNKQLESQL HRLRQLLEQP |
| | | QAEAKVNGTT VSSPSTSLQR SDSSQPMLLR VVGSQTSDSM |
| | | GEEDLLSPPQ DISTGLEEVM EQLNNSFPSS RGRNTPGKPM |
| | | REDTM |

The present disclosure also contemplates variants of these sequences so long as the function of each domain and linker is substantially maintained and/or the therapeutic efficacy of microdystrophin comprising such variants is substantially maintained. Functional activity includes (1) binding to one of, a combination of, or all of actin, β-dystroglycan, α1-syntrophin, α-dystrobrevin, and nNOS; (2) improved muscle function in an animal model (for example, in the mdx mouse model described herein) or in human subjects; and/or (3) cardioprotective or improvement in cardiac muscle function in animal models or human patients. In particular, microdystrophin can comprise ABD consisting of SEQ ID NO:3 or an amino acid sequence with at least 80%, at least 85%, at least 90%, at least 95%, at least 96%, at least 97%, at least 98%, or at least 99% sequence identity to SEQ ID NO: 3; H1 consisting of SEQ ID NO:5 or an amino acid sequence with at least 80%, at least 85%, at least 90%, at least 95%, at least 96%, at least 97%, at least 98%, or at least 99% sequence identity to SEQ ID NO: 5; R1 consisting of SEQ ID NO: 7 or an amino acid sequence with at least 80%, at least 85%, at least 90%, at least 95%, at least 96%, at least 97%, at least 98%, or at least 99% sequence identity to SEQ ID NO: 7; R2 consisting of SEQ ID NO: 8 or an amino acid sequence with at least 80%, at least 85%, at least 90%, at least 95%, at least 96%, at least 97%, at least 98%, or at least 99% sequence identity to SEQ ID NO: 8; H3 consisting of SEQ ID NO: 11 or an amino acid sequence with at least 80%, at least 85%, at least 90%, at least 95%, at least 96%, at least 97%, at least 98%, or at least 99% sequence identity to SEQ ID NO: 11; R24 consisting of SEQ ID NO: 13 or an amino acid sequence with at least 80%, at least 85%, at least 90%, at least 95%, at least 96%, at least 97%, at least 98%, or at least 99% sequence identity to SEQ ID NO: 13; H4 consisting of SEQ ID NO: 14 or an amino acid sequence with at least 80%, at least 85%, at least 90%, at least 95%, at least 96%, at least 97%, at least 98%, or at least 99% sequence identity to SEQ ID NO: 14; CR consisting of SEQ ID NO: 15 or 90 or an amino acid sequence with at least 80%, at least 85%, at least 90%, at least 95%, at least 96%, at least 97%, at least 98%, or at least 99% sequence identity to SEQ ID NO: 15 or 90; CT consisting of SEQ ID NO: 16 or 83 or an amino acid sequence with at least 80%, at least 85%, at least 90%, at least 95%, at least 96%, at least 97%, at least 98%, or at least 99% sequence identity to SEQ ID NO: 16 or 83, or CT comprising SEQ ID NO: 84. In addition to the foregoing, microdystrophin can comprise linkers in the locations described above that comprise or consist of sequences as follows: L1 consisting of SEQ ID NO: 4 or an amino acid sequence with at least 80%, at least 85%, at least 90%, at least 95%, at least 98%, or at least 99% sequence identity to SEQ ID NO: 4; L2 consisting of SEQ ID NO: 6 or an amino acid sequence with at least 80%, at least 85%, at least 90%, at least 95%, at least 98%, or at least 99% sequence identity to SEQ ID NO: 6; L3 consisting of SEQ ID NO: 9 or an amino acid sequence with at least 50% identity to SEQ ID NO: 9 or a variant with conservative substitutions for both L3 residues; and L4 consisting of SEQ ID NO: 12, 17, or 18 or an amino acid sequence with at least 50%, at least 75% sequence identity to SEQ ID NO: 12, 17, or 18.

Table 2 provides the amino acid sequences of the microdystrophin embodiments in accordance with the present disclosure. It is also contemplated that other embodiments are substituted variant of microdystrophin as defined by SEQ ID NOs: 1 (RGX-DYS1), 2 (RGX-DYS3), or 79 (RGX-DYS5). For example, conservative substitutions can be made to SEQ ID NOs: 1, 2, or 79 and substantially maintain its functional activity. In embodiments, microdystrophin may have at least 60%, at least 70%, at least 80%, at least 85%, at least 90%, at least 95%, at least 96%, at least 97%, at least 98%, or at least 99% sequence identity to the amino acid sequence of SEQ ID NOs: 1, 2, or 79 and maintain functional microdystrophin activity, as determined, for example, by one or more of the in vitro assays or in vivo assays in animal models disclosed in Section 5.4, infra. RGX-DYS2 and RGX-DYS4 of the disclosure also encode microdystrophin proteins comprising SEQ ID NO: 1.

TABLE 2
Amino acid sequences of RGX-DYS proteins

| | SEQ | |
|---|---|---|
| | ID | |
| Structure | NO: | Amino Acid Sequence |
| DYS1 | 1 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQQPDLAPGLTTIGASPTQTVTLVTQPVVTKETAI |
| | | SKLEMPSSLMLEVPTLERLQELQEATDELDLKLRQAEVIKGSWQPVGDLL |
| | | IDSLQDHLEKVKALRGEIAPLKENVSHVNDLARQLTTLGIQLSPYNLSTL |
| | | EDLNTRWKLLQVAVEDRVRQLHEAHRDFGPASQHELSTSVQGPWERAISP |
| | | NKVPYYINHETQTTCWDHPKMTELYQSLADLNNVRESAYRTAMKLRRLQK |
| | | ALCLDLLSLSAACDALDQHNLKQNDQPMDILQIINCLTTIYDRLEQEHNN |
| | | LVNVPLCVDMCLNWLLNVYDTGRTGRIRVLSFKTGIISLCKAHLEDKYRY |
| | | LFKQVASSTGFCDQRRLGLLLHDSIQIPRQLGEVASEGGSNIEPSVRSCF |
| | | QFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGE |
| | | DVRDFAKVLKNKFRTKRYFAKHPRMGYLPVQTVLEGDNMETPVTLINFWP |
| | | VDSAPASSPQLSHDDTHSRIEHYASRLAEMENSNGSYLNDSISPNESIDD |
| | | EHLLIQHYCQSLNQDSPLSQPRSPAQILISLESEERGELERILADLEEEN |
| | | RNLQAEYDRLKQQHEHKGLSPLPSPPEMMPTSPQSPR |
| DYS3 | 2 | MLWWEEVEDCYEREDVQKKTFTKWVNAQFSKFGKQHIENLFSDLQDGRRLLDLLE |
| | | GLTGQKLPKEKGSTRVHALNNVNKALRVLQNNNVDLVNIGSTDIVDGNHKLTLGL |
| | | IWNIILHWQVKNVMKNIMAGLQQTNSEKILLSWVRQSTRNYPQVNVINFTTSWSD |
| | | GLALNALIHSHRPDLFDWNSVVCQQSATQRLEHAFNIARYQLGIEKLLDPEDVDT |
| | | TYPDKKSILMYITSLFQVLPQQVSIEAIQEVEMLPRPPKVTKEEHFQLHHQMHYS |
| | | QQITVSLAQGYERTSSPKPRFKSYAYTQAAYVTTSDPTRSPFPSQHLEAPEDKSF |
| | | GSSLMESEVNLDRYQTALEEVLSWLLSAEDTLQAQGEISNDVEVVKDQFHTHEGY |
| | | MMDLTAHQGRVGNILQLGSKLIGTGKLSEDEETEVQEQMNLLNSRWECLRVASME |
| | | KQSNLHRVLMDLQNQKLKELNDWLTKTEERTRKMEEEPLGPDLEDLKRQVQQHKV |
| | | LQEDLEQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSLQKLAV |
| | | LKADLEKKKQSMGKLYSLKQDLLSTLKNKSVTQKTEAWLDNFARCWDNLVQKLEK |
| | | STAQISQQPDLAPGLTTIGASPTQTVTLVTQPVVTKETAISKLEMPSSLMLEVPT |
| | | LERLQELQEATDELDLKLRQAEVIKGSWQPVGDLLIDSLQDHLEKVKALRGEIAP |
| | | LKENVSHVNDLARQLTTLGIQLSPYNLSTLEDLNTRWKLLQVAVEDRVRQLHEAH |
| | | RDFGPASQHELSTSVQGPWERAISPNKVPYYINHETQTTCWDHPKMTELYQSLAD |
| | | LNNVRFSAYRTAMKLRRLQKALCLDLLSLSAACDALDQHNLKQNDQPMDILQIIN |
| | | CLTTIYDRLEQEHNNLVNVPLCVDMCLNWLLNVYDTGRIGRIRVLSFKTGIISLC |
| | | KAHLEDKYRYLFKQVASSTGFCDQRRLGLLLHDSIQIPRQLGEVASFGGSNIEPS |
| | | VRSCFQFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGEDVRDF |
| | | AKVLKNKFRTKRYFAKHPRMGYLPVQTVLEGDNMET |
| DYS5 | 79 | MLWWEEVEDCYEREDVQKKTFTKWVNAQFSKFGKQHIENLFSDLQDGRRLLDLLE |
| | | GLTGQKLPKEKGSTRVHALNNVNKALRVLQNNNVDLVNIGSTDIVDGNHKLTLGL |
| | | IWNIILHWQVKNVMKNIMAGLQQTNSEKILLSWVRQSTRNYPQVNVINFTTSWSD |
| | | GLALNALIHSHRPDLFDWNSVVCQQSATQRLEHAFNIARYQLGIEKLLDPEDVDT |
| | | TYPDKKSILMYITSLFQVLPQQVSIEAIQEVEMLPRPPKVTKEEHFQLHHQMHYS |
| | | QQITVSLAQGYERTSSPKPRFKSYAYTQAAYVTTSDPTRSPFPSQHLEAPEDKSF |
| | | GSSLMESEVNLDRYQTALEEVLSWLLSAEDTLQAQGEISNDVEVVKDQFHTHEGY |
| | | MMDLTAHQGRVGNILQLGSKLIGTGKLSEDEETEVQEQMNLLNSRWECLRVASME |
| | | KQSNLHRVLMDLQNQKLKELNDWLIKTEERTRKMEEEPLGPDLEDLKRQVQQHKV |
| | | LQEDLEQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSLQKLAV |
| | | LKADLEKKKQSMGKLYSLKQDLLSTLKNKSVTQKTEAWLDNFARCWDNLVQKLEK |
| | | STAQISQQPDLAPGLTTIGASPTQTVTLVTQPVVTKETAISKLEMPSSLMLEVPT |
| | | LERLQELQEATDELDLKLRQAEVIKGSWQPVGDLLIDSLQDHLEKVKALRGEIAP |
| | | LKENVSHVNDLARQLTTLGIQLSPYNLSTLEDLNTRWKLLQVAVEDRVRQLHEAH |
| | | RDFGPASQHELSTSVQGPWERAISPNKVPYYINHETQTTCWDHPKMTELYQSLAD |
| | | LNNVRESAYRTAMKLRRLQKALCLDLLSLSAACDALDQHNLKQNDQPMDILQIIN |
| | | CLTTIYDRLEQEHNNLVNVPLCVDMCLNWLLNVYDTGRIGRIRVLSFKTGIISLC |
| | | KAHLEDKYRYLFKQVASSTGFCDQRRLGLLLHDSIQIPRQLGEVASEGGSNIEPS |
| | | VRSCFQFANNKPEIEAALFLDWMRLEPQSMVWLPVLHRVAAAETAKHQAKCNICK |
| | | ECPIIGFRYRSLKHENYDICQSCFFSGRVAKGHKMHYPMVEYCTPTTSGEDVRDF |
| | | AKVLKNKFRTKRYFAKHPRMGYLPVQTVLEGDNMETPVTLINFWPVDSAPASSPQ |
| | | LSHDDTHSRIEHYASRLAEMENSNGSYLNDSISPNESIDDEHLLIQHYCQSLNQD |
| | | SPLSQPRSPAQILISLES |

### 5.2.2 Nucleic Acid Compositions Encoding Microdystrophin

Another aspect of the present disclosure are nucleic acids comprising a nucleotide sequence encoding a microdystrophin as described herein. Such nucleic acids comprise nucleotide sequences that encode the microdystrophin that has the domains arranged N-terminal to C-terminal as follows: ABD1-H1-R1-R2-R3-H3-R24-H4-CR-CT as detailed in Section 5.2.1, supra. The nucleotide sequence can be any nucleotide sequence that encodes the domains. The nucleotide sequence may be codon optimized and/or depleted of CpG islands for expression in the appropriate context. In particular embodiments, the nucleotide sequences encode a microdystrophin having an amino acid sequence of SEQ ID NO: 1, 2, or 79. The nucleotide sequence can be any sequence that encodes the microdystrophin, including the microdystrophin of SEQ ID NO: 1, SEQ ID NO: 2, of SEQ ID NO: 79, which nucleotide sequence may vary due to the degeneracy of the code. Tables 3 and 4 provide exemplary nucleotide sequences that encode the DMD domains. Table 3 provides the wild type DMD nucleotide sequence for the component and Table 4 provides the nucleotide sequence for the DMD component used in the constructs herein, including sequences that have been codon optimized and/or CpG depleted of CpG islands as follows:

TABLE 3
Dystrophin segment nucleotide sequences

| | SEQ | |
|---|---|---|
| Structure | ID | Nucleic Acid Sequence |
| ABD1 | 22 | ATGCTTTGGTGGGAAGAAGTAGAGGACTGTTATGAAAGAGAAGAT |
| | | GTTCAAAAGAAAACATTCACAAAATGGGTAAATGCACAATTTTCT |
| | | AAGTTTGGGAAGCAGCATATTGAGAACCTCTTCAGTGACCTACAG |
| | | GATGGGAGGCGCCTCCTAGACCTCCTCGAAGGCCTGACAGGGCAA |
| | | AAACTGCCAAAAGAAAAAGGATCCACAAGAGTTCATGCCCTGAAC |
| | | AATGTCAACAAGGCACTGCGGGTTTTGCAGAACAATAATGTTGAT |
| | | TTAGTGAATATTGGAAGTACTGACATCGTAGATGGAAATCATAAA |
| | | CTGACTCTTGGTTTGATTTGGAATATAATCCTCCACTGGCAGGTC |
| | | AAAAATGTAATGAAAAATATCATGGCTGGATTGCAACAAACCAAC |
| | | AGTGAAAAGATTCTCCTGAGCTGGGTCCGACAATCAACTCGTAAT |
| | | TATCCACAGGTTAATGTAATCAACTTCACCACCAGCTGGTCTGAT |
| | | GGCCTGGCTTTGAATGCTCTCATCCATAGTCATAGGCCAGACCTA |
| | | TTTGACTGGAATAGTGTGGTTTGCCAGCAGTCAGCCACACAACGA |
| | | CTGGAACATGCATTCAACATCGCCAGATATCAATTAGGCATAGAG |
| | | AAACTACTCGATCCTGAAGATGTTGATACCACCTATCCAGATAAG |
| | | AAGTCCATCTTAATGTACATCACATCACTCTTCCAAGTTTTGCCT |
| L1 | 23 | CAACAAGTGAGCATTGAAGCCATCCAGGAAGTGGAA |
| H1 | 24 | ATGTTGCCAAGGCCACCTAAAGTGACTAAAGAAGAACATTTTCAG |
| | | TTACATCATCAAATGCACTATTCTCAACAGATCACGGTCAGTCTA |
| | | GCACAGGGATATGAGAGAACTTCTTCCCCTAAGCCTCGATTCAAG |
| | | AGCTATGCCTACACACAGGCTGCTTATGTCACCACCTCTGACCCT |
| | | ACACGGAGCCCATTTCCTTCACAGCATTTGGAAGCTCCTGAAGAC |
| L2 | 25 | AAGTCATTTGGCAGTTCATTGATGGAG |
| R1 | 26 | AGTGAAGTAAACCTGGACCGTTATCAAACAGCTTTAGAAGAAGTA |
| | | TTATCGTGGCTTCTTTCTGCTGAGGACACATTGCAAGCACAAGGA |
| | | GAGATTTCTAATGATGTGGAAGTGGTGAAAGACCAGTTTCATACT |
| | | CATGAGGGGTACATGATGGATTTGACAGCCCATCAGGGCCGGGTT |
| | | GGTAATATTCTACAATTGGGAAGTAAGCTGATTGGAACAGGAAAA |
| | | TTATCAGAAGATGAAGAAACTGAAGTACAAGAGCAGATGAATCTC |
| | | CTAAATTCAAGATGGGAATGCCTCAGGGTAGCTAGCATGGAAAAA |
| | | CAAAGCAATTTACATAGA |
| R2 | 27 | GTTTTAATGGATCTCCAGAATCAGAAACTGAAAGAGTTGAATGAC |
| | | TGGCTAACAAAAACAGAAGAAAGAACAAGGAAAATGGAGGAAGAG |
| | | CCTCTTGGACCTGATCTTGAAGACCTAAAACGCCAAGTACAACAA |
| | | CATAAGGTGCTTCAAGAAGATCTAGAACAAGAACAAGTCAGGGTC |
| | | AATTCTCTCACTCACATGGTGGTGGTAGTTGATGAATCTAGTGGA |
| | | GATCACGCAACTGCTGCTTTGGAAGAACAACTTAAGGTATTGGGA |
| | | GATCGATGGGCAAACATCTGTAGATGGACAGAAGACCGCTGGGTT |
| | | CTTTTACAAGAC |
| L3 | 28 | ATCCTT |
| R3 | 29 | CTCAAATGGCAACGTCTTACTGAAGAACAGTGCCTTTTTAGTGCA |
| | | TGGCTTTCAGAAAAAGAAGATGCAGTGAACAAGATTCACACAACT |
| | | GGCTTTAAAGATCAAAATGAAATGTTATCAAGTCTTCAAAAACTG |
| | | GCCGTTTTAAAAGCGGATCTAGAAAAGAAAAAGCAATCCATGGGC |
| | | AAACTGTATTCACTCAAACAAGATCTTCTTTCAACACTGAAGAAT |
| | | AAGTCAGTGACCCAGAAGACGGAAGCATGGCTGGATAACTTTGCC |
| | | CGGTGTTGGGATAATTTAGTCCAAAAACTTGAAAAGAGTACAGCA |
| | | CAGATTTCACAG |
| L4.2 | 46 | CAAACCCTTGAA |
| H3 | 30 | CAGCCTGACCTAGCTCCTGGACTGACCACTATTGGAGCCTCTCCT |
| | | ACTCAGACTGTTACTCTGGTGACACAACCTGTGGTTACTAAGGAA |
| | | ACTGCCATCTCCAAACTAGAAATGCCATCTTCCTTGATGTTGGAG |
| | | GTACCT |
| L4 | 31 | ACCCTTGAA |
| R24 | 32 | AGACTCCAACTTCAAGAGGCCACGGATGAGCTGGACCTCAAGCTG |
| | | CGCCAAGCTGAGGTGATCAAGGGATCCTGGCAGCCCGTGGGCGAT |
| | | CTCCTCATTGACTCTCTCCAAGATCACCTCGAGAAAGTCAAGGCA |
| | | CTTCGAGGAGAAATTGCGCCTCTGAAAGAGAACGTGAGCCACGTC |
| | | AATGACCTTGCTCGCCAGCTTACCACTTTGGGCATTCAGCTCTCA |
| | | CCGTATAACCTCAGCACTCTGGAAGACCTGAACACCAGATGGAAG |
| | | CTTCTGCAGGTGGCCGTCGAGGACCGAGTCAGGCAGCTGCATGAA |
| H4 | 33 | GCCCACAGGGACTTTGGTCCAGCATCTCAGCACTTTCTTTCCACG |
| | | TCTGTCCAGGGTCCCTGGGAGAGAGCCATCTCGCCAAACAAAGTG |
| | | CCCTACTATATCAACCACGAGACTCAAACAACTTGCTGGGACCAT |
| | | CCCAAAATGACAGAGCTCTACCAGTCTTTAGCTGACCTGAATAAT |
| | | GTCAGATTCTCAGCTTATAGGACTGCCATGAAACTC |
| Cysteine-rich | 34 | CGAAGACTGCAGAAGGCCCTTTGCTTGGATCTCTTGAGCCTGTCA |
| domain (CR) | | GCTGCATGTGATGCCTTGGACCAGCACAACCTCAAGCAAAATGAC |
| | | CAGCCCATGGATATCCTGCAGATTATTAATTGTTTGACCACTATT |
| | | TATGACCGCCTGGAGCAAGAGCACAACAATTTGGTCAACGTCCCT |
| | | CTCTGCGTGGATATGTGTCTGAACTGGCTGCTGAATGTTTATGAT |
| | | ACGGGACGAACAGGGAGGATCCGTGTCCTGTCTTTTAAAACTGGC |
| | | ATCATTTCCCTGTGTAAAGCACATTTGGAAGACAAGTACAGATAC |
| | | CTTTTCAAGCAAGTGGCAAGTTCAACAGGATTTTGTGACCAGCGC |
| | | AGGCTGGGCCTCCTTCTGCATGATTCTATCCAAATTCCAAGACAG |
| | | TTGGGTGAAGTTGCATCCTTTGGGGGCAGTAACATTGAGCCAAGT |
| | | GTCCGGAGCTGCTTCCAATTTGCTAATAATAAGCCAGAGATCGAA |
| | | GCGGCCCTCTTCCTAGACTGGATGAGACTGGAACCCCAGTCCATG |
| | | GTGTGGCTGCCCGTCCTGCACAGAGTGGCTGCTGCAGAAACTGCC |
| | | AAGCATCAGGCCAAATGTAACATCTGCAAAGAGTGTCCAATCATT |
| | | GGATTCAGGTACAGGAGTCTAAAGCACTTTAATTATGACATCTGC |
| | | CAAAGCTGCTTTTTTTCTGGTCGAGTTGCAAAAGGCCATAAAATG |
| | | CACTATCCCATGGTGGAATATTGC |
| CR short | 47 | gccaagcatcaggccaaatgtaacatctgcaaagagtgtccaatc |
| | | attggattcaggtacaggagtctaaagcactttaattatgacatc |
| | | tgccaaagctgctttttttctggtcgagttgcaaaaggccataaa |
| | | atgcactatcccatggtggaatattgc |
| C-terminal | 35 | ACTCCGACTACATCAGGAGAAGATGTTCGAGACTTTGCCAAGGTA |
| (CT) Domain | | CTAAAAAACAAATTTCGAACCAAAAGGTATTTTGCGAAGCATCCC |
| | | CGAATGGGCTACCTGCCAGTGCAGACTGTCTTAGAGGGGGACAAC |
| | | ATGGAAACTCCCGTTACTCTGATCAACTTCTGGCCAGTAGATTCT |
| | | GCGCCTGCCTCGTCCCCTCAGCTTTCACACGATGATACTCATTCA |
| | | CGCATTGAACATTATGCTAGCAGGCTAGCAGAAATGGAAAACAGC |
| | | AATGGATCTTATCTAAATGATAGCATCTCTCCTAATGAGAGCATA |
| | | GATGATGAACATTTGTTAATCCAGCATTACTGCCAAAGTTTGAAC |
| | | CAGGACTCCCCCCTGAGCCAGCCTCGTAGTCCTGCCCAGATCTTG |
| | | ATTTCCTTAGAGAGTGAGGAAAGAGGGGAGCTAGAGAGAATCCTA |
| | | GCAGATCTTGAGGAAGAAAACAGGAATCTGCAAGCAGAATATGAC |
| | | CGTCTAAAGCAGCAGCACGAACATAAAGGCCTGTCCCCACTGCCG |
| | | TCCCCTCCTGAAATGATGCCCACCTCTCCCCAGAGTCCCCGG |
| L4 | 36 | GAGACCCTTGAA |
| L4 | 37 | CTTGAA |

TABLE 4
RGX-DYS segment nucleotide sequences (codon optimized and CpG
depleted

| | SEQ | |
|---|---|---|
| Structure | ID | Nucleic Acid Sequence |
| ABD | 57 | ATGCTTTGGTGGGAAGAGGTGGAAGATTGCTATGAGAGGGAAGA |
| | | TGTGCAGAAGAAAACCTTCACCAAATGGGTCAATGCCCAGTTCA |
| | | GCAAGTTTGGCAAGCAGCACATTGAGAACCTGTTCAGTGACCTG |
| | | CAGGATGGCAGAAGGCTGCTGGATCTGCTGGAAGGCCTGACAGG |
| | | CCAGAAGCTGCCTAAAGAGAAGGGCAGCACAAGAGTGCATGCCC |
| | | TGAACAATGTGAACAAGGCCCTGAGAGTGCTGCAGAACAACAAT |
| | | GTGGACCTGGTCAATATTGGCAGCACAGACATTGTGGATGGCAA |
| | | CCACAAGCTGACCCTGGGCCTGATCTGGAACATCATCCTGCACT |
| | | GGCAAGTGAAGAATGTGATGAAGAACATCATGGCTGGCCTGCAG |
| | | CAGACCAACTCTGAGAAGATCCTGCTGAGCTGGGTCAGACAGAG |
| | | CACCAGAAACTACCCTCAAGTGAATGTGATCAACTTCACCACCT |
| | | CTTGGAGTGATGGACTGGCCCTGAATGCCCTGATCCACAGCCAC |
| | | AGACCTGACCTGTTTGACTGGAACTCTGTTGTGTGCCAGCAGTC |
| | | TGCCACACAGAGACTGGAACATGCCTTCAACATTGCCAGATACC |
| | | AGCTGGGAATTGAGAAACTGCTGGACCCTGAGGATGTGGACACC |
| | | ACCTATCCTGACAAGAAATCCATCCTCATGTACATCACCAGCCT |
| | | GTTCCAGGTGCTGCCC |
| L1 | 58 | CAGCAAGTGTCCATTGAGGCCATTCAAGAGGTTGAG |
| H1 | 59 | ATGCTGCCCAGACCTCCTAAAGTGACCAAAGAGGAACACTTCCA |
| | | GCTGCACCACCAGATGCACTACTCTCAGCAGATCACAGTGTCTC |
| | | TGGCCCAGGGATATGAGAGAACAAGCAGCCCCAAGCCTAGGTTC |
| | | AAGAGCTATGCCTACACACAGGCTGCCTATGTGACCACATCTGA |
| | | CCCCACAAGAAGCCCATTTCCAAGCCAGCATCTGGAAGCCCCTG |
| | | AGGAC |
| L2 | 60 | AAGAGCTTTGGCAGCAGCCTGATGGAA |
| R1 | 61 | TCTGAAGTGAACCTGGATAGATACCAGACAGCCCTGGAAGAAGT |
| | | GCTGTCCTGGCTGCTGTCTGCTGAGGATACACTGCAGGCTCAGG |
| | | GTGAAATCAGCAATGATGTGGAAGTGGTCAAGGACCAGTTTCAC |
| | | ACCCATGAGGGCTACATGATGGACCTGACAGCCCACCAGGGCAG |
| | | AGTGGGAAATATCCTGCAGCTGGGCTCCAAGCTGATTGGCACAG |
| | | GCAAGCTGTCTGAGGATGAAGAGACAGAGGTGCAAGAGCAGATG |
| | | AACCTGCTGAACAGCAGATGGGAGTGTCTGAGAGTGGCCAGCAT |
| | | GGAAAAGCAGAGCAACCTGCACAGA |
| R2 | 62 | GTGCTCATGGACCTGCAGAATCAGAAACTGAAAGAACTGAATGA |
| | | CTGGCTGACCAAGACAGAAGAAAGGACTAGGAAGATGGAAGAGG |
| | | AACCTCTGGGACCAGACCTGGAAGATCTGAAAAGACAGGTGCAG |
| | | CAGCATAAGGTGCTGCAAGAGGACCTTGAGCAAGAGCAAGTCAG |
| | | AGTGAACAGCCTGACACACATGGTGGTGGTTGTGGATGAGTCCT |
| | | CTGGGGATCATGCCACAGCTGCTCTGGAAGAACAGCTGAAGGTG |
| | | CTGGGAGACAGATGGGCCAACATCTGTAGGTGGACAGAGGATAG |
| | | ATGGGTGCTGCTCCAGGAC |
| L3 | 63 | ATTCTG |
| R3 | 64 | CTGAAGTGGCAGAGACTGACAGAGGAACAGTGCCTGTTTTCTGC |
| | | CTGGCTCTCTGAGAAAGAGGATGCTGTCAACAAGATCCATACCA |
| | | CAGGCTTCAAGGATCAGAATGAGATGCTCAGCTCCCTGCAGAAA |
| | | CTGGCTGTGCTGAAGGCTGACCTGGAAAAGAAAAAGCAGTCCAT |
| | | GGGCAAGCTCTACAGCCTGAAGCAGGACCTGCTGTCTACCCTGA |
| | | AGAACAAGTCTGTGACCCAGAAAACTGAGGCCTGGCTGGACAAC |
| | | TTTGCTAGATGCTGGGACAACCTGGTGCAGAAGCTGGAAAAGTC |
| | | TACAGCCCAGATCAGCCAG |
| H3 | 65 | CAACCTGATCTTGCCCCTGGCCTGACCACAATTGGAGCCTCTCC |
| | | AACACAGACTGTGACCCTGGTTACCCAGCCAGTGGTCACCAAAG |
| | | AGACAGCCATCAGCAAACTGGAAATGCCCAGCTCTCTGATGCTG |
| | | GAAGTCCCC |
| L4 | 66 | ACACTGGAA |
| L4.1 | 19 | AGTGTG |
| L4.2 | 38 | CAGACACTGGAA |
| R24 | 67 | AGGCTGCAAGAACTTCAAGAGGCCACAGATGAGCTGGACCTGAA |
| | | GCTGAGACAGGCTGAAGTGATCAAAGGCAGCTGGCAGCCAGTTG |
| | | GGGACCTGCTCATTGATAGCCTGCAGGACCATCTGGAAAAAGTG |
| | | AAAGCCCTGAGGGGAGAGATTGCCCCTCTGAAAGAAAATGTGTC |
| | | CCATGTGAATGACCTGGCCAGACAGCTGACCACACTGGGAATCC |
| | | AGCTGAGCCCCTACAACCTGAGCACCCTTGAGGACCTGAACACC |
| | | AGGTGGAAGCTCCTCCAGGTGGCAGTGGAAGATAGAGTCAGGCA |
| | | GCTGCATGAG |
| H4 | 68 | GCCCACAGAGATTTTGGACCAGCCAGCCAGCACTTTCTGTCTAC |
| | | CTCTGTGCAAGGCCCCTGGGAGAGAGCTATCTCTCCTAACAAGG |
| | | TGCCCTACTACATCAACCATGAGACACAGACCACCTGTTGGGAT |
| | | CACCCCAAGATGACAGAGCTGTACCAGAGTCTGGCAGACCTCAA |
| | | CAATGTCAGATTCAGTGCCTACAGGACTGCCATGAAGCTC |
| Cysteine-rich | 69 | AGAAGGCTCCAGAAAGCTCTGTGCCTGGACCTGCTTTCCCTGAG |
| domain (CR) | | TGCAGCTTGTGATGCCCTGGACCAGCACAATCTGAAGCAGAATG |
| | | ACCAGCCTATGGACATCCTCCAGATCATCAACTGCCTCACCACC |
| | | ATCTATGATAGGCTGGAACAAGAGCACAACAATCTGGTCAATGT |
| | | GCCCCTGTGTGTGGACATGTGCCTGAATTGGCTGCTGAATGTGT |
| | | ATGACACAGGCAGAACAGGCAGGATCAGAGTCCTGTCCTTCAAG |
| | | ACAGGCATCATCTCCCTGTGCAAAGCCCACTTGGAGGACAAGTA |
| | | CAGATACCTGTTCAAGCAAGTGGCCTCCAGCACAGGCTTTTGTG |
| | | ACCAGAGAAGGCTGGGCCTGCTCCTGCATGACAGCATTCAGATC |
| | | CCTAGACAGCTGGGAGAAGTGGCTTCCTTTGGAGGCAGCAATAT |
| | | TGAGCCATCAGTCAGGTCCTGTTTTCAGTTTGCCAACAACAAGC |
| | | CTGAGATTGAGGCTGCCCTGTTCCTGGACTGGATGAGACTTGAG |
| | | CCTCAGAGCATGGTCTGGCTGCCTGTGCTTCATAGAGTGGCTGC |
| | | TGCTGAGACTGCCAAGCACCAGGCCAAGTGCAACATCTGCAAAG |
| | | AGTGCCCCATCATTGGCTTCAGATACAGATCCCTGAAGCACTTC |
| | | AACTATGATATCTGCCAGAGCTGCTTCTTTAGTGGCAGGGTTGC |
| | | CAAGGGCCACAAAATGCACTACCCCATGGTGGAATACTGC |
| CR short | 96 | GCCAAGCACCAGGCCAAGTGCAACATCTGCAAAGAGTGCCCCAT |
| (DYS6) | | CATTGGCTTCAGATACAGATCCCTGAAGCACTTCAACTATGATA |
| | | TCTGCCAGAGCTGCTTCTTTAGTGGCAGGGTTGCCAAGGGCCAC |
| | | AAAATGCACTACCCCATGGTGGAATACTGC |
| C-terminal | 70 | ACCCCAACAACCTCTGGGGAAGATGTTAGAGACTTTGCCAAGGT |
| (CT) Domain | | GCTGAAAAACAAGTTCAGGACCAAGAGATACTTTGCTAAGCACC |
| (DYS1) | | CCAGAATGGGCTACCTGCCTGTCCAGACAGTGCTTGAGGGTGAC |
| | | AACATGGAAACCCCTGTGACACTGATCAATTTCTGGCCAGTGGA |
| | | CTCTGCCCCTGCCTCAAGTCCACAGCTGTCCCATGATGACACCC |
| | | ACAGCAGAATTGAGCACTATGCCTCCAGACTGGCAGAGATGGAA |
| | | AACAGCAATGGCAGCTACCTGAATGATAGCATCAGCCCCAATGA |
| | | GAGCATTGATGATGAGCATCTGCTGATCCAGCACTACTGTCAGT |
| | | CCCTGAACCAGGACTCTCCACTGAGCCAGCCTAGAAGCCCTGCT |
| | | CAGATCCTGATCAGCCTTGAGTCTGAGGAAAGGGGAGAGCTGGA |
| | | AAGAATCCTGGCAGATCTTGAGGAAGAGAACAGAAACCTGCAGG |
| | | CAGAGTATGACAGGCTCAAACAGCAGCATGAGCACAAGGGACTG |
| | | AGCCCTCTGCCTTCTCCTCCTGAAATGATGCCCACCTCTCCACA |
| | | GTCTCCAAGGTGATGA (stop codons underlined) |
| Minimal C- | 80 | ACCCCAACAACCTCTGGGGAAGATGTTAGAGACTTTGCCAAGGT |
| terminal | | GCTGAAAAACAAGTTCAGGACCAAGAGATACTTTGCTAAGCACC |
| (CT1.5) | | CCAGAATGGGCTACCTGCCTGTCCAGACAGTGCTTGAGGGTGAC |
| Domain | | AACATGGAAACCCCTGTGACACTGATCAATTTCTGGCCAGTGGA |
| (DYS5,) | | CTCTGCCCCTGCCTCAAGTCCACAGCTGTCCCATGATGACACCC |
| | | ACAGCAGAATTGAGCACTATGCCTCCAGACTGGCAGAGATGGAA |
| | | AACAGCAATGGCAGCTACCTGAATGATAGCATCAGCCCCAATGA |
| | | GAGCATTGATGATGAGCATCTGCTGATCCAGCACTACTGTCAGT |
| | | CCCTGAACCAGGACTCTCCACTGAGCCAGCCTAGAAGCCCTGCT |
| | | CAGATCCTGATCAGCCTTGAGTCTTGATGA (stop codons |
| | | underlined) |
| LA | 71 | GAAACACTGGAA or GAGACACTGGAA |
| LA | 72 | CTGGAA |

In some embodiments, such compositions comprise a nucleic acid sequence encoding ABD1 that consists of SEQ ID NO: 22 or a sequence with at least 75%, at least 80%, at least 85%, at least 90%, at least 95%, at least 98% or at least 99% identity to SEQ ID NO: 22; a nucleic acid sequence encoding H1 that consists of SEQ ID NO: 24 or a sequence with at least 75%, at least 80%, at least 85%, at least 90%, at least 95%, at least 98% or at least 99% identity to SEQ ID NO: 24; a nucleic acid sequence encoding R1 that consists of SEQ ID NO: 26 or a sequence with at least 75%, at least 80%, at least 85%, at least 90%, at least 95%, at least 98% or at least 99% identity to SEQ ID NO: 26; a nucleic acid sequence encoding R2 that consists of SEQ ID NO: 27 or a sequence with at least 75%, at least 80%, at least 85%, at least 90%, at least 95%, at least 98% or at least 99% identity to SEQ ID NO: 27; a nucleic acid sequence encoding R3 that consists of SEQ ID NO: 29 or a sequence with at least 75%, at least 80%, at least 85%, at least 90%, at least 95%, at least 98% or at least 99% identity to SEQ ID NO: 29; a nucleic acid sequence encoding H3 that consists of SEQ ID NO: 30 or a sequence with at least 75%, at least 80%, at least 85%, at least 90%, at least 95%, at least 98% or at least 99% identity to SEQ ID NO: 30; a nucleic acid sequence encoding R24 that consists of SEQ ID NO: 32 or a sequence with at least 75%, at least 80%, at least 85%, at least 90%, at least 95%, at least 98% or at least 99% identity to SEQ ID NO: 32; a nucleic acid sequence encoding H4 that consists of SEQ ID NO: 33 or a sequence with at least 75%, at least 80%, at least 85%, at least 90%, at least 95%, at least 98%, or at least 99% identity to SEQ ID NO: 33; a nucleic acid sequence encoding CR that consists of SEQ ID NO: 34 or 47 or a sequence with at least 75%, at least 80%, at least 85%, at least 90%, at least 95%, at least 98% or at least 99% identity to SEQ ID NO: 34 or 47; and/or a nucleic acid sequence encoding CT that consists of SEQ ID NO: 35 or a sequence with at least 75%, at least 80%, at least 85%, at least 90%, at least 95%, at least 98% or at least 99% identity to SEQ ID NO: 35, encoding a microdystrophin that has functional activity.

In some embodiments, such compositions comprise a nucleic acid sequence encoding ABD1 that consists of SEQ ID NO: 22 or a sequence with at least 75%, at least 80%, at least 85%, at least 90%, at least 95%, at least 98% or at least 99% identity to SEQ ID NO: 22 and encodes for the ABD1 domain of SEQ ID NO: 3; a nucleic acid sequence encoding H1 that consists of SEQ ID NO: 24 or a sequence with at least 75%, at least 80%, at least 85%, at least 90%, at least 95%, at least 98% or at least 99% identity to SEQ ID NO: 24 and encodes for the H1 domain of SEQ ID NO: 5; a nucleic acid sequence encoding R1 that consists of SEQ ID NO: 26 or a sequence with at least 75%, at least 80%, at least 85%, at least 90%, at least 95%, at least 98% or at least 99% identity to SEQ ID NO: 26 and encodes for the R1 domain of SEQ ID NO: 7; a nucleic acid sequence encoding R2 that consists of SEQ ID NO: 27 or a sequence with at least 75%, at least 80%, at least 85%, at least 90%, at least 95%, at least 98% or at least 99% identity to SEQ ID NO: 27 and encodes for the R2 domain of SEQ ID NO: 8; a nucleic acid sequence encoding R3 that consists of SEQ ID NO: 29 or a sequence with at least 75%, at least 80%, at least 85%, at least 90%, at least 95%, at least 98% or at least 99% identity to SEQ ID NO: 29 and encodes for the R3 domain of SEQ ID NO: 10; a nucleic acid sequence encoding H3 that consists of SEQ ID NO: 30 or a sequence with at least 75%, at least 80%, at least 85%, at least 90%, at least 95%, at least 98% or at least 99% identity to SEQ ID NO: 30 and encodes for the H3 domain of SEQ ID NO: 11; a nucleic acid sequence encoding R24 that consists of SEQ ID NO: 32 or a sequence with at least 75%, at least 80%, at least 85%, at least 90%, at least 95%, at least 98% or at least 99% identity to SEQ ID NO: 32 and encodes for the R24 domain of SEQ ID NO: 13; a nucleic acid sequence encoding H4 that consists of SEQ ID NO: 33 or a sequence with at least 75%, at least 80%, at least 85%, at least 90%, at least 95%, at least 98%, or at least 99% identity to SEQ ID NO: 33 and encodes for the H4 domain of SEQ ID NO: 14; a nucleic acid sequence encoding CR that consists of SEQ ID NO: 34 or 47 or a sequence with at least 75%, at least 80%, at least 85%, at least 90%, at least 95%, at least 98% or at least 99% identity to SEQ ID NO: 34 or 47 and encodes for the CR domain of SEQ ID NO: 15 or 90; and/or a nucleic acid sequence encoding CT that consists of SEQ ID NO: 35 or 80 or a sequence with at least 75%, at least 80%, at least 85%, at least 90%, at least 95%, at least 98% or at least 99% identity to SEQ ID NO: 35 or 80 and encodes for the CT domain of SEQ ID NO: 16 or 83.

In addition to the foregoing, the nucleic acid compositions can optionally comprise nucleotide sequences encoding linkers in the locations described above that comprise or consist of sequences as follows: a nucleic acid sequence encoding L1 consisting of SEQ ID NO: 23 or a sequence with at least 80%, at least 85%, at least 90%, at least 95%, at least 98%, or at least 99% sequence identity to SEQ ID NO: 23 (e.g. encoding the L1 domain of SEQ ID NO: 4); a nucleic acid sequence encoding L2 consisting of SEQ ID NO: 25 or sequence with at least 80%, at least 85%, at least 90%, at least 95%, at least 98%, or at least 99% sequence identity to SEQ ID NO: 25 (e.g. encoding the L2 domain of SEQ ID NO: 6); a nucleic acid sequence encoding L3 consisting of SEQ ID NO: 28 or a sequence with at least 50% identity to SEQ ID NO: 28, encoding the L3 domain of SEQ ID NO: 9 or a variant with conservative substitutions for both L3 residues; and a nucleic acid sequence encoding L4 consisting of SEQ ID NO: 31, 36, or 37 or a sequence with at least 50%, at least 75% sequence identity to SEQ ID NO: 31, 36, or 37 (e.g. encoding the L4 domain of SEQ ID NO: 12, 17, or 18 or a variant with conservative substitutions for any of the L4 residues).

In various embodiments, the nucleic acid comprises a nucleotide sequence encoding the microdystrophin having the amino acid sequence of SEQ ID NO: 1, SEQ ID NO:2, or SEQ ID NO: 79. In embodiments, the nucleic acid comprises a nucleotide sequence which is SEQ ID NO: 20, SEQ ID NO: 21, or SEQ ID NO: 81, (encoding the microdystrophins of SEQ ID NO: 1, SEQ ID NO:2, or SEQ ID NO: 79, respectively). In various embodiments, the nucleotide sequence encoding a microdystrophin may have at least 50%, at least 60%, at least 70%, at least 75%, at least 80%, at least 85%, at least 90%, at least 95%, at least 98% or at least 99% sequence identity to the nucleotide sequence of SEQ ID NO: 20, 21, or 83 (Table 5) or the reverse complement thereof and encode a therapeutically effective microdystrophin.

TABLE 5
RGX-DYS Construct nucleotide sequences

| | SEQ | |
|---|---|---|
| Structure | ID | Nucleic Acid Sequence |
| DYS1 | 20 | ATGCTTTGGTGGGAAGAGGTGGAAGATTGCTATGAGAGGGAAGATGTGCA |
| | | GAAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCTG |
| | | CTGGATCTGCTGGAAGGCCTGACAGGCCAGAAGCTGCCTAAAGAGAAGGG |
| | | CAGCACAAGAGTGCATGCCCTGAACAATGTGAACAAGGCCCTGAGAGTGC |
| | | TGCAGAACAACAATGTGGACCTGGTCAATATTGGCAGCACAGACATTGTG |
| | | GATGGCAACCACAAGCTGACCCTGGGCCTGATCTGGAACATCATCCTGCA |
| | | CTGGCAAGTGAAGAATGTGATGAAGAACATCATGGCTGGCCTGCAGCAGA |
| | | CCAACTCTGAGAAGATCCTGCTGAGCTGGGTCAGACAGAGCACCAGAAAC |
| | | TACCCTCAAGTGAATGTGATCAACTTCACCACCTCTTGGAGTGATGGACT |
| | | GGCCCTGAATGCCCTGATCCACAGCCACAGACCTGACCTGTTTGACTGGA |
| | | ACTCTGTTGTGTGCCAGCAGTCTGCCACACAGAGACTGGAACATGCCTTC |
| | | AACATTGCCAGATACCAGCTGGGAATTGAGAAACTGCTGGACCCTGAGGA |
| | | TGTGGACACCACCTATCCTGACAAGAAATCCATCCTCATGTACATCACCA |
| | | GCCTGTTCCAGGTGCTGCCCCAGCAAGTGTCCATTGAGGCCATTCAAGAG |
| | | GTTGAGATGCTGCCCAGACCTCCTAAAGTGACCAAAGAGGAACACTTCCA |
| | | GCTGCACCACCAGATGCACTACTCTCAGCAGATCACAGTGTCTCTGGCCC |
| | | AGGGATATGAGAGAACAAGCAGCCCCAAGCCTAGGTTCAAGAGCTATGCC |
| | | TACACACAGGCTGCCTATGTGACCACATCTGACCCCACAAGAAGCCCATT |
| | | TCCAAGCCAGCATCTGGAAGCCCCTGAGGACAAGAGCTTTGGCAGCAGCC |
| | | TGATGGAATCTGAAGTGAACCTGGATAGATACCAGACAGCCCTGGAAGAA |
| | | GTGCTGTCCTGGCTGCTGTCTGCTGAGGATACACTGCAGGCTCAGGGTGA |
| | | AATCAGCAATGATGTGGAAGTGGTCAAGGACCAGTTTCACACCCATGAGG |
| | | GCTACATGATGGACCTGACAGCCCACCAGGGCAGAGTGGGAAATATCCTG |
| | | CAGCTGGGCTCCAAGCTGATTGGCACAGGCAAGCTGTCTGAGGATGAAGA |
| | | GACAGAGGTGCAAGAGCAGATGAACCTGCTGAACAGCAGATGGGAGTGTC |
| | | TGAGAGTGGCCAGCATGGAAAAGCAGAGCAACCTGCACAGAGTGCTCATG |
| | | GACCTGCAGAATCAGAAACTGAAAGAACTGAATGACTGGCTGACCAAGAC |
| | | AGAAGAAAGGACTAGGAAGATGGAAGAGGAACCTCTGGGACCAGACCTGG |
| | | AAGATCTGAAAAGACAGGTGCAGCAGCATAAGGTGCTGCAAGAGGACCTT |
| | | GAGCAAGAGCAAGTCAGAGTGAACAGCCTGACACACATGGTGGTGGTTGT |
| | | GGATGAGTCCTCTGGGGATCATGCCACAGCTGCTCTGGAAGAACAGCTGA |
| | | AGGTGCTGGGAGACAGATGGGCCAACATCTGTAGGTGGACAGAGGATAGA |
| | | TGGGTGCTGCTCCAGGACATTCTGCTGAAGTGGCAGAGACTGACAGAGGA |
| | | ACAGTGCCTGTTTTCTGCCTGGCTCTCTGAGAAAGAGGATGCTGTCAACA |
| | | AGATCCATACCACAGGCTTCAAGGATCAGAATGAGATGCTCAGCTCCCTG |
| | | CAGAAACTGGCTGTGCTGAAGGCTGACCTGGAAAAGAAAAAGCAGTCCAT |
| | | GGGCAAGCTCTACAGCCTGAAGCAGGACCTGCTGTCTACCCTGAAGAACA |
| | | AGTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCCA |
| | | GCAACCTGATCTTGCCCCTGGCCTGACCACAATTGGAGCCTCTCCAACAC |
| | | AGACTGTGACCCTGGTTACCCAGCCAGTGGTCACCAAAGAGACAGCCATC |
| | | AGCAAACTGGAAATGCCCAGCTCTCTGATGCTGGAAGTCCCCACACTGGA |
| | | AAGGCTGCAAGAACTTCAAGAGGCCACAGATGAGCTGGACCTGAAGCTGA |
| | | GACAGGCTGAAGTGATCAAAGGCAGCTGGCAGCCAGTTGGGGACCTGCTC |
| | | ATTGATAGCCTGCAGGACCATCTGGAAAAAGTGAAAGCCCTGAGGGGAGA |
| | | GATTGCCCCTCTGAAAGAAAATGTGTCCCATGTGAATGACCTGGCCAGAC |
| | | AGCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATAG |
| | | AGTCAGGCAGCTGCATGAGGCCCACAGAGATTTTGGACCAGCCAGCCAGC |
| | | ACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGA |
| | | TCACCCCAAGATGACAGAGCTGTACCAGAGTCTGGCAGACCTCAACAATG |
| | | TCAGATTCAGTGCCTACAGGACTGCCATGAAGCTCAGAAGGCTCCAGAAA |
| | | GCTCTGTGCCTGGACCTGCTTTCCCTGAGTGCAGCTTGTGATGCCCTGGA |
| | | CCAGCACAATCTGAAGCAGAATGACCAGCCTATGGACATCCTCCAGATCA |
| | | TCAACTGCCTCACCACCATCTATGATAGGCTGGAACAAGAGCACAACAAT |
| | | CTGGTCAATGTGCCCCTGTGTGTGGACATGTGCCTGAATTGGCTGCTGAA |
| | | TGTGTATGACACAGGCAGAACAGGCAGGATCAGAGTCCTGTCCTTCAAGA |
| | | CAGGCATCATCTCCCTGTGCAAAGCCCACTTGGAGGACAAGTACAGATAC |
| | | CTGTTCAAGCAAGTGGCCTCCAGCACAGGCTTTTGTGACCAGAGAAGGCT |
| | | GGGCCTGCTCCTGCATGACAGCATTCAGATCCCTAGACAGCTGGGAGAAG |
| | | TGGCTTCCTTTGGAGGCAGCAATATTGAGCCATCAGTCAGGTCCTGTTTT |
| | | CAGTTTGCCAACAACAAGCCTGAGATTGAGGCTGCCCTGTTCCTGGACTG |
| | | GATGAGACTTGAGCCTCAGAGCATGGTCTGGCTGCCTGTGCTTCATAGAG |
| | | TGGCTGCTGCTGAGACTGCCAAGCACCAGGCCAAGTGCAACATCTGCAAA |
| | | GAGTGCCCCATCATTGGCTTCAGATACAGATCCCTGAAGCACTTCAACTA |
| | | TGATATCTGCCAGAGCTGCTTCTTTAGTGGCAGGGTTGCCAAGGGCCACA |
| | | AAATGCACTACCCCATGGTGGAATACTGCACCCCAACAACCTCTGGGGAA |
| | | GATGTTAGAGACTTTGCCAAGGTGCTGAAAAACAAGTTCAGGACCAAGAG |
| | | ATACTTTGCTAAGCACCCCAGAATGGGCTACCTGCCTGTCCAGACAGTGC |
| | | TTGAGGGTGACAACATGGAAACCCCTGTGACACTGATCAATTTCTGGCCA |
| | | GTGGACTCTGCCCCTGCCTCAAGTCCACAGCTGTCCCATGATGACACCCA |
| | | CAGCAGAATTGAGCACTATGCCTCCAGACTGGCAGAGATGGAAAACAGCA |
| | | ATGGCAGCTACCTGAATGATAGCATCAGCCCCAATGAGAGCATTGATGAT |
| | | GAGCATCTGCTGATCCAGCACTACTGTCAGTCCCTGAACCAGGACTCTCC |
| | | ACTGAGCCAGCCTAGAAGCCCTGCTCAGATCCTGATCAGCCTTGAGTCTG |
| | | AGGAAAGGGGAGAGCTGGAAAGAATCCTGGCAGATCTTGAGGAAGAGAAC |
| | | AGAAACCTGCAGGCAGAGTATGACAGGCTCAAACAGCAGCATGAGCACAA |
| | | GGGACTGAGCCCTCTGCCTTCTCCTCCTGAAATGATGCCCACCTCTCCAC |
| | | AGTCTCCAAGGTGATGA |
| DYS3 | 21 | ATGCTTTGGTGGGAAGAGGTGGAAGATTGCTATGAGAGGGAAGATGTGCA |
| | | GAAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCTG |
| | | CTGGATCTGCTGGAAGGCCTGACAGGCCAGAAGCTGCCTAAAGAGAAGGG |
| | | CAGCACAAGAGTGCATGCCCTGAACAATGTGAACAAGGCCCTGAGAGTGC |
| | | TGCAGAACAACAATGTGGACCTGGTCAATATTGGCAGCACAGACATTGTG |
| | | GATGGCAACCACAAGCTGACCCTGGGCCTGATCTGGAACATCATCCTGCA |
| | | CTGGCAAGTGAAGAATGTGATGAAGAACATCATGGCTGGCCTGCAGCAGA |
| | | CCAACTCTGAGAAGATCCTGCTGAGCTGGGTCAGACAGAGCACCAGAAAC |
| | | TACCCTCAAGTGAATGTGATCAACTTCACCACCTCTTGGAGTGATGGACT |
| | | GGCCCTGAATGCCCTGATCCACAGCCACAGACCTGACCTGTTTGACTGGA |
| | | ACTCTGTTGTGTGCCAGCAGTCTGCCACACAGAGACTGGAACATGCCTTC |
| | | AACATTGCCAGATACCAGCTGGGAATTGAGAAACTGCTGGACCCTGAGGA |
| | | TGTGGACACCACCTATCCTGACAAGAAATCCATCCTCATGTACATCACCA |
| | | GCCTGTTCCAGGTGCTGCCCCAGCAAGTGTCCATTGAGGCCATTCAAGAG |
| | | GTTGAGATGCTGCCCAGACCTCCTAAAGTGACCAAAGAGGAACACTTCCA |
| | | GCTGCACCACCAGATGCACTACTCTCAGCAGATCACAGTGTCTCTGGCCC |
| | | AGGGATATGAGAGAACAAGCAGCCCCAAGCCTAGGTTCAAGAGCTATGCC |
| | | TACACACAGGCTGCCTATGTGACCACATCTGACCCCACAAGAAGCCCATT |
| | | TCCAAGCCAGCATCTGGAAGCCCCTGAGGACAAGAGCTTTGGCAGCAGCC |
| | | TGATGGAATCTGAAGTGAACCTGGATAGATACCAGACAGCCCTGGAAGAA |
| | | GTGCTGTCCTGGCTGCTGTCTGCTGAGGATACACTGCAGGCTCAGGGTGA |
| | | AATCAGCAATGATGTGGAAGTGGTCAAGGACCAGTTTCACACCCATGAGG |
| | | GCTACATGATGGACCTGACAGCCCACCAGGGCAGAGTGGGAAATATCCTG |
| | | CAGCTGGGCTCCAAGCTGATTGGCACAGGCAAGCTGTCTGAGGATGAAGA |
| | | GACAGAGGTGCAAGAGCAGATGAACCTGCTGAACAGCAGATGGGAGTGTC |
| | | TGAGAGTGGCCAGCATGGAAAAGCAGAGCAACCTGCACAGAGTGCTCATG |
| | | GACCTGCAGAATCAGAAACTGAAAGAACTGAATGACTGGCTGACCAAGAC |
| | | AGAAGAAAGGACTAGGAAGATGGAAGAGGAACCTCTGGGACCAGACCTGG |
| | | AAGATCTGAAAAGACAGGTGCAGCAGCATAAGGTGCTGCAAGAGGACCTT |
| | | GAGCAAGAGCAAGTCAGAGTGAACAGCCTGACACACATGGTGGTGGTTGT |
| | | GGATGAGTCCTCTGGGGATCATGCCACAGCTGCTCTGGAAGAACAGCTGA |
| | | AGGTGCTGGGAGACAGATGGGCCAACATCTGTAGGTGGACAGAGGATAGA |
| | | TGGGTGCTGCTCCAGGACATTCTGCTGAAGTGGCAGAGACTGACAGAGGA |
| | | ACAGTGCCTGTTTTCTGCCTGGCTCTCTGAGAAAGAGGATGCTGTCAACA |
| | | AGATCCATACCACAGGCTTCAAGGATCAGAATGAGATGCTCAGCTCCCTG |
| | | CAGAAACTGGCTGTGCTGAAGGCTGACCTGGAAAAGAAAAAGCAGTCCAT |
| | | GGGCAAGCTCTACAGCCTGAAGCAGGACCTGCTGTCTACCCTGAAGAACA |
| | | AGTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCCA |
| | | GCAACCTGATCTTGCCCCTGGCCTGACCACAATTGGAGCCTCTCCAACAC |
| | | AGACTGTGACCCTGGTTACCCAGCCAGTGGTCACCAAAGAGACAGCCATC |
| | | AGCAAACTGGAAATGCCCAGCTCTCTGATGCTGGAAGTCCCCACACTGGA |
| | | AAGGCTGCAAGAACTTCAAGAGGCCACAGATGAGCTGGACCTGAAGCTGA |
| | | GACAGGCTGAAGTGATCAAAGGCAGCTGGCAGCCAGTTGGGGACCTGCTC |
| | | ATTGATAGCCTGCAGGACCATCTGGAAAAAGTGAAAGCCCTGAGGGGAGA |
| | | GATTGCCCCTCTGAAAGAAAATGTGTCCCATGTGAATGACCTGGCCAGAC |
| | | AGCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATAG |
| | | AGTCAGGCAGCTGCATGAGGCCCACAGAGATTTTGGACCAGCCAGCCAGC |
| | | ACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGA |
| | | TCACCCCAAGATGACAGAGCTGTACCAGAGTCTGGCAGACCTCAACAATG |
| | | TCAGATTCAGTGCCTACAGGACTGCCATGAAGCTCAGAAGGCTCCAGAAA |
| | | GCTCTGTGCCTGGACCTGCTTTCCCTGAGTGCAGCTTGTGATGCCCTGGA |
| | | CCAGCACAATCTGAAGCAGAATGACCAGCCTATGGACATCCTCCAGATCA |
| | | TCAACTGCCTCACCACCATCTATGATAGGCTGGAACAAGAGCACAACAAT |
| | | CTGGTCAATGTGCCCCTGTGTGTGGACATGTGCCTGAATTGGCTGCTGAA |
| | | TGTGTATGACACAGGCAGAACAGGCAGGATCAGAGTCCTGTCCTTCAAGA |
| | | CAGGCATCATCTCCCTGTGCAAAGCCCACTTGGAGGACAAGTACAGATAC |
| | | CTGTTCAAGCAAGTGGCCTCCAGCACAGGCTTTTGTGACCAGAGAAGGCT |
| | | GGGCCTGCTCCTGCATGACAGCATTCAGATCCCTAGACAGCTGGGAGAAG |
| | | TGGCTTCCTTTGGAGGCAGCAATATTGAGCCATCAGTCAGGTCCTGTTTT |
| | | CAGTTTGCCAACAACAAGCCTGAGATTGAGGCTGCCCTGTTCCTGGACTG |
| | | GATGAGACTTGAGCCTCAGAGCATGGTCTGGCTGCCTGTGCTTCATAGAG |
| | | TGGCTGCTGCTGAGACTGCCAAGCACCAGGCCAAGTGCAACATCTGCAAA |
| | | GAGTGCCCCATCATTGGCTTCAGATACAGATCCCTGAAGCACTTCAACTA |
| | | TGATATCTGCCAGAGCTGCTTCTTTAGTGGCAGGGTTGCCAAGGGCCACA |
| | | AAATGCACTACCCCATGGTGGAATACTGCACCCCAACAACCTCTGGGGAA |
| | | GATGTTAGAGACTTTGCCAAGGTGCTGAAAAACAAGTTCAGGACCAAGAG |
| | | ATACTTTGCTAAGCACCCCAGAATGGGCTACCTGCCTGTCCAGACAGTGC |
| | | TTGAGGGTGACAACATGGAAACC |
| DYS5 | 81 | ATGCTTTGGTGGGAAGAGGTGGAAGATTGCTATGAGAGGGAAGATGTGCAG |
| | | AAGAAAACCTTCACCAAATGGGTCAATGCCCAGTTCAGCAAGTTTGGCAAG |
| | | CAGCACATTGAGAACCTGTTCAGTGACCTGCAGGATGGCAGAAGGCTGCTG |
| | | GATCTGCTGGAAGGCCTGACAGGCCAGAAGCTGCCTAAAGAGAAGGGCAGC |
| | | ACAAGAGTGCATGCCCTGAACAATGTGAACAAGGCCCTGAGAGTGCTGCAG |
| | | AACAACAATGTGGACCTGGTCAATATTGGCAGCACAGACATTGTGGATGGC |
| | | AACCACAAGCTGACCCTGGGCCTGATCTGGAACATCATCCTGCACTGGCAA |
| | | GTGAAGAATGTGATGAAGAACATCATGGCTGGCCTGCAGCAGACCAACTCT |
| | | GAGAAGATCCTGCTGAGCTGGGTCAGACAGAGCACCAGAAACTACCCTCAA |
| | | GTGAATGTGATCAACTTCACCACCTCTTGGAGTGATGGACTGGCCCTGAAT |
| | | GCCCTGATCCACAGCCACAGACCTGACCTGTTTGACTGGAACTCTGTTGTG |
| | | TGCCAGCAGTCTGCCACACAGAGACTGGAACATGCCTTCAACATTGCCAGA |
| | | TACCAGCTGGGAATTGAGAAACTGCTGGACCCTGAGGATGTGGACACCACC |
| | | TATCCTGACAAGAAATCCATCCTCATGTACATCACCAGCCTGTTCCAGGTG |
| | | CTGCCCCAGCAAGTGTCCATTGAGGCCATTCAAGAGGTTGAGATGCTGCCC |
| | | AGACCTCCTAAAGTGACCAAAGAGGAACACTTCCAGCTGCACCACCAGATG |
| | | CACTACTCTCAGCAGATCACAGTGTCTCTGGCCCAGGGATATGAGAGAACA |
| | | AGCAGCCCCAAGCCTAGGTTCAAGAGCTATGCCTACACACAGGCTGCCTAT |
| | | GTGACCACATCTGACCCCACAAGAAGCCCATTTCCAAGCCAGCATCTGGAA |
| | | GCCCCTGAGGACAAGAGCTTTGGCAGCAGCCTGATGGAATCTGAAGTGAAC |
| | | CTGGATAGATACCAGACAGCCCTGGAAGAAGTGCTGTCCTGGCTGCTGTCT |
| | | GCTGAGGATACACTGCAGGCTCAGGGTGAAATCAGCAATGATGTGGAAGTG |
| | | GTCAAGGACCAGTTTCACACCCATGAGGGCTACATGATGGACCTGACAGCC |
| | | CACCAGGGCAGAGTGGGAAATATCCTGCAGCTGGGCTCCAAGCTGATTGGC |
| | | ACAGGCAAGCTGTCTGAGGATGAAGAGACAGAGGTGCAAGAGCAGATGAAC |
| | | CTGCTGAACAGCAGATGGGAGTGTCTGAGAGTGGCCAGCATGGAAAAGCAG |
| | | AGCAACCTGCACAGAGTGCTCATGGACCTGCAGAATCAGAAACTGAAAGAA |
| | | CTGAATGACTGGCTGACCAAGACAGAAGAAAGGACTAGGAAGATGGAAGAG |
| | | GAACCTCTGGGACCAGACCTGGAAGATCTGAAAAGACAGGTGCAGCAGCAT |
| | | AAGGTGCTGCAAGAGGACCTTGAGCAAGAGCAAGTCAGAGTGAACAGCCTG |
| | | ACACACATGGTGGTGGTTGTGGATGAGTCCTCTGGGGATCATGCCACAGCT |
| | | GCTCTGGAAGAACAGCTGAAGGTGCTGGGAGACAGATGGGCCAACATCTGT |
| | | AGGTGGACAGAGGATAGATGGGTGCTGCTCCAGGACATTCTGCTGAAGTGG |
| | | CAGAGACTGACAGAGGAACAGTGCCTGTTTTCTGCCTGGCTCTCTGAGAAA |
| | | GAGGATGCTGTCAACAAGATCCATACCACAGGCTTCAAGGATCAGAATGAG |
| | | ATGCTCAGCTCCCTGCAGAAACTGGCTGTGCTGAAGGCTGACCTGGAAAAG |
| | | AAAAAGCAGTCCATGGGCAAGCTCTACAGCCTGAAGCAGGACCTGCTGTCT |
| | | ACCCTGAAGAACAAGTCTGTGACCCAGAAAACTGAGGCCTGGCTGGACAAC |
| | | TTTGCTAGATGCTGGGACAACCTGGTGCAGAAGCTGGAAAAGTCTACAGCC |
| | | CAGATCAGCCAGCAACCTGATCTTGCCCCTGGCCTGACCACAATTGGAGCC |
| | | TCTCCAACACAGACTGTGACCCTGGTTACCCAGCCAGTGGTCACCAAAGAG |
| | | ACAGCCATCAGCAAACTGGAAATGCCCAGCTCTCTGATGCTGGAAGTCCCC |
| | | ACACTGGAAAGGCTGCAAGAACTTCAAGAGGCCACAGATGAGCTGGACCTG |
| | | AAGCTGAGACAGGCTGAAGTGATCAAAGGCAGCTGGCAGCCAGTTGGGGAC |
| | | CTGCTCATTGATAGCCTGCAGGACCATCTGGAAAAAGTGAAAGCCCTGAGG |
| | | GGAGAGATTGCCCCTCTGAAAGAAAATGTGTCCCATGTGAATGACCTGGCC |
| | | AGACAGCTGACCACACTGGGAATCCAGCTGAGCCCCTACAACCTGAGCACC |
| | | CTTGAGGACCTGAACACCAGGTGGAAGCTCCTCCAGGTGGCAGTGGAAGAT |
| | | AGAGTCAGGCAGCTGCATGAGGCCCACAGAGATTTTGGACCAGCCAGCCAG |
| | | CACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGAT |
| | | CACCCCAAGATGACAGAGCTGTACCAGAGTCTGGCAGACCTCAACAATGTC |
| | | AGATTCAGTGCCTACAGGACTGCCATGAAGCTCAGAAGGCTCCAGAAAGCT |
| | | CTGTGCCTGGACCTGCTTTCCCTGAGTGCAGCTTGTGATGCCCTGGACCAG |
| | | CACAATCTGAAGCAGAATGACCAGCCTATGGACATCCTCCAGATCATCAAC |
| | | TGCCTCACCACCATCTATGATAGGCTGGAACAAGAGCACAACAATCTGGTC |
| | | AATGTGCCCCTGTGTGTGGACATGTGCCTGAATTGGCTGCTGAATGTGTAT |
| | | GACACAGGCAGAACAGGCAGGATCAGAGTCCTGTCCTTCAAGACAGGCATC |
| | | ATCTCCCTGTGCAAAGCCCACTTGGAGGACAAGTACAGATACCTGTTCAAG |
| | | CAAGTGGCCTCCAGCACAGGCTTTTGTGACCAGAGAAGGCTGGGCCTGCTC |
| | | CTGCATGACAGCATTCAGATCCCTAGACAGCTGGGAGAAGTGGCTTCCTTT |
| | | GGAGGCAGCAATATTGAGCCATCAGTCAGGTCCTGTTTTCAGTTTGCCAAC |
| | | AACAAGCCTGAGATTGAGGCTGCCCTGTTCCTGGACTGGATGAGACTTGAG |
| | | CCTCAGAGCATGGTCTGGCTGCCTGTGCTTCATAGAGTGGCTGCTGCTGAG |
| | | ACTGCCAAGCACCAGGCCAAGTGCAACATCTGCAAAGAGTGCCCCATCATT |
| | | GGCTTCAGATACAGATCCCTGAAGCACTTCAACTATGATATCTGCCAGAGC |
| | | TGCTTCTTTAGTGGCAGGGTTGCCAAGGGCCACAAAATGCACTACCCCATG |
| | | GTGGAATACTGCACCCCAACAACCTCTGGGGAAGATGTTAGAGACTTTGCC |
| | | AAGGTGCTGAAAAACAAGTTCAGGACCAAGAGATACTTTGCTAAGCACCCC |
| | | AGAATGGGCTACCTGCCTGTCCAGACAGTGCTTGAGGGTGACAACATGGAA |
| | | ACCCCTGTGACACTGATCAATTTCTGGCCAGTGGACTCTGCCCCTGCCTCA |
| | | AGTCCACAGCTGTCCCATGATGACACCCACAGCAGAATTGAGCACTATGCC |
| | | TCCAGACTGGCAGAGATGGAAAACAGCAATGGCAGCTACCTGAATGATAGC |
| | | ATCAGCCCCAATGAGAGCATTGATGATGAGCATCTGCTGATCCAGCACTAC |
| | | TGTCAGTCCCTGAACCAGGACTCTCCACTGAGCCAGCCTAGAAGCCCTGCT |
| | | CAGATCCTGATCAGCCTTGAGTCTTGATGA |

### 5.2.2.1 Codon Optimization and CpG Depletion

In one aspect the nucleotide sequence encoding the microdystrophin cassette is modified by codon optimization and CpG dinucleotide and CpG island depletion. Immune response against microdystrophin transgene is a concern for human clinical application, as evidenced in the first Duchenne Muscular Dystrophy (DMD) gene therapy clinical trials and in several adeno-associated vial (AAV)-minidystrophin gene therapy in canine models [Mendell, J.R., et al., Dystrophin immunity in Duchenne's muscular dystrophy. N Engl J Med, 2010. 363(15): p. 1429-37; and Kornegay, J. N., et al., Widespread muscle expression of an AAV9 human mini-dystrophin vector after intravenous injection in neonatal dystrophin-deficient dogs. Mol Ther, 2010. 18(8): p. 1501-8].

AAV-directed immune responses can be inhibited by reducing the number of CpG di-nucleotides in the AAV genome [Faust, S. M., et al., CpG-depleted adeno-associated virus vectors evade immune detection. J Clin Invest, 2013. 123(7): p. 2994-3001]. Depleting the transgene sequence of CpG motifs may diminish the role of TLR9 in activation of innate immunity upon recognition of the transgene as non-self, and thus provide stable and prolonged transgene expression. [See also Wang, D., P. W.L. Tai, and G. Gao, Adeno-associated virus vector as a platform for gene therapy delivery. Nat Rev Drug Discov, 2019. 18(5): p. 358-378.; and Rabinowitz, J., Y. K. Chan, and R. J. Samulski, Adeno-associated Virus (AAV) versus Immune Response. Viruses, 2019. 11(2)]. In embodiments, the microdystrophin cassette is human codon-optimized with CpG depletion. Codon-optimized and CpG depleted nucleotide sequences may be designed by any method known in the art, including for example, by Thermo Fisher Scientific GeneArt Gene Synthesis tools utilizing GeneOptimizer (Waltham, MA USA)). Nucleotide sequences SEQ ID NOs: 20, 21, 57-72, 80, 81, and 101-103 described herein represent codon-optimized and CpG depleted sequences.

Provided are microdystrophin transgenes that have reduced numbers of CpG dinucleotide sequences and, as a result, have reduced number of CpG islands. In certain embodiments, the microdystrophin nucleotide sequence has fewer than two (2) CpG islands, or one (1) CpG island or zero (0) CpG islands. In embodiments, provided are microdystrophin transgenes having fewer than 2, or 1 CpG islands, or 0 CpG islands that have reduced immunogenicity, as measured by anti-drug antibody titer compared to a microdystrophin transgene having more than 2 CpG islands. In certain embodiments, the microdystrophin nucleotide sequence consisting essentially of SEQ ID NO: 20, 21, or 81 has zero (0) CpG islands. In other embodiments, the microdystrophin transgene nucleotide sequence consisting essentially of a microdystrophin gene operably linked to a promoter, wherein the microdystrophin consists of SEQ ID NO: 20, 21, or 81, has less than two (2) CpG islands. In still other embodiments, the microdystrophin transgene nucleotide sequence consisting essentially of a microdystrophin gene operably linked to a promoter, wherein the microdystrophin consists of SEQ ID NO: 20, 21, or 81, has one (1) CpG island.

### 5.3. Gene Cassettes and Regulatory Elements

Another aspect of the present invention relates to nucleic acid expression cassettes comprising regulatory elements designed to confer or enhance expression of the microdystrophins. The invention further involves engineering regulatory elements, including promoter elements, and optionally enhancer elements and/or introns, to enhance or facilitate expression of the transgene. In some embodiments, the rAAV vector also includes such regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue.

### 5.3.1 Promoters

### 5.3.1.1 Tissue-Specific Promoters

In specific embodiments, the expression cassette of an AAV vector comprises a regulatory sequence, such as a promoter, operably linked to the transgene that allows for expression in target tissues. The promoter may be a muscle promoter. In certain embodiments, the promoter is a muscle-specific promoter. The phrase “muscle-specific”, “muscle-selective” or “muscle-directed” refers to nucleic acid elements that have adapted their activity in muscle cells or tissue due to the interaction of such elements with the intracellular environment of the muscle cells. Such muscle cells may include myocytes, myotubes, cardiomyocytes, and the like. Specialized forms of myocytes with distinct properties such as cardiac, skeletal, and smooth muscle cells are included. Various therapeutics may benefit from muscle-specific expression of a transgene. In particular, gene therapies that treat various forms of muscular dystrophy delivered to and enabling high transduction efficiency in muscle cells have the added benefit of directing expression of the transgene in the cells where the transgene is most needed. Cardiac tissue will also benefit from muscle-directed expression of the transgene. Muscle-specific promoters may be operably linked to the transgenes of the invention. In some embodiments, the muscle-specific promoter is selected from an SPc5-12 promoter (SEQ ID NO: 39), a muscle creatine kinase myosin light chain (MLC) promoter, a myosin heavy chain (MHC) promoter, a desmin promoter (SEQ ID NO: 89), a MHCK7 promoter (SEQ ID NO: 94), a CK6 promoter, a CK8 promoter (SEQ ID NO: 51), a MCK promoter (or a truncated form thereof) (SEQ ID NO: 86), an alpha actin promoter, an beta actin promoter, an gamma actin promoter, an E-syn promoter, a cardiac troponin C promoter, a troponin I promoter, a myoD gene family promoter, or a muscle-selective promoter residing within intron 1 of the ocular form of Pitx3.

Synthetic promoter c5-12 (Li, X. et al. Nature Biotechnology Vol. 17, pp. 241-245, MARCH 1999), known as the SPc5-12 promoter, has been shown to have cell type restricted expression, specifically muscle-cell specific expression. At less than 350 bp in length, the SPc5-12 promoter is smaller in length than most endogenous promoters, which can be advantageous when the length of the nucleic acid encoding the therapeutic protein is relatively long.

In order to further reduce the length of a vector, regulatory elements can be a reduced or shortened version (referred to herein as a “minimal promoter”) of any one of the promoters described herein. A minimal promoter comprises at least the transcriptionally active domain of the full-length version and is therefore still capable of driving expression. For example, in some embodiments, an AAV vector can comprise the transcriptionally active domain of a muscle-specific promoter, e.g., a minimal SPc5-12 promoter (e.g., SEQ ID NO: 40), operably linked to a therapeutic protein transgene. In embodiments, the therapeutic protein is microdystrophin as described herein. A minimal promoter of the present disclosure may or may not contain the portion of the promoter sequence that contributes to regulating expression in a tissue-specific manner.

Accordingly, in embodiments, provided are gene therapy cassettes with an SPc5-12 promoter (SEQ ID NO: 39). For example, RGX-DYS1 and RGX-DYS5 (FIG. 2) have the Spc5-12 promoter. Sequences of these promoters are provided in Table 6.

Alternatively, the promoter may be a constitutive promoter, for example, the CB7 promoter. Additional promoters include: cytomegalovirus (CMV) promoter, Rous sarcoma virus (RSV) promoter, MMT promoter, EF-1 alpha promoter (SEQ ID NO: 54), UB6 promoter, chicken beta-actin promoter, CAG promoter (SEQ ID NO: 52), RPE65 promoter, opsin promoter, the TBG (Thyroxine-binding Globulin) promoter, the APOA2 promoter, SERPINA1 (hAAT) promoter, or MIR122 promoter. In some embodiments, particularly where it may be desirable to turn off transgene expression, an inducible promoter is used, e.g., hypoxia-inducible or rapamycin-inducible promoter.

TABLE 6
Promoter sequences

| | SEQ | |
|---|---|---|
| Promoter | ID | Nucleic Acid Sequence |
| SPc5-12 | 39 | GGCCGTCCGCCCTCGGCACCATCCTCACGACACCCAAATATG |
| | | GCGACGGGTGAGGAATGGTGGGGAGTTATTTTTAGAGCGGTG |
| | | AGGAAGGTGGGCAGGCAGCAGGTGTTGGCGCTCTAAAAATAA |
| | | CTCCCGGGAGTTATTTTTAGAGCGGAGGAATGGTGGACACCC |
| | | AAATATGGCGACGGTTCCTCACCCGTCGCCATATTTGGGTGT |
| | | CCGCCCTCGGCCGGGGCCGCATTCCTGGGGGCCGGGCGGTGC |
| | | TCCCGCCCGCCTCGATAAAAGGCTCCGGGGCCGGCGGCGGCC |
| | | CACGAGCTACCCGGAGGAGCGGGAGGCGCCAAGC |
| minSPc5- | 40 | GAATGGTGGACACCCAAATATGGCGACGGTTCCTCACCCGTC |
| 12 | | GCCATATTTGGGTGTCCGCCCTCGGCCGGGGCCGCATTCCTG |
| | | GGGGCCGGGCGGTGCTCCCGCCCGCCTCGATAAAAGGCTCCG |
| | | GGGCCGGCGGCGGCCCACGAGCTACCCGGAGGAGCGGGAGGC |
| | | GCCAAG |
| CK8 | 51 | ccactacgggtttaggctgcccatgtaaggaggcaaggcctg |
| | | gggacacccgagatgcctggttataattaacccagacatgtg |
| | | gctgccccccccccccccaacacctgctgcctctaaaaataa |
| | | ccctgtccctggtggatcccactacgggtttaggctgcccat |
| | | gtaaggaggcaaggcctggggacacccgagatgcctggttat |
| | | aattaacccagacatgtggctgccccccccccccccaacacc |
| | | tgctgcctctaaaaataaccctgtccctggtggatcccacta |
| | | cgggtttaggctgcccatgtaaggaggcaaggcctggggaca |
| | | cccgagatgcctggttataattaacccagacatgtggctgcc |
| | | ccccccccccccaacacctgctgcctctaaaaataaccctgt |
| | | ccctggtggatcccctgcatgcgaagatcttcgaacaaggct |
| | | gtgggggactgagggcaggctgtaacaggcttgggggccagg |
| | | gcttatacgtgcctgggactcccaaagtattactgttccatg |
| | | ttcccggcgaagggccagctgtcccccgccagctagactcag |
| | | cacttagtttaggaaccagtgagcaagtcagcccttggggca |
| | | gcccatacaaggccatggggctgggcaagctgcacgcctggg |
| | | tccggggtgggcacggtgcccgggcaacgagctgaaagctca |
| | | tctgctctcaggggcccctccctggggacagcccctcctggc |
| | | tagtcacaccctgtaggctcctctatataacccaggggcaca |
| | | ggggctgccctcattctaccaccacctccacagcacagacag |
| | | acactcaggagccagccagcgtcga |
| CAG | 52 | gacattgattattgactagttattaatagtaatcaattacgg |
| | | ggtcattagttcatagcccatatatggagttccgcgttacat |
| | | aacttacggtaaatggcccgcctggctgaccgcccaacgacc |
| | | cccgcccattgacgtcaataatgacgtatgttcccatagtaa |
| | | cgccaatagggactttccattgacgtcaatgggtggagtatt |
| | | tacggtaaactgcccacttggcagtacatcaagtgtatcata |
| | | tgccaagtacgccccctattgacgtcaatgacggtaaatggc |
| | | ccgcctggcattatgcccagtacatgaccttatgggactttc |
| | | ctacttggcagtacatctacgtattagtcatcgctattacca |
| | | tggtcgaggtgagccccacgttctgcttcactctccccatct |
| | | cccccccctccccacccccaattttgtatttatttatttttt |
| | | aattattttgtgcagcgatgggggcggggggggggggggggc |
| | | gcgcgccaggcggggcggggcggggcgaggggcggggcgggg |
| | | cgaggcggagaggtgcggcggcagccaatcagagcggcgcgc |
| | | tccgaaagtttccttttatggcgaggcggcggcggcggcggc |
| | | cctataaaaagcgaagcgcgcggcgggcgggagtcgctgcgc |
| | | gctgccttcgccccgtgccccgctccgccgccgcctcgcgcc |
| | | gcccgccccggctctgactgaccgcgttactcccacaggtga |
| | | gcgggcgggacggcccttctcctccgggctgtaattagcgct |
| | | tggtttaatgacggcttgtttcttttctgtggctgcgtgaaa |
| | | gccttgaggggctccgggagggccctttgtgcggggggagcg |
| | | gctcggggggtgcgtgcgtgtgtgtgtgcgtggggagcgccg |
| | | cgtgcggctccgcgctgcccggcggctgtgagcgctgcgggc |
| | | gcggcgcggggctttgtgcgctccgcagtgtgcgcgagggga |
| | | gcgcggccgggggcggtgccccgcggtgcggggggggctgcg |
| | | aggggaacaaaggctgcgtgcggggtgtgtgcgtgggggggt |
| | | gagcagggggtgtgggcgcgtcggtcgggctgcaaccccccc |
| | | tgcacccccctccccgagttgctgagcacggcccggcttcgg |
| | | gtgcggggctccgtacggggcgtggcgcggggctcgccgtgc |
| | | cgggcggggggtggcggcaggtgggggtgccgggcggggcgg |
| | | ggccgcctcgggccggggagggctcgggggaggggcgcggcg |
| | | gcccccggagcgccggcggctgtcgaggcgcggcgagccgca |
| | | gccattgccttttatggtaatcgtgcgagagggcgcagggac |
| | | ttcctttgtcccaaatctgtgcggagccgaaatctgggaggc |
| | | gccgccgcaccccctctagcgggcgcggggcgaagcggtgcg |
| | | gcgccggcaggaaggaaatgggcggggagggccttcgtgcgt |
| | | cgccgcgccgccgtccccttctccctctccagcctcggggct |
| | | gtccgcggggggacggctgccttcgggggggacggggcaggg |
| | | cggggttcggcttctggcgtgtgaccggcggctctagagcct |
| | | ctgctaaccatgttcatgccttcttctttttcctacagctcc |
| | | tgggcaacgtgctggttattgtgctgtctcatcattttggca |
| | | aag |
| mU1a | 56 | atggaggcggtactatgtagatgagaattcaggagcaaactg |
| | | ggaaaagcaactgcttccaaatatttgtgatttttacagtgt |
| | | agttttggaaaaactcttagcctaccaattcttctaagtgtt |
| | | ttaaaatgtgggagccagtacacatgaagttatagagtgttt |
| | | taatgaggcttaaatatttaccgtaactatgaaatgctacgc |
| | | atatcatgctgttcaggctccgtggccacgcaactcatact |
| EF-1a | 54 | gggcagagcgcacatcgcccacagtccccgagaagttggggg |
| | | gaggggtcggcaattgaacgggtgcctagagaaggtggcgcg |
| | | gggtaaactgggaaagtgatgtcgtgtactggctccgccttt |
| | | ttcccgagggtgggggagaaccgtatataagtgcagtagtcg |
| | | ccgtgaacgttctttttcgcaacgggtttgccgccagaacac |
| | | ag |
| Human | 89 | ctgcagacatgcttgctgcctgccctggcgtgccctggcgaggc |
| desmin | | ttgccgtcacaggacccccgctggctgactcaggggcgcaggct |
| | | cttgcgggggagctggcctcccgcccccacggccacgggccctt |
| | | tcctggcaggacagcgggatcttgcagctgtcaggggaggggat |
| | | gacgggggactgatgtcaggaggggatacaaatagtgccgaaca |
| | | aggaccggattagatctacc |
| MHCK7 | 94 | aagcttgcat gtctaagcta gacccttcag |
| | | attaaaaata actgaggtaa gggcctgggt |
| | | aggggaggtg gtgtgagacg ctcctgtctc |
| | | tcctctatct gcccatcggc cctttgggga |
| | | ggaggaatgt gcccaaggac taaaaaaagg |
| | | ccatggagcc agaggggcga gggcaacaga |
| | | cctttcatgg gcaaaccttg gggccctgct |
| | | gtctagcatg ccccactacg ggtctaggct |
| | | gcccatgtaa ggaggcaagg cctggggaca |
| | | cccgagatgc ctggttataa ttaacccaga |
| | | catgtggctg cccccccccc cccaacacct |
| | | gctgcctcta aaaataaccc tgtccctggt |
| | | ggatcccctg catgcgaaga tcttcgaaca |
| | | aggctgtggg ggactgaggg caggctgtaa |
| | | caggcttggg ggccagggct tatacgtgcc |
| | | tgggactccc aaagtattac tgttccatgt |
| | | tcccggcgaa gggccagctg tcccccgcca |
| | | gctagactca gcacttagtt taggaaccag |
| | | tgagcaagtc agcccttggg gcagcccata |
| | | caaggccatg gggctgggca agctgcacgc |
| | | ctgggtccgg ggtgggcacg gtgcccgggc |
| | | aacgagctga aagctcatct gctctcaggg |
| | | gcccctccct ggggacagcc cctcctggct |
| | | agtcacaccc tgtaggetcc tctatataac |
| | | ccaggggcac aggggctgcc ctcattctac |
| | | caccacctcc acagcacaga cagacactca |
| | | ggagcagcca gc |
| Truncated | 86 | ccactacggg tctaggctgc ccatgtaagg |
| MCK | | aggcaaggcc tggggacacc cgagatgcct |
| | | ggttataatt aaccccaaca cctgctgccc |
| | | cccccccccc aacacctgct gcctgagcct |
| | | gagcggttac cccaccccgg tgcctgggtc |
| | | ttaggctctg tacaccatgg aggagaagct |
| | | cgctctaaaa ataaccctgt ccctggtgga |
| | | tccactacgg gtctatgctg cccatgtaag |
| | | gaggcaaggc ctggggacac ccgagatgcc |
| | | tggttataat taaccccaac acctgctgcc |
| | | cccccccccc caacacctgc tgcctgagcc |
| | | tgagcggtta ccccaccccg gtgcctgggt |
| | | cttaggctct gtacaccatg gaggagaagc |
| | | tcgctctaaa aataaccctg tccctggtgg |
| | | accactacgg gtctaggctg cccatgtaag |
| | | gaggcaaggc ctggggacac ccgagatgcc |
| | | tggttataat taaccccaac acctgctgcc |
| | | cccccccccc aacacctgct gcctgagcct |
| | | gagcggttac cccaccccgg tgcctgggtc |
| | | ttaggctctg tacaccatgg aggagaagct |
| | | cgctctaaaa ataaccctgt ccctggtcct |
| | | ccctggggac agcccctect ggctagtcac |
| | | accctgtagg ctcctctata taacccaggg |
| | | gcacaggggc tgcccccggg tcac |
| Spc | 92 | AGAGGCCGTCCGCCCTCGGCACCATCCTCACGACACCCAAAT |
| version 1 | | ATGGCGACGGGTGAGGAATGGTGGGGAGTTATTTTTAGAGCG |
| ((Spc5v1) | | GTGAGGAAGGTGGGCAGGCAGCAGGTGTTGGCGCTCCATATT |
| mutant of | | TGGCGGGAGTTATTTTTAGAGCGGAGGAATGGTGGACACCCA |
| Spc5-12) | | AATATGGCGACGGTTCCTCACCCGTCGCTAAAAATAACTCCG |
| | | TGTCCGCCCTCGGCCGGGGCCGCATTCCTGGGGGCCGGGCGG |
| | | TGCTCCCGCCCGCCTCGATAAAAGGCTCCGGGGCCGGCGGCG |
| | | GCCCACGAGCTACCCGGAGGAGCGGGAGGCGCCAAGCGGAA |
| Spc | 93 | GGCCGTCCGCCCTCGGCACCATCCTCACGACACCCAAATATG |
| version 2 | | GCGACGGGTGAGGAATGGTGGGGAGTTATTTTTAGAGCGGTG |
| ((Spc5v2) | | AGGAAGGTGGGCAGGCAGCAGGTGTTGGGGGAGTTATTTTTA |
| mutant of | | GAGCGGGGAGTTATTTTTAGAGCGGAGGAATGGTGGACACCC |
| Spc5-12) | | AAATATGGCGACGGTTCCTCACGGACACCCAAATATGGCGAC |
| | | GGGCCCTCGGCCGGGGCCGCATTCCTGGGGGCCGGGCGGTGC |
| | | TCCCGCCCGCCTCGATAAAAGGCTCCGGGGCCGGCGGCGGCC |
| | | CACGAGCTACCCGGAGGAGCGGGAGGCGCCAAGC |

In certain embodiments, the promoter is a CNS-specific promoter. For example, an expression cassette can comprise a promoter selected from a promoter isolated from the genes of neuron specific enolase (NSE), any neuronal promoter such as the promoter of Dopamine-1 receptor or Dopamine-2 receptor, the synapsin promoter, CB7 promoter (a chicken R-actin promoter and CMV enhancer), RSV promoter, GFAP promoter (glial fibrillary acidic protein), MBP promoter (myelin basic protein), MMT promoter, EF-1α, U86 promoter, RPE65 promoter or opsin promoter, an inducible promoter, for example, a hypoxia-inducible promoter, and a drug inducible promoter, such as a promoter induced by rapamycin and related agents.

In still other embodiments, expression cassettes can comprise multiple promoters which may be placed in tandem in the expression cassette comprising a microdystrophin transgene. As such, tandem or hybrid promoters may be employed in order to enhance expression and/or direct expression to multiple tissue types, (see, e.g. PCT International Publication No. WO2019154939A1, published Aug. 15, 2019, incorporated herein by reference) and, in particular, LMTP6, LMTP13, LMTP14, LMTP15, LMTP18, LMTP19, or LMTP20 as disclosed in PCT International Application No. PCT/US2020/043578, filed Jul. 24, 2020, hereby incorporated by reference).

### 5.3.2 Introns

Certain gene expression cassettes further include an intron, for example, 5′ of the microdystrophin coding sequence which may enhances proper splicing and, thus, microdystrophin expression. Accordingly, in some embodiments, an intron is coupled to the 5′ end of a sequence encoding a microdystrophin protein. In particular, the intron nucleotide sequence can be linked to the nucleotide sequence attached to the actin-binding domain. In other embodiments, the intron is less than 100 nucleotides in length.

In embodiments, the intron is a VH4 intron. The VH4 intron nucleic acid can comprise SEQ ID NO: 41 as shown in Table 7 below.

TABLE 7
Nucleotide sequences for different introns

| | SEQ | |
|---|---|---|
| Structure | ID | Sequence |
| VH4 | 41 | GTGAGTATCTCAGGGATCCAGACATGGGGATATGGGAGGTGCCTCT |
| intron | | GATC |
| | | CCAGGGCTCACTGTGGGTCTCTCTGTTCACAG |
| Chimeric | 75 | GTAAGTATCAAGGTTACAAGACAGGTTTAAGGAGACCAATAGAAAC |
| intron | | TGGGCTTGTCGAGACAGAGAAGACTCTTGCGTTTCTGATAGGCACC |
| | | TATTGGTCTTACTGACATCCACTTTGCCTTTCTCTCCACAG |
| SV40 | 76 | GTAAGTTTAGTCTTTTTGTCTTTTATTTCAGGTCCCGGATCCGGTG |
| intron | | GTGGTGCAAATCAAAGAACTGCTCCTCAGTGGATGTTGCCTTTACT |
| | | TCTAG |

In other embodiments, the intron is a chimeric intron derived from human β-globin and Ig heavy chain (also known as β-globin splice donor/immunoglobulin heavy chain splice acceptor intron, or β-globin/IgG chimeric intron) (Table 7, SEQ ID NO: 75). Other introns well known to the skilled person may be employed, such as the chicken R-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron (Table 7, SEQ ID NO: 76).

### 5.3.3 Other Regulatory Elements

### 5.3.3.1 polyA

Another aspect of the present disclosure relates to expression cassettes comprising a polyadenylation (polyA) site downstream of the coding region of the microdystrophin transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene, the bovine growth hormone (BPH) gene, the human growth hormone (hGH) gene, and the synthetic polyA (SPA) site. In one embodiment, the polyA signal comprises SEQ ID NO: 42 as shown in Table 8.

TABLE 8
Nucleotide sequence of the polyA signal

| | SEQ | |
|---|---|---|
| Structure | ID | Sequence |
| polyA | 42 | AGGCCTAATAAAGAGCTCAGATGCATCGATCAGAGTGTGTTGGT |
| | | TTTTTG |

### 5.3.4 Viral Vectors

The microdystrophin transgene in accordance with the present disclosure can be included in an AAV vector for gene therapy administration to a human subject. In some embodiments, recombinant AAV (rAAV) vectors can comprise an AAV viral capsid and a viral or artificial genome comprising an expression cassette flanked by AAV inverted terminal repeats (ITRs) wherein the expression cassette comprises a microdystrophin transgene, operably linked to one or more regulatory sequences that control expression of the transgene in human muscle or CNS cells to express and deliver the microdystrophin. The provided methods are suitable for use in the production of any isolated recombinant AAV particles for delivery of a microdystrophins described herein, in the production of a composition comprising any isolated recombinant AAV particles encoding a microdystrophin, or in the method for treating a disease or disorder amenable for treatment with a microdystrophin in a subject in need thereof comprising the administration of any isolated recombinant AAV particles encoding a microdystrophin described herein. As such, the rAAV can be of any serotype, variant, modification, hybrid, or derivative thereof, known in the art, or any combination thereof (collectively referred to as “serotype”). In particular embodiments, the AAV serotype has a tropism for muscle tissue. And, in other embodiments, the AAV serotype has a tropism for the liver, in which case the liver cells transduced with the AAV form a depot of microdystrophin secreting cells, secretin the microdystrophin into the circulation.

In some embodiments, rAAV particles have a capsid protein from an AAV8 or AAV9 serotype. Provided herein are the RGX-DYS1 construct in an rAAV particle having an AAV8 capsid and the RGX-DYS1 construct in an rAAV particle having an AAV9 capsid. Also provided are the RGX-DYS5 construct in an rAAV particle having an AAV8 capsid and the RGX-DYS5 construct in an rAAV particle having an AAV9 capsid. In some embodiments, the rAAV particles comprise a capsid protein from an AAV capsid serotype selected from the group consisting of AAV7, AAV.rh8, AAV.rh10, AAV.rh20, AAV.rh39, AAV.Rh74, AAV.RHM4-1, AAV.hu31, AAV.hu32, AAV.hu37, AAV.PHP.B, AAV.PHP.eB, and AAV.7m8. In some embodiments, the rAAV particles comprise a capsid protein with high sequence homology to AAV8 or AAV9 such as, AAV.rh10, AAV.rh20, AAV.rh39, AAV.Rh74, AAV.RHM4-1, AAV.hu31, AAV.hu32, and AAV.hu37. In some embodiments, the rAAV particles have an AAV capsid serotype of AAV1 or a derivative, modification, or pseudotype thereof. In some embodiments, the rAAV particles have an AAV capsid serotype of AAV4 or a derivative, modification, or pseudotype thereof. In some embodiments, the rAAV particles have an AAV capsid serotype of AAV5 or a derivative, modification, or pseudotype thereof. In some embodiments, the rAAV particles have an AAV capsid serotype of AAV8 or a derivative, modification, or pseudotype thereof. In some embodiments, the rAAV particles have an AAV capsid serotype of AAV9 or a derivative, modification, or pseudotype thereof.

In some embodiments, rAAV particles comprise a capsid protein that is a derivative, modification, or pseudotype of AAV8 or AAV9 capsid protein. In some embodiments, rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein. In some embodiments, rAAV particles comprise a capsid protein that is a derivative, modification, or pseudotype of AAV9 capsid protein. In some embodiments, rAAV particles comprise a capsid protein that has an AAV8 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein.

In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of AAV7, AAV8, AAV9, AAV.rh8, AAV.rh10, AAV.rh20, AAV.rh39, AAV.Rh74, AAV.RHM4-1, AAV.hu31, AAV.hu32, AAV.hu37, AAV.PHP.B, AAV.PHP.eB, or AAV.7m8 capsid protein. In some embodiments, the rAAV particles comprise a capsid protein that has at least 80% or more identity, e.g., 85%, 85%, 87%, 88%, 89%, 90%, 91%, 92%, 93%, 94%, 95%, 96%, 97%, 98%, 99%, 99.5%, etc., i.e. up to 100% identity, to the VP1, VP2 and/or VP3 sequence of an AAV capsid protein with high sequence homology to AAV8 or AAV9 such as, AAV.rh10, AAV.rh20, AAV.rh39, AAV.Rh74, AAV.RHM4-1, AAV.hu31, AAV.hu32, and AAV.hu37.

Alternatively, the rAAV particles have a capsid protein of a serotype selected from AAV1, AAV2, AAV3, AAV4, AAV5, AAV6, AAV7, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

For example, a population of rAAV particles can comprise two or more serotypes, e.g., comprising two or more of AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.)

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP (SEQ ID NO: 87) or LALGETTRP (SEQ ID NO: 88), as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAVPHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689, (see, e.g., SEQ ID NOs: 5-38 of '689) WO2009/104964 (see, e.g., SEQ ID NOs: 1-5, 7, 9, 20, 22, 24 and 31 of '964), WO 2010/127097 (see, e.g., SEQ ID NOs: 5-38 of '097), and WO 2015/191508 (see, e.g., SEQ ID NOs: 80-294 of '508), and U.S. Appl. Publ. No. 20150023924 (see, e.g., SEQ ID NOs: 1, 5-10 of '924), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051), WO 2005/033321 (see, e.g., SEQ ID NOs: 123 and 88 of '321), WO 03/042397 (see, e.g., SEQ ID NOs: 2, 81, 85, and 97 of '397), WO 2006/068888 (see, e.g., SEQ ID NOs: 1 and 3-6 of '888), WO 2006/110689 (see, e.g., SEQ ID NOs: 5-38 of '689) WO2009/104964 (see, e.g., SEQ ID NOs: 1-5, 7, 9, 20, 22, 24 and 31 of 964), WO 2010/127097 (see, e.g., SEQ ID NOs: 5-38 of '097), and WO 2015/191508 (see, e.g., SEQ ID NOs: 80-294 of '508), and U.S. Appl. Publ. No. 20150023924 (see, e.g., SEQ ID NOs: 1, 5-10 of '924).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In additional embodiments, rAAV particles comprise a mosaic capsid. Mosaic AAV particles are composed of a mixture of viral capsid proteins from different serotypes of AAV. In some embodiments, rAAV particles comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles comprise a mosaic capsid containing capsid proteins of a serotype selected from AAV1, AAV2, AAV5, AAV6, AAV7, AAV8, AAV9, AAV10, AAVrh.8, and AAVrh.10.

In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV3, AAV4, AAV5, AAV6, AAV7, AAV8, AAV9, AAV10 AAV11, AAV12, AAV13, AAV14, AAV15 and AAV16). In additional embodiments, rAAV particles comprise a pseudotyped rAAV particle comprised of a capsid protein of an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu31, AAV.hu32, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In additional embodiments, rAAV particles comprise a pseudotyped rAAV particle containing AAV8 capsid protein. In additional embodiments, rAAV particles comprise a pseudotyped rAAV particle is comprised of AAV9 capsid protein. In some embodiments, the pseudotyped rAAV8 or rAAV9 particles are rAAV2/8 or rAAV2/9 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In further embodiments, the capsid protein is a chimeric of 2 or more AAV capsid proteins from AAV serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV8 capsid protein and one or more AAV capsid proteins from an AAV serotype selected from AAV1, AAV2, AAV5, AAV6, AAV7, AAV9, AAV10, AAVrh.8, and AAVrh.10.

In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV9 capsid protein the capsid protein of one or more AAV capsid serotypes selected from AAV1, AAV2, AAV3, AAV4, AAV5, AA6, AAV7, AAV8, AAV9, AAVrh.8, and AAVrh.10.

In some embodiments the rAAV particles comprises a Clade A, B, E, or F AAV capsid protein. In some embodiments, the rAAV particles comprises a Clade F AAV capsid protein. In some embodiments the rAAV particles comprises a Clade E AAV capsid protein.

Table 9 below provides examples of amino acid sequences for an AAV8, AAV9, AAV.rh74, AAV.hu31, AAV.hu32, and AAV.hu37 capsid proteins and the nucleic acid sequence of AAV2 5′- and 3′ ITRs.

TABLE 9

| | SEQ | |
|---|---|---|
| Structure | ID | Sequence |
| 5′-ITR | 73 | cgcgcgctcgctcgctcactgaggccgcccgggcaaagcccgggcgt |
| | | cgggcgacctttggtcgcccggcctcagtgagcgagcgagcgcgcag |
| | | agagggagtggccaactccatcactaggggttcct |
| | | Rep protein binding site (rps) is underlined. |
| 3′-ITR | 74 | aggaacccctagtgatggagttggccactccctctctgcgcgctcgc |
| | | tcgctcactgaggccgggcgaccaaaggtcgcccgacgcccgggctt |
| | | tgcccgggcggcctcagtgagcgagcgagcgcgcag |
| | | Rep protein binding site (rps) is underlined. |
| AAV8 | 77 | MAADGYLPDW LEDNLSEGIR EWWALKPGAP KPKANQQKQD |
| Capsid | | DGRGLVLPGY KYLGPFNGLD KGEPVNAADA AALEHDKAYD |
| | | QQLQAGDNPY LRYNHADAEF QERLQEDTSF GGNLGRAVFQ |
| | | AKKRVLEPLG LVEEGAKTAP GKKRPVEPSP QRSPDSSTGI |
| | | GKKGQQPARK RLNFGQTGDS ESVPDPQPLG EPPAAPSGVG |
| | | PNTMAAGGGA PMADNNEGAD GVGSSSGNWH CDSTWLGDRV |
| | | ITTSTRTWAL PTYNNHLYKQ ISNGTSGGAT NDNTYFGYST |
| | | PWGYFDFNRF HCHFSPRDWQ RLINNNWGFR PKRLSFKLFN |
| | | IQVKEVTQNE GTKTIANNLT STIQVFTDSE YQLPYVLGSA |
| | | HQGCLPPFPA DVFMIPQYGY LTLNNGSQAV GRSSFYCLEY |
| | | FPSQMLRTGN NFQFTYTFED VPFHSSYAHS QSLDRLMNPL |
| | | IDQYLYYLSR TQTTGGTANT QTLGFSQGGP NTMANQAKNW |
| | | LPGPCYRQQR VSTTTGQNNN SNFAWTAGTK YHLNGRNSLA |
| | | NPGIAMATHK DDEERFFPSN GILIFGKQNA ARDNADYSDV |
| | | MLTSEEEIKT TNPVATEEYG IVADNLQQQN TAPQIGTVNS |
| | | QGALPGMVWQ NRDVYLQGPI WAKIPHTDGN FHPSPLMGGF |
| | | GLKHPPPQIL IKNTPVPADP PTTFNQSKLN SFITQYSTGQ |
| | | VSVEIEWELQ KENSKRWNPE IQYTSNYYKS TSVDFAVNTE |
| | | GVYSEPRPIG TRYLTRNL |
| AAV9 | 78 | MAADGYLPDW LEDNLSEGIR EWWALKPGAP QPKANQQHQD |
| Capsid | | NARGLVLPGY KYLGPGNGLD KGEPVNAADA AALEHDKAYD |
| | | QQLKAGDNPY LKYNHADAEF QERLKEDTSF GGNLGRAVFQ |
| | | AKKRLLEPLG LVEEAAKTAP GKKRPVEQSP QEPDSSAGIG |
| | | KSGAQPAKKR LNFGQTGDTE SVPDPQPIGE PPAAPSGVGS |
| | | LTMASGGGAP VADNNEGADG VGSSSGNWHC DSQWLGDRVI |
| | | TTSTRTWALP TYNNHLYKQI SNSTSGGSSN DNAYFGYSTP |
| | | WGYFDFNRFH CHFSPRDWQR LINNNWGFRP KRLNFKLFNI |
| | | QVKEVTDNNG VKTIANNLTS TVQVFTDSDY QLPYVLGSAH |
| | | EGCLPPFPAD VFMIPQYGYL TLNDGSQAVG RSSFYCLEYF |
| | | PSQMLRTGNN FQFSYEFENV PFHSSYAHSQ SLDRLMNPLI |
| | | DQYLYYLSKT INGSGQNQQT LKFSVAGPSN MAVQGRNYIP |
| | | GPSYRQQRVS TTVTQNNNSE FAWPGASSWA LNGRNSLMNP |
| | | GPAMASHKEG EDRFFPLSGS LIFGKQGTGR DNVDADKVMI |
| | | TNEEEIKTTN PVATESYGQV ATNHQSAQAQ AQTGWVQNQG |
| | | ILPGMVWQDR DVYLQGPIWA KIPHTDGNFH PSPLMGGFGM |
| | | KHPPPQILIK NTPVPADPPT AFNKDKLNSF ITQYSTGQVS |
| | | VEIEWELQKE NSKRWNPEIQ YTSNYYKSNN VEFAVNTEGV |
| | | YSEPRPIGTR YLTRNL |
| hu.37 | 48 | MAADGYLPDW LEDNLSEGIR EWWDLKPGAP KPKANQQKQD |
| Capsid | | DGRGLVLPGY KYLGPFNGLD KGEPVNAADA AALEHDKAYD |
| | | QQLKAGDNPY LRYNHADAEF QERLQEDTSF GGNLGRAVFQ |
| | | AKKRVLEPLG LVEEAAKTAP GKKRPVEPSP QRSPDSSTGI |
| | | GKKGQQPAKK RLNFGQTGDS ESVPDPQPIG EPPAGPSGLG |
| | | SGTMAAGGGA PMADNNEGAD GVGSSSGNWH CDSTWLGDRV |
| | | ITTSTRTWAL PTYNNHLYKQ ISNGTSGGST NDNTYFGYST |
| | | PWGYFDFNRF HCHFSPRDWQ RLINNNWGFR PKRLSFKLFN |
| | | IQVKEVTQNE GTKTIANNLT STIQVFTDSE YQLPYVLGSA |
| | | HQGCLPPFPA DVFMIPQYGY LTLNNGSQAV GRSSFYCLEY |
| | | FPSQMLRTGN NFEFSYTFED VPFHSSYAHS QSLDRLMNPL |
| | | IDQYLYYLSR TQSTGGTQGT QQLLFSQAGP ANMSAQAKNW |
| | | LPGPCYRQQR VSTILSQNNN SNFAWTGATK YHLNGRDSLV |
| | | NPGVAMATHK DDEERFFPSS GVLMFGKQGA GRDNVDYSSV |
| | | MLTSEEEIKT TNPVATEQYG VVADNLQQTN TGPIVGNVNS |
| | | QGALPGMVWQ NRDVYLQGPI WAKIPHTDGN FHPSPLMGGF |
| | | GLKHPPPQIL IKNTPVPADP PTTFSQAKLA SFITQYSTGQ |
| | | VSVEIEWELQ KENSKRWNPE IQYTSNYYKS TNVDFAVNTE |
| | | GTYSEPRPIG TRYLTRNL |
| hu.31 | 49 | MAADGYLPDW LEDTLSEGIR QWWKLKPGPP PPKPAERHKD |
| Capsid | | DSRGLVLPGY KYLGPGNGLD KGEPVNAADA AALEHDKAYD |
| | | QQLKAGDNPY LKYNHADAEF QERLKEDTSF GGNLGRAVFQ |
| | | AKKRLLEPLG LVEEAAKTAP GKKRPVEQSP QEPDSSAGIG |
| | | KSGSQPAKKK LNFGQTGDTE SVPDPQPIGE PPAAPSGVGS |
| | | LTMASGGGAP VADNNEGADG VGSSSGNWHC DSQWLGDRVI |
| | | TTSTRTWALP TYNNHLYKQI SNSTSGGSSN DNAYFGYSTP |
| | | WGYFDFNRFH CHFSPRDWQR LINNNWGFRP KRLNFKLFNI |
| | | QVKEVTDNNG VKTIANNLTS TVQVFTDSDY QLPYVLGSAH |
| | | EGCLPPFPAD VFMIPQYGYL TLNDGGQAVG RSSFYCLEYF |
| | | PSQMLRTGNN FQFSYEFENV PFHSSYAHSQ SLDRLMNPLI |
| | | DQYLYYLSKT INGSGQNQQT LKFSVAGPSN MAVQGRNYIP |
| | | GPSYRQQRVS TTVTQNNNSE FAWPGASSWA LNGRNSLMNP |
| | | GPAMASHKEG EDRFFPLSGS LIFGKQGTGR DNVDADKVMI |
| | | TNEEEIKTTN PVATESYGQV ATNHQSAQAQ AQTGWVQNQG |
| | | ILPGMVWQDR DVYLQGPIWA KIPHTDGNFH PSPLMGGFGM |
| | | KHPPPQILIK NTPVPADPPT AFNKDKLNSF ITQYSTGQVS |
| | | VEIEWELQKE NSKRWNPEIQ YTSNYYKSNN VEFAVSTEGV |
| | | YSEPRPIGTR YLTRNL |
| hu.32 | 50 | MAADGYLPDW LEDTLSEGIR QWWKLKPGPP PPKPAERHKD |
| Capsid | | DSRGLVLPGY KYLGPGNGLD KGEPVNAADA AALEHDKAYD |
| | | QQLKAGDNPY LKYNHADAEF QERLKEDTSF GGNLGRAVFQ |
| | | AKKRLLEPLG LVEEAAKTAP GKKRPVEQSP QEPDSSAGIG |
| | | KSGSQPAKKK LNFGQTGDTE SVPDPGQPIG EPPAAPSGVG |
| | | SLTMASGGGA PVADNNEGAD GVGSSSGNWH CDSQWLGDRV |
| | | ITTSTRTWAL PTYNNHLYKQ ISNSTSGGSS NDNAYFGYST |
| | | PWGYFDFNRF HCHFSPRDWQ RLINNNWGFR PKRLNFKLFN |
| | | IQVKEVTDNN GVKTIANNLT STVQVFTDSD YQLPYVLGSA |
| | | HEGCLPPFPA DVFMIPQYGY LTLNDGSQAV GRSSFYCLEY |
| | | FPSQMLRTGN NFQFSYEFEN VPFHSSYAHS QSLDRLMNPL |
| | | IDQYLYYLSK TINGSGQNQQ TLKFSVAGPS NMAVQGRNYI |
| | | PGPSYRQQRV STTVTQNNNS EFAWPGASSW ALNGRNSLMN |
| | | PGPAMASHKE GEDRFFPLSG SLIFGKQGTG RDNVDADKVM |
| | | ITNEEEIKTT NPVATESYGQ VATNHQSAQA QAQTGWVQNQ |
| | | GILPGMVWQD RDVYLQGPIW AKIPHTDGNF HPSPLMGGFG |
| | | MKHPPPQILI KNTPVPADPP TAFNKDKLNS FITQYSTGQV |
| | | SVEIEWELQK ENSKRWNPEI QYTSNYYKSN NVEFAVNTEG |
| | | VYSEPRPIGT RYLTRNL |
| Rh.74 | 45 | MAADGYLPD WLEDNLSEG IREWWDLKP GAPKPKANQ |
| version 1 | | QKQDNGRGL VLPGYKYLG PFNGLDKGE PVNAADAAA |
| | | LEHDKAYDQ QLQAGDNPY LRYNHADAE FQERLQEDT |
| | | SFGGNLGRA VFQAKKRVL EPLGLVESP VKTAPGKKR |
| | | PVEPSPQRS PDSSTGIGK KGQQPAKKR LNFGQTGDS |
| | | ESVPDPQPI GEPPAGPSG LGSGTMAAG GGAPMADNN |
| | | EGADGVGSS SGNWHCDST WLGDRVITT STRTWALPT |
| | | YNNHLYKQI SNGTSGGST NDNTYFGYS TPWGYFDFN |
| | | RFHCHFSPR DWQRLINNN WGFRPKRLN FKLFNIQVK |
| | | EVTQNEGTK TIANNLIST IQVFTDSEY QLPYVLGSA |
| | | HQGCLPPFP ADVFMIPQY GYLTLNNGS QAVGRSSFY |
| | | CLEYFPSQM LRTGNNFEF SYNFEDVPF HSSYAHSQS |
| | | LDRLMNPLI DQYLYYLSR TQSTGGTAG TQQLLFSQA |
| | | GPNNMSAQA KNWLPGPCY RQQRVSTTL SQNNNSNFA |
| | | WTGATKYHL NGRDSLVNP GVAMATHKD DEERFFPSS |
| | | GVLMFGKQG AGKDNVDYS SVMLTSEEE IKTTNPVAT |
| | | EQYGVVADN LQQQNAAPI VGAVNSQGA LPGMVWQNR |
| | | DVYLQGPIW AKIPHTDGN FHPSPLMGG FGLKHPPPQ |
| | | ILIKNTPVP ADPPTTFNQ AKLASFITQ YSTGQVSVE |
| | | IEWELQKEN SKRWNPEIQ YTSNYYKST NVDFAVNTE |
| | | GTYSEPRPI GTRYLTRNL |
| Rh.74 | 85 | MAADGYLPD WLEDNLSEG IREWWDLKP GAPKPKANQ |
| version 2 | | QKQDNGRGL VLPGYKYLG PFNGLDKGE PVNAADAAA |
| | | LEHDKAYDQ QLQAGDNPY LRYNHADAE FQERLQEDT |
| | | SFGGNLGRA VFQAKKRVL EPLGLVESP VKTAPGKKR |
| | | PVEPSPQRS PDSSTGIGK KGQQPAKKR LNFGQTGDS |
| | | ESVPDPQPI GEPPAAPSG VGPNTMAAG GGAPMADNN |
| | | EGADGVGSS SGNWHCDST WLGDRVITT STRTWALPT |
| | | YNNHLYKQI SNGTSGGST NDNTYFGYS TPWGYFDFN |
| | | RFHCHFSPR DWQRLINNN WGFRPKRLN FKLFNIQVK |
| | | EVTQNEGTK TIANNLIST IQVFTDSEY QLPYVLGSA |
| | | HQGCLPPFP ADVFMIPQY GYLTLNNGS QAVGRSSFY |
| | | CLEYFPSQM LRTGNNFEF SYNFEDVPF HSSYAHSQS |
| | | LDRLMNPLI DQYLYYLSR TQSTGGTAG TQQLLFSQA |
| | | GPNNMSAQA KNWLPGPCY RQQRVSTTL SQNNNSNFA |
| | | WTGATKYHL NGRDSLVNP GVAMATHKD DEERFFPSS |
| | | GVLMFGKQG AGKDNVDYS SVMLTSEEE IKTTNPVAT |
| | | EQYGVVADN LQQQNAAPI VGAVNSQGA LPGMVWQNR |
| | | DVYLQGPIW AKIPHTDGN FHPSPLMGG FGLKHPPPQ |
| | | ILIKNTPVP ADPPTTFNQ AKLASFITQ YSTGQVSVE |
| | | IEWELQKEN SKRWNPEIQ YTSNYYKST NVDFAVNTE |
| | | GTYSEPRPI GTRYLTRNL |

The provided methods are suitable for use in the production of recombinant AAV encoding a transgene. In certain embodiments, the transgene is a microdystrophin as described herein. In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the described transgene. In a specific embodiment, the constructs described herein comprise the following components: (1) AAV2 or AAV8 inverted terminal repeats (ITRs) that flank the expression cassette; (2) control elements, which include a muscle-specific SPc5.12 promoter and a small poly A signal; and (3) transgene providing (e.g., coding for) a nucleic acid encoding microdystrophin as described herein, including the microdystrophin coding sequence of the RGX-DYS1 transgene (SEQ ID NO:20) or the RGX-DYS5 transgene (SEQ ID NO:81). In a specific embodiment, the constructs described herein comprise the following components: (1) AAV2 or AAV8 ITRs that flank the expression cassette; (2) control elements, which include a) the muscle-specific SPc5.12 promoter, b) a small poly A signal; and (3) microdystrophin cassette, which includes from the N-terminus to the C-terminus, ABD1-H1-R1-R2-R3-H3-R24-H4-CR-CT, wherein CT comprises at least the portion of the CT comprising an α1-syntrophin binding site, including the CT having an amino acid sequence of SEQ ID NO:16 or 83. In a specific embodiment, the constructs described herein comprise the following components: (1) AAV2 or AAV8 ITRs that flank the expression cassette; (2) control elements, which include a) the muscle-specific SPc5.12 promoter, b) an intron (e.g., VH4) and c) a small poly A signal; and (3) microdystrophin cassette, which includes from the N-terminus to the C-terminus ABD1-H1-R1-R2-R3-H3-R24-H4-CR-CT, wherein the CT comprises at least the portion of the CT comprising an α1-syntrophin binding site, including the CT having an amino acid sequence of SEQ ID NO:16 or 83, ABD1 being directly coupled to VH4.

In a specific embodiment, the constructs described herein comprise the following components: (1) AAV2 ITRs that flank the expression cassette; (2) control elements, which include the muscle-specific SPc5.12 promoter, and b) a small poly A signal; and (3) the nucleic acid encoding the RGX-DYS1 microdystrophin having an amino acid sequence of SEQ ID NO:1, including encoded by a nucleotide sequence of SEQ ID NO:20. In a specific embodiment, the constructs described herein comprise the following components: (1) AAV2 ITRs that flank the expression cassette; (2) control elements, which include the muscle-specific SPc5.12 promoter, and b) a small poly A signal; and (3) the nucleic acid encoding the RXG-DYS5 microdystrophin having an amino acid sequence of SEQ ID NO:79, including encoded by a nucleotide sequence of SEQ ID NO:81. In some embodiments, constructs described herein comprising AAV ITRs flanking a microdystrophin expression cassette, which includes from the N-terminus to the C-terminus ABD1-H1-R1-R2-R3-H2-R24-H4-CR-CT, wherein the CT comprises at least the portion of the CT comprising an α1-syntrophin binding site, including the CT having an amino acid sequence of SEQ ID NO:16 or 83, can be between 4000 nt and 5000 nt in length. In some embodiments, such constructs are less than 4900 nt, 4800 nt, 4700 nt, 4600 nt, 4500 nt, 4400 nt, or 4300 nt in length.

Some nucleic acid embodiments of the present disclosure comprise rAAV vectors encoding microdystrophin comprising or consisting of a nucleotide sequence of SEQ ID NO: 53, 55, or 82 provided in Table 10 below. In various embodiments, an rAAV vector comprising a nucleotide sequence that has at least 50%, at least 60%, at least 70%, at least 75%, at least 80%, at least 85%, at least 90%, at least 95%, at least 98% or at least 99% sequence identity to the nucleotide sequence of SEQ ID NO: 53, 55, or 82 or the reverse complement thereof and encodes a rAAV vector suitable for expression of a therapeutically effective microdystrophin in muscle cells. In embodiments, the constructs having the nucleotide sequence of SEQ ID NO: 53, 55 or 82 are in a recombinant rAAV8 or rAAV9 particle.

TABLE 10
RGX-DYS cassette nucleotide sequences

| | SEQ | |
|---|---|---|
| Structure | ID | Nucleic Acid Sequence |
| RGX-DYS1 | 53 | ctgcgcgctcgctcgctcactgaggccgcccgggcaaagcccgg |
| (full cassette | | gcgtcgggcgacctttggtcgcccggcctcagtgagcgagcgag |
| SPc5-12 to | | cgcgcagagagggagtggccaactccatcactaggggttcctCA |
| polyA | | TATGcagggtaatggggatcctCTAGAGGCCGTCCGCCCTCGGC |
| including | | ACCATCCTCACGACACCCAAATATGGCGACGGGTGAGGAATGGT |
| intervening | | GGGGAGTTATTTTTAGAGCGGTGAGGAAGGTGGGCAGGCAGCAG |
| seqs) | | GTGTTGGCGCTCTAAAAATAACTCCCGGGAGTTATTTTTAGAGC |
| 4734 bp | | GGAGGAATGGTGGACACCCAAATATGGCGACGGTTCCTCACCCG |
| ITRs shown in | | TCGCCATATTTGGGTGTCCGCCCTCGGCCGGGGCCGCATTCCTG |
| lower case | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGCGgAATTCGCCACCATGCTTTGGTGGGAAGAGGTGGAAGATT |
| | | GCTATGAGAGGGAAGATGTGCAGAAGAAAACCTTCACCAAATGG |
| | | GTCAATGCCCAGTTCAGCAAGTTTGGCAAGCAGCACATTGAGAA |
| | | CCTGTTCAGTGACCTGCAGGATGGCAGAAGGCTGCTGGATCTGC |
| | | TGGAAGGCCTGACAGGCCAGAAGCTGCCTAAAGAGAAGGGCAGC |
| | | ACAAGAGTGCATGCCCTGAACAATGTGAACAAGGCCCTGAGAGT |
| | | GCTGCAGAACAACAATGTGGACCTGGTCAATATTGGCAGCACAG |
| | | ACATTGTGGATGGCAACCACAAGCTGACCCTGGGCCTGATCTGG |
| | | AACATCATCCTGCACTGGCAAGTGAAGAATGTGATGAAGAACAT |
| | | CATGGCTGGCCTGCAGCAGACCAACTCTGAGAAGATCCTGCTGA |
| | | GCTGGGTCAGACAGAGCACCAGAAACTACCCTCAAGTGAATGTG |
| | | ATCAACTTCACCACCTCTTGGAGTGATGGACTGGCCCTGAATGC |
| | | CCTGATCCACAGCCACAGACCTGACCTGTTTGACTGGAACTCTG |
| | | TTGTGTGCCAGCAGTCTGCCACACAGAGACTGGAACATGCCTTC |
| | | AACATTGCCAGATACCAGCTGGGAATTGAGAAACTGCTGGACCC |
| | | TGAGGATGTGGACACCACCTATCCTGACAAGAAATCCATCCTCA |
| | | TGTACATCACCAGCCTGTTCCAGGTGCTGCCCCAGCAAGTGTCC |
| | | ATTGAGGCCATTCAAGAGGTTGAGATGCTGCCCAGACCTCCTAA |
| | | AGTGACCAAAGAGGAACACTTCCAGCTGCACCACCAGATGCACT |
| | | ACTCTCAGCAGATCACAGTGTCTCTGGCCCAGGGATATGAGAGA |
| | | ACAAGCAGCCCCAAGCCTAGGTTCAAGAGCTATGCCTACACACA |
| | | GGCTGCCTATGTGACCACATCTGACCCCACAAGAAGCCCATTTC |
| | | CAAGCCAGCATCTGGAAGCCCCTGAGGACAAGAGCTTTGGCAGC |
| | | AGCCTGATGGAATCTGAAGTGAACCTGGATAGATACCAGACAGC |
| | | CCTGGAAGAAGTGCTGTCCTGGCTGCTGTCTGCTGAGGATACAC |
| | | TGCAGGCTCAGGGTGAAATCAGCAATGATGTGGAAGTGGTCAAG |
| | | GACCAGTTTCACACCCATGAGGGCTACATGATGGACCTGACAGC |
| | | CCACCAGGGCAGAGTGGGAAATATCCTGCAGCTGGGCTCCAAGC |
| | | TGATTGGCACAGGCAAGCTGTCTGAGGATGAAGAGACAGAGGTG |
| | | CAAGAGCAGATGAACCTGCTGAACAGCAGATGGGAGTGTCTGAG |
| | | AGTGGCCAGCATGGAAAAGCAGAGCAACCTGCACAGAGTGCTCA |
| | | TGGACCTGCAGAATCAGAAACTGAAAGAACTGAATGACTGGCTG |
| | | ACCAAGACAGAAGAAAGGACTAGGAAGATGGAAGAGGAACCTCT |
| | | GGGACCAGACCTGGAAGATCTGAAAAGACAGGTGCAGCAGCATA |
| | | AGGTGCTGCAAGAGGACCTTGAGCAAGAGCAAGTCAGAGTGAAC |
| | | AGCCTGACACACATGGTGGTGGTTGTGGATGAGTCCTCTGGGGA |
| | | TCATGCCACAGCTGCTCTGGAAGAACAGCTGAAGGTGCTGGGAG |
| | | ACAGATGGGCCAACATCTGTAGGTGGACAGAGGATAGATGGGTG |
| | | CTGCTCCAGGACATTCTGCTGAAGTGGCAGAGACTGACAGAGGA |
| | | ACAGTGCCTGTTTTCTGCCTGGCTCTCTGAGAAAGAGGATGCTG |
| | | TCAACAAGATCCATACCACAGGCTTCAAGGATCAGAATGAGATG |
| | | CTCAGCTCCCTGCAGAAACTGGCTGTGCTGAAGGCTGACCTGGA |
| | | AAAGAAAAAGCAGTCCATGGGCAAGCTCTACAGCCTGAAGCAGG |
| | | ACCTGCTGTCTACCCTGAAGAACAAGTCTGTGACCCAGAAAACT |
| | | GAGGCCTGGCTGGACAACTTTGCTAGATGCTGGGACAACCTGGT |
| | | GCAGAAGCTGGAAAAGTCTACAGCCCAGATCAGCCAGCAACCTG |
| | | ATCTTGCCCCTGGCCTGACCACAATTGGAGCCTCTCCAACACAG |
| | | ACTGTGACCCTGGTTACCCAGCCAGTGGTCACCAAAGAGACAGC |
| | | CATCAGCAAACTGGAAATGCCCAGCTCTCTGATGCTGGAAGTCC |
| | | CCACACTGGAAAGGCTGCAAGAACTTCAAGAGGCCACAGATGAG |
| | | CTGGACCTGAAGCTGAGACAGGCTGAAGTGATCAAAGGCAGCTG |
| | | GCAGCCAGTTGGGGACCTGCTCATTGATAGCCTGCAGGACCATC |
| | | TGGAAAAAGTGAAAGCCCTGAGGGGAGAGATTGCCCCTCTGAAA |
| | | GAAAATGTGTCCCATGTGAATGACCTGGCCAGACAGCTGACCAC |
| | | ACTGGGAATCCAGCTGAGCCCCTACAACCTGAGCACCCTTGAGG |
| | | ACCTGAACACCAGGTGGAAGCTCCTCCAGGTGGCAGTGGAAGAT |
| | | AGAGTCAGGCAGCTGCATGAGGCCCACAGAGATTTTGGACCAGC |
| | | CAGCCAGCACTTTCTGTCTACCTCTGTGCAAGGCCCCTGGGAGA |
| | | GAGCTATCTCTCCTAACAAGGTGCCCTACTACATCAACCATGAG |
| | | ACACAGACCACCTGTTGGGATCACCCCAAGATGACAGAGCTGTA |
| | | CCAGAGTCTGGCAGACCTCAACAATGTCAGATTCAGTGCCTACA |
| | | GGACTGCCATGAAGCTCAGAAGGCTCCAGAAAGCTCTGTGCCTG |
| | | GACCTGCTTTCCCTGAGTGCAGCTTGTGATGCCCTGGACCAGCA |
| | | CAATCTGAAGCAGAATGACCAGCCTATGGACATCCTCCAGATCA |
| | | TCAACTGCCTCACCACCATCTATGATAGGCTGGAACAAGAGCAC |
| | | AACAATCTGGTCAATGTGCCCCTGTGTGTGGACATGTGCCTGAA |
| | | TTGGCTGCTGAATGTGTATGACACAGGCAGAACAGGCAGGATCA |
| | | GAGTCCTGTCCTTCAAGACAGGCATCATCTCCCTGTGCAAAGCC |
| | | CACTTGGAGGACAAGTACAGATACCTGTTCAAGCAAGTGGCCTC |
| | | CAGCACAGGCTTTTGTGACCAGAGAAGGCTGGGCCTGCTCCTGC |
| | | ATGACAGCATTCAGATCCCTAGACAGCTGGGAGAAGTGGCTTCC |
| | | TTTGGAGGCAGCAATATTGAGCCATCAGTCAGGTCCTGTTTTCA |
| | | GTTTGCCAACAACAAGCCTGAGATTGAGGCTGCCCTGTTCCTGG |
| | | ACTGGATGAGACTTGAGCCTCAGAGCATGGTCTGGCTGCCTGTG |
| | | CTTCATAGAGTGGCTGCTGCTGAGACTGCCAAGCACCAGGCCAA |
| | | GTGCAACATCTGCAAAGAGTGCCCCATCATTGGCTTCAGATACA |
| | | GATCCCTGAAGCACTTCAACTATGATATCTGCCAGAGCTGCTTC |
| | | TTTAGTGGCAGGGTTGCCAAGGGCCACAAAATGCACTACCCCAT |
| | | GGTGGAATACTGCACCCCAACAACCTCTGGGGAAGATGTTAGAG |
| | | ACTTTGCCAAGGTGCTGAAAAACAAGTTCAGGACCAAGAGATAC |
| | | TTTGCTAAGCACCCCAGAATGGGCTACCTGCCTGTCCAGACAGT |
| | | GCTTGAGGGTGACAACATGGAAACCCCTGTGACACTGATCAATT |
| | | TCTGGCCAGTGGACTCTGCCCCTGCCTCAAGTCCACAGCTGTCC |
| | | CATGATGACACCCACAGCAGAATTGAGCACTATGCCTCCAGACT |
| | | GGCAGAGATGGAAAACAGCAATGGCAGCTACCTGAATGATAGCA |
| | | TCAGCCCCAATGAGAGCATTGATGATGAGCATCTGCTGATCCAG |
| | | CACTACTGTCAGTCCCTGAACCAGGACTCTCCACTGAGCCAGCC |
| | | TAGAAGCCCTGCTCAGATCCTGATCAGCCTTGAGTCTGAGGAAA |
| | | GGGGAGAGCTGGAAAGAATCCTGGCAGATCTTGAGGAAGAGAAC |
| | | AGAAACCTGCAGGCAGAGTATGACAGGCTCAAACAGCAGCATGA |
| | | GCACAAGGGACTGAGCCCTCTGCCTTCTCCTCCTGAAATGATGC |
| | | CCACCTCTCCACAGTCTCCAAGGTGATGACTCGAGAGGCCTAAT |
| | | AAAGAGCTCAGATGCATCGATCAGAGTGTGTTGGTTTTTTGTGT |
| | | GCCAGGGTAATGGGCTAGCTGCGGCCGCaggaacccctagtgat |
| | | ggagttggccactccctctctgcgcgctcgctcgctcactgagg |
| | | ccgggcgaccaaaggtcgcccgacgcccgggctttgcccgggcg |
| | | gcctcagtgagcgagcgagcgcgcag |
| RGX-DYS3 | 55 | ctgcgcgctcgctcgctcactgaggccgcccgggcaaagcccgg |
| (full cassette | | gcgtcgggcgacctttggtcgcccggcctcagtgagcgagcgag |
| SPc5-12 to | | cgcgcagagagggagtggccaactccatcactaggggttcctCA |
| polyA | | TATGCAGGGTAATGGGGATCCTCTAGAGGCCGTCCGCCCTCGGC |
| including | | ACCATCCTCACGACACCCAAATATGGCGACGGGTGAGGAATGGT |
| intervening | | GGGGAGTTATTTTTAGAGCGGTGAGGAAGGTGGGCAGGCAGCAG |
| seqs) | | GTGTTGGCGCTCTAAAAATAACTCCCGGGAGTTATTTTTAGAGC |
| 4364 bp) | | GGAGGAATGGTGGACACCCAAATATGGCGACGGTTCCTCACCCG |
| ITRs shown in | | TCGCCATATTTGGGTGTCCGCCCTCGGCCGGGGCCGCATTCCTG |
| lower case | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGGTGAGTATCTCAGGGATCCAGACATGGGGATATGGGAGGTGC |
| | | CTCTGATCCCAGGGCTCACTGTGGGTCTCTCTGTTCACAGGAAT |
| | | TCGCCACCATGCTTTGGTGGGAAGAGGTGGAAGATTGCTATGAG |
| | | AGGGAAGATGTGCAGAAGAAAACCTTCACCAAATGGGTCAATGC |
| | | CCAGTTCAGCAAGTTTGGCAAGCAGCACATTGAGAACCTGTTCA |
| | | GTGACCTGCAGGATGGCAGAAGGCTGCTGGATCTGCTGGAAGGC |
| | | CTGACAGGCCAGAAGCTGCCTAAAGAGAAGGGCAGCACAAGAGT |
| | | GCATGCCCTGAACAATGTGAACAAGGCCCTGAGAGTGCTGCAGA |
| | | ACAACAATGTGGACCTGGTCAATATTGGCAGCACAGACATTGTG |
| | | GATGGCAACCACAAGCTGACCCTGGGCCTGATCTGGAACATCAT |
| | | CCTGCACTGGCAAGTGAAGAATGTGATGAAGAACATCATGGCTG |
| | | GCCTGCAGCAGACCAACTCTGAGAAGATCCTGCTGAGCTGGGTC |
| | | AGACAGAGCACCAGAAACTACCCTCAAGTGAATGTGATCAACTT |
| | | CACCACCTCTTGGAGTGATGGACTGGCCCTGAATGCCCTGATCC |
| | | ACAGCCACAGACCTGACCTGTTTGACTGGAACTCTGTTGTGTGC |
| | | CAGCAGTCTGCCACACAGAGACTGGAACATGCCTTCAACATTGC |
| | | CAGATACCAGCTGGGAATTGAGAAACTGCTGGACCCTGAGGATG |
| | | TGGACACCACCTATCCTGACAAGAAATCCATCCTCATGTACATC |
| | | ACCAGCCTGTTCCAGGTGCTGCCCCAGCAAGTGTCCATTGAGGC |
| | | CATTCAAGAGGTTGAGATGCTGCCCAGACCTCCTAAAGTGACCA |
| | | AAGAGGAACACTTCCAGCTGCACCACCAGATGCACTACTCTCAG |
| | | CAGATCACAGTGTCTCTGGCCCAGGGATATGAGAGAACAAGCAG |
| | | CCCCAAGCCTAGGTTCAAGAGCTATGCCTACACACAGGCTGCCT |
| | | ATGTGACCACATCTGACCCCACAAGAAGCCCATTTCCAAGCCAG |
| | | CATCTGGAAGCCCCTGAGGACAAGAGCTTTGGCAGCAGCCTGAT |
| | | GGAATCTGAAGTGAACCTGGATAGATACCAGACAGCCCTGGAAG |
| | | AAGTGCTGTCCTGGCTGCTGTCTGCTGAGGATACACTGCAGGCT |
| | | CAGGGTGAAATCAGCAATGATGTGGAAGTGGTCAAGGACCAGTT |
| | | TCACACCCATGAGGGCTACATGATGGACCTGACAGCCCACCAGG |
| | | GCAGAGTGGGAAATATCCTGCAGCTGGGCTCCAAGCTGATTGGC |
| | | ACAGGCAAGCTGTCTGAGGATGAAGAGACAGAGGTGCAAGAGCA |
| | | GATGAACCTGCTGAACAGCAGATGGGAGTGTCTGAGAGTGGCCA |
| | | GCATGGAAAAGCAGAGCAACCTGCACAGAGTGCTCATGGACCTG |
| | | CAGAATCAGAAACTGAAAGAACTGAATGACTGGCTGACCAAGAC |
| | | AGAAGAAAGGACTAGGAAGATGGAAGAGGAACCTCTGGGACCAG |
| | | ACCTGGAAGATCTGAAAAGACAGGTGCAGCAGCATAAGGTGCTG |
| | | CAAGAGGACCTTGAGCAAGAGCAAGTCAGAGTGAACAGCCTGAC |
| | | ACACATGGTGGTGGTTGTGGATGAGTCCTCTGGGGATCATGCCA |
| | | CAGCTGCTCTGGAAGAACAGCTGAAGGTGCTGGGAGACAGATGG |
| | | GCCAACATCTGTAGGTGGACAGAGGATAGATGGGTGCTGCTCCA |
| | | GGACATTCTGCTGAAGTGGCAGAGACTGACAGAGGAACAGTGCC |
| | | TGTTTTCTGCCTGGCTCTCTGAGAAAGAGGATGCTGTCAACAAG |
| | | ATCCATACCACAGGCTTCAAGGATCAGAATGAGATGCTCAGCTC |
| | | CCTGCAGAAACTGGCTGTGCTGAAGGCTGACCTGGAAAAGAAAA |
| | | AGCAGTCCATGGGCAAGCTCTACAGCCTGAAGCAGGACCTGCTG |
| | | TCTACCCTGAAGAACAAGTCTGTGACCCAGAAAACTGAGGCCTG |
| | | GCTGGACAACTTTGCTAGATGCTGGGACAACCTGGTGCAGAAGC |
| | | TGGAAAAGTCTACAGCCCAGATCAGCCAGCAACCTGATCTTGCC |
| | | CCTGGCCTGACCACAATTGGAGCCTCTCCAACACAGACTGTGAC |
| | | CCTGGTTACCCAGCCAGTGGTCACCAAAGAGACAGCCATCAGCA |
| | | AACTGGAAATGCCCAGCTCTCTGATGCTGGAAGTCCCCACACTG |
| | | GAAAGGCTGCAAGAACTTCAAGAGGCCACAGATGAGCTGGACCT |
| | | GAAGCTGAGACAGGCTGAAGTGATCAAAGGCAGCTGGCAGCCAG |
| | | TTGGGGACCTGCTCATTGATAGCCTGCAGGACCATCTGGAAAAA |
| | | GTGAAAGCCCTGAGGGGAGAGATTGCCCCTCTGAAAGAAAATGT |
| | | GTCCCATGTGAATGACCTGGCCAGACAGCTGACCACACTGGGAA |
| | | TCCAGCTGAGCCCCTACAACCTGAGCACCCTTGAGGACCTGAAC |
| | | ACCAGGTGGAAGCTCCTCCAGGTGGCAGTGGAAGATAGAGTCAG |
| | | GCAGCTGCATGAGGCCCACAGAGATTTTGGACCAGCCAGCCAGC |
| | | ACTTTCTGTCTACCTCTGTGCAAGGCCCCTGGGAGAGAGCTATC |
| | | TCTCCTAACAAGGTGCCCTACTACATCAACCATGAGACACAGAC |
| | | CACCTGTTGGGATCACCCCAAGATGACAGAGCTGTACCAGAGTC |
| | | TGGCAGACCTCAACAATGTCAGATTCAGTGCCTACAGGACTGCC |
| | | ATGAAGCTCAGAAGGCTCCAGAAAGCTCTGTGCCTGGACCTGCT |
| | | TTCCCTGAGTGCAGCTTGTGATGCCCTGGACCAGCACAATCTGA |
| | | AGCAGAATGACCAGCCTATGGACATCCTCCAGATCATCAACTGC |
| | | CTCACCACCATCTATGATAGGCTGGAACAAGAGCACAACAATCT |
| | | GGTCAATGTGCCCCTGTGTGTGGACATGTGCCTGAATTGGCTGC |
| | | TGAATGTGTATGACACAGGCAGAACAGGCAGGATCAGAGTCCTG |
| | | TCCTTCAAGACAGGCATCATCTCCCTGTGCAAAGCCCACTTGGA |
| | | GGACAAGTACAGATACCTGTTCAAGCAAGTGGCCTCCAGCACAG |
| | | GCTTTTGTGACCAGAGAAGGCTGGGCCTGCTCCTGCATGACAGC |
| | | ATTCAGATCCCTAGACAGCTGGGAGAAGTGGCTTCCTTTGGAGG |
| | | CAGCAATATTGAGCCATCAGTCAGGTCCTGTTTTCAGTTTGCCA |
| | | ACAACAAGCCTGAGATTGAGGCTGCCCTGTTCCTGGACTGGATG |
| | | AGACTTGAGCCTCAGAGCATGGTCTGGCTGCCTGTGCTTCATAG |
| | | AGTGGCTGCTGCTGAGACTGCCAAGCACCAGGCCAAGTGCAACA |
| | | TCTGCAAAGAGTGCCCCATCATTGGCTTCAGATACAGATCCCTG |
| | | AAGCACTTCAACTATGATATCTGCCAGAGCTGCTTCTTTAGTGG |
| | | CAGGGTTGCCAAGGGCCACAAAATGCACTACCCCATGGTGGAAT |
| | | ACTGCACCCCAACAACCTCTGGGGAAGATGTTAGAGACTTTGCC |
| | | AAGGTGCTGAAAAACAAGTTCAGGACCAAGAGATACTTTGCTAA |
| | | GCACCCCAGAATGGGCTACCTGCCTGTCCAGACAGTGCTTGAGG |
| | | GTGACAACATGGAAACCTGATGAGTCGACAGGCCTAATAAAGAG |
| | | CTCAGATGCATCGATCAGAGTGTGTTGGTTTTTTGTGTGGCTAG |
| | | CTGCGGCCGCaggaacccctagtgatggagttggccactccctc |
| | | tctgcgcgctcgctcgctcactgaggccgggcgaccaaaggtcg |
| | | cccgacgcccgggctttgcccgggcggcctcagtgagcgagcga |
| | | gcgcgcag |
| RGX-DYS5 | 82 | ctgcgcgctcgctcgctcactgaggccgcccgggcaaagcccgg |
| (full cassette | | gcgtcgggcgacctttggtcgcccggcctcagtgagcgagcgag |
| SPc5-12 to | | cgcgcagagagggagtggccaactccatcactaggggttcctCA |
| polyA | | TATGcagggtaatggggatcctCTAGAGGCCGTCCGCCCTCGGC |
| including | | ACCATCCTCACGACACCCAAATATGGCGACGGGTGAGGAATGGT |
| intervening | | GGGGAGTTATTTTTAGAGCGGTGAGGAAGGTGGGCAGGCAGCAG |
| seqs) | | GTGTTGGCGCTCTAAAAATAACTCCCGGGAGTTATTTTTAGAGC |
| 4560 bp | | GGAGGAATGGTGGACACCCAAATATGGCGACGGTTCCTCACCCG |
| ITRs shown in | | TCGCCATATTTGGGTGTCCGCCCTCGGCCGGGGCCGCATTCCTG |
| lower case | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGCGGAATTCGCCACCATGCTTTGGTGGGAAGAGGTGGAAGATT |
| | | GCTATGAGAGGGAAGATGTGCAGAAGAAAACCTTCACCAAATGG |
| | | GTCAATGCCCAGTTCAGCAAGTTTGGCAAGCAGCACATTGAGAA |
| | | CCTGTTCAGTGACCTGCAGGATGGCAGAAGGCTGCTGGATCTGC |
| | | TGGAAGGCCTGACAGGCCAGAAGCTGCCTAAAGAGAAGGGCAGC |
| | | ACAAGAGTGCATGCCCTGAACAATGTGAACAAGGCCCTGAGAGT |
| | | GCTGCAGAACAACAATGTGGACCTGGTCAATATTGGCAGCACAG |
| | | ACATTGTGGATGGCAACCACAAGCTGACCCTGGGCCTGATCTGG |
| | | AACATCATCCTGCACTGGCAAGTGAAGAATGTGATGAAGAACAT |
| | | CATGGCTGGCCTGCAGCAGACCAACTCTGAGAAGATCCTGCTGA |
| | | GCTGGGTCAGACAGAGCACCAGAAACTACCCTCAAGTGAATGTG |
| | | ATCAACTTCACCACCTCTTGGAGTGATGGACTGGCCCTGAATGC |
| | | CCTGATCCACAGCCACAGACCTGACCTGTTTGACTGGAACTCTG |
| | | TTGTGTGCCAGCAGTCTGCCACACAGAGACTGGAACATGCCTTC |
| | | AACATTGCCAGATACCAGCTGGGAATTGAGAAACTGCTGGACCC |
| | | TGAGGATGTGGACACCACCTATCCTGACAAGAAATCCATCCTCA |
| | | TGTACATCACCAGCCTGTTCCAGGTGCTGCCCCAGCAAGTGTCC |
| | | ATTGAGGCCATTCAAGAGGTTGAGATGCTGCCCAGACCTCCTAA |
| | | AGTGACCAAAGAGGAACACTTCCAGCTGCACCACCAGATGCACT |
| | | ACTCTCAGCAGATCACAGTGTCTCTGGCCCAGGGATATGAGAGA |
| | | ACAAGCAGCCCCAAGCCTAGGTTCAAGAGCTATGCCTACACACA |
| | | GGCTGCCTATGTGACCACATCTGACCCCACAAGAAGCCCATTTC |
| | | CAAGCCAGCATCTGGAAGCCCCTGAGGACAAGAGCTTTGGCAGC |
| | | AGCCTGATGGAATCTGAAGTGAACCTGGATAGATACCAGACAGC |
| | | CCTGGAAGAAGTGCTGTCCTGGCTGCTGTCTGCTGAGGATACAC |
| | | TGCAGGCTCAGGGTGAAATCAGCAATGATGTGGAAGTGGTCAAG |
| | | GACCAGTTTCACACCCATGAGGGCTACATGATGGACCTGACAGC |
| | | CCACCAGGGCAGAGTGGGAAATATCCTGCAGCTGGGCTCCAAGC |
| | | TGATTGGCACAGGCAAGCTGTCTGAGGATGAAGAGACAGAGGTG |
| | | CAAGAGCAGATGAACCTGCTGAACAGCAGATGGGAGTGTCTGAG |
| | | AGTGGCCAGCATGGAAAAGCAGAGCAACCTGCACAGAGTGCTCA |
| | | TGGACCTGCAGAATCAGAAACTGAAAGAACTGAATGACTGGCTG |
| | | ACCAAGACAGAAGAAAGGACTAGGAAGATGGAAGAGGAACCTCT |
| | | GGGACCAGACCTGGAAGATCTGAAAAGACAGGTGCAGCAGCATA |
| | | AGGTGCTGCAAGAGGACCTTGAGCAAGAGCAAGTCAGAGTGAAC |
| | | AGCCTGACACACATGGTGGTGGTTGTGGATGAGTCCTCTGGGGA |
| | | TCATGCCACAGCTGCTCTGGAAGAACAGCTGAAGGTGCTGGGAG |
| | | ACAGATGGGCCAACATCTGTAGGTGGACAGAGGATAGATGGGTG |
| | | CTGCTCCAGGACATTCTGCTGAAGTGGCAGAGACTGACAGAGGA |
| | | ACAGTGCCTGTTTTCTGCCTGGCTCTCTGAGAAAGAGGATGCTG |
| | | TCAACAAGATCCATACCACAGGCTTCAAGGATCAGAATGAGATG |
| | | CTCAGCTCCCTGCAGAAACTGGCTGTGCTGAAGGCTGACCTGGA |
| | | AAAGAAAAAGCAGTCCATGGGCAAGCTCTACAGCCTGAAGCAGG |
| | | ACCTGCTGTCTACCCTGAAGAACAAGTCTGTGACCCAGAAAACT |
| | | GAGGCCTGGCTGGACAACTTTGCTAGATGCTGGGACAACCTGGT |
| | | GCAGAAGCTGGAAAAGTCTACAGCCCAGATCAGCCAGCAACCTG |
| | | ATCTTGCCCCTGGCCTGACCACAATTGGAGCCTCTCCAACACAG |
| | | ACTGTGACCCTGGTTACCCAGCCAGTGGTCACCAAAGAGACAGC |
| | | CATCAGCAAACTGGAAATGCCCAGCTCTCTGATGCTGGAAGTCC |
| | | CCACACTGGAAAGGCTGCAAGAACTTCAAGAGGCCACAGATGAG |
| | | CTGGACCTGAAGCTGAGACAGGCTGAAGTGATCAAAGGCAGCTG |
| | | GCAGCCAGTTGGGGACCTGCTCATTGATAGCCTGCAGGACCATC |
| | | TGGAAAAAGTGAAAGCCCTGAGGGGAGAGATTGCCCCTCTGAAA |
| | | GAAAATGTGTCCCATGTGAATGACCTGGCCAGACAGCTGACCAC |
| | | ACTGGGAATCCAGCTGAGCCCCTACAACCTGAGCACCCTTGAGG |
| | | ACCTGAACACCAGGTGGAAGCTCCTCCAGGTGGCAGTGGAAGAT |
| | | AGAGTCAGGCAGCTGCATGAGGCCCACAGAGATTTTGGACCAGC |
| | | CAGCCAGCACTTTCTGTCTACCTCTGTGCAAGGCCCCTGGGAGA |
| | | GAGCTATCTCTCCTAACAAGGTGCCCTACTACATCAACCATGAG |
| | | ACACAGACCACCTGTTGGGATCACCCCAAGATGACAGAGCTGTA |
| | | CCAGAGTCTGGCAGACCTCAACAATGTCAGATTCAGTGCCTACA |
| | | GGACTGCCATGAAGCTCAGAAGGCTCCAGAAAGCTCTGTGCCTG |
| | | GACCTGCTTTCCCTGAGTGCAGCTTGTGATGCCCTGGACCAGCA |
| | | CAATCTGAAGCAGAATGACCAGCCTATGGACATCCTCCAGATCA |
| | | TCAACTGCCTCACCACCATCTATGATAGGCTGGAACAAGAGCAC |
| | | AACAATCTGGTCAATGTGCCCCTGTGTGTGGACATGTGCCTGAA |
| | | TTGGCTGCTGAATGTGTATGACACAGGCAGAACAGGCAGGATCA |
| | | GAGTCCTGTCCTTCAAGACAGGCATCATCTCCCTGTGCAAAGCC |
| | | CACTTGGAGGACAAGTACAGATACCTGTTCAAGCAAGTGGCCTC |
| | | CAGCACAGGCTTTTGTGACCAGAGAAGGCTGGGCCTGCTCCTGC |
| | | ATGACAGCATTCAGATCCCTAGACAGCTGGGAGAAGTGGCTTCC |
| | | TTTGGAGGCAGCAATATTGAGCCATCAGTCAGGTCCTGTTTTCA |
| | | GTTTGCCAACAACAAGCCTGAGATTGAGGCTGCCCTGTTCCTGG |
| | | ACTGGATGAGACTTGAGCCTCAGAGCATGGTCTGGCTGCCTGTG |
| | | CTTCATAGAGTGGCTGCTGCTGAGACTGCCAAGCACCAGGCCAA |
| | | GTGCAACATCTGCAAAGAGTGCCCCATCATTGGCTTCAGATACA |
| | | GATCCCTGAAGCACTTCAACTATGATATCTGCCAGAGCTGCTTC |
| | | TTTAGTGGCAGGGTTGCCAAGGGCCACAAAATGCACTACCCCAT |
| | | GGTGGAATACTGCACCCCAACAACCTCTGGGGAAGATGTTAGAG |
| | | ACTTTGCCAAGGTGCTGAAAAACAAGTTCAGGACCAAGAGATAC |
| | | TTTGCTAAGCACCCCAGAATGGGCTACCTGCCTGTCCAGACAGT |
| | | GCTTGAGGGTGACAACATGGAAACCCCTGTGACACTGATCAATT |
| | | TCTGGCCAGTGGACTCTGCCCCTGCCTCAAGTCCACAGCTGTCC |
| | | CATGATGACACCCACAGCAGAATTGAGCACTATGCCTCCAGACT |
| | | GGCAGAGATGGAAAACAGCAATGGCAGCTACCTGAATGATAGCA |
| | | TCAGCCCCAATGAGAGCATTGATGATGAGCATCTGCTGATCCAG |
| | | CACTACTGTCAGTCCCTGAACCAGGACTCTCCACTGAGCCAGCC |
| | | TAGAAGCCCTGCTCAGATCCTGATCAGCCTTGAGTCTTGATGAG |
| | | TCGACAGGCCTAATAAAGAGCTCAGATGCATCGATCAGAGTGTG |
| | | TTGGTTTTTTGTGTGGCTAGCTGCGGCCGCaggaacccctagtg |
| | | atggagttggccactccctctctgcgcgctcgctcgctcactga |
| | | ggccgggcgaccaaaggtcgcccgacgcccgggctttgcccggg |
| | | cggcctcagtgagcgagcgagcgcgcag |
| Encodes RGX- | 97 | ctgcgcgctcgctcgctcactgaggccgcccgggcaaagc |
| DYS1 | | ccgggcgtcgggcgacctttggtcgcccggcctcagtgag |
| (full genome | | cgagcgagcgcgcagagagggagtggccaactccatcact |
| SPc5-12v1 to | | aggggttcctCATATGcagggtaatggggatcctCTAGAA |
| polyA | | GAGGCCGTCCGCCCTCGGCACCATCCTCACGACACCC |
| including | | AAATATGGCGACGGGTGAGGAATGGTGGGGAGTTATT |
| intervening | | TTTAGAGCGGTGAGGAAGGTGGGCAGGCAGCAGGTGT |
| sequences and | | TGGCGCTCCATATTTGGCGGGAGTTATTTTTAGAGCG |
| flanking ITR | | GAGGAATGGTGGACACCCAAATATGGCGACGGTTCCT |
| sequences) | | CACCCGTCGCTAAAAATAACTCCGTGTCCGCCCTCGG |
| ITRs shown in | | CCGGGGCCGCATTCCTGGGGGCCGGGCGGTGCTCCCG |
| lower case | | CCCGCCTCGATAAAAGGCTCCGGGGCCGGCGGCGGCC |
| | | CACGAGCTACCCGGAGGAGCGGGAGGCGCCAAGCGgA |
| | | AGgAATTCGCCACCATGCTTTGGTGGGAAGAGGTGGAAGA |
| | | TTGCTATGAGAGGGAAGATGTGCAGAAGAAAACCTTCACC |
| | | AAATGGGTCAATGCCCAGTTCAGCAAGTTTGGCAAGCAGC |
| | | ACATTGAGAACCTGTTCAGTGACCTGCAGGATGGCAGAAG |
| | | GCTGCTGGATCTGCTGGAAGGCCTGACAGGCCAGAAGCTG |
| | | CCTAAAGAGAAGGGCAGCACAAGAGTGCATGCCCTGAACA |
| | | ATGTGAACAAGGCCCTGAGAGTGCTGCAGAACAACAATGT |
| | | GGACCTGGTCAATATTGGCAGCACAGACATTGTGGATGGC |
| | | AACCACAAGCTGACCCTGGGCCTGATCTGGAACATCATCC |
| | | TGCACTGGCAAGTGAAGAATGTGATGAAGAACATCATGGC |
| | | TGGCCTGCAGCAGACCAACTCTGAGAAGATCCTGCTGAGC |
| | | TGGGTCAGACAGAGCACCAGAAACTACCCTCAAGTGAATG |
| | | TGATCAACTTCACCACCTCTTGGAGTGATGGACTGGCCCT |
| | | GAATGCCCTGATCCACAGCCACAGACCTGACCTGTTTGAC |
| | | TGGAACTCTGTTGTGTGCCAGCAGTCTGCCACACAGAGAC |
| | | TGGAACATGCCTTCAACATTGCCAGATACCAGCTGGGAAT |
| | | TGAGAAACTGCTGGACCCTGAGGATGTGGACACCACCTAT |
| | | CCTGACAAGAAATCCATCCTCATGTACATCACCAGCCTGT |
| | | TCCAGGTGCTGCCCCAGCAAGTGTCCATTGAGGCCATTCA |
| | | AGAGGTTGAGATGCTGCCCAGACCTCCTAAAGTGACCAAA |
| | | GAGGAACACTTCCAGCTGCACCACCAGATGCACTACTCTC |
| | | AGCAGATCACAGTGTCTCTGGCCCAGGGATATGAGAGAAC |
| | | AAGCAGCCCCAAGCCTAGGTTCAAGAGCTATGCCTACACA |
| | | CAGGCTGCCTATGTGACCACATCTGACCCCACAAGAAGCC |
| | | CATTTCCAAGCCAGCATCTGGAAGCCCCTGAGGACAAGAG |
| | | CTTTGGCAGCAGCCTGATGGAATCTGAAGTGAACCTGGAT |
| | | AGATACCAGACAGCCCTGGAAGAAGTGCTGTCCTGGCTGC |
| | | TGTCTGCTGAGGATACACTGCAGGCTCAGGGTGAAATCAG |
| | | CAATGATGTGGAAGTGGTCAAGGACCAGTTTCACACCCAT |
| | | GAGGGCTACATGATGGACCTGACAGCCCACCAGGGCAGAG |
| | | TGGGAAATATCCTGCAGCTGGGCTCCAAGCTGATTGGCAC |
| | | AGGCAAGCTGTCTGAGGATGAAGAGACAGAGGTGCAAGAG |
| | | CAGATGAACCTGCTGAACAGCAGATGGGAGTGTCTGAGAG |
| | | TGGCCAGCATGGAAAAGCAGAGCAACCTGCACAGAGTGCT |
| | | CATGGACCTGCAGAATCAGAAACTGAAAGAACTGAATGAC |
| | | TGGCTGACCAAGACAGAAGAAAGGACTAGGAAGATGGAAG |
| | | AGGAACCTCTGGGACCAGACCTGGAAGATCTGAAAAGACA |
| | | GGTGCAGCAGCATAAGGTGCTGCAAGAGGACCTTGAGCAA |
| | | GAGCAAGTCAGAGTGAACAGCCTGACACACATGGTGGTGG |
| | | TTGTGGATGAGTCCTCTGGGGATCATGCCACAGCTGCTCT |
| | | GGAAGAACAGCTGAAGGTGCTGGGAGACAGATGGGCCAAC |
| | | ATCTGTAGGTGGACAGAGGATAGATGGGTGCTGCTCCAGG |
| | | ACATTCTGCTGAAGTGGCAGAGACTGACAGAGGAACAGTG |
| | | CCTGTTTTCTGCCTGGCTCTCTGAGAAAGAGGATGCTGTC |
| | | AACAAGATCCATACCACAGGCTTCAAGGATCAGAATGAGA |
| | | TGCTCAGCTCCCTGCAGAAACTGGCTGTGCTGAAGGCTGA |
| | | CCTGGAAAAGAAAAAGCAGTCCATGGGCAAGCTCTACAGC |
| | | CTGAAGCAGGACCTGCTGTCTACCCTGAAGAACAAGTCTG |
| | | TGACCCAGAAAACTGAGGCCTGGCTGGACAACTTTGCTAG |
| | | ATGCTGGGACAACCTGGTGCAGAAGCTGGAAAAGTCTACA |
| | | GCCCAGATCAGCCAGCAACCTGATCTTGCCCCTGGCCTGA |
| | | CCACAATTGGAGCCTCTCCAACACAGACTGTGACCCTGGT |
| | | TACCCAGCCAGTGGTCACCAAAGAGACAGCCATCAGCAAA |
| | | CTGGAAATGCCCAGCTCTCTGATGCTGGAAGTCCCCACAC |
| | | TGGAAAGGCTGCAAGAACTTCAAGAGGCCACAGATGAGCT |
| | | GGACCTGAAGCTGAGACAGGCTGAAGTGATCAAAGGCAGC |
| | | TGGCAGCCAGTTGGGGACCTGCTCATTGATAGCCTGCAGG |
| | | ACCATCTGGAAAAAGTGAAAGCCCTGAGGGGAGAGATTGC |
| | | CCCTCTGAAAGAAAATGTGTCCCATGTGAATGACCTGGCC |
| | | AGACAGCTGACCACACTGGGAATCCAGCTGAGCCCCTACA |
| | | ACCTGAGCACCCTTGAGGACCTGAACACCAGGTGGAAGCT |
| | | CCTCCAGGTGGCAGTGGAAGATAGAGTCAGGCAGCTGCAT |
| | | GAGGCCCACAGAGATTTTGGACCAGCCAGCCAGCACTTTC |
| | | TGTCTACCTCTGTGCAAGGCCCCTGGGAGAGAGCTATCTC |
| | | TCCTAACAAGGTGCCCTACTACATCAACCATGAGACACAG |
| | | ACCACCTGTTGGGATCACCCCAAGATGACAGAGCTGTACC |
| | | AGAGTCTGGCAGACCTCAACAATGTCAGATTCAGTGCCTA |
| | | CAGGACTGCCATGAAGCTCAGAAGGCTCCAGAAAGCTCTG |
| | | TGCCTGGACCTGCTTTCCCTGAGTGCAGCTTGTGATGCCC |
| | | TGGACCAGCACAATCTGAAGCAGAATGACCAGCCTATGGA |
| | | CATCCTCCAGATCATCAACTGCCTCACCACCATCTATGAT |
| | | AGGCTGGAACAAGAGCACAACAATCTGGTCAATGTGCCCC |
| | | TGTGTGTGGACATGTGCCTGAATTGGCTGCTGAATGTGTA |
| | | TGACACAGGCAGAACAGGCAGGATCAGAGTCCTGTCCTTC |
| | | AAGACAGGCATCATCTCCCTGTGCAAAGCCCACTTGGAGG |
| | | ACAAGTACAGATACCTGTTCAAGCAAGTGGCCTCCAGCAC |
| | | AGGCTTTTGTGACCAGAGAAGGCTGGGCCTGCTCCTGCAT |
| | | GACAGCATTCAGATCCCTAGACAGCTGGGAGAAGTGGCTT |
| | | CCTTTGGAGGCAGCAATATTGAGCCATCAGTCAGGTCCTG |
| | | TTTTCAGTTTGCCAACAACAAGCCTGAGATTGAGGCTGCC |
| | | CTGTTCCTGGACTGGATGAGACTTGAGCCTCAGAGCATGG |
| | | TCTGGCTGCCTGTGCTTCATAGAGTGGCTGCTGCTGAGAC |
| | | TGCCAAGCACCAGGCCAAGTGCAACATCTGCAAAGAGTGC |
| | | CCCATCATTGGCTTCAGATACAGATCCCTGAAGCACTTCA |
| | | ACTATGATATCTGCCAGAGCTGCTTCTTTAGTGGCAGGGT |
| | | TGCCAAGGGCCACAAAATGCACTACCCCATGGTGGAATAC |
| | | TGCACCCCAACAACCTCTGGGGAAGATGTTAGAGACTTTG |
| | | CCAAGGTGCTGAAAAACAAGTTCAGGACCAAGAGATACTT |
| | | TGCTAAGCACCCCAGAATGGGCTACCTGCCTGTCCAGACA |
| | | GTGCTTGAGGGTGACAACATGGAAACCCCTGTGACACTGA |
| | | TCAATTTCTGGCCAGTGGACTCTGCCCCTGCCTCAAGTCC |
| | | ACAGCTGTCCCATGATGACACCCACAGCAGAATTGAGCAC |
| | | TATGCCTCCAGACTGGCAGAGATGGAAAACAGCAATGGCA |
| | | GCTACCTGAATGATAGCATCAGCCCCAATGAGAGCATTGA |
| | | TGATGAGCATCTGCTGATCCAGCACTACTGTCAGTCCCTG |
| | | AACCAGGACTCTCCACTGAGCCAGCCTAGAAGCCCTGCTC |
| | | AGATCCTGATCAGCCTTGAGTCTGAGGAAAGGGGAGAGCT |
| | | GGAAAGAATCCTGGCAGATCTTGAGGAAGAGAACAGAAAC |
| | | CTGCAGGCAGAGTATGACAGGCTCAAACAGCAGCATGAGC |
| | | ACAAGGGACTGAGCCCTCTGCCTTCTCCTCCTGAAATGAT |
| | | GCCCACCTCTCCACAGTCTCCAAGGTGATGACTCGAGAGG |
| | | CCTAATAAAGAGCTCAGATGCATCGATCAGAGTGTGTTGG |
| | | TTTTTTGTGTGCCAGGGTAATGGGCTAGCTGCGGCCGCag |
| | | gaacccctagtgatggagttggccactccctctctgcgcg |
| | | ctcgctcgctcactgaggccgggcgaccaaaggtcgcccg |
| | | acgcccgggctttgcccgggcggcctcagtgagcgagcga |
| | | gcgcgcag |
| Encodes RGX- | 98 | ctgcgcgctcgctcgctcactgaggccgcccgggcaaagc |
| DYS1 | | ccgggcgtcgggcgacctttggtcgcccggcctcagtgag |
| (full genome | | cgagcgagcgcgcagagagggagtggccaactccatcact |
| SPc5-12v2 to | | aggggttcctCATATGcagggtaatggggatcctCTAGAG |
| polyA | | GCCGTCCGCCCTCGGCACCATCCTCACGACACCCAAA |
| including | | TATGGCGACGGGTGAGGAATGGTGGGGAGTTATTTTT |
| intervening | | AGAGCGGTGAGGAAGGTGGGCAGGCAGCAGGTGTTGG |
| sequences and | | GGGAGTTATTTTTAGAGCGGGGAGTTATTTTTAGAGC |
| flanking ITR | | GGAGGAATGGTGGACACCCAAATATGGCGACGGTTCC |
| sequences) | | TCACGGACACCCAAATATGGCGACGGGCCCTCGGCCG |
| ITRs shown in | | GGGCCGCATTCCTGGGGGCCGGGCGGTGCTCCCGCCC |
| lower case | | GCCTCGATAAAAGGCTCCGGGGCCGGCGGCGGCCCAC |
| | | GAGCTACCCGGAGGAGCGGGAGGCGCCAAGCGgAATT |
| | | CGCCACCATGCTTTGGTGGGAAGAGGTGGAAGATTGCTAT |
| | | GAGAGGGAAGATGTGCAGAAGAAAACCTTCACCAAATGGG |
| | | TCAATGCCCAGTTCAGCAAGTTTGGCAAGCAGCACATTGA |
| | | GAACCTGTTCAGTGACCTGCAGGATGGCAGAAGGCTGCTG |
| | | GATCTGCTGGAAGGCCTGACAGGCCAGAAGCTGCCTAAAG |
| | | AGAAGGGCAGCACAAGAGTGCATGCCCTGAACAATGTGAA |
| | | CAAGGCCCTGAGAGTGCTGCAGAACAACAATGTGGACCTG |
| | | GTCAATATTGGCAGCACAGACATTGTGGATGGCAACCACA |
| | | AGCTGACCCTGGGCCTGATCTGGAACATCATCCTGCACTG |
| | | GCAAGTGAAGAATGTGATGAAGAACATCATGGCTGGCCTG |
| | | CAGCAGACCAACTCTGAGAAGATCCTGCTGAGCTGGGTCA |
| | | GACAGAGCACCAGAAACTACCCTCAAGTGAATGTGATCAA |
| | | CTTCACCACCTCTTGGAGTGATGGACTGGCCCTGAATGCC |
| | | CTGATCCACAGCCACAGACCTGACCTGTTTGACTGGAACT |
| | | CTGTTGTGTGCCAGCAGTCTGCCACACAGAGACTGGAACA |
| | | TGCCTTCAACATTGCCAGATACCAGCTGGGAATTGAGAAA |
| | | CTGCTGGACCCTGAGGATGTGGACACCACCTATCCTGACA |
| | | AGAAATCCATCCTCATGTACATCACCAGCCTGTTCCAGGT |
| | | GCTGCCCCAGCAAGTGTCCATTGAGGCCATTCAAGAGGTT |
| | | GAGATGCTGCCCAGACCTCCTAAAGTGACCAAAGAGGAAC |
| | | ACTTCCAGCTGCACCACCAGATGCACTACTCTCAGCAGAT |
| | | CACAGTGTCTCTGGCCCAGGGATATGAGAGAACAAGCAGC |
| | | CCCAAGCCTAGGTTCAAGAGCTATGCCTACACACAGGCTG |
| | | CCTATGTGACCACATCTGACCCCACAAGAAGCCCATTTCC |
| | | AAGCCAGCATCTGGAAGCCCCTGAGGACAAGAGCTTTGGC |
| | | AGCAGCCTGATGGAATCTGAAGTGAACCTGGATAGATACC |
| | | AGACAGCCCTGGAAGAAGTGCTGTCCTGGCTGCTGTCTGC |
| | | TGAGGATACACTGCAGGCTCAGGGTGAAATCAGCAATGAT |
| | | GTGGAAGTGGTCAAGGACCAGTTTCACACCCATGAGGGCT |
| | | ACATGATGGACCTGACAGCCCACCAGGGCAGAGTGGGAAA |
| | | TATCCTGCAGCTGGGCTCCAAGCTGATTGGCACAGGCAAG |
| | | CTGTCTGAGGATGAAGAGACAGAGGTGCAAGAGCAGATGA |
| | | ACCTGCTGAACAGCAGATGGGAGTGTCTGAGAGTGGCCAG |
| | | CATGGAAAAGCAGAGCAACCTGCACAGAGTGCTCATGGAC |
| | | CTGCAGAATCAGAAACTGAAAGAACTGAATGACTGGCTGA |
| | | CCAAGACAGAAGAAAGGACTAGGAAGATGGAAGAGGAACC |
| | | TCTGGGACCAGACCTGGAAGATCTGAAAAGACAGGTGCAG |
| | | CAGCATAAGGTGCTGCAAGAGGACCTTGAGCAAGAGCAAG |
| | | TCAGAGTGAACAGCCTGACACACATGGTGGTGGTTGTGGA |
| | | TGAGTCCTCTGGGGATCATGCCACAGCTGCTCTGGAAGAA |
| | | CAGCTGAAGGTGCTGGGAGACAGATGGGCCAACATCTGTA |
| | | GGTGGACAGAGGATAGATGGGTGCTGCTCCAGGACATTCT |
| | | GCTGAAGTGGCAGAGACTGACAGAGGAACAGTGCCTGTTT |
| | | TCTGCCTGGCTCTCTGAGAAAGAGGATGCTGTCAACAAGA |
| | | TCCATACCACAGGCTTCAAGGATCAGAATGAGATGCTCAG |
| | | CTCCCTGCAGAAACTGGCTGTGCTGAAGGCTGACCTGGAA |
| | | AAGAAAAAGCAGTCCATGGGCAAGCTCTACAGCCTGAAGC |
| | | AGGACCTGCTGTCTACCCTGAAGAACAAGTCTGTGACCCA |
| | | GAAAACTGAGGCCTGGCTGGACAACTTTGCTAGATGCTGG |
| | | GACAACCTGGTGCAGAAGCTGGAAAAGTCTACAGCCCAGA |
| | | TCAGCCAGCAACCTGATCTTGCCCCTGGCCTGACCACAAT |
| | | TGGAGCCTCTCCAACACAGACTGTGACCCTGGTTACCCAG |
| | | CCAGTGGTCACCAAAGAGACAGCCATCAGCAAACTGGAAA |
| | | TGCCCAGCTCTCTGATGCTGGAAGTCCCCACACTGGAAAG |
| | | GCTGCAAGAACTTCAAGAGGCCACAGATGAGCTGGACCTG |
| | | AAGCTGAGACAGGCTGAAGTGATCAAAGGCAGCTGGCAGC |
| | | CAGTTGGGGACCTGCTCATTGATAGCCTGCAGGACCATCT |
| | | GGAAAAAGTGAAAGCCCTGAGGGGAGAGATTGCCCCTCTG |
| | | AAAGAAAATGTGTCCCATGTGAATGACCTGGCCAGACAGC |
| | | TGACCACACTGGGAATCCAGCTGAGCCCCTACAACCTGAG |
| | | CACCCTTGAGGACCTGAACACCAGGTGGAAGCTCCTCCAG |
| | | GTGGCAGTGGAAGATAGAGTCAGGCAGCTGCATGAGGCCC |
| | | ACAGAGATTTTGGACCAGCCAGCCAGCACTTTCTGTCTAC |
| | | CTCTGTGCAAGGCCCCTGGGAGAGAGCTATCTCTCCTAAC |
| | | AAGGTGCCCTACTACATCAACCATGAGACACAGACCACCT |
| | | GTTGGGATCACCCCAAGATGACAGAGCTGTACCAGAGTCT |
| | | GGCAGACCTCAACAATGTCAGATTCAGTGCCTACAGGACT |
| | | GCCATGAAGCTCAGAAGGCTCCAGAAAGCTCTGTGCCTGG |
| | | ACCTGCTTTCCCTGAGTGCAGCTTGTGATGCCCTGGACCA |
| | | GCACAATCTGAAGCAGAATGACCAGCCTATGGACATCCTC |
| | | CAGATCATCAACTGCCTCACCACCATCTATGATAGGCTGG |
| | | AACAAGAGCACAACAATCTGGTCAATGTGCCCCTGTGTGT |
| | | GGACATGTGCCTGAATTGGCTGCTGAATGTGTATGACACA |
| | | GGCAGAACAGGCAGGATCAGAGTCCTGTCCTTCAAGACAG |
| | | GCATCATCTCCCTGTGCAAAGCCCACTTGGAGGACAAGTA |
| | | CAGATACCTGTTCAAGCAAGTGGCCTCCAGCACAGGCTTT |
| | | TGTGACCAGAGAAGGCTGGGCCTGCTCCTGCATGACAGCA |
| | | TTCAGATCCCTAGACAGCTGGGAGAAGTGGCTTCCTTTGG |
| | | AGGCAGCAATATTGAGCCATCAGTCAGGTCCTGTTTTCAG |
| | | TTTGCCAACAACAAGCCTGAGATTGAGGCTGCCCTGTTCC |
| | | TGGACTGGATGAGACTTGAGCCTCAGAGCATGGTCTGGCT |
| | | GCCTGTGCTTCATAGAGTGGCTGCTGCTGAGACTGCCAAG |
| | | CACCAGGCCAAGTGCAACATCTGCAAAGAGTGCCCCATCA |
| | | TTGGCTTCAGATACAGATCCCTGAAGCACTTCAACTATGA |
| | | TATCTGCCAGAGCTGCTTCTTTAGTGGCAGGGTTGCCAAG |
| | | GGCCACAAAATGCACTACCCCATGGTGGAATACTGCACCC |
| | | CAACAACCTCTGGGGAAGATGTTAGAGACTTTGCCAAGGT |
| | | GCTGAAAAACAAGTTCAGGACCAAGAGATACTTTGCTAAG |
| | | CACCCCAGAATGGGCTACCTGCCTGTCCAGACAGTGCTTG |
| | | AGGGTGACAACATGGAAACCCCTGTGACACTGATCAATTT |
| | | CTGGCCAGTGGACTCTGCCCCTGCCTCAAGTCCACAGCTG |
| | | TCCCATGATGACACCCACAGCAGAATTGAGCACTATGCCT |
| | | CCAGACTGGCAGAGATGGAAAACAGCAATGGCAGCTACCT |
| | | GAATGATAGCATCAGCCCCAATGAGAGCATTGATGATGAG |
| | | CATCTGCTGATCCAGCACTACTGTCAGTCCCTGAACCAGG |
| | | ACTCTCCACTGAGCCAGCCTAGAAGCCCTGCTCAGATCCT |
| | | GATCAGCCTTGAGTCTGAGGAAAGGGGAGAGCTGGAAAGA |
| | | ATCCTGGCAGATCTTGAGGAAGAGAACAGAAACCTGCAGG |
| | | CAGAGTATGACAGGCTCAAACAGCAGCATGAGCACAAGGG |
| | | ACTGAGCCCTCTGCCTTCTCCTCCTGAAATGATGCCCACC |
| | | TCTCCACAGTCTCCAAGGTGATGACTCGAGAGGCCTAATA |
| | | AAGAGCTCAGATGCATCGATCAGAGTGTGTTGGTTTTTTG |
| | | TGTGCCAGGGTAATGGGCTAGCTGCGGCCGCaggaacccc |
| | | tagtgatggagttggccactccctctctgcgcgctcgctc |
| | | gctcactgaggccgggcgaccaaaggtcgcccgacgcccg |
| | | ggctttgcccgggcggcctcagtgagcgagcgagcgcgca |
| | | g |

### 5.3.5 Methods of Making rAAV Particles

Another aspect of the present invention involves making molecules disclosed herein. In some embodiments, a molecule according to the invention is made by providing a nucleotide comprising the nucleic acid sequence encoding any of the capsid protein molecules herein; and using a packaging cell system to prepare corresponding rAAV particles with capsid coats made up of the capsid protein. Such capsid proteins are described in Section 5.3.4, supra. In some embodiments, the nucleic acid sequence encodes a sequence having at least 60%, 70%, 80%, 85%, 90%, or 95%, including 96%, 97%, 98%, 99% or 99.9%, identity to the sequence of a capsid protein molecule described herein and retains (or substantially retains) biological function of the capsid protein and the inserted peptide from a heterologous protein or domain thereof. In some embodiments, the nucleic acid encodes a sequence having at least 60%, 70%, 80%, 85%, 90%, or 95%, including 96%, 97%, 98%, 99% or 99.9%, identity to the sequence of the AAV8 capsid protein, while retaining (or substantially retaining) biological function of the AAV8 capsid protein and the inserted peptide.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In embodiments, the cap and rep genes are provided by a packaging cell and not present in the viral genome.

In some embodiments, the nucleic acid encoding the engineered capsid protein is cloned into an AAV Rep-Cap plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging.

Numerous cell culture-based systems are known in the art for production of rAAV particles, any of which can be used to practice a method disclosed herein. The cell culture-based systems include transfection, stable cell line production, and infectious hybrid virus production systems which include, but are not limited to, adenovirus-AAV hybrids, herpesvirus-AAV hybrids and baculovirus-AAV hybrids. rAAV production cultures for the production of rAAV virus particles require: (1) suitable host cells, including, for example, human-derived cell lines, mammalian cell lines, or insect-derived cell lines; (2) suitable helper virus function, provided by wild type or mutant adenovirus (such as temperature-sensitive adenovirus), herpes virus, baculovirus, or a plasmid construct providing helper functions; (3) AAV rep and cap genes and gene products; (4) a transgene (such as a therapeutic transgene) flanked by AAV ITR sequences and optionally regulatory elements; and (5) suitable media and media components (nutrients) to support cell growth/survival and rAAV production.

Nonlimiting examples of host cells include: A549, WEHI, 10T1/2, BHK, MDCK, COS1, COS7, BSC 1, BSC 40, BMT 10, VERO, W138, HeLa, HEK293 and their derivatives (HEK293T cells, HEK293F cells), Saos, C2C12, L, HT1080, HepG2, primary fibroblast, hepatocyte, myoblast cells, CHO cells or CHO-derived cells, or insect-derived cell lines such as SF-9 (e.g. in the case of baculovirus production systems). For a review, see Aponte-Ubillus et al., 2018, Appl. Microbiol. Biotechnol. 102:1045-1054, which is incorporated by reference herein in its entirety for manufacturing techniques.

In one aspect, provided herein is a method of producing rAAV particles, comprising (a) providing a cell culture comprising an insect cell; (b) introducing into the cell one or more baculovirus vectors encoding at least one of: i. an rAAV genome to be packaged, ii. an AAV rep protein sufficient for packaging, and iii. an AAV cap protein sufficient for packaging; (c) adding to the cell culture sufficient nutrients and maintaining the cell culture under conditions that allow production of the rAAV particles. In some embodiments, the method comprises using a first baculovirus vector encoding the rep and cap genes and a second baculovirus vector encoding the rAAV genome. In some embodiments, the method comprises using a baculovirus encoding the rAAV genome and an insect cell expressing the rep and cap genes. In some embodiments, the method comprises using a baculovirus vector encoding the rep and cap genes and the rAAV genome. In some embodiments, the insect cell is an Sf-9 cell. In some embodiments, the insect cell is an Sf-9 cell comprising one or more stably integrated heterologous polynucleotide encoding the rep and cap genes.

In some embodiments, a method disclosed herein uses a baculovirus production system. In some embodiments the baculovirus production system uses a first baculovirus encoding the rep and cap genes and a second baculovirus encoding the rAAV genome. In some embodiments the baculovirus production system uses a baculovirus encoding the rAAV genome and a host cell expressing the rep and cap genes. In some embodiments the baculovirus production system uses a baculovirus encoding the rep and cap genes and the rAAV genome. In some embodiments, the baculovirus production system uses insect cells, such as Sf-9 cells.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by inverted terminal repeats (ITRs)) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments of a method disclosed herein, AAV rep and cap genes, helper genes, and rAAV genomes are introduced into cells by transfection of one or more plasmid vectors encoding the AAV rep and cap genes, helper genes, and rAAV genome. In some embodiments of a method disclosed herein, AAV rep and cap genes, helper genes, and rAAV genomes can be introduced into cells by transduction with viral vectors, for example, rHSV vectors encoding the AAV rep and cap genes, helper genes, and rAAV genome. In some embodiments of a method disclosed herein, one or more of AAV rep and cap genes, helper genes, and rAAV genomes are introduced into the cells by transduction with an rHSV vector. In some embodiments, the rHSV vector encodes the AAV rep and cap genes. In some embodiments, the rHSV vector encodes the helper genes. In some embodiments, the rHSV vector encodes the rAAV genome. In some embodiments, the rHSV vector encodes the AAV rep and cap genes. In some embodiments, the rHSV vector encodes the helper genes and the rAAV genome. In some embodiments, the rHSV vector encodes the helper genes and the AAV rep and cap genes.

In one aspect, provided herein is a method of producing rAAV particles, comprising (a) providing a cell culture comprising a host cell; (b) introducing into the cell one or more rHSV vectors encoding at least one of: i. an rAAV genome to be packaged, ii. helper functions necessary for packaging the rAAV particles, iii. an AAV rep protein sufficient for packaging, and iv. an AAV cap protein sufficient for packaging; (c) adding to the cell culture sufficient nutrients and maintaining the cell culture under conditions that allow production of the rAAV particles. In some embodiments, the rHSV vector encodes the AAV rep and cap genes. In some embodiments, the rHSV vector encodes helper functions. In some embodiments, the rHSV vector comprises one or more endogenous genes that encode helper functions. In some embodiments, the rHSV vector comprises one or more heterogeneous genes that encode helper functions. In some embodiments, the rHSV vector encodes the rAAV genome. In some embodiments, the rHSV vector encodes the AAV rep and cap genes. In some embodiments, the rHSV vector encodes helper functions and the rAAV genome. In some embodiments, the rHSV vector encodes helper functions and the AAV rep and cap genes. In some embodiments, the cell comprises one or more stably integrated heterologous polynucleotide encoding the rep and cap genes.

In one aspect, provided herein is a method of producing rAAV particles, comprising (a) providing a cell culture comprising a mammalian cell; (b) introducing into the cell one or more polynucleotides encoding at least one of: i. an rAAV genome to be packaged, ii. helper functions necessary for packaging the rAAV particles, iii. an AAV rep protein sufficient for packaging, and iv. an AAV cap protein sufficient for packaging; (c) adding to the cell culture sufficient nutrients and maintaining the cell culture under conditions that allow production of the rAAV particles. In some embodiments, the helper functions are encoded by adenovirus genes. In some embodiments, the mammalian cell comprises one or more stably integrated heterologous polynucleotide encoding the rep and cap genes.

Molecular biology techniques to develop plasmid or viral vectors encoding the AAV rep and cap genes, helper genes, and/or rAAV genome are commonly known in the art. In some embodiments, AAV rep and cap genes are encoded by one plasmid vector. In some embodiments, AAV helper genes (e.g., adenovirus E1a gene, E1b gene, E4 gene, E2a gene, and VA gene) are encoded by one plasmid vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one plasmid vector. In some embodiments, one or more helper genes are stably expressed by the host cell, and one or more helper genes are introduced into the cell by transfection by one plasmid vector. In some embodiments, the helper genes are stably expressed by the host cell. In some embodiments, AAV rep and cap genes are encoded by one viral vector. In some embodiments, AAV helper genes (e.g., adenovirus E1a gene, E1b gene, E4 gene, E2a gene, and VA gene) are encoded by one viral vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one viral vector. In some embodiments, one or more helper genes are stably expressed by the host cell, and one or more helper genes are introduced into the cell by transfection by one viral vector. In some embodiments, the AAV rep and cap genes, the adenovirus helper functions necessary for packaging, and the rAAV genome to be packaged are introduced to the cells by transfection with one or more polynucleotides, e.g., vectors. In some embodiments, a method disclosed herein comprises transfecting the cells with a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the AAV cap gene is an AAV8 or AAV9 cap gene. In some embodiments, the AAV cap gene is an AAV.rh8, AAV.rh10, AAV.rh20, AAV.rh39, AAV.Rh74, AAV.RHM4-1, AAV.hu37, AAV.PHB, or AAV.7m8 cap gene. In some embodiments, the AAV cap gene encodes a capsid protein with high sequence homology to AAV8 or AAV9 such as, AAV.rh10, AAV.rh20, AAV.rh39, AAV.Rh74, AAV.RHM4-1, and AAV.hu37. In some embodiments, the vector encoding the rAAV genome to be packaged comprises a gene of interest flanked by AAV ITRs. In some embodiments, the AAV ITRs are from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes.

Any combination of vectors can be used to introduce AAV rep and cap genes, AAV helper genes, and rAAV genome to a cell in which rAAV particles are to be produced or packaged. In some embodiments of a method disclosed herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell. In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses Ela. In some embodiments, the cell comprises a stable transgene encoding the rAAV genome.

In some embodiments, AAV rep, cap, and helper genes (e.g., E1a gene, E1b gene, E4 gene, E2a gene, or VA gene) can be of any AAV serotype. Similarly, AAV ITRs can also be of any AAV serotype. For example, in some embodiments, AAV ITRs are from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In some embodiments, AAV cap gene is from AAV8 or AAV9 cap gene. In some embodiments, an AAV cap gene is from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.rh74, AAV.hu31, AAV.hu32, or AAV.hu37 or other AAV serotypes (e.g., a hybrid serotype harboring sequences from more than one serotype). In some embodiments, AAV rep and cap genes for the production of a rAAV particle are from different serotypes. For example, the rep gene is from AAV2 whereas the cap gene is from AAV8. In another example, the rep gene is from AAV2 whereas the cap gene is from AAV9.

In some embodiments, the rep gene is from AAV1, AAV2, AAV3, AAV4, AAV5, AAV6, AAV7, AAV8, AAV9, AAV10, AAV11, AAV12, AAV13, AAV14, AAV15 and AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other AAV serotypes (e.g., a hybrid serotype harboring sequences from more than one serotype). In other embodiments, the rep and the cap genes are from the same serotype. In still other embodiments, the rep and the cap genes are from the same serotype, and the rep gene comprises at least one modified protein domain or modified promoter domain. In certain embodiments, the at least one modified domain comprises a nucleotide sequence of a serotype that is different from the capsid serotype. The modified domain within the rep gene may be a hybrid nucleotide sequence consisting fragments different serotypes.

Hybrid rep genes provide improved packaging efficiency of rAAV particles, including packaging of a viral genome comprising a microdystrophin transgene greater than 4 kb, greater than 4.1 kb, greater than 4.2 kB, greater than 4.3 kb, greater than 4.4 kB, greater than 4.5 kb, or greater than 4.6 kb. AAV rep genes consist of nucleic acid sequences that encode the non-structural proteins needed for replication and production of virus. Transcription of the rep gene initiates from the p5 or p19 promoters to produce two large (Rep78 and Rep68) and two small (Rep52 and Rep40) nonstructural Rep proteins, respectively. Additionally, Rep78/68 domain contains a DNA-binding domain that recognizes specific ITR sequences within the ITR. All four Rep proteins have common helicase and ATPase domains that function in genome replication and/or encapsidation (Maurer A C, 2020, DOI: 10.1089/hum.2020.069). Transcription of the cap gene initiates from a p40 promoter, which sequence is within the C-terminus of the rep gene, and it has been suggested that other elements in the rep gene may induce p40 promoter activity. The p40 promoter domain includes transcription factor binding elements EF1A, MLTF, and ATF, Fos/Jun binding elements (AP-1), Sp1-like elements (Sp1 and GGT), and the TATA element (Pereira and Muzyczka, Journal of Virology, June 1997, 71(6):4300-4309). In some embodiments, the rep gene comprises a modified p40 promoter. In some embodiments, the p40 promoter is modified at any one or more of the EF1A binding element, MLTF binding element, ATF binding element, Fos/Jun binding elements (AP-1), Sp1-like elements (Sp1 or GGT), or the TATA element. In other embodiments, the rep gene is of serotype 1, 3, 4, 5, 6, 7, 8, 9, 10, 11, 12, 13, 14, 15, 16, rh8, rh10, rh20, rh39, rh.74, RHM4-1, or hu37, and the portion or element of the p40 promoter domain is modified to serotype 2. In still other embodiments, the rep gene is of serotype 8 or 9, and the portion or element of the p40 promoter domain is modified to serotype 2.

ITRs contain A and A′ complimentary sequences, B and B′ complimentary sequences, and C and C′ complimentary sequences; and the D sequence is contiguous with the ssDNA genome. The complimentary sequences of the ITRs form hairpin structures by self-annealing (Berns K I. The Unusual Properties of the AAV Inverted Terminal Repeat. Hum Gene Ther 2020). The D sequence contains a Rep Binding Element (RBE) and a terminal resolution site (TRS), which together constitute the AAV origin of replication. The ITRs are also required as packaging signals for genome encapsidation following replication. In some embodiments, the ITR sequences and the cap genes are from the same serotype, except that one or more of the A and A′ complimentary sequences, B and B′ complimentary sequences, C and C′ complimentary sequences, or the D sequence may be modified to contain sequences from a different serotype than the capsid. In some embodiments, the modified ITR sequences are from the same serotype as the rep gene. In other embodiments, the ITR sequences and the cap genes are from different serotypes, except that one or more of the ITR sequences selected from A and A′ complimentary sequences, B and B′ complimentary sequences, C and C′ complimentary sequences, or the D sequence are from the same serotype as the capsid (cap gene), and one or more of the ITR sequences are from the same serotype as the rep gene.

In some embodiments, the rep and the cap genes are from the same serotype, and the rep gene comprises a modified Rep78 domain, DNA binding domain, endonuclease domain, ATPase domain, helicase domain, p5 promoter domain, Rep68 domain, p5 promoter domain, Rep52 domain, p19 promoter domain, Rep40 domain or p40 promoter domain. In other embodiments, the rep and the cap genes are from the same serotype, and the rep gene comprises at least one protein domain or promoter domain from a different serotype. In one embodiment, an rAAV comprises a transgene flanked by AAV2 ITR sequences, an AAV8 cap, and a hybrid AAV2/8 rep. In another embodiment, the AAV2/8 rep comprises serotype 8 rep except for the p40 promoter domain or a portion thereof is from serotype 2 rep. In other embodiments, the AAV2/8 rep comprises serotype 2 rep except for the p40 promoter domain or a portion thereof is from serotype 8 rep. In some embodiments, more than two serotypes may be utilized to construct a hybrid rep/cap plasmid.

Any suitable method known in the art may be used for transfecting a cell may be used for the production of rAAV particles according to a method disclosed herein. In some embodiments, a method disclosed herein comprises transfecting a cell using a chemical based transfection method. In some embodiments, the chemical-based transfection method uses calcium phosphate, highly branched organic compounds (dendrimers), cationic polymers (e.g., DEAE dextran or polyethylenimine (PEI)), lipofection. In some embodiments, the chemical-based transfection method uses cationic polymers (e.g., DEAE dextran or polyethylenimine (PEI)). In some embodiments, the chemical-based transfection method uses polyethylenimine (PEI). In some embodiments, the chemical-based transfection method uses DEAE dextran. In some embodiments, the chemical-based transfection method uses calcium phosphate.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

Provided are host cell lines for production of the rAAV particles containing the constructs encoding the microdystrophins as disclosed herein, including the constructs of SEQ ID NO: 53 or 82 (RGX-DYS1 or RGX-DYS5).

In preferred embodiments, the rAAVs provide transgene delivery vectors that can be used in therapeutic and prophylactic applications, as discussed in more detail below.

### 5.4. Therapeutic Utility

Provided are methods of assaying the constructs, including recombinant gene therapy vectors, encoding microdystrophins, as disclosed herein, for therapeutic efficacy alone or in combination with one or more of the second therapeutics described herein. Methods include both in vitro and in vivo tests in animal models as described herein or using any other methods known in the art for testing the activity and efficacy of microdystrophins alone or in combination with a second therapeutic.

### 5.4.1 In Vitro Assays

### 5.4.1.1 In Vitro Infection System for Muscle Cells

Provided are methods of testing of the infectivity of a recombinant vector disclosed herein, for example rAAV particles. For example, the infectivity of recombinant gene therapy vectors in muscle cells can be tested in C2C12 myoblasts. Several muscle or heart cell lines may be utilized, including but not limited to T0034 (human), L6 (rat), MM14 (mouse), P19 (mouse), G-7 (mouse), G-8 (mouse), QM7 (quail), H9c2(2-1) (rat), Hs 74.Ht (human), and Hs 171.Ht (human) cell lines. Vector copy numbers may be assess using polymerase chain reaction techniques and level of microdystrophin expression may be tested by measuring levels of microdystrophin mRNA in the cells.

### 5.4.2 Animal Models

The efficacy of a viral vector containing a transgene encoding a microdystrophin as described herein may be tested by administering to an animal model to replace mutated dystrophin, for example, by using the mdx mouse and/or the golden retriever muscular dystrophy (GRMD) model and to assess the biodistribution, expression and therapeutic effect of the transgene expression. The therapeutic effect may be assessed, for example, by assessing change in muscle strength in the animal receiving the microdystrophin transgene. Animal models using larger mammals as well as nonmammalian vertebrates and invertebrates can also be used to assess pre-clinical therapeutic efficacy of a vector described herein. Accordingly, provided are compositions and methods for therapeutic administration comprising a dose of a microdystrophin encoding vector disclosed herein in an amount demonstrated to be effective according to the methods for assessing therapeutic efficacy disclosed here either alone or in combination with a second therapeutic described herein.

### 5.4.2.1 Murine Models

The efficacy of gene therapy vectors alone or in combination with the second therapeutics disclosed herein may be assessed in murine models of DMD. The mdx mouse model (Yucel, N., et al, Humanizing the mdx mouse model of DMD: the long and the short of it, Regenerative Medicine volume 3, Article number: 4 (2018)), carries a nonsense mutation in exon 23, resulting in an early termination codon and a truncated protein (mdx). Mdx mice have 3-fold higher blood levels of pyruvate kinase activity compared to littermate controls. Like the human DMD disease, mdx skeletal muscles exhibit active myofiber necrosis, cellular infiltration, a wide range of myofiber sizes and numerous centrally nucleated regenerating myofibers. This phenotype is enhanced in the diaphragm, which undergoes progressive degeneration and myofiber loss resulting in an approximately 5-fold reduction in muscle isometric strength. Necrosis and regeneration in hind-limb muscles peaks around 3-4 weeks of age, but plateaus thereafter. In mdx mice and mdx mice crossed onto other mouse backgrounds (for example DBA/2J), a mild but significant decrease in cardiac ejection fraction is observed (Van Westering, Molecules 2015, 20, 8823-8855). Such DMD model mice with cardiac functional defects may be used to assess the cardioprotective effects or improvement or maintenance of cardiac function or attenuation of cardiac dysfunction of the gene therapy vectors described herein alone or in combination with the second therapeutics disclosed herein. Examples 5-8 herein details use of the mdx mouse model to assess gene therapy vectors encoding microdystrophins.

Additional mdx mouse models: A number of alternative versions in different genetic backgrounds have been generated including the mdx2cv, mdx3cv, mdx4cv, and mdx5cv lines (C57BL/6 genetic background). These models were created by treating mice with N-ethyl-N-nitrosourea, a chemical mutagen. Each strain carries a different point mutation. As a whole, there are few differences in the presentation of disease phenotypes in the mdxcv models compared to the mdx mouse. Additional mouse models have been created by crossing the mdx line to various knock-out mouse models (e.g. Myod1^{-/-}, α-Integrin7^{-/-}, α-Dystrobrevin^{-/-}, and Utrophin^{-/-}). All mouse models which are currently used to study DMD have been described in detail by Yucel, N., et al, Humanizing the mdx mouse model of DMD: the long and the short of it, npj Regenerative Medicine volume 3, Article number: 4 (2018), which is incorporated herein by reference.

### Cardiac Function

Assessment of efficacy on cardiac function can be measured in mice, including mdx mice. To measure the blood pressure (BP) mice are sedated using 1.5% isofluorane with constant monitoring of the plane of anesthesia and maintenance of the body temperature at 36.5-37.58 C. The heart rate is maintained at 450-550 beats/min. A BP cuff is placed around the tail, and the tail is then placed in a sensor assembly for noninvasive BP monitoring during anesthesia. Ten consecutive BP measurements are taken. Qualitative and quantitative measurements of tail BP, including systolic pressure, diastolic pressure and mean pressure, are made offline using analytic software. See, for example, Wehling-Henricks et al, Human Molecular Genetics, 2005, Vol. 14, No. 14; Uaesoontrachoon et al, Human Molecular Genetics, 2014, Vol. 23, No. 12.

To monitor ECG wave heights and interval durations in awake, freely moving mice, radio telemetry devices are used. Transmitter units are implanted in the peritoneal cavity of anesthetized mice and the two electrical leads are secured near the apex of the heart and the right acromion in a lead II orientation. Mice are housed singly in cages over antenna receivers connected to a computer system for data recording. Unfiltered ECG data is collected for 10 seconds each hour for 35 days. The first 7 days of data are discarded to allow for recovery from the surgical procedure and ensure any effects of anesthesia has subsided. Data waveforms and parameters are analyzed with the DSI analysis packages (ART 3.01 and Physiostat 4.01) and measurements are compiled and averaged to determine heart rates, ECG wave heights and interval durations. Raw ECG waveforms are scanned for arrhythmias by two independent observers.

Picro-Sirius red staining is performed to measure the degree of fibrosis in the heart of trial mice. In brief, at the end of trial, directly following euthanasia, the heart muscle is removed and fixed in 10% formalin for later processing. The heart is sectioned and paraffin sections are deparaffinized in xylene followed by nuclear staining with Weigert's hematoxylin for 8 min. They are then washed and then stained with Picro-Sirius red (0.5 g of Sirius red F3B, saturated aqueous solution of picric acid) for an additional 30 min. The sections are cleared in three changes of xylene and mounted in Permount. Five random digital images are taken using an Eclipse E800 (Nikon, Japan) microscope, and blinded analysis is done using Image J (NIH).Blood samples are taken via cardiac puncture when the animals are euthanized, and the serum collected is used for the measurement of muscle CK levels.

### 5.4.2.2 Canine

Most canine studies are conducted in the golden retriever muscular dystrophy (GRMD) model (Korneygay, J. N., et al, The golden retriever model of Duchenne muscular dystrophy. Skelet Muscle. 2017; 7: 9, which is incorporated by reference in its entirety). Dogs with GRMD are afflicted with a progressive, fatal disease with skeletal and cardiac muscle phenotypes and selective muscle involvement—a severe phenotype that more closely mirrors that of DMD. GRMD dogs carry a single nucleotide change that leads to exon skipping and an out-of-frame DMD transcript. Phenotypic features in dogs include elevation of serum CK, CRDs on EMG, and histopathologic evidence of grouped muscle fiber necrosis and regeneration. Phenotypic variability is frequently observed in GRMD, as in humans. GRMD dogs develop paradoxical muscle hypertrophy which seems to play a role in the phenotype of affected dogs, with stiffness at gait, decreased joint range of motion, and trismus being common features. Objective biomarkers to evaluate disease progression include tetanic flexion, tibiotarsal joint angle, % eccentric contraction decrement, maximum hip flexion angle, pelvis angle, cranial sartorius circumference, and quadriceps femoris weight.

### 5.5. Methods of Treatment

Provided are methods of treating human subjects for any muscular dystrophy disease that can be treated by providing a functional dystrophin in combination with a second therapeutic, wherein the second therapeutic can treat a dystrophinopathy disease or ameliorate one or more symptoms thereof. DMD is the most common of such disease, but the gene therapy vectors that express microdystrophin provided herein can be administered in combination with a second therapeutic described herein to treat Becker muscular dystrophy (BMD), myotonic muscular dystrophy (Steinert's disease), Facioscapulohumeral disease (FSHD), limb-girdle muscular dystrophy, X-linked dilated cardiomyopathy, or oculopharyngeal muscular dystrophy. The microdystrophin of the present disclosure may be any microdystrophin described herein, including those that have the domains in an N-terminal to C-terminal order of ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, CR is a cysteine-rich region of dystrophin and CT is at least a portion of a C-terminal region of dystrophin comprising a α1-syntrophin binding site, in certain embodiments SEQ ID NO:16 or SEQ ID NO:83. In embodiments, the microdystrophin has an amino acid sequence of SEQ ID Nos: 1, 2, or 79. The vectors encoding the microdystrophin include those having a nucleic acid sequence of SEQ ID NO: 20, 21, or 81, in certain embodiments, operably linked to regulatory elements for constitutive, muscle-specific (including skeletal, smooth muscle and cardiac muscle-specific) expression, or CNS specific expression, and other regulatory elements such as poly A sites. Such nucleic acids may be in the context of an rAAV genome, for example, flanked by ITR sequences, particularly, AAV2 ITR sequences. In certain embodiments, the methods and compositions comprising administering to a subject in need thereof, an rAAV comprising the construct having a nucleic acid sequence of SEQ ID NO: 53, 55, or 82. In embodiments, the constructs are in an rAAV8 or rAAV9 particle. In embodiments, the therapeutic is AAV8-RGX-DYS1. In embodiments, the patient has been diagnosed with and/or has symptom(s) associated with DMD.

In embodiments, the second therapeutic is a mutation suppression therapy, an exon skipping therapy, a steroid therapy, an immunosuppressive/anti-inflammatory therapy, any therapy that treats one or more symptoms of the dystrophinopathy, or any combination thereof. Dosing for each second therapeutic can be any of the known doses for administering each of the second therapeutics.

In some embodiments, the second therapeutic can be administered to alleviate or further alleviate one or more symptoms or characteristics of dystrophinopathies which may be assessed by any of, but not limited to, the following assays on the subject: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. As an example, the publication of Manzur et al. (Manzur A Y et al, (2008), Glucocorticoid corticosteroids for Duchenne muscular dystrophy (review), Wiley publishers, The Cochrane collaboration.) gives an extensive explanation of each of these assays. For each of these assays, as soon as a detectable improvement or prolongation of a parameter measured in an assay has been found, it may indicate that one or more symptoms of Duchenne Muscular Dystrophy has been alleviated in an individual using a method of the invention. Detectable improvement or prolongation may be a statistically significant improvement or prolongation as described in Hodgetts et al (Hodgetts S., et al, (2006), Neuromuscular Disorders, 16: 591-602.2006). Alternatively, the alleviation of one or more symptom(s) of Duchenne Muscular Dystrophy may be assessed by measuring an improvement of a muscle fiber function, integrity and/or survival as later defined herein.

A treatment in a method according to the invention may have a duration of at least one week, at least one month, at least several months, at least one year, at least 2, 3, 4, 5, 6 years or more. The frequency of administration of any of the second therapeutics described herein may depend on several parameters such as the age of the patient, the type of mutation, the number of molecules (dose), the formulation of said molecule. The frequency may be ranged between at least once in a two weeks, or three weeks or four weeks or five weeks or a longer time period.

The first therapeutic and second therapeutic can be administered to an individual in any order. When more than one second therapeutic is administered those can also be administered in any order relevant to each other and to the first therapeutic. In one embodiment, said therapeutics are administered simultaneously (meaning that said therapeutics are administered within 10 hours, including within one hour). In another embodiment, said therapeutics are administered sequentially.

In some embodiments, the first and second therapeutics provide a synergistic therapeutic effect with respect to one or more clinical end points in the treatment of a dystrophinopathy in a subject, in particular, where the therapeutic effect is greater than the additive therapeutic effects of the first and second therapeutics when administered alone. In some embodiments, the first and second therapeutics provide a synergistic effect in that the therapeutics result in improvements in different sets of clinical endpoints such that the therapeutic benefit of the combination is greater than the therapeutic benefit of each therapeutic individually.

### 5.5.1 Mutation Suppression Therapy

Disclosed are methods of treating a dystrophinopathy in a subject in need thereof, comprising administering to the subject a first therapeutic and a second therapeutic, wherein the first therapeutic is an rAAV vector comprising a transgene encoding a microdystrophin disclosed herein and the second therapeutic is a mutation suppression therapy.

In some embodiments, the second therapeutic is ataluren. In some embodiments, ataluren is administered orally. In some embodiments, ataluren can be administered in a dose of 10 mg/kg/day to 200 mg/kg/day. In some embodiments, ataluren can be administered in a dose of 40 mg/kg. For example, the dosing can be 10 mg/kg in the morning, 10 mg/kg at midday, and 20 mg/kg in the evening. The length of time for ataluren administration can be weeks, months, or years. In some embodiments, treatment resulted in increased ability to walk/run longer distances and/or increased ability to climb stairs compared to pre-treatment levels.

In some embodiments, the second therapeutic is gentamicin. In some embodiments, gentamicin is administered intravenously. In some embodiments, gentamicin can be administered in a dose of 3 mg/kg/day to 25 mg/kg/day. In some embodiments, gentamicin can be administered in a dose of 7.5 mg/kg/day. The length of time for ataluren administration can be weeks, months, or years. In some embodiments, treatment resulted in increased hearing, kidney function and/or muscle strength compared to pre-treatment levels.

In some embodiments, the mutation suppressor therapy is a nonsense suppressor mutation. For example, the subject can have a nonsense mutation and the second therapeutic enables a ribosome to read through a premature nonsense mutation.

Nonsense suppressor therapies can be of two general classes. A first class includes compounds that disrupt codon-anticodon recognition during protein translation in a eukaryotic cell, so as to promote readthrough of a nonsense codon. These agents can act by, for example, binding to a ribosome so as to affect its activity in initiating translation or promoting polypeptide chain elongation, or both. For example, nonsense suppressor agents of this class can act by binding to rRNA (e.g., by reducing binding affinity to 18S rRNA). A second class are those that provide the eukaryotic translational machinery with a tRNA that provides for incorporation of an amino acid in a polypeptide where the mRNA normally encodes a stop codon, e.g., suppressor tRNAs.

### 5.5.2 Exon Skipping Therapy

Disclosed are methods of treating a dystrophinopathy in a subject in need thereof, comprising administering to the subject a first therapeutic and a second therapeutic, wherein the first therapeutic is an rAAV comprising a transgene encoding a microdystrophin disclosed herein and the second therapeutic is an exon skipping therapy. In some embodiments, the exon skipping therapy is an antisense oligonucleotide.

In some embodiments, a subject is first identified as being amenable to treatment with an exon skipping therapy.

Exon skipping refers to the induction in a cell of a mature mRNA that does not contain a particular exon that is normally present therein. Exon skipping is achieved by providing a cell expressing the pre-mRNA of said mRNA with a molecule (i.e. exon skipping therapy) capable of interfering with sequences such as, for example, the splice donor or splice acceptor sequence that are both required for allowing the enzymatic process of splicing, or a molecule (i.e. exon skipping therapy) that is capable of interfering with an exon inclusion signal required for recognition of a stretch of nucleotides as an exon to be included in the mRNA. The term pre-mRNA refers to a non-processed or partly processed precursor mRNA that is synthesized from a DNA template in the cell nucleus by transcription.

In some embodiments, a subject treated with the exon skipping therapy means that at least 1%, 10%, 20%, 30%, 40%, 50%, 60%, 70%, 80%, 90% or more of the DMD mRNA in one or more (muscle) cells of the subject will not contain said exon.

In some embodiments, the exon skipping therapy results in skipping of one or more exons of dystrophin. In some embodiments, one or more of exons 1-60 can be skipped. In some embodiments, one or more of exons 2, 43, 44, 45, 50, 51, 52, 53, or 55 of the human dystrophin gene can be skipped to express a form of dystrophin protein.

In some embodiments, the exon skipping therapy results in skipping exon 45. For example, in some embodiments, the exon skipping therapy can be casimersen. In some embodiments, casimersen can be administered intravenously. In some embodiments, administration can be daily, weekly, or monthly. In some embodiments, the length of treatment can be weeks, months or years. In some embodiments, casimersen can be administered in a dose of 10 mg/kg to 200 mg/kg. In some embodiments, casimersen can be administered in a dose of 30 mg/kg. For example, administration can be once weekly via intravenous (IV) infusions of 30 mg/kg. In some embodiments, the exon skipping therapy can be SRP-5045. In some embodiments, the exon skipping therapy can be DS-5141B. In some embodiments, DS-5141B can be administered subcutaneously. In some embodiments, administration can be daily, weekly, or monthly. In some embodiments, the length of treatment can be weeks, months or years. In some embodiments, DS-5141B can be administered in a dose of 0.1 mg/kg to 20 mg/kg. In some embodiments, DS-5141B can be administered in a dose of 2 mg/kg or 6 mg/kg. For example, administration can be subcutaneously once a week for 2 weeks at a dose of 2 to 6 mg/kg/week.

In some embodiments, the exon skipping therapy results in skipping exon 50. For example, in some embodiments, the exon skipping therapy can be SRP-5050. In some embodiments, SRP-5050 can be administered intravenously or subcutaneously. In some embodiments, administration can be daily, weekly, or monthly. In some embodiments, the length of treatment can be weeks, months or years. SRP-5050 is part of a peptide phosphorodiamidate morpholino oligomer (PPMO) technology that includes a cell-penetrating peptide that is conjugated to an oligomer backbone with the goal of increasing cellular uptake in the muscle tissue. In some embodiments, the PPMO technology used herein is similar to that described in Tsoumpra et al. EBioMedicine 45(2019):630-645 and/or Guidotti et al. Trends in Pharmacological Sciences, vol 38, issue 4, 406-424, 2017, both of which are incorporated herein by reference in their entirety.

In some embodiments, the exon skipping therapy results in skipping exon 51. For example, in some embodiments, the exon skipping therapy can be eteplirsen. in some embodiments, the exon skipping therapy can be SRP-5051. SRP-5050 is part of the PPMO technology that includes a cell-penetrating peptide that is conjugated to an oligomer backbone with the goal of increasing cellular uptake in the muscle tissue. In some embodiments, SRP-5051 can be administered intravenously. In some embodiments, administration can be daily, weekly, or monthly. In some embodiments, the length of treatment can be weeks, months or years. In some embodiments, SRP-5051 can be administered in a dose of 1 mg/kg to 200 mg/kg. In some embodiments, SRP-5051 can be administered in a dose of 4 mg/kg to 40 mg/kg. For example, administration can be once monthly via intravenous (IV) infusion at a dose of 20 mg/kg.

In some embodiments, the exon skipping therapy results in skipping exon 53. For example, in some embodiments, the exon skipping therapy can be golodirsen. In some embodiments, golodirsen can be administered intravenously. In some embodiments, administration can be daily, weekly, or monthly. In some embodiments, the length of treatment can be weeks, months or years. In some embodiments, golodirsen can be administered in a dose of 10 mg/kg/day to 200 mg/kg/day. In some embodiments, golodirsen can be administered in a dose of 30 mg/kg. For example, administration can be once weekly via intravenous (IV) infusions of 30 mg/kg.

In some embodiments, the exon skipping therapy can be SRP-5053. SRP-5053 is part of the PPMO technology that includes a cell-penetrating peptide that is conjugated to an oligomer backbone with the goal of increasing cellular uptake in the muscle tissue. In some embodiments, SRP-5053 can be administered intravenously or subcutaneously. In some embodiments, administration can be daily, weekly, or monthly. In some embodiments, the length of treatment can be weeks, months or years.

In some embodiments, the exon skipping therapy can be viltolarsen. In some embodiments, viltolarsen can be administered intravenously. In some embodiments, administration can be daily, weekly, or monthly. In some embodiments, the length of treatment can be weeks, months or years. In some embodiments, viltolarsen can be administered in a dose of 10 mg/kg to 200 mg/kg. In some embodiments, viltolarsen can be administered in a dose of 80 mg/kg. For example, administration can be once weekly via intravenous (IV) infusions of 80 mg/kg.

In some embodiments, the exon skipping therapy results in skipping exon 52. For example, in some embodiments, the exon skipping therapy can be SRP-5052. SRP-5052 is part of the PPMO technology that includes a cell-penetrating peptide that is conjugated to an oligomer backbone with the goal of increasing cellular uptake in the muscle tissue. In some embodiments, SRP-5052 can be administered intravenously or subcutaneously. In some embodiments, administration can be daily, weekly, or monthly. In some embodiments, the length of treatment can be weeks, months or years.

In some embodiments, the exon skipping therapy results in skipping exon 44. For example, in some embodiments, the exon skipping therapy can be SRP-5044. SRP-5044 is part of the PPMO technology that includes a cell-penetrating peptide that is conjugated to an oligomer backbone with the goal of increasing cellular uptake in the muscle tissue. In some embodiments, SRP-5044 can be administered intravenously or subcutaneously. In some embodiments, administration can be daily, weekly, or monthly. In some embodiments, the length of treatment can be weeks, months or years.

In some embodiments, the exon skipping therapy can be NS-089/NCNP-02. In some embodiments, NS-089/NCNP-02 can be administered intravenously. In some embodiments, administration can be daily, weekly, or monthly. In some embodiments, the length of treatment can be weeks, months or years. In some embodiments, NS-089/NCNP-02 can be administered in a dose of 0.5 mg/kg to 200 mg/kg. In some embodiments, NS-089/NCNP-02 can be administered in a dose of 1.62 mg/kg, 10 mg/kg, 40 mg/kg, or 80 mg/kg. For example, administration can be once weekly via intravenous (IV) infusions of 1.62 mg/kg, 10 mg/kg, 40 mg/kg, or 80 mg/kg.

In some embodiments, the exon skipping therapy results in skipping exon 2. For example, in some embodiments, the exon skipping therapy can be scAAV9.U7.ACCA. scAAV9.U7.ACCA is an AAV9 vector carrying U7snRNA to treat a duplicate of exon 2. In some embodiments, scAAV9.U7.ACCA can be administered intravenously. In some embodiments, administration can be daily, weekly, or monthly. In some embodiments, the length of treatment can be weeks, months or years. In some embodiments, scAAV9.U7.ACCA can be administered in a dose of 1×10¹² viral genomes/kilogram (vg/kg) to 1×10¹⁵ vg/kg. In some embodiments, NS-089/NCNP-02 can be administered in a dose of 3×10¹³ vg/kg to 8×10¹³ vg/kg. For example, administration can be once daily, weekly, monthly or yearly via intravenous (IV) infusions of 3×10¹³ vg/kg or 8×10¹³ vg/kg.

In some embodiments, the second therapeutic can be a combination of two or more of the exon skipping therapies described herein. For example, in some embodiments, the exon skipping therapy can be a combination of casimersen and golodiresen or casimersen, eteplirsen, and golodiresen.

### 5.5.3 Steroid Therapy

Disclosed are methods of treating a dystrophinopathy in a subject in need thereof, comprising administering to the subject a first therapeutic and a second therapeutic, wherein the first therapeutic is an rAAV comprising a transgene encoding a microdystrophin disclosed herein and the second therapeutic is a steroid therapy. In some embodiments, the steroid therapy is a glucocorticoid steroid

In some embodiments, the steroid therapy is prednisone, deflazacort, Vamorolone, or Spironolactone, or a combination thereof. Spironolactone is an aldosterone antagonist and although may not be considered a steroid, it is used in a similar manner to steroids and is often compared to corticosteroids.

In some embodiments, the daily dose of prednisone is 0.2 mg/kg/day to 10 mg/kg/day. In some embodiments, the daily dose of prednisone is 0.75 mg/kg/day. In some embodiments, the daily dose of deflazacort is 0.2 mg/kg/day to 40 mg/kg/day. In some embodiments, the daily dose of deflazacort is 0.9 mg/kg/day. In some embodiments, the daily dose of Vamorolone is 0.5 mg/kg to 40 mg/kg. In some embodiments, the daily dose of Vamorolone is 2 mg/kg, 6 mg/kg or 20 mg/kg. In some embodiments, the daily dose of Spironolactone is 5 mg to 40 mg. In some embodiments, the daily dose of Spironolactone is 12.5 mg or 25 mg.

The steroid dose can be increased or decreased based on growth, weight, and other side effects experienced. In some embodiments, dosing can be either daily or high dose weekends. For example, inn some embodiments, doses of twice weekly can go up to 250 mg/day of prednisone or 300 mg/day of deflazacort. In some embodiments, dosing can be 10 days on, 10 days off, etc.

### 5.5.4 Immunosuppressive/Anti-Inflammatory Therapy

Disclosed are methods of treating a dystrophinopathy in a subject in need thereof, comprising administering to the subject a first therapeutic and a second therapeutic, wherein the first therapeutic is an rAAV comprising a transgene encoding a microdystrophin disclosed herein and the second therapeutic is an immunosuppressive or anti-inflammatory therapy.

In some embodiments, the immunosuppressive or anti-inflammatory therapy is edasalonexent.

In some embodiments, the immunosuppressive or anti-inflammatory therapy is canakinumab. Canakinumab is a monoclonal antibody, targeting IL1b, which is a cytokine that plays a role in inflammation and immune responses. In some embodiments, canakinumab can be administered subcutaneously. In some embodiments, administration can be daily, weekly, or monthly. In some embodiments, the length of treatment can be weeks, months or years. In some embodiments, canakinumab can be administered in a dose of 0.5 mg/kg to 20 mg/kg. In some embodiments, canakinumab can be administered in a dose of 2 mg/kg or 4 mg/kg. For example, administration can be a single dose via subcutaneous injection of 2 or 4 mg/kg.

In some embodiments, the immunosuppressive or anti-inflammatory therapy is pamrevlumab. Pamrevlumab is an antibody therapy designed to block the activity of connective tissue growth factor (CTGF), a pro-inflammatory protein that promotes fibrosis (scarring) and is found at unusually high levels in the muscles of people with DMD. Fibrosis is a hallmark of muscular dystrophies, contributing to muscle weakness and injury, including to cardiac muscle. In some embodiments, inhibition of connective tissue growth factor (CTGF) by pamrevlumab could result in decreased fibrosis in muscles leading to increased muscle function. In some embodiments, Pamrevlumab can be administered intravenously. In some embodiments, administration can be daily, weekly, or monthly. In some embodiments, the length of treatment can be weeks, months or years. In some embodiments, Pamrevlumab can be administered in a dose of 10 mg/kg to 200 mg/kg. In some embodiments, Pamrevlumab can be administered in a dose of 35 mg/kg. For example, administration can be every two weeks via intravenous (IV) infusions of 35 mg/kg.

In some embodiments, the immunosuppressive or anti-inflammatory therapy is imlifidase. Imlifidase is an enzyme that rapidly cleaves IgG antibodies, thereby suppressing the immune response against AAVs. Thus, once the immune response against AAVs has been suppressed, gene therapy treatments using an AAV vector can be used more efficiently. In some embodiments, imlifidase can be administered intravenously. In some embodiments, administration can be daily, weekly, or monthly. In some embodiments, the length of treatment can be weeks, months or years. In some embodiments, imlifidase can be administered in a dose of 0.1 mg/kg to 10 mg/kg. In some embodiments, imlifidase can be administered in a dose of 0.25 mg/kg. For example, administration can a single dose via intravenous (IV) infusions of 0.25 mg/kg.

### 5.5.5 Therapies that Treat One or More Symptoms of the Dystrophinopathy

Disclosed are methods of treating a dystrophinopathy in a subject in need thereof, comprising administering to the subject a first therapeutic and a second therapeutic, wherein the first therapeutic is an rAAV comprising a transgene encoding a microdystrophin disclosed herein and the second therapeutic is a therapy that treats one or more symptoms of the dystrophinopathy. In some embodiments, a therapy that treats one or more symptoms of the dystrophinopathy can also include any of the mutation suppression therapies, exon skipping therapies, steroid therapies, and immunosuppressive/anti-inflammatory therapies described herein.

In some embodiments, the one or more symptoms of the dystrophinopathy is decreased muscle mass and/or strength, wherein the second therapeutic improves muscle mass and/or strength. For example, the second therapeutic can be spironolactone (same as described for steroid therapy), Follistatin, SERCA2a, EDG-5506, Tamoxifen, Givinostat, ASP0367, or a combination thereof.

In some embodiments, follistatin or follistatin variants can be used as the second therapeutic. In some embodiments, follistatin can be administered as a gene therapy in a viral vector such as AAV.

In some embodiments, SERCA2a can be used as the second therapeutic. In some embodiments, SERCA2a can be administered as a gene therapy in a viral vector such as AAV. In some embodiments, SERCA2a can be administered intravenously. In some embodiments, administration can be daily, weekly, or monthly. In some embodiments, the length of treatment can be weeks, months or years. In some embodiments, 1×10¹¹ to 1×10¹⁴ vg is administered. In some embodiments, 6×10¹² vg is administered.

EDG-5506 is a small molecule therapy that can stabilize skeletal muscle fibers (muscles under voluntary control) and protect them from damage during contractions. In some embodiments, SERCA2a can be administered orally. In some embodiments, administration can be daily, weekly, or monthly. In some embodiments, the length of treatment can be weeks, months or years.

In some embodiments, the second therapeutic is tamoxifen. In some embodiments, tamoxifen can be administered orally. In some embodiments, administration can be daily, weekly, or monthly. In some embodiments, the length of treatment can be weeks, months or years. In some embodiments, tamoxifen can be administered in a dose of 0.1 mg/kg to 20 mg/kg. In some embodiments, tamoxifen can be administered in a dose of 0.6 mg/kg. In some embodiments, tamoxifen can be administered in a dose of 5 mg to 100 mg. For example, administration can be a single oral dose of 0.6 mg/kg daily.

In some embodiments, Givinostat is a molecule that inhibits enzymes called histone deacetylases (HDACs) that turn off gene expression and can reduce a muscle's ability to regenerate. By inhibiting HDACs, givinostat may reduce fibrosis and the death of muscle cells while also enabling muscles to regenerate. In some embodiments, Givinostat is administered via oral suspension. In some embodiments, administration can be daily, weekly, or monthly. In some embodiments, the length of treatment can be weeks, months or years. In some embodiments, Givinostat can be administered in a dose of 1 mg/ml to 100 mg/ml. In some embodiments, Givinostat can be administered in a dose of 10 mg/ml. For example, administration can be twiaily via oral suspension of 10 mg/ml.

In some embodiments, ASP0367 is used turn on the PPAR delta (6) pathway. The PPAR-6 pathway regulates mitochondria by turning on different genes in the cell. When the pathway is on, the mitochondria use fatty acids more often and more mitochondria are made. Using more fatty acids for energy results in increased energy production. Thus, ASP0367 is a mitochondrial-directed medicine for the treatment of DMD, which is designed to treat DMD by increasing fatty acid oxidation and mitochondrial biogenesis in muscle cells.

In some embodiments, the second therapeutic is a cell based therapy. For example, the cell based therapy is one or more myoblasts. In some embodiments, the myoblast cell based therapy is NCT02196467. In some embodiments, 1-500 million myoblasts can be transplanted per centimeter cube in the Extensor carpi radialis of one of the patient's forearms, resuspended in saline. More specifically, 30 million myoblasts can be transplanted per centimeter cube can be transplanted.

In some embodiments, the cell based therapy is CAP-1002 and can improve respiratory, cardiac and upper limb function. Thus, in some embodiments, the cell based therapy is a cardiosphere derived cell.

In some embodiments, the one or more symptoms of the dystrophinopathy is a symptom related to a cardiac condition. In some embodiments, the cardiac condition is cardiomyopathy, decreased cardiac function, fibrosis in the heart, or a combination thereof. Thus, in some embodiments, the second therapeutic is Ifetroban, Bisoprolol fumarate, Eplerenone, or a combination thereof.

Ifetroban is a potent and selective thromboxane receptor antagonist. In some embodiments ifetroban can stop important molecular signals that mediate inflammation and fibrosis (tissue scaring) mechanisms in the heart, triggered by the loss of dystrophin protein—the hallmark feature of DMD. In some embodiments, ifetroban is administered orally. In some embodiments, administration can be daily, weekly, or monthly. In some embodiments, the length of treatment can be weeks, months or years. In some embodiments, ifetroban can be administered in a dose of 50 mg to 400 mg. In some embodiments, ifetroban can be administered in a dose of 200 mg. For example, administration can be once daily via capsule—four 50 mg capsules. In some embodiments, Bisoprolol is administered at a dose of 0.05 mg/kg to 20 mg/kg. In some embodiments, Bisoprolol is administered at a dose of 0.2 mg/kg. In some embodiments, Bisoprolol is administered at a dose of 1.25 mg every 24 hr and the subject is monitored for heart rate, blood pressure, and other heart related symptoms. The bisoprolol dose can be increased 1.25 mg progressively until a daily dose of 0.2 mg/kg or the maximum tolerated dose (he rest heart rate<75 bpm and systolic blood pressure<90 mmHg) is achieved. Dosing can be increased with an assessment of the subject's heart rate, blood pressure, symptoms and ECG.

In some embodiments, eplerenone is administered orally. In some embodiments, administration can be daily, weekly, or monthly. In some embodiments, the length of treatment can be weeks, months or years. In some embodiments, eplerenone can be administered in a dose of 10 mg to 200 mg. In some embodiments, eplerenone can be administered in a dose of 25 mg. For example, administration can be once daily via capsule in a single 25 mg capsule.

In some embodiments, the one or more symptoms of the dystrophinopathy is a respiratory symptom. Thus, the second therapeutic can be Idebenone. In some embodiments, Idebenone can be administered orally. In some embodiments, administration can be daily, weekly, or monthly. In some embodiments, the length of treatment can be weeks, months or years. In some embodiments, Idebenone can be administered in a dose of 250 mg/day to 2000 mg/day. In some embodiments, Idebenone can be administered in a dose of 900 mg/day. For example, administration can be three times a day, orally, wherein each oral administration is two tablets each of 150 mg.

In some embodiments, the second therapeutic is orthopedic management, endocrinologic management, gastrointestinal management, urologic management, or a combination thereof.

In some embodiments, the second therapeutic is transcutaneous electrical nerve stimulation (TENS). TENS can increase muscle strength, increase range of joint motions and/or improve sleep. In some embodiments, the TENS is applied using VECTTOR system. The VT-200, or VECTTOR system, delivers electrical stimulation via electrodes on the acupuncture points of a subject's feet/legs and hands/arms to provide symptomatic relief of chronic intractable pain and/or management of post-surgical pain. In some embodiments, nerve stimulator treatment (e.g. TENS) can be administered one time, two times, three times, four times, five times or more daily.

### 5.5.6 Dosing of Microdystrophin

Disclosed are methods of treatment of human patients having a dystrophinopathy (e.g. DMD or BMD) amenable to treatment with functional dystrophin and a second therapeutic effective to treat or ameliorate one or more symptoms of a dystrophinopathy, by peripheral, including intravenous, administration of an rAAV particle, including rAAV8 or rAAV9 particle, containing a construct encoding a microdystrophin described herein at a dosage of 5×10¹³ to 1×10¹⁵, including a dose of 2×10¹⁴ vg/kg. Doses can range from 1×10⁸ vector genomes per kg (vg/kg) to 1×10¹⁵ vg/kg. In some embodiments, the dose can be 3×10¹³, 1×10¹⁴, 3×10¹⁴, 5×10¹⁴ vg/kg. In some embodiments, the dose can be 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ vg/kg in combination with the second therapeutic. Therapeutically effective dosages are administered as a single dosage. Alternatively, multiple doses may be administered during the course of a treatment regimen (i.e., days, weeks, months, etc.).

The dosages are therapeutically effective, which can be assessed at appropriate times after the administration, including 12 weeks, 26 weeks, 52 weeks or more, and include assessments for improvement or amelioration of symptoms and/or biomarkers of the dystrophinopathy as known in the art and detailed herein. Recombinant vectors used for delivering the transgene encoding the microdystrophin are described herein. Such vectors should have a tropism for human muscle cells (including skeletal muscle, smooth muscle and/or cardiac muscle) and can include non-replicating rAAV, particularly those bearing an AAV8 capsid. The recombinant vectors, including vectors having the construct RGX-DYS1 or RGX-DYS5 (see FIG. 2) can be administered in any manner such that the recombinant vector enters the muscle tissue or CNS, including by introducing the recombinant vector into the bloodstream, including intravenous administration.

Subjects to whom such gene therapy is administered can be those responsive to gene therapy mediated delivery of a microdystrophin to muscles. In particular embodiments, the methods encompass treating patients who have been diagnosed with DMD or other muscular dystrophy disease, such as, Becker muscular dystrophy (BMD), myotonic muscular dystrophy (Steinert's disease), Facioscapulohumeral disease (FSHD), limb-girdle muscular dystrophy, X-linked dilated cardiomyopathy, or oculopharyngeal muscular dystrophy, or have one or more symptoms associated therewith, and identified as responsive to treatment with microdystrophin, or considered a good candidate for therapy with gene mediated delivery of microdystrophin. In specific embodiments, the patients have previously been treated with synthetic version of dystrophin and have been found to be responsive to one or more of synthetic versions of dystrophin. To determine responsiveness, the synthetic version of dystrophin (e.g., produced in human cell culture, bioreactors, etc.) may be administered directly to the subject.

Therapeutically effective doses of any such recombinant vector should be administered in any manner such that the recombinant vector enters the muscle (e.g., skeletal muscle or cardiac muscle), including by introducing the recombinant vector into the bloodstream. In specific embodiments, the vector is administered subcutaneously, intramuscularly or intravenously. Intramuscular, subcutaneous, or intravenous administration should result in expression of the soluble transgene product in cells of the muscle (including skeletal muscle, cardiac muscle, and/or smooth muscle). The expression of the transgene product results in delivery and maintenance of the transgene product in the muscle. Alternatively, the delivery may result in gene therapy delivery and expression of the microdystrophin in the liver, and the soluble microdystrophin product is then carried through the bloodstream to the muscles where it can impart its therapeutic effect.

Pharmaceutical compositions suitable for intravenous, intramuscular, subcutaneous or hepatic administration comprise a suspension of the recombinant vector comprising the transgene encoding microdystrophin in a formulation buffer comprising a physiologically compatible aqueous buffer. The formulation buffer can comprise one or more of a polysaccharide, a surfactant, polymer, or oil. The disclosed pharmaceutical compositions can comprise any of the microdystrophins, particularly the rAAV vectors comprising a transgene encoding the microdystrophins, disclosed herein and can be used in the disclosed methods.

For example, a pharmaceutical composition comprising a rAAV, including an rAAV8 comprising a transgene encoding RGX-DYS1, including the RGX-DYS1 construct having the nucleotide sequence of SEQ ID NO:20 (also known as RGX-DYS1) can be used in the disclosed methods. In some embodiments, a pharmaceutical composition can comprise a recombinant adeno-associated virus serotype 8 (AAV8) that contains a vector genome encoding a microdystrophin. The rAAV particles containing constructs encoding the microdystrophins disclosed herein, including RGX-DYS1 and RGX-DYS5 can be formulated in modified Dulbecco's phosphate buffered saline (DPBS) with sucrose buffer, which comprises 0.2 g/L potassium chloride, 0.2 g/L potassium phosphate monobasic, 1.2 g/L sodium phosphate dibasic anhydrous, 5.8 g/L sodium chloride, 40 g/L sucrose, and 0.01 g/L poloxamer 188, pH 7.4. The pharmaceutical composition can be supplied as a frozen suspension in sterile, single-use vials for intravenous (IV) administration. In some embodiments, the pharmaceutical composition can be filled into Crystal Zenith® (CZ) vials sealed with latex-free rubber stoppers and flip-off aluminum seals. In some embodiments, the pharmaceutical composition can be available in one configuration: 5.0 mL deliverable volume in a 10 mL vial.

The gene therapy vectors provided herein may be administered in combination with other treatments for muscular dystrophy, including corticosteroids, beta blockers and ACE inhibitors.

The disclosed methods of treatment can result in one of many endpoints indicative of therapeutic efficacy described herein. In some embodiments, the endpoints can be monitored 6 weeks, 12 weeks, 24 weeks, 30 weeks, 36 weeks, 42 weeks, 48 weeks, 1 year, 2 years, 3 years, 4 years or 5 years after the administration of a rAAV particle comprising a transgene that encodes one of the disclosed microdystrophins.

In some embodiments, creatine kinase activity can be used as an endpoint for therapeutic efficacy of the methods of treatment and administration disclosed herein. The creatine kinase activity can decrease in the subject relative to the level (of creatine kinase activity) prior to said administration. In some embodiments, the creatine kinase activity can decrease in the subject relative to the level (of creatine kinase activity) in the subject prior to treatment or relative to the level (of creatine kinase activity) in a non-treated subject having a dystrophinopathy (for example, a reference level identified in a natural history study). The creatine kinase activity measured in the human subject after administration of a rAAV with a transgene encoding microdystrophin can be to a control value which can be the creatine kinase activity in the subject prior to administration, creatine kinase activity in a subject with a dystrophinopathy that has not be treated, creatine kinase activity in a subject that does not have a dystrophinopathy, creatine kinase activity in a standard.

In some embodiments, provided are methods of treating a dystrophinopathy, including DMD and BMD, by peripheral, including intravenous administration of an rAAV vector containing a microdystrophin construct disclosed herein, including RGX-DYS1, at dosages disclosed herein, including dosages of 5×10¹³ genome copies/kg to 1×10¹⁵ genome copies/kg, including 1×10¹⁴ genome copies/kg, 2×10¹⁴ genome copies/kg or 3×10¹⁴ genome copies/kg genome copies/kg, wherein the in creatine kinase activity is reduced by 0.5 fold to 1.5 fold at least 12 weeks, 26 weeks, or 52 weeks after administration of the rAAV therapeutic. In some embodiments, a decrease in creatine kinase activity can be a decrease of 1000 to 10,000 units/liter compared to a control or the value measured in the subject amount prior to administration of the therapeutic. In some embodiments, an amount of 1000, 2000, 3000, 4000, or 5000 units/liter in the after administration endpoint is indicative of a decrease.

In some embodiments, reduction in lesions in a gastrocnemius muscle (or other muscle) can be used as an endpoint measure for therapeutic efficacy for the methods of treatment and administration disclosed herein. The lesions in a gastrocnemius muscle can decrease in the subject relative to the level (of lesions in the gastrocnemius muscle) prior to said administration of rAAV with a transgene encoding microdystrophin. In some embodiments, the lesions in the gastrocnemius muscle can decrease in the subject relative to the level (of lesions in the gastrocnemius muscle) in a non-treated subject having a dystrophinopathy. The comparison of lesions in the gastrocnemius muscle can be to a standard, wherein the standard is a number or set of numbers that represent the lesions in a subject that does not have a dystrophinopathy or the lesions in a non-treated subject having a dystrophinopathy. Thus, in some embodiments, the comparison of lesions in the gastrocnemius muscle after administration of a rAAV with a transgene encoding microdystrophin can be to a control subject. The control can be the lesions in the gastrocnemius muscle in the subject prior to administration lesions in the gastrocnemius muscle in a subject with a dystrophinopathy that has not be treated, lesions in the gastrocnemius muscle in a subject that does not have a dystrophinopathy, or lesions in the gastrocnemius muscle in a standard.

In some embodiments, the lesions in the gastrocnemius muscle of the subject are assessed using magnetic resonance imaging (MRI). MRI can be a good tool for imagine muscles, ligaments, and tendons, therefore, muscle disorders can be detected and/or characterized using MRI. In some embodiments, provided are methods of treating a dystrophinopathy, including DMD and BMD, by peripheral, including intravenous administration of an rAAV vector containing a microdystrophin construct disclosed herein, including RGX-DYS1, at dosages disclosed herein, including dosages of 5×10¹³ genome copies/kg to 1×10¹⁵ genome copies/kg including 1×10¹⁴ genome copies/kg, 2×10¹⁴ genome copies/kg or 3×10¹⁴ genome copies/kg, resulting in a decrease of lesions in gastrocnemius muscle after administration is about 1-100%, 2-50%, or 3-10% compared a control, for example, compared to the lesions in the gastrocnemius muscle of the subject prior to said administration. For example, a subject treated with a rAAV with a transgene encoding microdystrophin can have 1, 5, 10, 15, 20, 25, 30, 35, 40, 45, or 50% or greater decrease in lesions compared to a control.

In some embodiments, gastrocnemius muscle volume (or muscle volume of any other muscle) can be used as an endpoint for treatment efficacy. The gastrocnemius muscle volume can decrease in the subject relative to the level (of gastrocnemius muscle volume) prior to said administration of rAAV with a transgene encoding microdystrophin. In some embodiments, the gastrocnemius muscle volume can decrease in the subject relative to the level (of gastrocnemius muscle volume) in a subject that does not have a dystrophinopathy. In some embodiments, the gastrocnemius muscle volume can decrease in the subject relative to the level (of gastrocnemius muscle volume) in a non-treated subject having a dystrophinopathy. The comparison of gastrocnemius muscle volume can be to a standard, wherein the standard is a number or set of numbers that represent the volume in a subject that does not have a dystrophinopathy or the volume in a non-treated subject having a dystrophinopathy. Thus, in some embodiments, the comparison of gastrocnemius muscle volume after administration of a rAAV with a transgene encoding microdystrophin can be to a control. The control can be the gastrocnemius muscle volume in the subject prior to administration, gastrocnemius muscle volume in a subject with a dystrophinopathy that has not be treated, gastrocnemius muscle volume in a subject that does not have a dystrophinopathy, or gastrocnemius muscle volume in a standard.

In some embodiments, the gastrocnemius muscle volume of the subject can be assessed using MRI. In some embodiments, provided are methods of treating a dystrophinopathy, including DMD and BMD, by peripheral, including intravenous administration, of an rAAV vector containing a microdystrophin construct disclosed herein, including RGX-DYS1, at dosages disclosed herein, including dosages of 5×10¹³ genome copies/kg to 1×10¹⁵ genome copies/kg including 1×10¹⁴ genome copies/kg, 2×10¹⁴ genome copies/kg or 3×10¹⁴ genome copies/kg, resulting in a decrease in gastrocnemius muscle volume of about 1-100%, 2-50%, or 3-20% compared a control, for example, compared to the gastrocnemius muscle volume prior to said administration. In some embodiments, a decrease of gastrocnemius muscle volume after administration of a rAAV comprising a transgene that encodes microdystrophin can be about 2-400 mm³, 5-200 mm³, or 20-100 mm³ compared a control. For example, a subject treated with a rAAV with a transgene encoding microdystrophin can have 10, 20, 30, 40, 50, 60, 70, 80, 90,100,110, 120, 130, 140, or 150 mm³ or greater decrease in gastrocnemius muscle volume compared to a control.

In some embodiments, a fat fraction of muscle can be used as an endpoint for therapeutic efficacy of the methods of administering rAAV therapeutics disclosed herein. The muscle can be muscles in the pelvic girdle and thigh (gluteus maximus, adductor magnus, rectus femoris, vastus lateralis, vastus medialis, biceps femoris, semitendinosus, and gracilis). The fat fraction of muscle can decrease in the subject relative to the level (of fat fraction of muscle) prior to said administration of rAAV with a transgene encoding microdystrophin as disclosed herein. In some embodiments, the fat fraction of muscle can decrease in the subject relative to the level (of fat fraction of muscle) in a non-treated subject having a dystrophinopathy. The comparison of fat fraction of muscle can be to a standard, wherein the standard is a number or set of numbers that represent the amount or percent of fat fraction of muscle in a subject that does not have a dystrophinopathy or the amount or percent in a non-treated subject having a dystrophinopathy. Thus, in some embodiments, the comparison of fat fraction of muscle after administration of a rAAV with a transgene encoding microdystrophin can be to a control. The control can be the fat fraction of muscle in the subject prior to administration, fat fraction of muscle in a subject with a dystrophinopathy that has not be treated, fat fraction of muscle in a subject that does not have a dystrophinopathy, or fat fraction of muscle of a standard.

In some embodiments, the fat fraction of muscle of the subject are assessed using magnetic resonance imaging (MRI). In some embodiments, provided are methods of treating a dystrophinopathy, including DMD and BMD, by peripheral, including intravenous administration of an rAAV vector containing a microdystrophin construct disclosed herein, including RGX-DYS1, at dosages disclosed herein, including dosages of 5×10¹³ genome copies/kg to 1×10¹⁵ genome copies/kg including 1×10¹⁴ genome copies/kg, 2×10¹⁴ genome copies/kg, and 3×10¹⁴ genome copies/kg, results in a decrease of fat fraction of muscle after administration of a rAAV comprising a transgene that encodes microdystrophin can be about 1-100%, 2-50%, or 3-10% compared a control, for example, compared to the fat fraction of muscle prior to said administration. For example, a subject treated with a rAAV with a transgene encoding microdystrophin can have 1, 5, 10, 15, 20, 25, 30, 35, 40, 45, or 50% or greater decrease in fat fraction of muscle compared to a control.

In some embodiments, T2-relaxation time of lesions in muscle can be used as an endpoint for treatment. The muscle can be any muscle, for example, gastrocnemius. The T2-relaxation time of lesions in muscle can decrease in the subject relative to the level (of T2-relaxation time of lesions in muscle) prior to said administration of rAAV with a transgene encoding microdystrophin. In some embodiments, the T2-relaxation time of lesions in muscle can decrease in the subject relative to the level (of T2-relaxation time of lesions in muscle) in a subject that does not have a dystrophinopathy. In some embodiments, the T2-relaxation time of lesions in muscle can decrease in the subject relative to the level (of T2-relaxation time of lesions in muscle in a non-treated subject having a dystrophinopathy. The comparison of T2-relaxation time of lesions in muscle can be to a standard, wherein the standard is a number or set of numbers that represent the T2-relaxation time of lesions in muscle in a subject that does not have a dystrophinopathy or the T2-relaxation time of lesions in muscle in a non-treated subject having a dystrophinopathy. Thus, in some embodiments, the comparison of T2-relaxation time of lesions in muscle after administration of a rAAV with a transgene encoding microdystrophin can be to a control. The control can be the T2-relaxation time of lesions in muscle in the subject prior to administration, T2-relaxation time of lesions in muscle in a subject with a dystrophinopathy that has not be treated, T2-relaxation time of lesions in muscle in a subject that does not have a dystrophinopathy, or T2-relaxation time of lesions in muscle in a standard.

In some embodiments, the T2-relaxation time of lesions in muscle of the subject is assessed using magnetic resonance imaging (MRI). In some embodiments, a decrease of T2-relaxation time of lesions in muscle after administration of a rAAV comprising a transgene that encodes microdystrophin can be about 1-100%, 5-50%, or 10-30% compared a control, for example, compared to the T2-relaxation time of lesions in muscle prior to said administration. In some embodiments, provided are methods of treating a dystrophinopathy, including DMD and BMD, by peripheral, including intravenous administration, of an rAAV vector containing a microdystrophin construct disclosed herein, including RGX-DYS1, at dosages disclosed herein, including dosages of 5×10¹³ genome copies/kg to 1×10¹⁵ genome copies/kg, including 1×10¹⁴ genome copies/kg, 2×10¹⁴ genome copies/kg, and 3×10¹⁴ genome copies/kg, which results in a decrease of T2-relaxation time of lesions in muscle after administration of a rAAV comprising a transgene that encodes microdystrophin can be about 1-500 milliseconds (ms), 1-400 ms, 1-300 ms, 1-200 ms, 1-100 ms, 1-50 ms, 1-25 ms, 1-10 ms compared a control. In some embodiments, a decrease of T2-relaxation time of lesions in muscle after administration of a rAAV comprising a transgene that encodes microdystrophin can be about 2 to 8 ms. For example, a subject treated with a rAAV with a transgene encoding microdystrophin can have a decrease of T2-relaxation time of lesions in muscle compared to a control of 2, 4, 6, 8, 10, 12, 14, 16, 18, 20, 22, 24, 26, 28, 30, or more ms.

In some embodiments, gait score can be used as an endpoint for treatment. The gait score can be about −1 to 2 after administration of a rAAV comprising a transgene that encodes microdystrophin. In some embodiments, the gait score can be about 1 after administration of a rAAV comprising a transgene that encodes microdystrophin.

In some embodiments, the North Star Ambulatory Assessment (NSAA) can be used as an endpoint for treatment. The NSAA of the treated subject can be compared to NSAA prior to administration of rAAV comprising a transgene that encodes microdystrophin. The NSAA of the treated subject can be compared to NSAA in a subject that does not have a dystrophinopathy. The NSAA of the treated subject can be compared to a non-treated subject having a dystrophinopathy. The NSAA of the treated subject can be compared to a standard, wherein the standard is a score or set of scores that represent the NSAA in a subject that does not have a dystrophinopathy or the NSAA in a non-treated subject having a dystrophinopathy.

In some embodiments, the NSAA of the subject treated with rAAV comprising a transgene that encodes microdystrophin increased compared to the NSAA score prior to said administration or compared to any of the NSAA comparisons described above. In some embodiments, the increase can be from 0 to 1, 0 to 2 or from 1 to 2.

In some embodiments, any of the 17 items used in the NSAA can be used as an individual endpoint of treatment. For example, any of the following can be endpoints for treatment, stand, walk, stand up from chair, stand on one leg (right), stand on one leg (left), climb box step (right leg first), climb box step (left leg first), descend box step (right leg first), descend box step (left leg first), lying to sitting, rise from floor, lift head, stand on heels, jump, hop right leg, hop left leg, and run (10m). Each of these assessments are well known in the art. An improvement in one or more of these endpoints can be seen after administration of rAAV comprising a transgene that encodes microdystrophin. One of skill in the art would understand what is considered an improvement. For example, in some embodiments a decrease in the amount of time it takes the subject treated with of rAAV comprising a transgene that encodes microdystrophin to stand, run/walk a determined distance, and/or climb a set number of stairs can be achieved.

A decrease in the amount of time it takes a subject administered rAAV comprising a transgene that encodes microdystrophin to run/walk a determined distance can be 1, 2, 3, 4, 5, 6, 7, 8, 9, 10, 20, 30, 40, 50% or more compared to a control, for example, the amount of time it took the subject prior to administration of the rAAV. In some embodiments, the determined distance to run and/or walk can be 10 meters.

A decrease in the amount of time it takes a subject administered rAAV comprising a transgene that encodes microdystrophin to stand can be 1, 2, 3, 4, 5, 6, 7, 8, 9, 10, 20, 30, 40, 50% or more compared to a control, for example, the amount of time it took the subject prior to administration of the rAAV.

A decrease in the amount of time it takes a subject administered rAAV comprising a transgene that encodes microdystrophin to climb a set number of stairs can be 1, 2, 3, 4, 5, 6, 7, 8, 9, 10, 20, 30, 40, 50% or more compared to a control, for example, the amount of time it took the subject prior to administration of the rAAV. In some embodiments, the set number of stairs can be, but is not limited to, 1, 2, 3, 4, 5, 6, 7, 8, 9, or 10.

In some embodiments, questionnaires can be used as an endpoint for treatment. For example, Pediatric Outcomes Data Collection Instrument (PODCI) questionnaire can be used to quantify functional abilities of a subject before and after treatment with rAAV comprising a transgene encoding microdystrophin.

### 5.5.7 Cardiac Output

Although skeletal muscle symptoms are considered the defining characteristic of DMD, patients most commonly die of respiratory or cardiac failure. DMD patients develop dilated cardiomyopathy (DCM) due to the absence of dystrophin in cardiomyocytes, which is required for contractile function. This leads to an influx of extracellular calcium, triggering protease activation, cardiomyocyte death, tissue necrosis, and inflammation, ultimately leading to accumulation of fat and fibrosis. This process first affects the left ventricle (LV), which is responsible for pumping blood to most of the body and is thicker and therefore experiences a greater workload. Atrophic cardiomyocytes exhibit a loss of striations, vacuolization, fragmentation, and nuclear degeneration. Functionally, atrophy and scarring leads to structural instability and hypo kinesis of the LV, ultimately progressing to general DCM. DMD may be associated with various ECG changes like sinus tachycardia, reduction of circadian index, decreased heart rate variability, short PR interval, right ventricular hypertrophy, S-T segment depression and prolonged QTc.

Gene therapy treatment provided herein can slow or arrest the progression of DMD and other dystrophinopathies, particularly to reduce the progression of or attenuate cardiac dysfunction and/or maintain or improve cardiac function. Efficacy may be monitored by periodic evaluation of signs and symptoms of cardiac involvement or heart failure that are appropriate for the age and disease stage of the trial population, using serial electrocardiograms, and serial noninvasive imaging studies (e.g., echocardiography or cardiac magnetic resonance imaging (CMR)). CMR may be used to monitor changes from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), inflammation, and fibrosis. ECG may be used to monitor conduction abnormalities and arrhythmias. In particular, ECG may be used to assess normalization of the PR interval, R waves in V1, Q waves in V6, ventricular repolarization, QS waves in inferior and/or upper lateral wall, conduction disturbances in right bundle branch, QT C, and QRS.

Accordingly, provided are nucleic acid compositions, including compositions comprising gene expression cassettes and viral vectors, comprising a nucleic acid encoding a microdystrophin protein disclosed herein, and methods of administering those compositions that improve or maintain cardiac function or slow the loss of cardiac function, for example, by preventing reductions in decreasing LVEF below 45% and/or normalization of function (LVFS≥28%) as measured by serial electrocardiograms, and/or serial noninvasive imaging studies (e.g., echocardiography or cardiac magnetic resonance imaging (CMR)). Measurements may be compared to an untreated control or to the subject prior to treatment with the nucleic acid composition. Alternatively, the nucleic acid compositions described here in and the methods of administering nucleic acid compositions results in an improvement in cardiac function or reduction in the loss of cardiac function as assessed by monitoring changes from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), inflammation, and fibrosis. ECG may be used to monitor conduction abnormalities and arrhythmias. In particular, ECG may be used to assess normalization of the PR interval, R waves in V1, Q waves in V6, ventricular repolarization, QS waves in inferior and/or upper lateral wall, conduction disturbances in right bundle branch, QT C, and QRS.

In some embodiments, cardiac function and/or pulmonary function can be used as an endpoint for assessment of therapeutic efficacy of the administration. The cardiac function and/or pulmonary function can improve or increase in the subject relative to the level (of cardiac function and/or pulmonary function) prior to said administration. In some embodiments, the cardiac function and/or pulmonary function can improve or increase in the subject relative to the level (of cardiac function and/or pulmonary function) in a subject that does not have a dystrophinopathy. In some embodiments, the cardiac function and/or pulmonary function can decrease in the subject relative to the level (of cardiac function and/or pulmonary function) in a non-treated subject having a dystrophinopathy. The comparison of cardiac function and/or pulmonary function can be to a standard, wherein the standard is a number or set of numbers that represent the cardiac function and/or pulmonary function in a subject that does not have a dystrophinopathy or the cardiac function and/or pulmonary function in a non-treated subject having a dystrophinopathy. Thus, in some embodiments, the comparison of cardiac function and/or pulmonary function after administration of a rAAV with a transgene encoding microdystrophin can be to a control. The control can be the cardiac function and/or pulmonary function in the subject prior to administration, cardiac function and/or pulmonary function in a subject with a dystrophinopathy that has not be treated, cardiac function and/or pulmonary function in a subject that does not have a dystrophinopathy, cardiac function and/or pulmonary function in a standard.

In some embodiments, an improvement or increase in cardiac function and/or pulmonary function is 1 to 100% compared to a control, for example, compared to the subject prior to administration of rAAV comprising a transgene encoding microdystrophin. In some embodiments, cardiac function can be measured using impedance, electric activities, and calcium handling.

### 5.5.8 Central Nervous System

A portion of patients with DMD can also have epilepsy, learning and cognitive impairment, dyslexia, neurodevelopment disorders such as attention deficit hyperactive disorder (ADHD), autism, and/or psychiatric disorders, such as obsessive-compulsive disorder, anxiety or sleep disorders.

The goal of gene therapy treatments disclosed herein can be to improve cognitive function or alleviate symptoms of epilepsy and/or psychiatric disorders. Efficacy may be assessed by periodic evaluation of behavior and cognitive function that are appropriate for the age and disease stage of the trial population and or by quantifying and qualifying seizure events.

Accordingly, provided are nucleic acid compositions and methods of administering the microdystrophin gene therapy compositions that improve cognitive function, reduce the occurrence or severity of seizures, alleviate symptoms of ADHD, obsessive-compulsive disorder, anxiety and/or sleep disorders.

### 5.5.9 Patient Primary Endpoints

The efficacy of the compositions, including the dosage of the composition, and methods described herein may be assessed in clinical evaluation of subjects being treated. Patient primary endpoints may include monitoring the change from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), change from baseline in the NSAA, change from baseline in the Performance of Upper Limp (PUL) score, and change from baseline in the Brooke Upper Extremity Scale score (Brooke score), change from baseline in grip strength, pinch strength, change in cardiac fibrosis score by MRI, change in upper arm (bicep) muscle fat and fibrosis assessed by MRI, measurement of leg strength using a dynamometer, walk test 6-minutes, walk test 10-minutes, walk analysis—3D recording of walking, change in utrophin membrane staining via quantifiable imaging of immunostained biopsy sections, and a change in regenerating fibers by measuring (via muscle biopsy) a combination of fiber size and neonatal myosin positivity. See, for example, Mazzone E et al, North Star Ambulatory Assessment, 6-minute walk test and timed items in ambulant boys with Duchenne muscular dystrophy. Neuromuscular Disorders 20 (2010) 712-716.; Abdelrahim Abdrabou Sadek, et al, Evaluation of cardiac functions in children with Duchenne Muscular Dystrophy: A prospective case-control study. Electron Physician (2017) November; 9(11): 5732-5739; Magrath, P. et al, Cardiac MRI biomarkers for Duchenne muscular dystrophy. BIOMARKERS IN MEDICINE (2018) VOL. 12, NO. 11.; Pane, M. et al, Upper limb function in Duchenne muscular dystrophy: 24 month longitudinal data. PLoS One. 2018 Jun. 20; 13(6):e0199223.

### 6. EXAMPLES

### 6.1 Example 1—Construction of Microdystrophin (DMD) Gene Expression Cassettes for Insertion of Cis Plasmids

DMD constructs encode microdystrophins with the core backbone: 5′(N-terminus)-ABD-H1-R1-R2-R3-H3-R24-H4-CR-3′ (C-terminus) (FIG. 2), but which differ in the presence and length of the C-terminus (CT). The microdystrophin encoded by RGX-DYS1 has as the C terminus the proximal 194 amino acids of the wild type DMD protein C-terminus domain (wild type DMD amino acid sequence is SEQ ID NO: 95), the RGX-DYS3 encodes a microdystrophin that does not have a C-terminus, and RGX-DYS5 encodes a microdystrophin with 140 amino acids of the C-terminal domain (SEQ ID NO:83), which contains an α1-syntrophin binding site but not a dystrobrevin binding site (see FIGS. 1A and 1B). The constructs include the Spc5-12 promoter (SEQ ID NO:39) and smPA regulatory sequences, and RGX-DYS3 includes the VH4 intron sequence (SEQ ID NO:41). All were cloned into Cis plasmids flanked by ITRs. All DNA sequences encoding the DMD genes are codon-optimized and CpG depleted.

### 6.1.1. Recombinant Engineering of RGX-DYS1, RGX-DYS3 and RGX-DYS5 Transgene

In brief, the human codon-optimized and CpG depleted nucleotide sequence of a microdystrophin construct in RGX-DYS1 (SEQ ID NO:1) as shown in FIG. 2 encoding N-terminal-ABD1-H1-R1-R2-R3-H3-R24-H4-CR-CT-C-terminal was synthesized using GeneArt Gene Synthesis (Invitrogen, Thermo Fisher, Waltham, MA). The desired C-terminus was made by site directed mutagenesis using the following two primers: 5′: TGA CTC GAG AGG CCT AAT AAA GAG C (SEQ ID NO: 43), 3′: CCT TGG AGA CTG TGG AGA GGT G (SEQ ID NO: 44). The construct includes a synthetic muscle promoter (e.g. spc5-12; SEQ ID NO:39), and a small poly(A) signal sequence (sm pA (SEQ ID NO:42) and has a nucleotide sequence of SEQ ID NO:53.

The construct RGX-DYS3 (FIG. 2) was engineered encoding the microdystrophin of the RGX-DYS1 construct detailed above with a small portion (48 amino acids; SEQ ID NO:91) of the CT domain (the microdystrophin having the amino acid sequence of SEQ ID NO:2). This construct includes the Spc5-12 promoter the sm pA poly A sequence and the VH4 intron at the 5′end of the microdystrophin coding sequence. The transgene construct has a nucleotide sequence of SEQ ID NO:21.

The construct RGX-DYS5 (FIG. 2) was engineered to encode the microdystrophin DYS5 (amino acid sequence of SEQ ID NO: 79), which is the DYS1 microdystrophin except that the C-terminal domain is truncated and is 140 amino acids in length (SEQ ID NO: 83). The construct includes the spc5-12 promoter and sm pA signal sequence and has a nucleotide sequence of SEQ ID NO: 82.

Plasmid RGX-DYS5 was created by replacing the long version of C-terminus of DYS1 in plasmid RGX-DYS1 with an intermediate length version of the C-terminus tail. In brief, a gBlock-DMD-1.5 tail was synthesized from Integrated DNA technologies containing the intermediate version of the C-terminus flanked by EcoRV and NheI sites and 17 bp of the overlapping sequence of the RGX-DYS1 plasmid. The source plasmid RGX-DYS1 was digested with restriction enzymes NheI and EcoRV (New England Biolabs), and then in-fusion ligated with the gBlock-DMD1.5 Tail. The final plasmid RGX-DYS5 was confirmed by enzyme digestion and subsequent sequencing. RGX-DYS2 and RGX-DYS4 were constructed similarly, each encoding the same microdystrophin protein as RGX-DYS1, with RGX-DYS2 containing the VH4 intron downstream of the promoter and RGX-DYS4 having a truncated muscle-specific promoter.

The constructs were all inserted into cis plasmids such that they are positioned to be flanked by ITRs(nucleotide sequence of SEQ ID NO. 82). The RGX-DYS1 cassette comprises a nucleotide sequence of SEQ ID NO:20 encoding the DYS1 microdystrophin, the RGX-DYS3 cassette comprises a nucleotide sequence of SEQ ID NO:21 encoding the DYS3 microdystrophin and the RGX-DYS5 cassette comprises a nucleotide sequence of SEQ ID NO:81 encoding the DYS5 microdystrophin (see also Table 5). Table 10 provides the nucleotide sequences of the artificial genomes (including the flanking ITR sequences which are indicated in lower case letters) of RGX-DYS1 (SEQ ID NO: 53), RGS-DYS3 (SEQ ID NO: 55) and RGX-DYS5 (SEQ ID NO 82).

The length and expression of the protein was confirmed by expression of the different plasmids in C2C12 cells and assaying cell lysates by western blot.

To examine the packaging efficiency of RGX-DYS5, RGX-DYS5 was packaged into AAV8 vector using HEK293 cells, and the titer of the AAV8 packaged vector RGX-DYS5 was determined following shake flask culture and affinity purification. Average titer was higher than AAV8 packaged RGX-DYS1 and comparable to AAV8 packaged RGX-DYS3 in these benchtop production runs. (Data not shown.)

### 6.2 Example 2: Comparative Study of Construct Expression in Mdx Mice

### 6.2.1 p-Dys Expression Comparisons by Western Blot, mRNA Expression and DNA Vector Copy Numbers

Data and samples described in this example related to RGX-DYS1 experiments were collected following treatment as described in Section 6.4 (Proof of Concept; Example 5) infra (n=13 mice dosed with AAV8-RGX-DYS1). In vivo testing of AAV8-RGX-DYS3 and AAV8-RGX-DYS5 vectors was performed in 13 male C57BL/10ScSn-Dmd^{mdx}/J (mdx) mice. All vectors were systemically delivered into the 5-weeks-old mdx mice by tail vein injection at 2E14 vg/kg dosage (n=5 for group 1, AAV8-RGX-DYS3; n=5 for group 2, AAV8-RGX-DYS5; n=3, mdx negative (no dosing) control). Animals ranged from 15.9 g to 22.0 g in weight on the day of dosing. At 6 weeks post-vector administration, blood was collected for serum and animals were euthanized and underwent necropsy for collection of tissues. Major skeletal muscles including gastrocnemius (Gas), tibialis anterior (TA), diaphragm, triceps, quadriceps, heart, liver and major organs were collected and snap frozen in isopentane/liquid nitrogen double bath and placed into pre-chilled cryotubes.

The body weights for each animal were recorded two times weekly, and the average change in weight for each group was calculated. All animals gained weight, as expected, over the 7 week period except for one animal.

Experiments with the RGX-DYS1 treated mice were performed at different facilities from the experiments for the RGX-DYS3 and RGX-DYS5 treated mice.

Microdystrophin protein expression from gastrocnemius muscle, as collected from treated mdx mice, was examined by western blot. Briefly, 20 to 30 mg of tissues were homogenized in protein lysis buffer (15% SDS, 75 mM Tri-HCl pH6.8, proteinase inhibitor, 20% glycerol, 5% beta-mercaptoethanol) (Bead Mill homogenizer Bead Ruptor 12, SKU:19050A, OMNI International). After homogenizing, the samples were spun down for 5 mins at top speed at room temperature, and the supernatants were subjected to protein quantification. The protein stock supernatants were quantified using Qubit protein assay kit (Catalog #Q33211, ThermoFisher Scientific). Total protein concentration per stock was calculated, then 20 μg of protein stock supernatant was loaded onto an SDS-PAGE gel. Western blot was performed using a primary anti-dystrophin antibody (MANEX1011B(1C7), Developmental Studies Hybridoma Bank) at 1:1000 dilution, and the secondary antibody applied was goat anti-mouse IgG2a conjugate to horseradish peroxidase (HRP) (Thermo Fisher Scientific, Cat. No. 62-6520). α1-actin serves as the loading control in each lane of the gel. For anti-al-actin blot, rabbit polyclonal anti-α1-actin antibody (PA5-78715, Thermo Fisher) was used at a dilution factor of 1:10,000, and the secondary goat anti-rabbit antibody (Thermo Fisher Scientific, Cat. No. 31460) was used at 1:20,000. Protein signal was detected using ECL Prime Western Blotting Detection Reagent (per Manufacturer's instructions; AMERSHAM, RPN2232) and quantified by densitometry guided by Image Lab software (Bio-Rad).

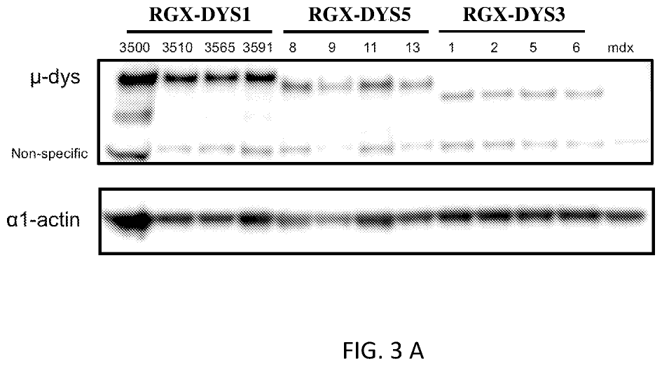

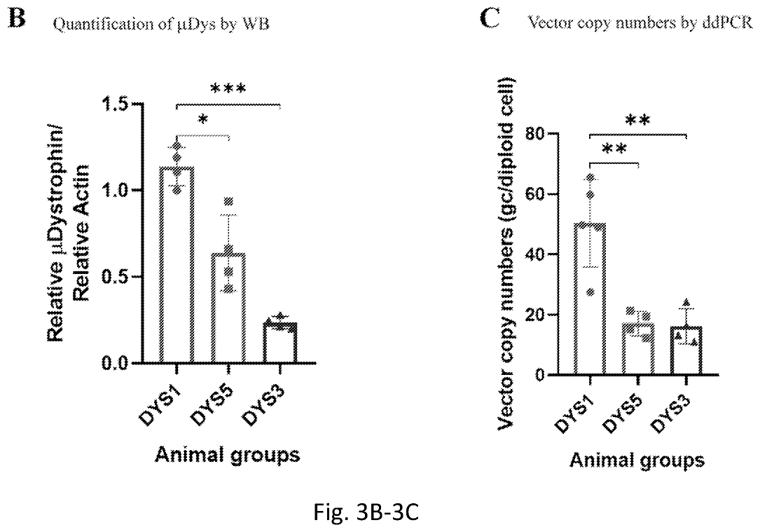

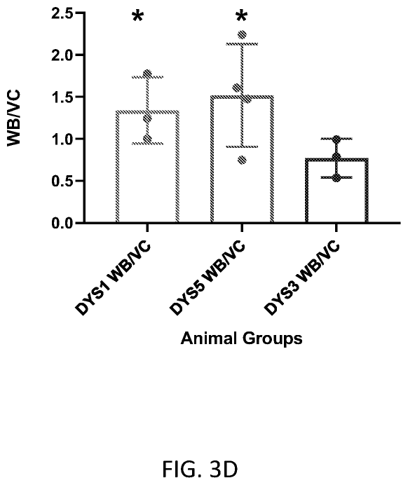

Western blot results (FIG. 3A) revealed several observations: First, the estimated size of each microdystrophin protein corresponds well to its observed migration on the gel, e.g. RGX-DYS1 microdystrophin protein was 148 kDa, while the size of RGX-DYS5 and RGX-DYS3 proteins were 142 kDa and 132 kDa, respectively. Second, the intensity of the bands was different for each protein present in the gastrocnemius muscle tissue. The longer version microdystrophin, RGX-DYS1 vector, displayed the strongest transgene expression, followed by the intermediate version RGX-DYS5 and shorter version RGX-DYS3 (and FIGS. 3A and 3B). The difference in microdystrophin expression level among those three constructs could be due to either variation in AAV vector genome level or protein stability of different lengths of microdystrophin constructs.

To elucidate genome copies per cell, ddPCR was performed to examine AAV-μ-dys vector genome copy numbers in those tissues, wherein the copy number of delivered vector in a specific tissue per diploid cell was calculated as:

$$\frac{ve ctor copy number}{endogenous control} \times 2 ..$$

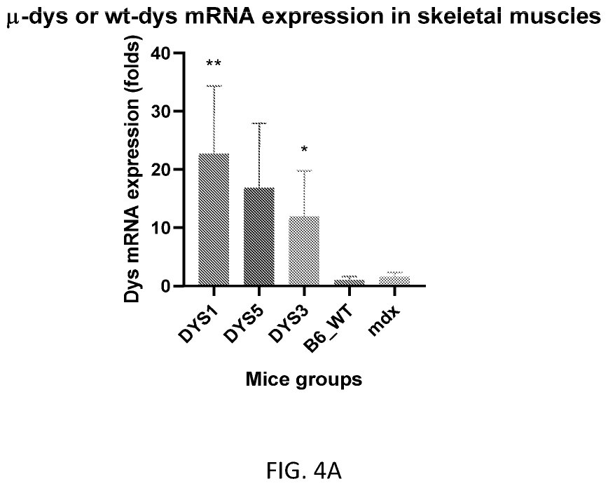

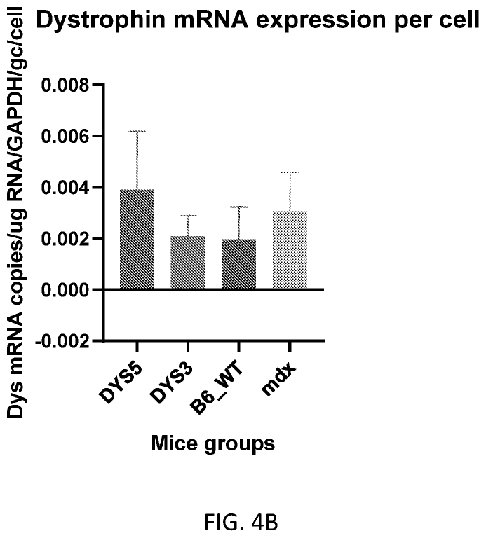

As displayed in FIG. 3C, the RGX-DYS1 vector-delivered endogenous control tissues indeed had higher vector genome copy numbers (50±14 gc/cell) than RGX-DYS5 (17±4 gc/cell) and RGX-DYS3 (16±5 gc/cell) vector-delivered tissues (values were normalized to glucagon genome copies). The relative microdystrophin expression was then compared to vector copy numbers. As shown in FIG. 4, the expression of relative microdystrophin in RGX-DYS1-treated muscle (1.33±0.39) and RGX-DYS5-treated muscle (1.774±0.40) were all significantly higher than the RGX-DYS3-treated muscle (0.77±0.22, p<0.05, n=3 to 5). This data indicates that the longer versions of microdystrophin (having a C-terminus) generated by RGX-DYS1 and RGX-DYS5 vectors render better stability of microdystrophin protein in muscle cells in vivo.

Additionally, the mRNA expression of micro and wild-type (WT)-dystrophin in skeletal muscle in untreated wild-type B6 and mdx mice, compared to treated mice, was measured with ddPCR. Total RNA were extracted from the muscle tissue using RNeasy Fibrous Tissue Mini Kit (REF 74704, Qiagen). cDNA was synthesized using High-capacity cDNA reverse transcription kit with RNAse inhibitor (Ref 4374966, Applied Biosystems by Thermo Fisher Scientific). The RNA concentration was measured using a Nanodrop spectrophotometer. The copy numbers of microdystrophin, WT-dystrophin, and endogenous control Glyceraldehyde 3-phosphate dehydrogenase (GAPDH) mRNA were measured using digital PCR (Naica Crystal Digital PCR system, Stilla technologies). Primers and probe against mouse WT-dystrophin (mm01216951_m1, Thermo Fisher Scientific)(also described in the biodistribution study above in Section 6.5 (Example 5)), and mouse GAPDH (mm99999915_g1, Thermo Fisher Scientific) were commercially available. As shown in FIG. 4A, the relative WT-dystrophin transcript in the naïve B6 mice was 1±0.64, and the WT-dystrophin mRNA expression in mdx mice was 1.55±0.77 (p=0.15, n=4). The relative microdystrophin mRNA in treated animals were as follows: RGX-DYS1-treated muscle, 22.66±11.6 (p<0.01, n=5); RGX-DYS5-treated, 16.83±11.07 (p=0.06, n=3) and RGX-DYS3 treated muscle, 11.87±7.90 (p<0.05, n=4). This data indicated that delivery of the microdystrophin vectors in RGX-DYS1, RGX-DYS5, and RGX-DYS3 groups all generated much higher microdystrophin transcripts than the wild-type level. Furthermore, microdystrophin mRNA copy numbers were normalized to AAV vector genome copy numbers per cell, and WT-dystrophin mRNA was normalized to genome copy numbers per cell (2 copies/cell), in addition to GAPDH normalization. As shown in FIG. 4B, all groups displayed essentially similar levels of mRNA expression on a per genome basis (n=3 to 5, p>0.05). This indicated that the muscle-specific Spc5-12 promoter driving expression of the AAV-microdystrophin transgenes was as potent as the native dystrophin promoter in mouse skeletal muscle cells.

### 6.2.2 Microdystrophin Expression by Immunofluorescence (IF) Staining and Dystrophin-Associated Protein Complex (DAPC) Association

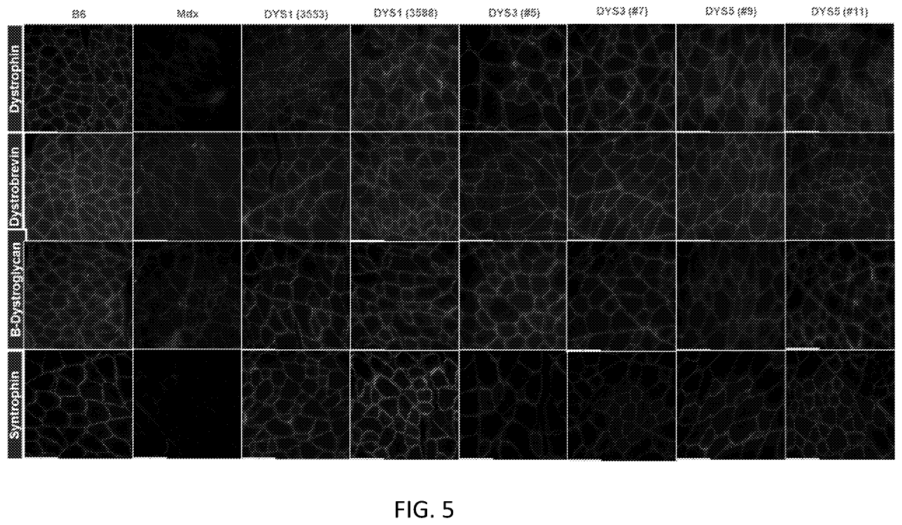

Next, immunofluorescent (IF) staining was performed to examine expression of dystrophin and dystrophin associated protein complexes including dystrobrevin, β-dystroglycan, syntrophin, and nNos on gastrocnemius muscles from different groups. The IF staining protocol and antibodies applied were as previously described in Section 6.2 hereinabove (Example 2). As shown in FIG. 5, the dystrophin protein and examined DAPC proteins were all absent in the untreated mdx muscle, while they were strongly present on the wild-type B6 muscle membrane. For all three treated groups, microdystrophin protein was expressed on nearly 100% muscle fibers and they were indistinguishable amongst the different treatment groups. The three treatment groups displayed restoration of dystrobrevin expression on muscle membranes with a very similar pattern observed. For 0-dystroglycan staining, the muscles in the RGX-DYS1-treated group displayed a more uniform and more intense β-dystroglycan staining (expression).

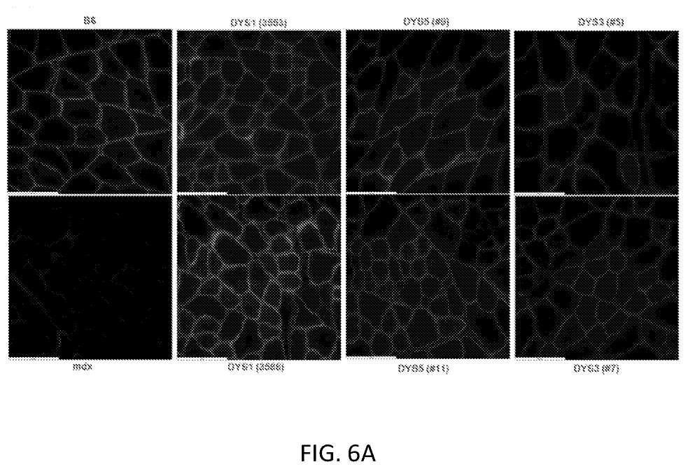

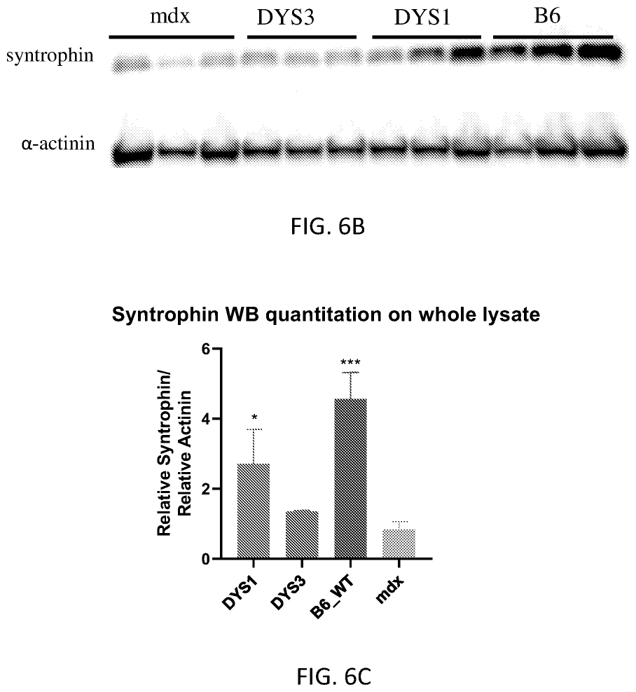

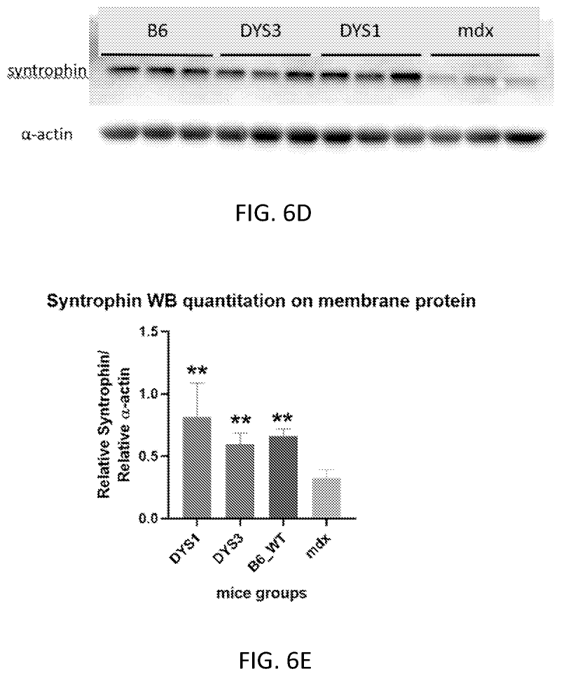

The more dramatic difference amongst the treatment groups was observed in syntrophin staining. The expression of syntrophin on muscle membrane was much enhanced in RGX-DYS1 group which contains longer length of microdystrophin, followed by RGX-DYS5 and RGX-DYS3 (FIG. 5 and FIG. 6A). The same trend was further substantiated by western blot analysis on muscle lysates (FIG. 6B). Western blot against syntrophin was performed on skeletal muscle tissue lysate (gastrocnemius muscle tissue from 3 each of the mdx treated and untreated groups, and one gastrocnemius and two triceps were from the B6 mice group). The polyclonal anti-syntrophin antibody (Abcam, ab11187) was used at 1:10,000, incubation at room temperature for 1 hour. Rabbit monoclonal against α-actinin (ab68167, Abcam) was applied at 1:5000 dilution. Secondary goat anti-rabbit antibody (Thermo Fisher Scientific, Cat. No. A-10685) was applied. The ratio of syntrophin expression to the endogenous control actinin expression in WT muscle was 4.56±0.76 (n=3, p<0.001 by one-way ANOVA) as compared with mdx group (0.84±0.22). The ratio in RGX-DYS1 and RGX-DYS5 groups were 2.72±0.97 (n=3, p<0.05 as compared with mdx group) and 1.35±0.03, respectively (FIG. 6C). The level of syntrophin expression in skeletal muscle was additionally examined on total muscle membrane extracts by western blot. Total skeletal muscle protein was extracted using Mem-Per Plus membrane protein extraction kit (Cat #89842, Thermo Fisher) (gastrocnemius muscle tissue from each of the mdx treated and untreated groups, and quadriceps from the B6 mice group). 20 μg of total membrane protein was loaded into each lane (FIG. 6D). The polyclonal anti-syntrophin antibody (Abcam, ab11187) was used at 1:10,000 incubation at 4° C. overnight. The loading control polyclonal anti-actin (PA5-78715, Thermo Fisher) was applied at 1:10,000 dilution for overnight incubation at 4° C. Slightly different from the whole lysate western experiment where WT muscle displayed the highest syntrophin expression level, the total membrane protein western blot displayed highest relative syntrophin expression in RGX-DYS1 group (0.81±0.26, n=3), followed by B6_WT group (0.6623±0.05, n=3), RGX-DYS3 group (0.59±0.08), and mdx group (0.32±0.07, n=3), as seen in FIG. 6E. These results clearly indicated that the microdystrophins generated by the microdystrophin vectors were able to restore muscle membrane syntrophin expression, and the longer version of RGX-DYS1 had superior ability to anchor syntrophin to muscle membrane than the shorter version RGX-DYS3.

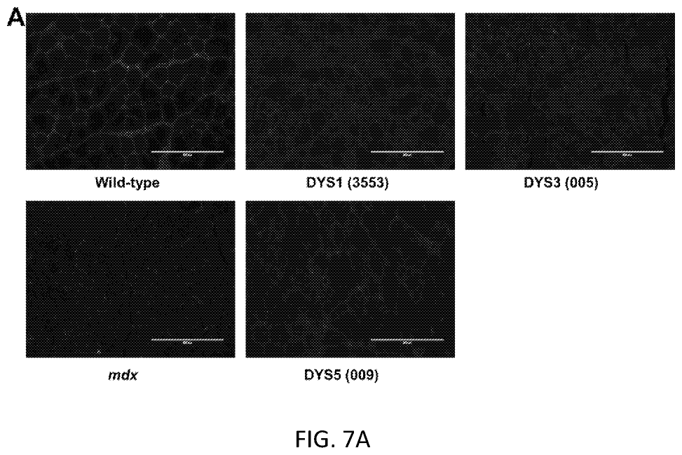

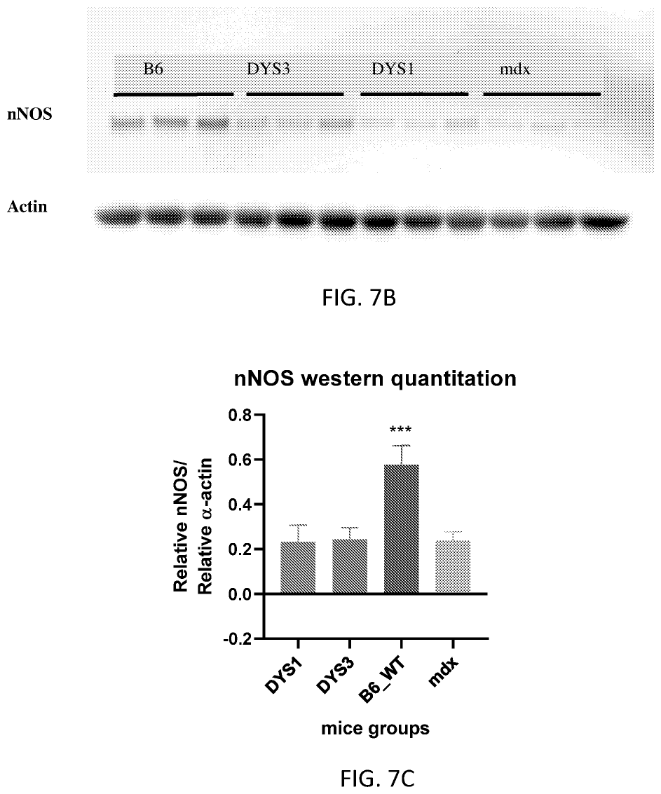

nNOS western blots were prepared analogously using muscle membranes (gastrocnemius muscle tissue/mdx, and quadriceps/B6 groups). Total muscle membrane protein was extracted using Mem-Per Plus membrane protein extraction kit (Cat #89842, Thermo Fisher). 20 μg of total membrane protein was loaded into each lane of an SDS-PAGE gel. The primary antibody against nNOS (SC-5302, Santa Cruz Biotechnology) was used at 1:500, and polyclonal anti-actin (PA5-78715, Thermo Fisher) was applied at 1:10,000 dilution. Secondary goat anti-Mouse IgG antibody, HRP (62-6520, ThermoFisher) was applied. With respect to nNOS expression, we observed a noticeable difference between the RGX-DYS1 and RGX-DYS3 group images following IF staining (FIG. 7A). However, western blot results did not reveal any significant difference among RGX-DYS1, RGX-DYS3, and untreated mdx group (FIGS. 7B-C), indicating the restoration of nNOS by RGX-DYS1 vector was low.

Overall, delivery of RGX-DYS1, RGX-DYS3, and RGX-DYS5 vectors in mdx mice all resulted in robust microdystrophin expression and restoration of dystrophin associated protein complexes (DAPCs). The longer version of RGX-DYS1 vector enhanced restoration of DAPCs particularly for syntrophin and 0-dystroglycan. The ability of restoration of nNOS to the membrane DAPC by RGX-DYS1 vector was low but visible upon IF staining.

### 6.2.3 Transduction of Satellite Cells and Amelioration of Regeneration of Muscular Dystrophic Muscle by RGX-DYS1 Vector

Skeletal muscle stem cells, or satellite cells (SCs), are normally quiescent and located between the basal lamina and sarcolemma of the myofiber. During growth and after muscle damage, a myogenic program of SCs is activated, and SCs self-renew to maintain their pool and/or differentiate to form myoblasts and eventually myofibers. Adeno-associated viral (AAV) vectors are well-known for transduction of differentiated myofibers, so we investigated whether satellite cells could also be transduced by AAV vectors. Satellite cells are small with very little cytoplasm, so it is technically challenging to study transgene expression in these cells. Here, we applied RNAscope® to investigate whether AAV could transduce satellite cells. RNAscope® is in situ hybridization (ISH) technology that enables simultaneous signal amplification and background noise suppression, which allows for the visualization of single molecule gene expression directly in intact tissue with single cell resolution. The co-localization of three microdystrophin proteins (DYS1, DYS3 or DYS5) and Pax7 mRNA in skeletal muscle of untreated mdx, RGX-DYS1 treated mdx and wild type C57BL/6 mice. RNAscope® multiplex fluorescent analysis was utilized with AAV microdystrophin probe labelled with fluorophore, Opal 570 (red), and muscle satellite cell marker, pax7, labelled with fluorophore, Opal 520 (green). The RNAscope® multiplex fluorescent analysis of AAV transgene and Pax7 mRNA expression was performed at Advanced Cell Diagnostics Inc (Newark, CA). Total RNA was extracted from skeletal muscles using RNeasy® Fibrous Tissue Mini Kit (Qiagen Cat. No. 74704), and cDNA was synthesized with High-Capacity cDNA Reverse Transcription Kit with RNase Inhibitor (Applied Biosystems Cat. No. 4374966). The absolute copy numbers of microdystrophin mRNA and endogenous control GAPDH mRNA were measured using digital PCR (Naica Crystal Digital PCR system, Stilla technologies). The primers and probe against microdystrophin was the same as previously described. The mouse pax7 primers and probe set (TaqMan™ MGB Probe, Applied Biosystems Cat. No. 4316034) was bought commercially.

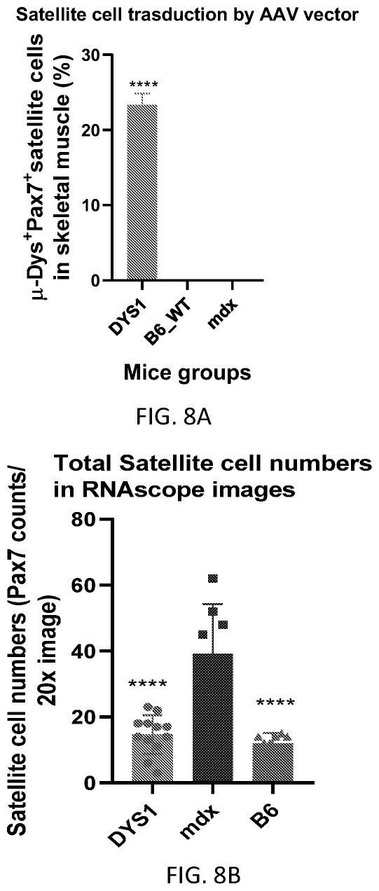

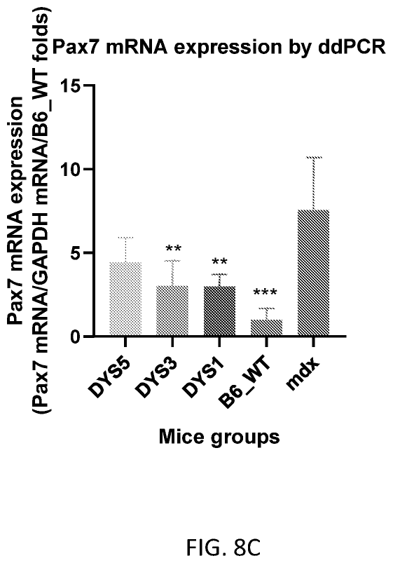

The microdystrophin transduced satellite cells were counted, and the satellite cell transduction rate was calculated. In AAV-μ-dys transduced skeletal muscles, the transduction rate of satellite cells was 23±1.5% (FIG. 8A). This indicated AAV vector was able to transduce muscle satellite cells although at much lower transduction rate than mature myofibers.

Total pax7+ satellite cell numbers were then counted in the RNAscope images to investigate whether the numbers of satellite cells were similar in the different treatment groups. As shown in FIG. 8B, pax7 positive cell counts per image in the untreated mdx was 39.12±15.14, and the positive cell counts in the wild-type B6 mice and DMD vector treated mice were 11.87±3.23 (8 images were counted, p<0.0001 by one way ANOVA) and 14.66±5.91 (12 images were counted, p<0.0001 by one way ANOVA), respectively. The increase of satellite cell numbers in the untreated mdx muscle indicated the regenerative nature of muscular dystrophic muscle. Delivery of microdystrophin with the RGX-DYS1 vector reversed this pathology and alleviated muscle regeneration.

In addition to RNAscope technology analysis, we extracted total muscle RNA and performed cDNA synthesis. Total RNA was extracted from skeletal muscles using RNeasy® Fibrous Tissue Mini Kit (Qiagen Cat. No. 74704), and cDNA was synthesized with High-Capacity cDNA Reverse Transcription Kit with RNase Inhibitor (Applied Biosystems Cat. No. 4374966). The samples were subjected to ddPCR analysis using mouse pax7 specific primers and probe sets (available commercially: mm01354484_m1 Pax7, Thermo Fisher Scientific; and TaqMan™ MGB Probe from Applied Biosystems Cat. No. 4316034, respectively). The mouse GAPDH primers and probe set were used to normalize the RNA and cDNA input. The absolute copy numbers of microdystrophin mRNA and endogenous control GAPDH mRNA were measured using digital PCR (Naica Crystal Digital PCR system, Stilla technologies). The ratio of pax7 mRNA copy numbers to GAPDH mRNA copy numbers were compared among groups (FIG. 8C). As expected, the relative expression of pax7 expression in mdx mice was 7.56±3.14, which was much higher than the WT-B6 mice (1±0.68, n=5, p<0.001 by one-way ANOVA). The relative pax7 expression in three different microdystrophin vector-treated groups were much reduced (4.40±1.50 for RGX-DYS5 (n=3, p=0.06), 3.12±0.74 for RGX-DYS3 group (n=5, p<0.01), 2.98±0.68 for RGX-DYS1 (n=5, p<0.01). Pax+ satellite cell count is elevated in mdx, consistent with active cycle of muscle degeneration and regeneration in this dystrophic model. The reduction of pax7 mRNA expression in satellite cells of microdystrophin-treated mdx mice indicates that the present microdystrophin vectors correct the satellite cell hyperplasia in muscular dystrophic muscle through amelioration of muscle regeneration.

DYS1 treatment significantly reduces the satellite cell hyperplasia in mdx, as measured by both satellite cell counting and Pax7 mRNA expression (FIGS. 8B and 8C).

### 6.3 Example 3 AAV8 Compared to AAV9 RNA/DNA Studies

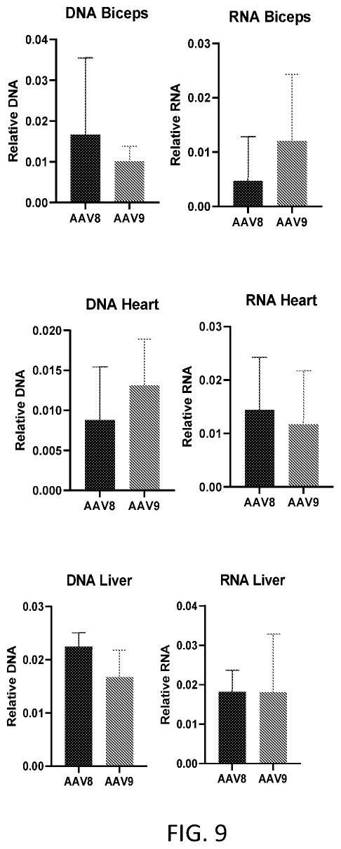

AAV8 and AAV9 have similar transduction efficiency in skeletal and cardiac muscles of Non-human primates (NHP) via systemic delivery (FIG. 9). AAV8 and AAV9 packaging CAG-GFP cassette with a unique barcode were produced individually and pooled together with other capsids in approximately equal concentration to generate a library of 118 barcoded AAVs. This library (PAVE118) was administered intravenously to three cynomolgus macaques at a dose of 1.77e13 GC/kg. DNA and RNA isolated from various NHP tissues at 3 weeks post dosing were subjected to NGS analysis for relative abundance. There was no significant difference between DNA and RNA levels from AAV8 and AAV9 capsid in skeletal muscle, cardiac muscle, and liver of NHP.

### 6.4 Example 4—In Vitro Studies (DMD Patient-Derived Induced Pluripotent Stem Cells (iPSC))

Although mdx/BL10 is a well-established murine model of DMD, cardiomyopathy—a leading cause of death in patients with DMD—is either not exhibited or the phenotype is limited to mild ventricular dilation upon aging (Yucel et al, 2018). Recent publications have indicated that iPSC-derived cardiomyocytes (iPSC-CMs) from DMD patients can be used for modeling dilated cardiomyopathy (Laurila et al, 2016; Lin et al, 2015).

This study can establish an in vitro cardiac model of DMD using patient-derived iPSC-CMs and evaluate the bioactivity of RGX-DYS1 in dystrophin-deficient human cardiomyocyte cells. iPSCs from DMD patients obtained from the European Bank for Induced Pluripotent Stem Cells (EBiSC) and healthy donors can be differentiated into cardiomyocytes. Functional phenotypes of DMD and healthy control human cell lines can be characterized once mature cardiomyocytes are generated.

Upon the establishment of functional phenotypes, RGX-DYS1 can be incubated with DMD iPSC-CMs. RGX-DYS1 vector (DNA) and RGX-DYS1 microdystrophin will be determined by qPCR and immunocytochemistry, respectively. Additionally, to assess the benefit of RGX-DYS1 in DMD cardiomyocytes, cardiac-functional endpoints (i.e., impedance, electric activities, and calcium handling) can be evaluated.

### 6.5 Example 5—Proof of Concept-6 Week Study in Mdx Mice

### 6.5.1 6-Week Study in Mdx Mice

To evaluate the bioactivity of AAV8-RGX-DYS1 in mdx mice, AAV8-RGX-DYS1 was administered intravenously to 5-week-old mdx male mice (C57BL/10ScSn-Dmdmdx/J; n=13 per group) at doses of 0 (vehicle) or 2×10¹⁴ GC/kg. The following parameters and endpoints were included in this study: mortality, clinical observations, body weights, forelimb grip strength, and in vitro force on the Extender Digitorum Longus (EDL). At necropsy (6 weeks post-dose), gross examination of tissues, including tissue weights was conducted. Muscle tissue was separately collected for evaluation of muscle pathology. AAV8-RGX-DYS1 microdystrophin expression was evaluated by Western blot and immunofluorescence, and RGX-DYS1 vector DNA biodistribution was also assessed. Finally, expression and localization of DAPC proteins were also assessed in tibialis anterior (TA) and diaphragm tissues using immunofluorescence.

AAV8-RGX-DYS1 was well tolerated at 2×10¹⁴ GC/kg. There were no RGX-DYS1-related mortalities or adverse clinical observations. One mouse was euthanized due to hydrocephalus 3 weeks after RGX-DYS1 administration. However, this finding was not considered test article-related as hydrocephalus is commonly seen in mdx mice and was also seen in vehicle control mdx mice in the 12-week pharmacology study (Xu et al, 2015; Example 6)

Consistent with the natural history data of mdx mice (Coley et al, 2016), mean body weight in vehicle control mdx mice was significantly higher than the age-matched historical controls (+11%). Also, the absolute and normalized muscle tissue weights were significantly higher compared to the wild-type historical control data (HCD) at the testing facility (AGAD Biosciences) (+18% to 53%, +7% to +36%, respectively). RGX-DYS1 administration decreased body weight in mdx mice (−13%), but this was comparable to the testing facility's historical wild-type control data. Concomitant with this, the absolute and normalized weights of all skeletal muscles (including the diaphragm) were lower than the vehicle control mdx mice (−17% to −29% and −4% to −18%, respectively).

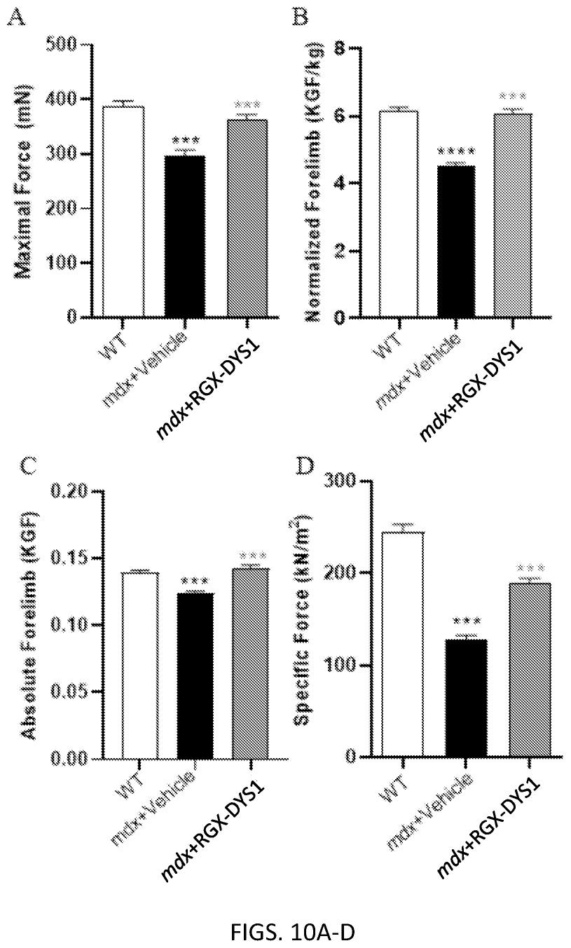

Muscle function was assessed by grip strength at Week 5, and in vitro force of the EDL muscle was assessed at necropsy (Week 6). The vehicle control mdx mice showed significant reduction in the absolute and normalized forelimb grip strength compared to the age-matched historical wild-type control data. AAV8-RGX-DYS1 administration increased the absolute and normalized forelimb grip strength in mdx mice compared to the vehicle control mdx mice (+14.5% and +33.7%, respectively), and these data were comparable to the historical wild-type control data at the testing facility. As shown in FIGS. 10A-10D, both maximal and specific force output were significantly decreased in the vehicle control mdx mice compared to wild-type HCD. In contrast, AAV8-RGX-DYS1 administration to mdx mice led to significant increases in the maximal and specific force output of the EDL muscle compared to vehicle control mdx mice (+21% and 47.2%, respectively). To determine grip strength, the mouse was gently placed on top of the forelimb wire grid so that only its front paws were allowed to grip one of the horizontal bars. After ensuring both the front paws were grasping the same bar and the torso horizontal to the ground and parallel to the bar, the mouse was pulled back steadily with uniform force down the complete length of the grid until the grip was released. 5 good pulls for each animal over five consecutive days for acclimation and testing. The single best-recorded value (maximal force) was calculated for analysis of maximal strength of individual mice. Normalized strength (KGF/kg) was calculated based on the body weight. To determine in vitro force, the EDL muscle of the right hindlimb were removed from each mouse and immersed in an oxygenated bath (95% 02, 5% CO2) that contains Ringer's solution (pH 7.4) at 25° C. Using non-fatiguing twitches, the muscle was adjusted to the optimal length for force generation. The muscles were stimulated with electrode to elicit tetanic contractions that were separated by 2-minute rest intervals. With each subsequent tetanus, the stimulation frequency was increased in steps of 20, 30 or 50 Hz until the force reached a plateau which usually occurred around 250 Hz. The cross-sectional area of the muscles was measured based on muscle mass, fiber length, and tissue density. Finally, the muscle specific force (kN/m2) was calculated based on the cross-sectional area of the muscle.

To examine whether AAV8-RGX-DYS1 administration not only improves muscle function, but also attenuates the dystrophic phenotype in mdx mice, muscle pathology (i.e., inflammation, degeneration, regeneration, and central nucleation) was examined in the TA and diaphragm at the end of the study (Week 6) in mdx mice administered AAV8-RGX-DYS1. The wild-type control tissue from the testing facility's (AGADA Biosciences) tissue bank was used (n=2-3) as a comparator for the TA muscle and aged-matched wild-type HCD from the test facility was used for the diaphragm.

Inflammation was examined using Hematoxylin and Eosin (H&E) staining. Regenerating and degenerating fibers in muscles were determined by immunostaining of embryonic myosin heavy chain (eMHC) and IgM, respectively. Central nucleation, another indicator of muscle regeneration, was also measured by H&E staining.

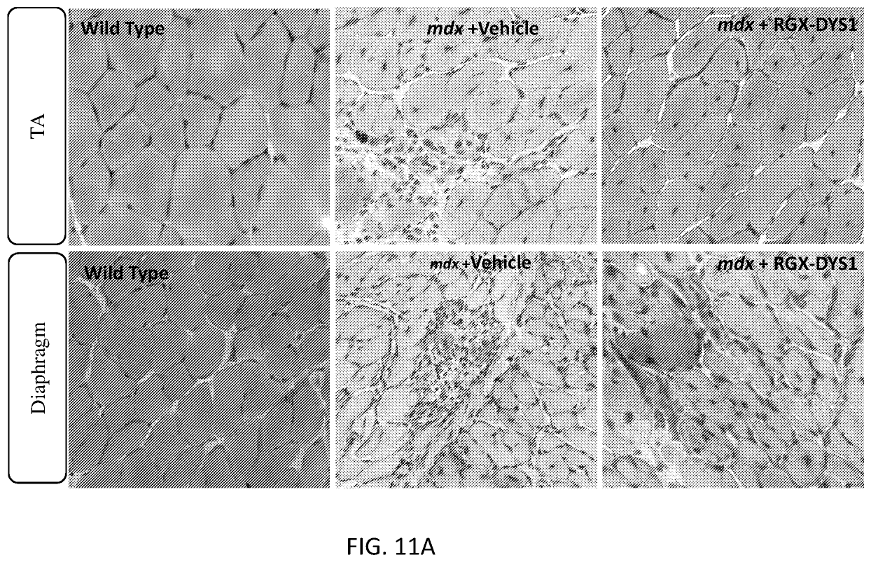

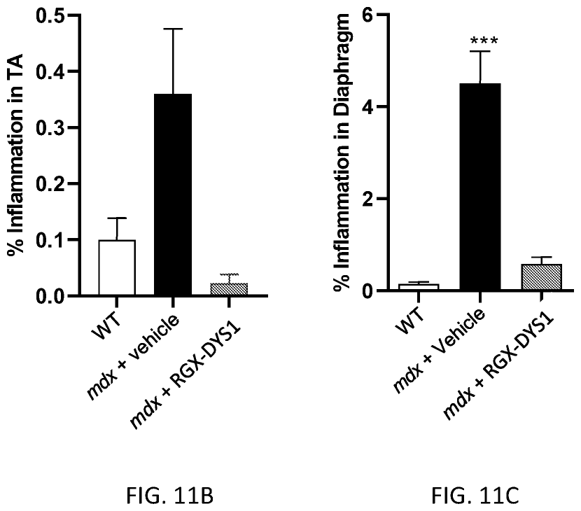

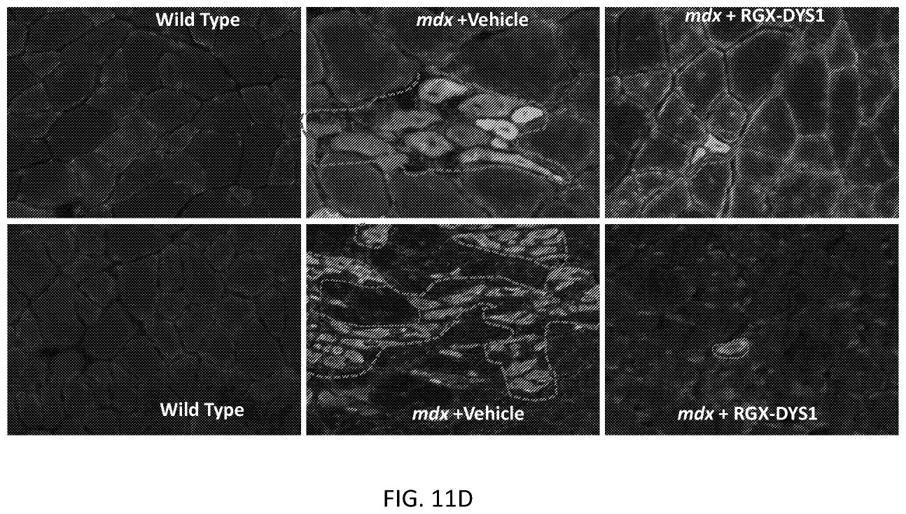

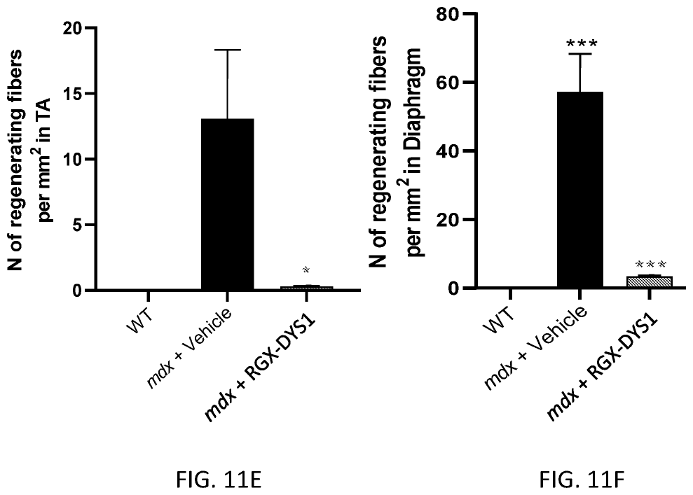

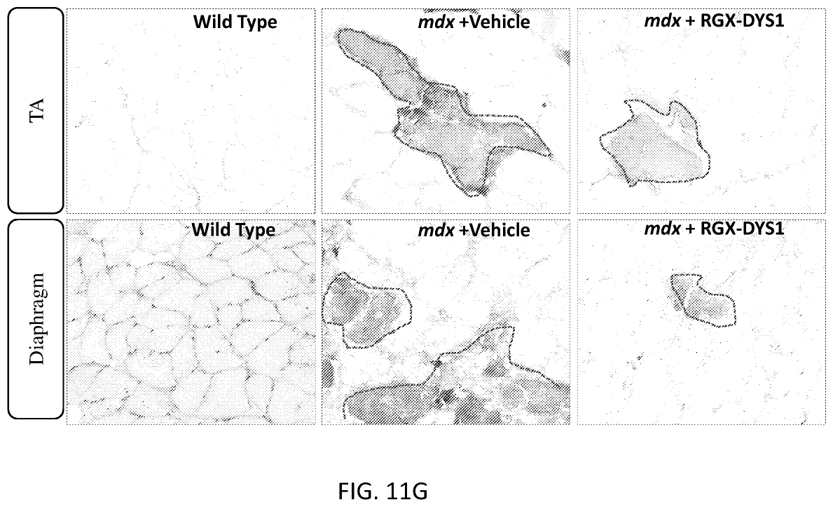

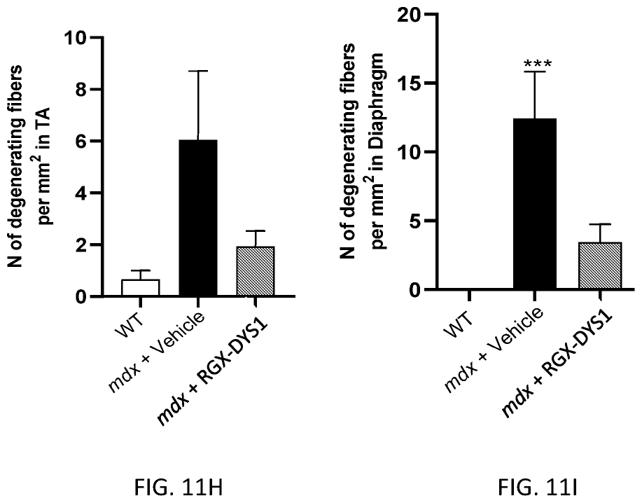

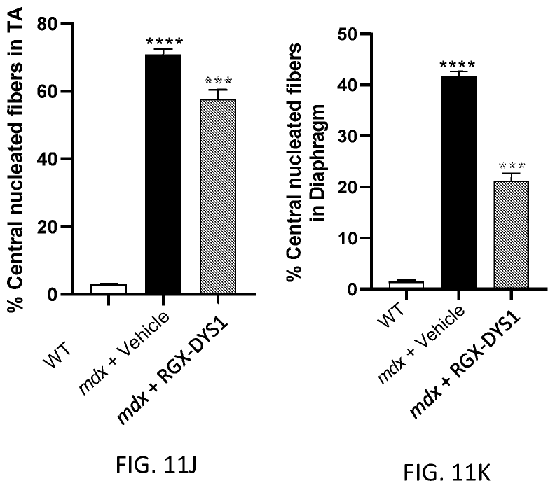

As shown in FIGS. 11A-11K, dystrophic pathology (inflammation, degeneration, regeneration) was apparent in the TA and diaphragm of vehicle control mdx mice compared to the wild-type mice. In addition, centrally nucleated fibers (CNFs), recognized as regenerated fibers, in the vehicle control mdx were significantly higher than the historical wild-type control data (TA: 2.92% in wild type vs 70.81% in mdx; diaphragm: 1.46% in wild type vs 41.63% in mdx). AAV8-RGX-DYS1 administration attenuated dystrophic changes in mdx mice (FIGS. 11A-11K); significant reductions in inflammation, regeneration, and degeneration were observed in both the TA and diaphragm tissues, which were similar to the wild-type HCD at the testing facility. AAV8-RGX-DYS1 administration also significantly decreased the percentage of CNFs in the TA (−18.4%) and diaphragm (−48.9%) in mdx mice, but the percentages of CNFs in both tissues were higher than the historical wild-type control data (FIGS. 11A-11K). These observations are likely due to the early onset of muscle cell replication (˜3 weeks of age) in mdx mice prior to AAV8-RGX-DYS1 administration and the excellent regenerative capacity of mdx-BL10 background mice, unlike DMD patients (Turk et al, 2005).

To confirm the successful transduction of AAV8-RGX-DYS1 in mdx mice the RGX-DYS1 biodistribution (vector DNA) was examined by ddPCR, and transgene levels of RGX-DYS1 microdystrophin (protein) was determined by immunofluorescence and Western blot. Dystrophin levels were also measured as a control.

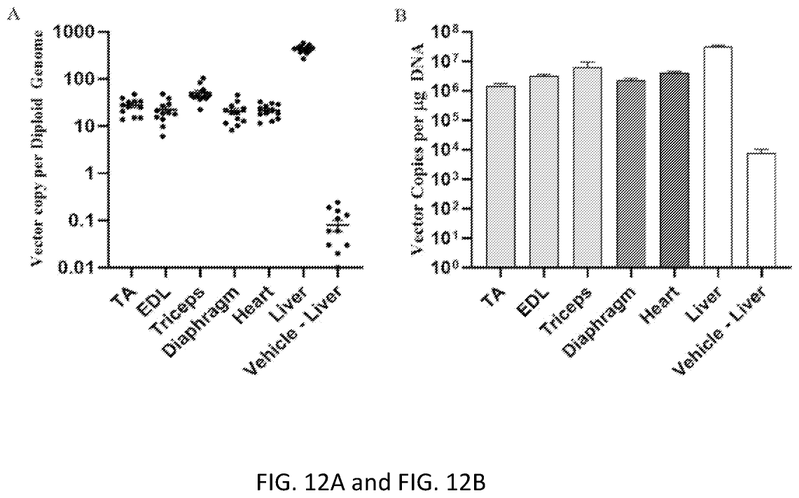

Vector DNA levels were quantifiable by ddPCR in all tissues (liver, heart, diaphragm, TA, EDL, and triceps) and were collected from all AAV8-RGX-DYS1-administered animals (FIGS. 12A and 12B)). The liver had the highest vector DNA level, whereas levels in all other muscle tissues were comparable. In the vehicle control mdx mice, the liver and a few selected animal tissues for each muscle group were analyzed to confirm that vector DNA levels were absent or close to LLOQ (−0.08 GC/dg).

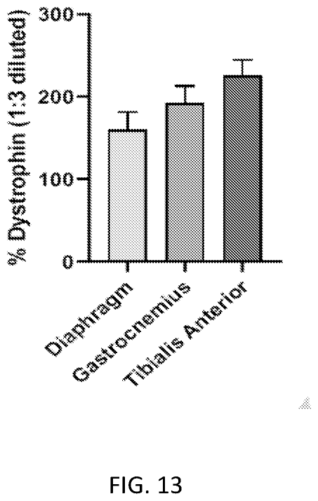

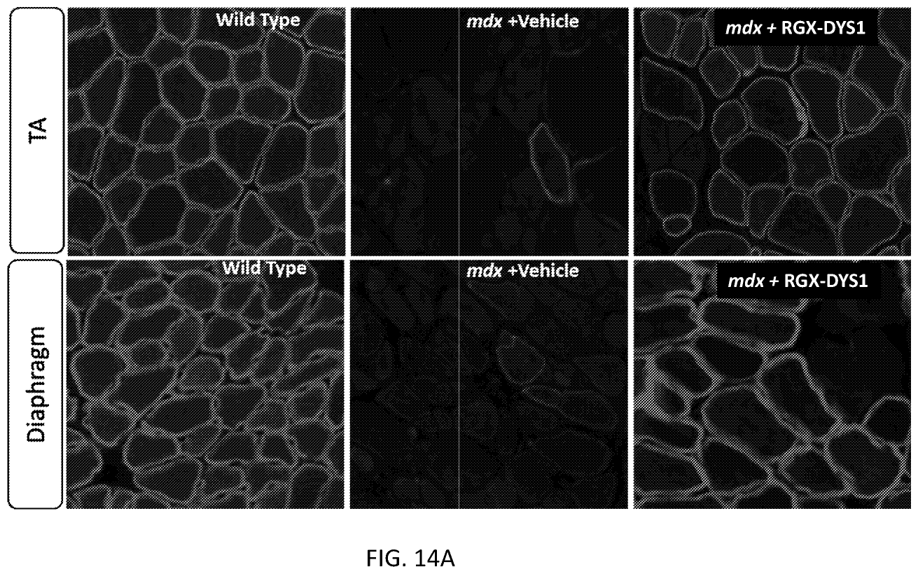

For Western blot analysis, protein was extracted from the diaphragm, gastrocnemius, and TA muscles collected from AAV8-RGX-DYS1-administered mdx mice. The level of microdystrophin in the samples was calculated as a percent of the normal dystrophin based on the standard curve derived from measurement of dystrophin from a mixture of BL10 mouse and German Shorthair Pointer Muscular Dystrophy (GSHPMD) dog muscle lysates. AAV8-RGX-DYS1-administered mdx mice showed an expression of AAV8-RGX-DYS1 microdystrophin, reported as percent of dystrophin, of 159.5% in diaphragm, 191.8% in gastrocnemius, and 225.2% in TA muscles (FIG. 13). AAV8-RGX-DYS1 microdystrophin expression in diaphragm, gastrocnemius, and TA muscles were consistent with the wide distribution of vector DNA across all muscle tissues at Week 6.

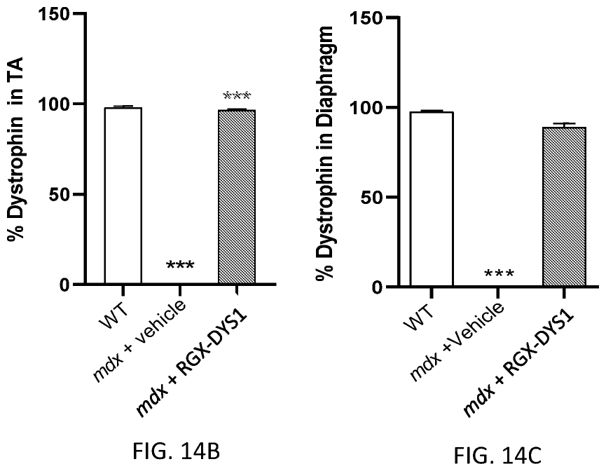

To further confirm the RGX-DYS1 microdystrophin expression in myofibers, immunofluorescence was performed in the TA and diaphragm. Six weeks post administration, the vehicle control mdx mice had no dystrophin-positive fibers (i.e., no dystrophin expression at sarcolemma membrane) in the TA and diaphragm except for very few somatic revertant myofibers. In contrast, the AAV8-RGX-DYS1-administered mdx mice showed robust and correct microdystrophin expression at the sarcolemma membrane of the TA (96%) and diaphragm (89.1%) muscle tissues (FIGS. 14A-14C); these results were similar to that of wild-type control. As previously reported, a uniform dystrophin expression is required to stabilize myofiber turnover and attenuate pathology in dystrophic muscle (van Westering et al, 2020).

Dystrophin deficiency results in the disassembly of the entire DAPC, which is responsible for maintaining muscle integrity and cellular signaling during repetitive contraction and relaxation of muscle (Sancar et al, 2011; Duan et al, 2018). Thus, the absence of dystrophin and destabilization of DAPC is thought to increase susceptibility to muscle damage and accumulate intracellular calcium influx, leading to a severe dystrophic phenotype (Cirak et al, 2012).

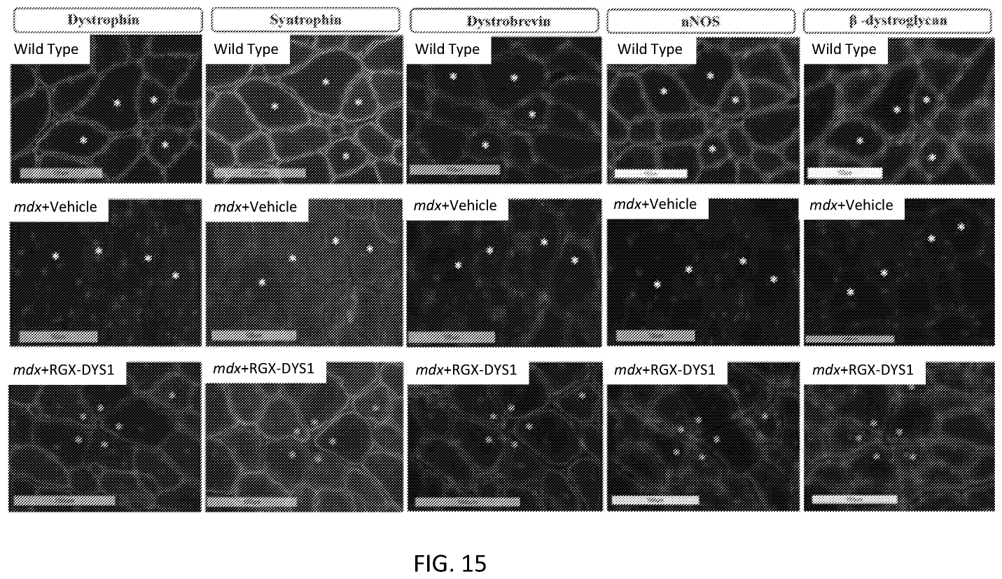

To evaluate whether AAV8-RGX-DYS1 administration could also restore the stabilization of DAPC proteins, immunofluorescence was performed in the TA and diaphragm muscles with anti-α1-syntrophin, dystrobrevin, nNOS-1, and β-dystroglycan (FIG. 15).

The vehicle control mdx mice showed negligible/undetectable DAPC proteins in the sarcolemma of muscle fibers in the TA and diaphragm compared to the wild-type controls. AAV8-RGX-DYS1 administration fully restored sarcolemma expression of α1-syntrophin ( 9/10 in TA and 10/10 in diaphragm) and dystrobrevin ( 8/10 in TA and 10/10 in diaphragm) in both tissues. More importantly, both α1-syntrophin and dystrobrevin expressions in AAV8-RGX-DYS1-administered mdx mice appeared to co-localize with anti-dystrophin staining and were similar to the wild-type mice. These results demonstrated that the C-terminal domain (CT146) of RGX-DYS1 microdystrophin could recruit the α-dystrobrevin and α-syntrophin, as previously reported (Adams et al, 2000; Dowling et al, 2021). β-dystroglycan presence at the sarcolemma was also restored in AAV8-RGX-DYS1-administered mdx mice compared to the vehicle control mdx mice. However, AAV8-RGX-DYS1-administered mdx mice showed large areas of robust expression ( 6/10) and low expression ( 4/10) in the TA when compared to wild-type. A similar pattern was also noted in the diaphragm tissues from AAV8-RGX-DYS1-administered mdx mice. AAV9-RGX-DYS1 administration did not appear to fully restore nNOS presence at the sarcolemma but nNOS was detectable at the sarcolemma of the TA and diaphragm, and at higher levels than the vehicle control mdx mice. Taken together, AAV8-RGX-DYS1 administration increased the expression of DAPC proteins, including those proteins specific to the CT domain, at the sarcolemma of the TA and diaphragm muscles, suggesting an improvement of the structural integrity of muscle fibers.

To further elucidate the pharmacology of AAV8-RGX-DYS1 in mdx mice, AAV8-RGX-DYS1 was administered intravenously to 5-week-old mdx mice (5 males per group) at doses of 0 (vehicle), 3×10¹³, 1×10¹⁴, and 3×10¹⁴ GC/kg. An additional group of wild-type mice (C57BL/10ScSn) was included as a control. Animals will be sacrificed at 6 weeks post-dose. In vitro force measurement, including eccentric contraction, will be assessed in EDL and diaphragm muscles as a functional muscle endpoint. The following endpoints will also be included: mortality, clinical observations, weekly body weights, transgene expression (protein), gross examination, tissue weights, and muscle pathology.

### 6.6. Example 6: 12 Week Pharmacology Study in Mdx Mice

### 6.6.1 12-Week Study in Mdx Mice

The pharmacology of AAV8-RGX-DYS1 in mdx mice following a single IV injection was evaluated.

Groups of mdx male mice (n=10 per group) were administered a single IV injection of AAV8-RGX-DYS1 at 0 (vehicle), 3×10¹³, 1×10¹⁴, 3×10¹⁴, or 5×10¹⁴ GC/kg (maximum feasible dose). An additional group of wild-type mice (C57BL/10ScSn) was included as a control. The following parameters and endpoints were included: mortality, clinical observations, body weights, in vivo muscle function (grip strength, automated gait analysis), biomarkers (T2-MRI imaging and CK from serum), AAV8-RGX-DYS1 biodistribution (vector DNA), RGX-DYS1 microdystrophin expression (protein), gross examination, tissue weights, and histopathology, including spermatogenesis. In vivo endpoints (grip strength, motor gait analysis and T2-MRI imaging) were conducted at Week 6 and 12. An additional time point (Week 9) was added to conduct the grip strength measurement. Serum for CK analysis was collected at Week 7 after examining in vivo endpoints, and at terminal necropsy. 12 weeks after AAV8-RGX-DYS1 administration, terminal necropsy was conducted. Thus, all animals were sacrificed 12 weeks post administration.

AAV8-RGX-DYS1 was well tolerated up to the 5×10¹⁴ GC/kg dose, and there was no AAV8-RGX-DYS1-related mortality. There were four premature deaths due to hydrocephalus, consisting of one male in the vehicle control group, two males administered 3×10¹³ GC/kg and one male administered 1×10¹⁴ GC/kg AAV8-RGX-DYS1. However, as previously stated, this finding was not considered test-article related as hydrocephalus is associated with the mdx mouse phenotype (Xu et al, 2015). There were no AAV8-RGX-DYS1-related clinical observations during the study period.

### Fine Motor Kinematic Gait Analysis (In Vivo Functional Test)

Fine motor kinematic analysis was used to demonstrate the functional effect of AAV8-RGX-DYS1. Briefly, the movement of mice was captured using a high-speed camera (300 frames/s) from three different views, from below, right side, and left side. Fine motor skills and gait properties were then assessed using a high precision kinematic analysis method (MotoRater; TSE Systems, Homburg, Germany) using the walking mode. When vehicle control mdx mice are observed using fine motor kinematic analysis, the phenotype is associated with a lower body posture which is observed as increased hip, knee and ankle extensions as well as increased overall hip height and decreased forelimb toe clearance compared to wild-type mice.

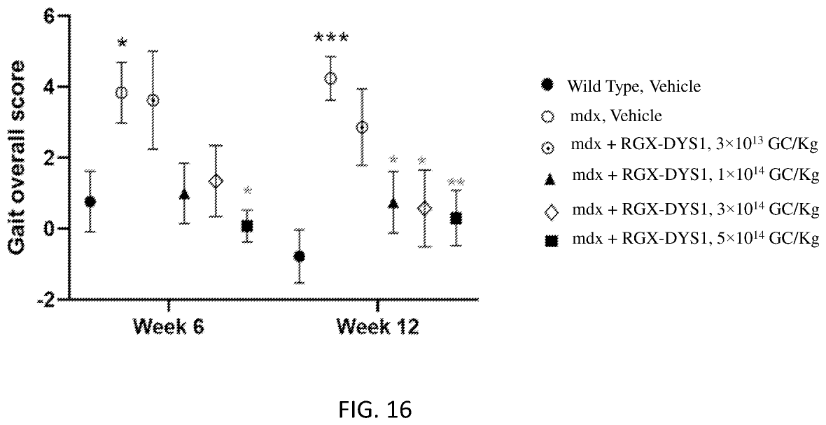

As shown in the FIG. 16, the overall gait score, which combined kinematic parameters into one single score, in the vehicle control mdx mice was significantly higher than the wild-type mice at Week 6 (0.77 in wild type vs 3.84 in vehicle control mdx), and a clearer difference was noted between the vehicle control mdx mice and wild-type mice at week 12 (−0.77 in wild type vs 4.25 in vehicle control mdx). At Week 6 (11-12 weeks of age), the effects of AAV8-RGX-DYS1 were prominent at doses of 1×10¹⁴ GC/kg and 3×10¹⁴ GC/kg; the overall gait score at a AAV8-RGX-DYS1 dose of 5×10¹⁴ GC/kg was similar to that of the wild type. At Week 12 (17-18 weeks of age), the overall gait score at a AAV8-RGX-DYS1 dose of >1×10¹⁴ GC/kg was significantly improved and normalized to the wild type level (−0.77 in wild type vs 0.76, 0.57, and 0.30 at 1×10¹⁴, 3×10¹⁴, 5×10¹⁴ GC/kg, respectively).

### T2-Magnetic Resonance Imaging (Biomarker)

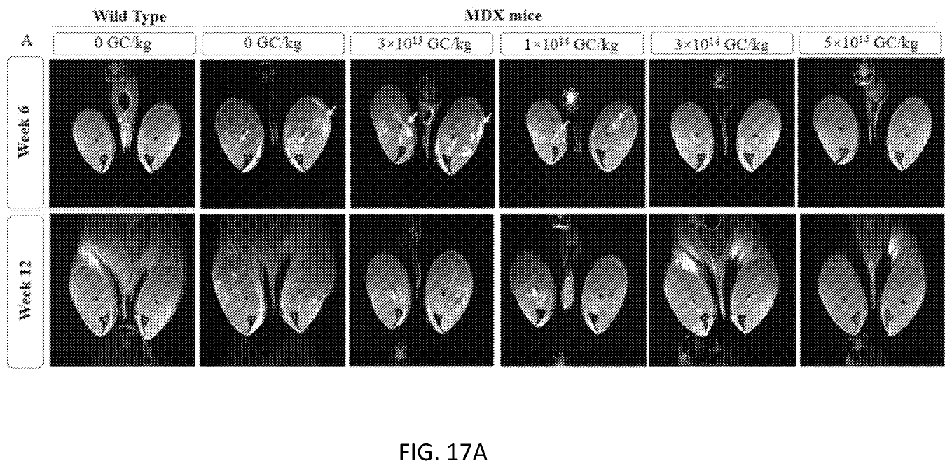

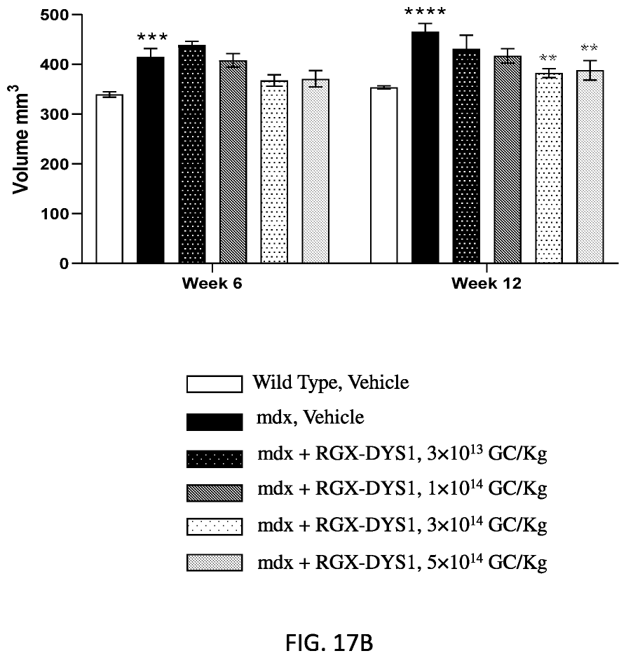

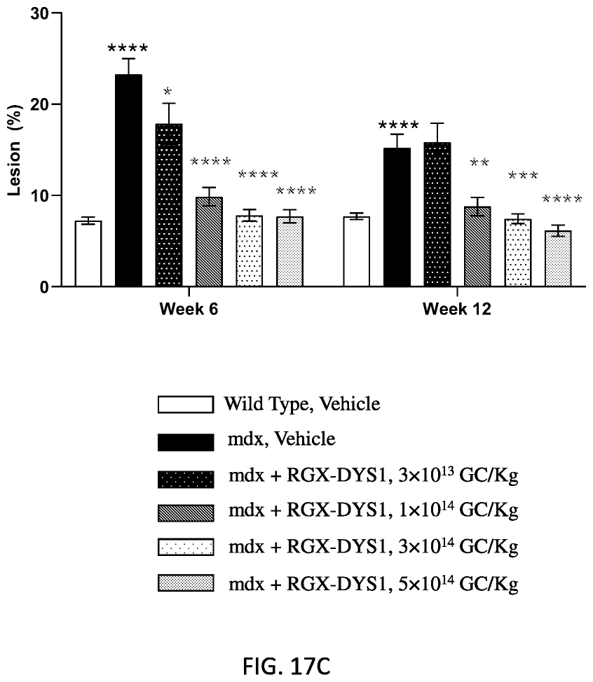

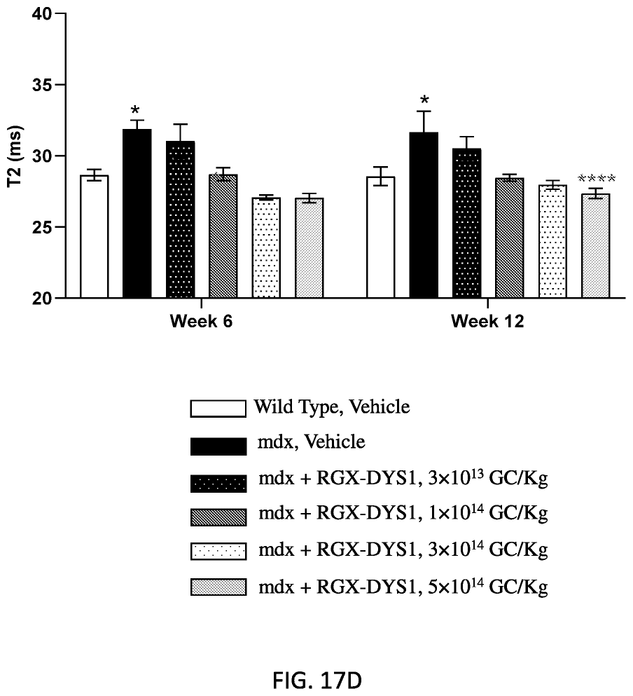

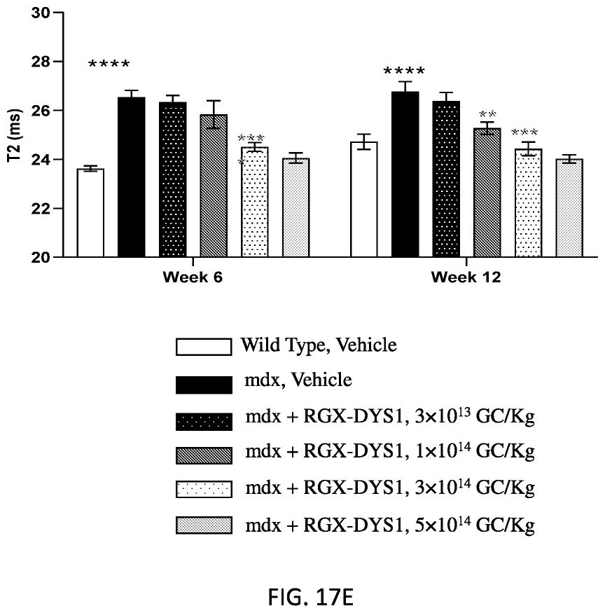

In DMD patients, muscle MRI is emerging as a powerful tool to assess muscle damage and inflammation (Forbes et al, 2020). In this study, a T2-mapping MRI was performed 6 and 12 weeks after dosing to evaluate gastrocnemius muscle volumes, percent hyperintense lesion, and T2-relaxation times in gastrocnemius lesions (hyperintense) and non-lesions (normal-appearing muscle) (FIG. 17A). Gastrocnemius volume (both legs combined) was significantly increased in the vehicle control mdx mice compared to the wild-type mice at both Week 6 and 12 time points due to compensatory hypertrophy. At Week 6, AAV8-RGX-DYS1 administration reduced gastrocnemius volume at doses of 3×10¹⁴ and 5×10¹⁴ GC/kg compared to the vehicle control mdx mice. At Week 12, the dose-response bioactivity of AAV8-RGX-DYS1 in gastrocnemius volume was clearly observed in mdx mice administered AAV8-RGX-DYS1 at doses of 3×10¹⁴ and 5×10¹⁴ GC/kg.

Hyperintense lesions, as a marker of muscle edema, were quantified based on automated threshold analysis from both legs. Increased hyperintense lesions (represented as % lesions) were clearly observed in the vehicle control mdx when compared to the wild-type controls at Week 6 and 12. At Week 6, reduced lesions were already evident at a AAV8-RGX-DYS1 dose of 3×10¹³ GC/kg, and were significantly improved at doses of 1×10¹⁴, 3×10¹⁴, and 5×10¹⁴ GC/kg. At Week 12, clear differences were noted in the mdx mice administered AAV8-RGX-DYS1 at doses of 1×10¹⁴, 3×10¹⁴, and 5×10¹⁴ GC/kg, and were comparable to wild type.

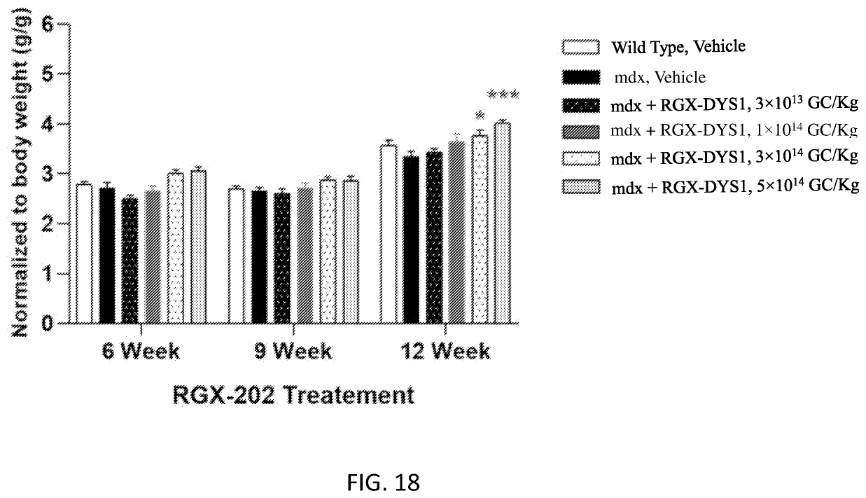

T2 time is normally increased in pathological process involving water environmental changes such as edema, inflammation, and to some extent formation of fibrosis (Hogrel et al, 2016; Wokke et al, 2016). Therefore, T2-relaxation time was assessed for both hyperintense lesions and normal-appearing gastrocnemius muscle (non-lesion) (FIGS. 18 D and E). Although there were no lesions observed in the images of the wild-type animals, the small percentage reported in FIG. 18A should be considered background levels. Increased T2-relaxation time was observed in the vehicle control mdx mice at both time points (Week 6 and 12) when compared to wild type. AAV8-RGX-DYS1 administration significantly decreased T2-relaxation time in mdx mice at doses of 1×10¹⁴, 3×10¹⁴, and 5×10¹⁴ GC/kg, and these times were comparable to wild type at Week 6 and 12. Thus, in AAV8-RGX-DYS1-administered mice, T2-relaxation time was comparable to wild-type animals at doses >1×10¹⁴ GC/kg by Week 12.

In AAV8-RGX-DYS1-administered mice, T2-relaxation time was comparable to wild-type animals at doses of >1×10¹⁴ GC/kg by Week 12.

### Grip Strength (In Vivo Functional Test)

Grip strength measurement at Week 6 and 9 did not clearly reveal differences between vehicle control mdx mice and wild-type mice (FIG. 18). At Week 12, a minimal difference in the grip strength between the vehicle control mdx and wild-type mice was noted with no statistical significance. The grip strength in mdx mice administered AAV8-RGX-DYS1 at doses of 3×10¹⁴ and 5×10¹⁴ GC/kg was significantly increased when compared to the vehicle control mdx mice. These inconsistent observations are likely due to the fact that grip strength testing in rodents can be influenced by a variety of factors other than motor function, (Maurissen et al, 2003; Nagaraju et al, 2008).

### Creatine Kinase

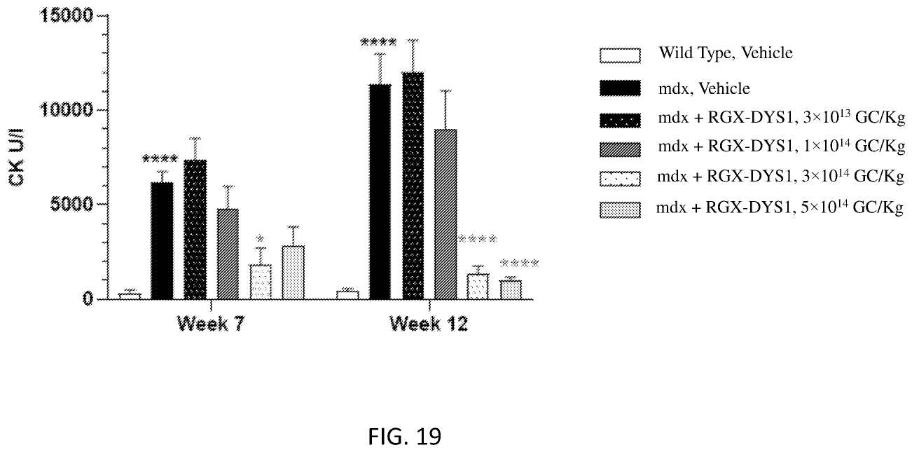

As expected, mean CK levels were 21-fold and 30-fold greater in the vehicle control mdx mice compared to wild type control at week 7 and week 12, respectively. In the AAV8-RGX-DYS1-administered mdx mice, CK levels were reduced at doses >1×10¹⁴ GC/kg, reaching significance at doses of >3×10¹⁴ GC/kg (FIG. 19).

### RGX-DYS1 Biodistribution (Vector DNA)

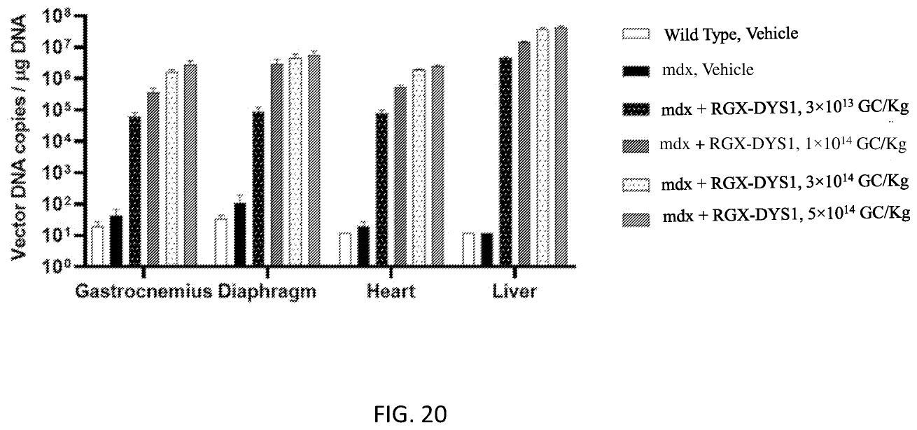

DNA vector biodistribution was assessed using the qPCR method. Gastrocnemius, diaphragm, heart, and liver tissues from AAV8-RGX-DYS1-administered mdx mice had high levels of vector DNA at the end of the study (Week 12). A trend for dose-proportional increase of vector DNA levels in the examined tissues of all AAV8-RGX-DYS1 treated mice was observed, although no significance was reached (FIG. 20). The liver had a higher vector DNA level compared to muscle tissues in all AAV8-RGX-DYS1-administered mice. Tissues collected from wild-type BL10 mice and mdx vehicle control mice showed vector DNA levels at either below quantitation limit (BQL=50 copies/μg DNA) or limit of detection (LOD=11.96 copies/μg DNA).

### RGX-DYS1 Microdystrophin Expression (Protein)

To examine the RGX-DYS1 microdystrophin expression in mdx mice, Western blot analysis was performed.

At Week 12, mdx mice administered AAV8-RGX-DYS1 at doses of 1×10¹⁴, 3×10¹⁴, and 5×10¹⁴ GC/kg showed significantly higher RGX-DYS1 microdystrophin expression in all three muscles (gastrocnemius, diaphragm, and heart) when compared to vehicle control mdx mice (p<0.05-0.001). At the lowest AAV8-RGX-DYS1 dose (3×10¹³ GC/kg), RGX-DYS1 microdystrophin levels were higher than vehicle control mdx mice but were not significant.

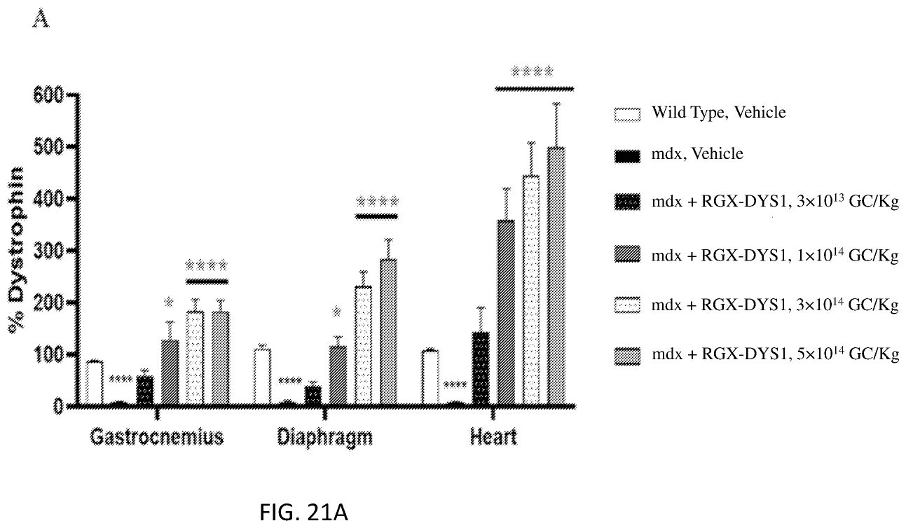

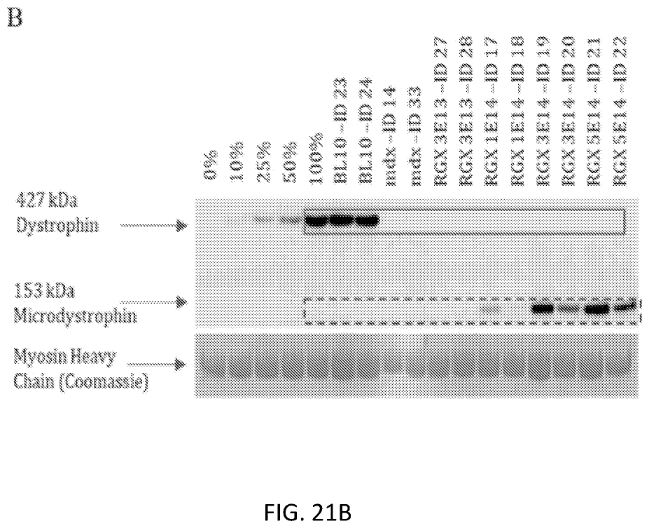

In all AAV8-RGX-DYS1-administered mdx mice, expression of RGX-DYS1 microdystrophin in heart tissue was higher when compared to gastrocnemius and diaphragm, whereas expression in the gastrocnemius and diaphragm were generally comparable (FIGS. 21A and 21B). In comparison to the dystrophin protein expression in wild-type mice, the mdx mice that received AAV8-RGX-DYS1 at 3×10¹⁴ and 5×10¹⁴ GC/kg showed significantly higher RGX-DYS1 microdystrophin expression in gastrocnemius, diaphragm, and heart.

Overall, the presence of RGX-DYS1 microdystrophin protein in muscles from AAV8-RGX-DYS1-administered mdx mice was consistent with the detection of vector DNA levels. Despite the fact that RGX-DYS1 vector DNA levels across all muscles were comparable in each dose group, RGX-DYS1 microdystrophin in heart tissue was generally higher when compared to gastrocnemius and diaphragm, whereas expression in the gastrocnemius and diaphragm were generally comparable.

In this study, the minimum effective dose following IV administration of AAV8-RGX-DYS1 to mdx mice is currently considered to be 1×10¹⁴ GC/kg, based on significant improvement in muscle function as measured by fine motor kinematic gait analysis and improvement in muscle preservation as measured by MRI.

### 6.7 Example 7: 26-Week Pharmacology Study in Mdx Mice

The long-term bioactivity of AAV8-RGX-DYS1 in mdx mice following a single IV injection is being evaluated.

Groups of male mdx mice (n=10 per group) were administered a single IV injection of AAV8-RGX-DYS1 at 0 (vehicle), 3×10¹³, 1×10¹⁴, 3×10¹⁴, or 5×10¹⁴ GC/kg. An additional group of wild-type mice (C57BL/10ScSn) was included as a control. Animals will be euthanized at 26 weeks post-dose. The following parameters and endpoints are included: mortality, clinical observations, weekly body weights, in vivo muscle function (grip strength at Week 6, 9, 17, and 26; automated gait analysis at Week 9, 17, and 26), biomarkers (T2-MRI imaging at Week 6, 17, and 26, and CK from serum at Week 17 and 26), biodistribution (vector DNA), transgene expression (protein), gross examination, tissue weights, and histopathology including spermatogenesis and muscle pathology.

The long-term efficacy of AAV8-RGX-DYS1 in mdx mice following a single IV injection and the long-term toxicity of AAV8-RGX-DYS1 in a relevant model of DMD were also studied.

Groups of mdx male mice (n=10 per group per time point) were administered a single IV injection of AAV8-RGX-DYS1 at 0 (vehicle), 3×10¹³, 1×10¹⁴, 3×10¹⁴, or 5×10¹⁴ GC/kg. An additional group of wild type mice (C57BL/10ScSn) was included as a control. Animals will be sacrificed at 26 weeks post-dose. The following parameters and endpoints included: mortality, clinical observation, grip strength, gait analysis, MRI, Creatine Kinase (CK) analysis, weekly body weights, gross examination, tissue weights, and histopathology including spermatogenesis.

### 6.8: Example 8: 6 Week Pharmacology Study in MDX Mice

To further elucidate the pharmacology of RGX-DYS1 in mdx mice, AAV8-RGX-DYS1 was administered intravenously to 5-week-old mdx mice (5 males per group) at doses of 0 (vehicle), 3×1013, 1×1014, and 3×1014 GC/kg. An additional group of wild-type mice (C57BL/10ScSn) was included as a control. Animals will be sacrificed at 6 weeks post-dose. In vitro force measurement, including eccentric contraction, will be assessed in EDL and diaphragm muscles as a functional muscle endpoint. The following endpoints will also be included: mortality, clinical observations, weekly body weights, transgene expression (protein), gross examination, tissue weights, and muscle pathology.

### 6.9 Summary and Conclusion of Pharmacology Studies

A series of in vivo pharmacology studies were conducted using mdx mice, a biologically relevant model for DMD. This DMD murine model (mdx) exhibits many of the clinical and pathological manifestations humans experience with DMD (Rodrigues et al, 2016).

When AAV8-RGX-DYS1 was intravenously administrated to mdx mice, an improvement in muscle function (grip strength and in vitro force measurement at 2×10¹⁴ GC/kg; gait analysis at ≥1×10¹⁴ GC/kg) was evident 6 weeks post-dosing. Moreover, the effect of RGX-DYS1 on muscle function, as evidenced by automated gait analysis, was more prominent at 12 weeks post-dosing at doses ≥1×10¹⁴ GC/kg. An assessment of dystrophic lesions with T2-MRI showed improvement at doses of ≥1×10¹⁴ GC/kg and were comparable to the wild-type levels at these doses.

AAV8-RGX-DYS1 administration significantly reduced dystrophic pathology (inflammation, degeneration, and regeneration) in mdx mice at a dose of 2×10¹⁴ GC/kg and will be further assessed after 6 or 26 weeks to further characterize the dose-relationship. These RGX-DYS1 effects on muscle function, biomarkers, and pathology were associated with increased levels of RGX-DYS1 microdystrophin and vector DNA. In the AAV8-RGX-DYS1-administered mdx mice, high levels of RGX-DYS1 microdystrophin and RGX-DYS1 vector DNA in skeletal muscles were already observed at a dose of 2×10¹⁴ GC/kg after 6 weeks post dosing. Furthermore, highly sustained RGX-DYS1 microdystrophin and vector DNA levels were observed in skeletal and cardiac muscles after 12 weeks post-dosing.

An additional examination of RGX-DYS1 microdystrophin by immunofluorescence following AAV8-RGX-DYS1 administration confirmed a robust and correct localization of RGX-DYS1 microdystrophin at the sarcolemma of the TA and diaphragm muscles similar to wild type. A uniform dystrophin expression is required to stabilize myofiber turnover and attenuate pathology in dystrophic muscle (van Westering et al, 2020). In addition to RGX-DYS1 microdystrophin expression, other dystrophin-associated proteins were also restored and correctly expressed at the sarcolemma of the TA and diaphragm muscles, suggesting improved structural integrity in muscle.

In summary, a single dose of AAV8-RGX-DYS1 in a relevant animal model of DMD disease has provided remarkable benefits for muscle function, biomarkers associated with muscle damage, dystrophic muscle pathology, and other dystrophin-associated proteins. At AAV8-RGX-DYS1 doses >1×10¹⁴ GC/kg, there was significant improvement in muscle function as measured by fine motor kinematic gait analysis and improvement in muscle preservation as measured by MRI in the 12-week study. At a AAV8-RGX-DYS1 dose of 2×10¹⁴ GC/kg in the 6-week POC study, in addition to significant improvement in muscle function there was also significant improvement in dystrophic pathology and DAPC protein expression. Moreover, high levels of vector DNA and increased RGX-DYS1 microdystrophin protein expression were observed in skeletal and cardiac muscles at doses ≥1×10¹⁴ GC/kg. Therefore, the minimum effective dose following IV administration of AAV8-RGX-DYS1 to mdx mice, a murine model of DMD disease, is currently considered to be 1×10¹⁴ GC/kg.

### 6.10 Example 9: Toxicology Studies in Mice

The toxicity of AAV8-RGX-DYS1 has been evaluated in both the 12-week and 26-week pharmacology studies (non-GLP) in mdx mice.

### 6.10.1 12 Week Mdx Mouse Toxicology Study

As described above in the 12 week study in mdx mice for the pharmacology study, the same study protocol was used for toxicity studies (Example 4 herein). Groups of mdx male mice (n=10 per group) were administered a single IV injection of AAV8-RGX-DYS1 at 0 (vehicle), 3×10¹³, 1×10¹⁴, 3×10¹⁴, or 5×10¹⁴ GC/kg. An additional group of wild type mice (C57BL/10ScSn) was included as a control. Animals were sacrificed at 12 weeks post AAV8-RGX-DYS1 administration. The following parameters and endpoints included: mortality, clinical observation, body weights, gross examination, tissue weights, and histopathology, including spermatogenesis.

There were no test-article-related clinical observations and mean body weights increased over the time of the study in all dose groups. Mean body weights of mdx mice administered AAV8-RGX-DYS1 doses of >3×10¹⁴ GC/kg significantly decreased compared to the vehicle control mdx mice from Week 3 onward. Although their body weights were lower than the wild type, the difference was minimal (less than 10%) with no statistical difference between wild-type mice and mdx mice administered AAV8-RGX-DYS1 at the two high doses (3×10¹⁴ and 5×10¹⁴ GC/kg).

There were no AAV8-RGX-DYS1-related mortalities or clinical observations during the study period. At necropsy, brain, pituitary gland, spinal cord (cervical, thoracic, and lumbar), adrenal gland, kidney, liver, lung, mandibular and mesenteric lymph nodes, pancreas, spleen, thymus, prostate gland, seminal vesicle gland, testis, and epididymis were collected and were examined microscopically. The administration of a single IV dose of AAV8-RGX-DYS1 to male mdx mice up to 5×10¹⁴ GC/kg was not associated with any gross lesions, organ weight differences or microscopic findings in the tissues examined, including the male reproductive organs.

Therefore, when AAV8-RGX-DYS1 was administered to groups of mdx mice, the no-observed-adverse-effect level (NOAEL) was 5×10¹⁴ GC/kg, the highest dose tested.

### 6.10.2. 26 Week Toxicology Study in Mdx Mice

A toxicology study was conducted in connection with the 26 week in mdx mice pharmacology study described in Example 5 herein. Groups of mdx male mice (n=10 per group per time point) were administered a single IV injection of AAV8-RGX-DYS1 at 0 (vehicle), 3×1013, 1×1014, 3×1014, or 5×1014 GC/kg. An additional group of wild type mice (C57BL/10ScSn) was included as a control. Animals will be sacrificed at 26 weeks post-dose. The following parameters and endpoints included: mortality, clinical observation, weekly body weights, gross examination, tissue weights, and histopathology including spermatogenesis.

### 6.11 Example 10—Clinical Trial Protocol

### 6.11.1 Study Rationale

Duchenne muscular dystrophy (DMD) is an X-linked form of muscular dystrophy that results in progressive muscle weakness usually leading to death by young adulthood. DMD affects approximately 1 in 3,600 to 9,300 male births worldwide (Mah et al, 2014). The disease is caused by mutations in the DMD gene, which is located on the X chromosome and codes for a protein (dystrophin) that provides structural stability to skeletal and cardiac muscle fibers via the DAPC on muscle cell membranes (Hoffman et al, 1987). The lack of functional dystrophin in patients with DMD gene mutations reduces muscle cells' plasma membrane stability. Membrane destabilization results in altered mechanical properties and aberrant signaling, which contribute to membrane fragility, necrosis, inflammation, and progressive muscle wasting (Evans et al, 2009).

AAV8-RGX-DYS1 is a recombinant adeno-associated virus type 8 containing a human microdystrophin expression cassette designed to express microdystrophin from a muscle-specific promoter, and potentially prevent muscle degeneration in patients with DMD irrespective of the DMD mutation.

### 6.11.2 Dose

A dose of AAV8-RGX-DYS1 of 2×10¹⁴ GC/kg body mass will be administered as a single IV dose.

### 6.11.3. Study Design

This is a Phase 1/2 open-label, one-time dose study to evaluate the safety, tolerability, PD (RGX-DYS1 microdystrophin expression levels), PK (vector concentration), and clinical efficacy of AAV8-RGX-DYS1 in at least 6 ambulant male participants with DMD over the course of a 52-week study period. Safety will be the primary focus, with a secondary focus on expression levels of RGX-DYS1 microdystrophin. At least 6 participants will be enrolled to receive AAV8-RGX-DYS1 at a single dose level. Participants will be enrolled at the time written informed consent is given and will receive intervention only after completion of all Pretreatment Screening and Baseline assessments.

In this trial, two groups will assess data accumulated from the trial, an external IDMC and the Sponsor's ISC. The primary role of the IDMC is to assess safety at periodic intervals. The primary role of the ISC is to monitor safety on an ongoing basis.

Participants will receive AAV8-RGX-DYS1 at a dose of 2×10¹⁴ GC/kg. The first 3 participants must weigh less than 30 kg to receive AAV8-RGX-DYS1 and dosing will be staggered by at least 4 weeks. Safety data will be reviewed by the ISC after each of the participants has completed 4 weeks of follow-up. After the third participant has been followed for 4 weeks post-dosing and safety data have been reviewed by the IDMC and ISC, subsequent participants may be dosed in parallel. The ISC will make a recommendation after review of each participant's safety data. If the ISC recommends continuing, the next participant will be dosed.

After each participant has completed 12 weeks post-dosing of AAV8-RGX-DYS1, RGX-DYS1 microdystrophin expression levels will be determined in muscle biopsies, and participants will be evaluated for clinical efficacy by functional tests.

Following completion of Week 12, participants will continue to be assessed for safety and efficacy for 52 weeks following administration of AAV8-RGX-DYS1. At the end of the study, all participants will be invited to participate in a long-term follow-up study.

Participants will be assessed for ambulatory function, timed tasks, and strength throughout the 52-week follow-up periods using validated outcome measures (McDonald et al, 2013; McDonald et al, 2018; Mutoni et al, 2019), including the North Star Ambulatory Assessment (NSAA) linear score. Additional efficacy outcomes will be measured, including Time to Stand (TTSTAND), Time to Run/Walk 10 meters (TTRW), Time to Climb four stairs (TTCLIMB), myometry; as well as assessment of muscle using MRI imaging, cardiac and pulmonary function, creatine kinase levels, and patient-reported outcomes.

Sample Size and Power Calculation: At least 6 participants will be enrolled to assess the safety and tolerability of AAV8-RGX-DYS1 and explore the effect of AAV8-RGX-DYS1 on biomarker and clinical efficacy endpoints. Sample size is not based on any statistical justification.

Statistical Methods: All data will be summarized using descriptive statistics. Categorical variables will be analyzed using frequencies and percentages, and continuous variables will be summarized using descriptive statistics (number of non-missing observations, mean, standard deviation, median, minimum, and maximum). Subject listings and graphical displays will be presented as appropriate.

### 6.11.4 Eligibility Criteria

Participants in this study will be males who have a diagnosis of DMD based on clinical manifestations; family history, if applicable; and confirmed by skeletal muscle biopsy for dystrophin analysis by immunofluorescence or Western blot, or genotyping demonstrating a mutation consistent with DMD. Participants must be able to walk at least 100 meters without assistive devices and be able to rise to standing from supine (Time-to-Stand Test [TTSTAND]) in ≥3 and <9 seconds.

### 6.11.5 Inclusion

Male Participant's parent(s) or legal guardian(s) has (have) provided written informed consent and Health Insurance Portability and Accountability Act (HIPAA) authorization, where applicable, prior to any study-related procedures; participants will be asked to give written or verbal assent according to local requirements.

Participants must be at least 4 years of age and less than 12 years of age.

Participant has previous diagnosis of DMD, as defined as: Dystrophin immunofluorescence and/or Western blot analysis of skeletal muscle biopsy showing dystrophin deficiency, and clinical picture consistent with typical DMD, OR Identifiable mutation within the DMD gene (deletion/duplication of one or more exons), where reading frame can be predicted as ‘out-of-frame,’ and clinical picture consistent with typical DMD, OR Complete DMD gene sequencing showing an alteration (point mutation, duplication, other) that is expected to preclude production of the dystrophin protein (i.e., nonsense mutation, deletion/duplication leading to a downstream stop codon), with a clinical picture consistent with typical DMD.

Participant is able to walk 100 meters independently without assistive devices, as assessed at the Screening Visit.

Participant is able to complete the TTSTAND without assistance in ≥3 and <9 seconds, as assessed at the Screening Visit.

Clinical laboratory test results, including hepatic and renal function, are within the normal range at the Screening Visit, or if abnormal, are not clinically significant, in the opinion of the Investigator.

Documentation is provided at the Screening Visit that the participant has had 2 doses of measles, mumps, rubella, and varicella vaccine, with or without serologic evidence of immunity.

Participant has been on a stable daily dose of systemic glucocorticoids, ≥0.5 mg/kg/day prednisone or prednisolone or ≥0.75 mg/kg/day deflazacort, for at least 12 weeks prior to the Screening Visit.

Participant and parent(s)/guardian(s) are willing and able to comply with scheduled visits, study intervention administration plan, and study procedures.

### 6.11.6 Exclusion

Patients are excluded if one or more of the following are true:

Participant has a serious or unstable medical or psychological condition that, in the opinion of the PI, would compromise the subject's safety or successful participation in the study or interpretation of the study results.

Participant has evidence of symptomatic cardiomyopathy.

Participant has severe behavioral or cognitive problems that preclude participation in the study, in the opinion of the Investigator;

Participant has detectable AAV8 total binding antibodies in serum;

Participant has any non-healed injury or surgery that could impact functional testing (e.g., NSAA).

Participant has received any investigational or commercial gene therapy product over his lifetime.

Participant has a history of human immunodeficiency virus (HIV) or hepatitis B or hepatitis C virus infection, or positive screening tests for hepatitis B (hepatitis B surface antigen, hepatitis B surface antibody, hepatitis B core antibody [IgG]), or hepatitis C (either hepatitis C antibody or HCV RNA), or HIV antibodies.

Participant is a first-degree family member of a clinical site employee or any other individual involved with the conduct of the study.

Participant is currently taking any other investigational intervention or has taken any other investigational intervention within 3 months prior to the scheduled Day 1 intervention.

### 6.11.7 Study Objectives and Endpoints

| | | |
|---|---|---|
| Type | Objectives | Endpoints |

Primary

| | | |
|---|---|---|
| Safety | To evaluate the safety and | AEs and SAEs |
| | tolerability of AAV8-RGX- | Vital signs, Physicial |
| | DYS1 administered as a | exam, clinical laboratory |
| | single IV dose of 2 × 10¹⁴ | tests, 12-lead |
| | GC/kg body mass in | electrocardiogram, 2D- |
| | ambulant boys with DMD | echocardiography, |
| | | immune responses to |
| | | AAV8 vector |

Secondary

| | | |
|---|---|---|
| Pharma- | To investigate the effects | Quantification of RGX- |
| codynamics | on microdystrophin | DYS1 microdystrophin |
| (PD) | expression levels in muscle | at baseline and Week 12: |
| | of AAV8-RGX-DYS1 | RGX-DYS1 Microdystrophin |
| | administered as a single IV | expression levels determined |
| | dose of 2 × 10¹⁴ GC/kg | in biceps muscle biopsy |
| | body mass in ambulant boys | |
| | with DMD | |
| Pharma- | To investigate the single- | Blood and urine at 24-hour |
| cokinetics | dose PK and vector shedding | post-dose, Week 1, Week 2, |
| (PK) and | of AAV8-RGX-DYS1 | Week 4, Week 12, and |
| Vector | administered as a single IV | Months 6 and 12. |
| Shedding | dose of 2 × 10¹⁴ GC/kg | |
| | body mass in ambulant | |
| | boys with DMD | |
| Clinical | To investigate the clinical | Longitudinal trajectory of the |
| Efficacy | efficacy of AAV8-RGX- | following endpoints and |
| | DYS1 administered as a | change from baseline to the |
| | single IV dose of | Week 12, and Months 6, 9, |
| | 2 × 10¹⁴ GC/kg body mass in | and 12 assessment time points in: |
| | ambulant boys with DMD | NSAA linear score (Primary |
| | | efficacy outcome measure) and |
| | | raw Total score of NSAA |
| | | TTSTAND velocity (rise/second) |
| | | TTCLIMB velocity (tasks/second) |
| | | TTRW velocity (meters/second) |
| | | to complete 10 meters |
| | | Parameters from hand-held |
| | | myometry |
| | | Parameters from POSNA PODCI |
| | | Parameters from Peds QL |
| | | Neuromuscular Core ADL |
| | | and FIM |
| Clinical | To investigate the clinical | Longitudinal trajectory of the |
| Efficacy | efficacy of AAV8-RGX- | following endpoints: |
| | DYS1 administered as a | Parameters from MRI imaging |
| | single IV dose of | of skeletal muscle at baseline, |
| | 2 × 10¹⁴ GC/kg body mass | Month 6, and Month 12 |
| | in ambulant boys with DMD | Parameters from Cardiac MRI |
| | | at baseline and Month 12 |
| | | Parameters from pulmonary |
| | | function testing at baseline, |
| | | Month 6, and Month 12 |

### 6.11.8 Study Assessments and Procedures

Participants will receive AAV8-RGX-DYS1 at a dose of 2×10¹⁴ GC/kg. Dosing of the first 3 participants will be staggered by at least 4 weeks with a review of safety data by the ISC after each of the participants have completed 4 weeks of follow-up. After the third participant has been followed for 4 weeks post-dosing and safety data have been reviewed by the ISC and IDMC, subsequent participants may be dosed in parallel. The ISC will make a recommendation after review of each participant's safety data. If the ISC recommends continuing, the next participant will be dosed.

After each participant has completed 12 weeks post-dosing, RGX-DYS1 microdystrophin expression levels will be determined in muscle biopsies, and participants will be evaluated for clinical efficacy by functional tests. Following completion of Week 12, participants will continue to be assessed for safety and efficacy for 52 weeks following administration of AAV8-RGX-DYS1. At the end of the study, all participants will be invited to participate in a long-term follow-up study.

### 6.11.9 Bioanalytical Approach

### Muscle Biopsies—RGX-DYS1 Microdystrophin Distribution

An immunohistochemistry assay and western blots will be employed to detect fibers expressing RGX-DYS1 microdystrophin, and its localization. Immunogenicity Assays

### Anti-AAV8 Antibodies Assay

An electrochemiluminescence-based assay utilizing the Mesoscale platform will be validated to detect total antibodies to AAV8 in serum, and it will be used to monitor potential immune responses to AAV8-RGX-DYS1. This assay will not be used to determine subjects' eligibility to enroll in the study. A separate assay is being validated to identify eligible subjects based on anti-AAV8 antibodies status.

### Elispot

An interferon gamma ELISPOT assay will be developed to detect potential cellular responses to AAV8-RGX-DYS1, directed to either the AAV8 capsid proteins or RGX-DYS1 microdystrophin.

### RGX-DYS1 Vector Assays

### Vector Shedding

A qPCR assay that measures RGX-DYS1 in blood (or serum) and urine will be developed to measure shedding of the RGX-DYS1 vector after administration.

### Biodistribution in Muscle Biopsies

A qPCR assay that measures RGX-DYS1 vector DNA in muscle biopsies will be developed to measure the vector biodistribution to the target tissue after administration.

### 6.12 Methods of Administering AAV8-RGX-DYS1 in Combination with a Therapy for Dystrophinopathy

Clinical studies may be carried out to assess the therapeutic benefit of administering AAV8-RGX-DYS1 as described in Example 10 (Section 6.11, supra) in combination with a second therapeutic effective for treatment or amelioration of symptoms of a dystrophinopathy. The protocol described in Example 10 above can be used for administration of AAV8-RGX-DYS1. The dosing for a second therapeutic used in combination with the microdystrophin can be any of the clinical protocols known for the second therapeutics described herein. The combination treatments can last for at least a 6 months, one year, two years, three years, four years, five years, or up to at least 10 years. The combination may provide a synergistic therapeutic benefit for one or more of the monitored clinical endpoints as compared to each therapeutic on its own or, alternatively, the therapeutics may each ameliorate a different set of therapeutic end points such that the therapeutic benefit is greater than each therapeutic administered on its own.

For the combination of microdystrophin and casimersen, a subject can receive at least one dose of at least 1×10¹⁴ GC/kg or in some methods 2×10¹⁴ GC/kg of AAV8-RGX-DYS1 and 30 mg/kg once weekly of casimersen.

For the combination of microdystrophin and eteplirsen, a subject can receive at least one dose of at least 1×10¹⁴ GC/kg or in some methods 2×10¹⁴ GC/kg of AAV8-RGX-DYS1 and 30 mg/kg once weekly of eteplirsen.

For the combination of microdystrophin and golodirsen, a subject can receive at least one dose of at least 1×10¹⁴ GC/kg or in some methods 2×10¹⁴ GC/kg of AAV8-RGX-DYS1 and 30 mg/kg once weekly of golodirsen.

For the combination of microdystrophin and viltolarsen, a subject can receive at least one dose of at least 1×10¹⁴ GC/kg or in some methods 2×10¹⁴ GC/kg of AAV8-RGX-DYS1 and 80 mg/kg once weekly of viltolarsen.

For the combination of microdystrophin and ataluren, a subject can receive at least one dose of at least 1×10¹⁴ GC/kg or in some methods 2×10¹⁴ GC/kg of AAV8-RGX-DYS1 and ataluren suspension orally TID, 10 milligrams/kilogram (mg/kg) at morning, 10 mg/kg at midday, and 20 mg/kg at evening (total daily dose 40 mg/kg).

For the combination of microdystrophin and prednisone, a subject can receive at least one dose of at least 1×10¹⁴ GC/kg or in some methods 2×10¹⁴ GC/kg of AAV8-RGX-DYS1 and a daily dose of prednisone at 0.75 mg/kg/day.

For the combination of microdystrophin and deflazacort, a subject can receive at least one dose of at least 1×10¹⁴ GC/kg or in some methods 2×10¹⁴ GC/kg of AAV8-RGX-DYS1 and a daily dose of deflazacort at 0.9 mg/kg/day.

### REFERENCES

Adams M E, Kramarcy N, Krall S P, Rossi S G, Rotundo R L, Sealock R, Froehner S C. Absence of alpha-syntrophin leads to structurally aberrant neuromuscular synapses deficient in utrophin. J Cell Biol. 2000 Sep. 18; 150(6):1385-98. doi: 10.1083/jcb.150.6.1385.
Ahi Y S, Mittal S K. Components of Adenovirus Genome Packaging. Front Microbiol. 2016 Sep. 23; 7:1503. doi: 10.3389/fmicb.2016.01503.
Athanasopoulos T, Foster H, Foster K, Dickson G. Codon optimization of the microdystrophin gene for Duchene muscular dystrophy gene therapy. Methods Mol Biol. 2011; 709:21-37. doi: 10.1007/978-1-61737-982-6_2.
Banks G B, Judge L M, Allen J M, Chamberlain J S. The polyproline site in hinge 2 influences the functional capacity of truncated dystrophins. PLoS Genet. 2010; 6(5):e1000958. Published 2010 May 20. doi:10.1371/journal.pgen.1000958
Beekman C, Janson A A, Baghat A, van Deutekom J C, Datson N A. Use of capillary Western immunoassay (Wes) for quantification of dystrophin levels in skeletal muscle of healthy controls and individuals with Becker and Duchenne muscular dystrophy. PLoS One. 2018; 13(4):e0195850. Published 2018 Apr. 11. doi:10.1371/journal.pone.0195850
Binks M. Parent Project Muscular Dystrophy Annual Conference 2019
BioNews. Translarna (ataluren). Muscular Dystrophy News Today.
https://musculardystrophynews.com/translarna-ataluren/ Updated Nov. 26, 2019. Accessed Feb. 5, 2020.
BioNews. Vyondys 53 (golodirsen) Muscular Dystrophy News Today. https://musculardystrophynews.com/golodirsen-srp-4053/ Updated December 13,
Birnkrant D J, Bushby K, Bann C M, et al. Diagnosis and management of Duchenne muscular dystrophy, part 2: respiratory, cardiac, bone health, and orthopaedic management. Lancet Neurol. 2018; 17(4):347-361. doi:10.1016/S1474-4422(18)30025-5
Brooke M H, Fenichel G M, Griggs R C, et al. Duchenne muscular dystrophy: patterns of clinical progression and effects of supportive therapy. Neurology. 1989; 39(4):475-481. doi:10.1212/wnl.39.4.475
Bulfield G, Siller W G, Wight P A, Moore K J. X chromosome-linked muscular dystrophy (mdx) in the mouse. Proc Natl Acad Sci USA. 1984; 81(4):1189-1192. doi:10.1073/pnas.81.4.1189
Bushby K, Finkel R, Birnkrant D J, et al. Diagnosis and management of Duchenne muscular dystrophy, part 1: diagnosis, and pharmacological and psychosocial management. Lancet Neurol. 2010; 9(1):77-93. doi:10.1016/S1474-4422(09)70271-6
Chan S, Head S I, Morley J W. Branched fibers in dystrophic mdx muscle are associated with a loss of force following lengthening contractions. Am J Physiol Cell Physiol. 2007; 293(3):C985-C992. doi:10.1152/ajpcell.00128.2007
Chroboczek J, Bieber F, Jacrot B. The sequence of the genome of adenovirus type 5 and its comparison with the genome of adenovirus type 2. Virology. 1992; 186(1):280-285. doi:10.1016/0042-6822(92)90082-z
Cirak S, Feng L, Anthony K, et al. Restoration of the dystrophin-associated glycoprotein complex after exon skipping therapy in Duchenne muscular dystrophy. Mol Ther. 2012; 20(2):462-467. doi:10.1038/mt.2011.248
Coley W D, Bogdanik L, Vila M C, et al. Effect of genetic background on the dystrophic phenotype in mdx mice. Hum Mol Genet. 2016; 25(1):130-145. doi:10.1093/hmg/ddv460
CureDuchenne: What is Duchenne? https://www.cureduchenne.org/about/what-is-duchenne/Accessed 15 Mar. 2020.
Datson N A, Bijl S, Janson A, et al. Using a State-of-the-Art Toolbox to Evaluate Molecular and Functional Readouts of Antisense Oligonucleotide-Induced Exon Skipping in mdx Mice. Nucleic Acid Ther. 2020; 30(1):50-65. doi:10.1089/nat.2019.0824
Donovan-Banfield I, Turnell A S, Hiscox J A, Leppard K N, Matthews D A. Deep splicing plasticity of the human adenovirus type 5 transcriptome drives virus evolution. Commun Biol. 2020; 3(1):124. Published 2020 Mar. 13. doi:10.1038/s42003-020-0849-9
Dowling P, Gargan S, Murphy S, et al. The Dystrophin Node as Integrator of Cytoskeletal Organization, Lateral Force Transmission, Fiber Stability and Cellular Signaling in Skeletal Muscle. Proteomes. 2021; 9(1):9. Published 2021 Feb. 2. doi:10.3390/proteomes9010009
Duan D, Goemans N, Takeda S, Mercuri E, Aartsma-Rus A. Duchenne muscular dystrophy. Nat Rev Dis Primers. 2021; 7(1):13. Published 2021 Feb. 18. doi:10.1038/s41572-021-00248-3
Ehmsen J, Poon E, Davies K. The dystrophin-associated protein complex. J Cell Sci. 2002; 115(Pt 14):2801-2803.
Emery A E H. Duchenne Muscular Dystrophy. (1993) Oxford University Press, Oxford.
Evans N P, Misyak S A, Robertson J L, Bassaganya-Riera J, Grange R W. Immune-mediated mechanisms potentially regulate the disease time-course of duchenne muscular dystrophy and provide targets for therapeutic intervention. P M R. 2009; 1(8):755-768. doi:10.1016/j.pmrj.2009.04.010
Falzarano M S, Scotton C, Passarelli C, Ferlini A. Duchenne Muscular Dystrophy: From Diagnosis to Therapy. Molecules. 2015; 20(10):18168-18184. Published 2015 Oct. 7. doi:10.3390/molecules201018168
Faust S M, Bell P, Cutler B J, et al. CpG-depleted adeno-associated virus vectors evade immune detection. J Clin Invest. 2013; 123(7):2994-3001. doi:10.1172/JCI68205
Ferlini A, Flanigan K M, Lochmuller H, Muntoni F, Hoen P A, McNally E. 204th ENMC International Workshop on Biomarkers in Duchenne Muscular Dystrophy 24-26 Jan. 2014.
Forbes S C, Arora H, Willcocks R J, et al. Upper and Lower Extremities in Duchenne Muscular Dystrophy Evaluated with Quantitative MRI and Proton M R Spectroscopy in a Multicenter Cohort. Radiology. 2020; 295(3):616-625. doi:10.1148/radiol.2020192210
Gao G P, Alvira M R, Wang L, Calcedo R, Johnston J, Wilson J M. Novel adeno-associated viruses from rhesus monkeys as vectors for human gene therapy. Proc Natl Acad Sci USA. 2002; 99(18):11854-11859. doi:10.1073/pnas.182412299
Grady R M, Grange R W, Lau K S, et al. Role for alpha-dystrobrevin in the pathogenesis of dystrophin-dependent muscular dystrophies. Nat Cell Biol. 1999; 1(4):215-220. doi:10.1038/12034
Graham F L, Smiley J, Russell W C, Nairn R. Characteristics of a human cell line transformed by DNA from human adenovirus type 5. J Gen Virol. 1977; 36(1):59-74. doi:10.1099/0022-1317-36-1-59
Granchelli J A, Pollina C, Hudecki M S. Pre-clinical screening of drugs using the mdx mouse. Neuromuscul Disord. 2000; 10(4-5):235-239. doi:10.1016/s0960-8966(99)00126-1
Hathout Y, Marathi R L, Rayavarapu S, et al. Discovery of serum protein biomarkers in the mdx mouse model and cross-species comparison to Duchenne muscular dystrophy patients. Hum Mol Genet. 2014; 23(24):6458-6469. doi:10.1093/hmg/ddu366
Heier C R, Guerron A D, Korotcov A, et al. Non-invasive MRI and spectroscopy of mdx mice reveal temporal changes in dystrophic muscle imaging and in energy deficits. PLoS One. 2014; 9(11):e112477. Published 2014 Nov. 12. doi:10.1371/journal.pone.0112477
Hoffman E P, Brown R H Jr, Kunkel L M. Dystrophin: the protein product of the Duchenne muscular dystrophy locus. Cell. 1987; 51(6):919-928. doi:10.1016/0092-8674(87)90579-4
Hoffman E P, Brown R H Jr, Kunkel L M. Dystrophin: the protein product of the Duchenne muscular dystrophy locus. Cell. 1987; 51(6):919-928. doi:10.1016/0092-8674(87)90579-4
Hoffman E P. Pharmacotherapy of Duchenne Muscular Dystrophy. Handb Exp Pharmacol. 2020; 261:25-37. doi:10.1007/164_2019_256
Hogrel J Y, Wary C, Moraux A, et al. Longitudinal functional and NMR assessment of upper limbs in Duchenne muscular dystrophy. Neurology. 2016; 86(11):1022-1030. doi:10.1212/WNL.0000000000002464
Hordeaux J, Hinderer C, Goode T, et al. Toxicology Study of Intra-Cisterna Magna Adeno-Associated Virus 9 Expressing Human Alpha-L-Iduronidase in Rhesus Macaques. Mol Ther Methods Clin Dev. 2018; 10:79-88. Published 2018 Jul. 14. doi:10.1016/j.omtm.2018.06.003
Hosaka Y, Yokota T, Miyagoe-Suzuki Y, et al. Alpha1-syntrophin-deficient skeletal muscle exhibits hypertrophy and aberrant formation of neuromuscular junctions during regeneration. J Cell Biol. 2002; 158(6):1097-1107. doi:10.1083/jcb.200204076
Jiang H, Couto L B, Patarroyo-White S, et al. Effects of transient immunosuppression on adenoassociated, virus-mediated, liver-directed gene transfer in rhesus macaques and implications for human gene therapy. Blood. 2006; 108(10):3321-3328. doi:10.1182/blood-2006-04-017913
Kieny P, Chollet S, Delalande P, et al. Evolution of life expectancy of patients with Duchenne muscular dystrophy at AFM Yolaine de Kepper centre between 1981 and 2011. Ann Phys Rehabil Med. 2013; 56(6):443-454. doi:10.1016/j.rehab.2013.06.002
Koenig M, Hoffman E P, Bertelson C J, Monaco A P, Feener C, Kunkel L M. Complete cloning of the Duchenne muscular dystrophy (DMD) cDNA and preliminary genomic organization of the DMD gene in normal and affected individuals. Cell. 1987; 50(3):509-517. doi:10.1016/0092-8674(87)90504-6
Koo T, Malerba A, Athanasopoulos T, et al. Delivery of AAV2/9-microdystrophin genes incorporating helix 1 of the coiled-coil motif in the C-terminal domain of dystrophin improves muscle pathology and restores the level of α1-syntrophin and α-dystrobrevin in skeletal muscles of mdx mice. Hum Gene Ther. 2011; 22(11):1379-1388. doi:10.1089/hum.2011.020
Laurila E, Ahola A, Hyttinen J, Aalto-Setala K. Methods for in vitro functional analysis of iPSC derived cardiomyocytes—Special focus on analyzing the mechanical beating behavior. Biochim Biophys Acta. 2016; 1863(7 Pt B):1864-1872. doi:10.1016/j.bbamcr.2015.12.013
Le Guiner C, Servais L, Montus M, et al. Long-term microdystrophin gene therapy is effective in a canine model of Duchenne muscular dystrophy. Nat Commun. 2017; 8:16105. Published 2017 Jul. 25. doi:10.1038/ncomms16105
Li X, Eastman E M, Schwartz R J, Draghia-Akli R. Synthetic muscle promoters: activities exceeding naturally occurring regulatory sequences. Nat Biotechnol. 1999; 17(3):241-245. doi:10.1038/6981
Lim K R, Maruyama R, Yokota T. Eteplirsen in the treatment of Duchenne muscular dystrophy. Drug Des Devel Ther. 2017; 11:533-545. Published 2017 Feb. 28. doi:10.2147/DDDT.S97635
Lin B, Li Y, Han L, et al. Modeling and study of the mechanism of dilated cardiomyopathy using induced pluripotent stem cells derived from individuals with Duchenne muscular dystrophy. Dis Model Mech. 2015; 8(5):457-466. doi:10.1242/dmm.019505
Mah J K, Korngut L, Dykeman J, Day L, Pringsheim T, Jette N. A systematic review and meta-analysis on the epidemiology of Duchenne and Becker muscular dystrophy. Neuromuscul Disord. 2014; 24(6):482-491. doi:10.1016/j.nmd.2014.03.008
Manno C S, Pierce G F, Arruda V R, et al. Successful transduction of liver in hemophilia by AAV-Factor IX and limitations imposed by the host immune response [published correction appears in Nat Med. 2006 May; 12(5):592. Rasko, John [corrected to Rasko, John J E]; Rustagi, Pradip K [added]]. Nat Med. 2006; 12(3):342-347. doi:10.1038/nm1358
Matthews D J, James K A, Miller L A, et al. Use of corticosteroids in a population-based cohort of boys with duchenne and becker muscular dystrophy. J Child Neurol. 2010; 25(11):1319-1324. doi:10.1177/0883073810362762
Maurissen J P, Marable B R, Andrus A K, Stebbins K E. Factors affecting grip strength testing. Neurotoxicol Teratol. 2003; 25(5):543-553. doi:10.1016/s0892-0362(03)00073-4
McDonald C M, Henricson E K, Abresch R T, et al. Long-term effects of glucocorticoids on function, quality of life, and survival in patients with Duchenne muscular dystrophy: a prospective cohort study. Lancet. 2018; 391(10119):451-461. doi:10.1016/50140-6736(17)32160-8
McDonald C M, Henricson E K, Abresch R T, et al. The cooperative international neuromuscular research group Duchenne natural history study—a longitudinal investigation in the era of glucocorticoid therapy: design of protocol and the methods used. Muscle Nerve. 2013; 48(1):32-54. doi:10.1002/mus.23807
Meyer O A, Tilson H A, Byrd W C, Riley M T. A method for the routine assessment of fore- and hindlimb grip strength of rats and mice. Neurobehav Toxicol. 1979; 1(3):233-236.
Moens P, Baatsen P H, Mardchal G. Increased susceptibility of EDL muscles from mdx mice to damage induced by contractions with stretch. J Muscle Res Cell Motil. 1993 August; 14(4):446-51. doi: 10.1007/BF00121296. PMID: 7693747.
Moorwood C, Liu M, Tian Z, Barton E R. Isometric and eccentric force generation assessment of skeletal muscles isolated from murine models of muscular dystrophies. J Vis Exp. 2013; (71):e50036. Published 2013 Jan. 31. doi:10.3791/50036
Muntoni F, Domingos J, Manzur A Y, et al. Categorising trajectories and individual item changes of the North Star Ambulatory Assessment in patients with Duchenne muscular dystrophy. PLoS One. 2019; 14(9):e0221097. Published 2019 Sep. 3. doi:10.1371/journal.pone.0221097
Nagaraju K, Willmann R; TREAT-NMD Network and the Wellstone Muscular Dystrophy Cooperative Research Network. Developing standard procedures for murine and canine efficacy studies of DMD therapeutics: report of two expert workshops on “Pre-clinical testing for Duchenne dystrophy”: Washington D.C., Oct. 27-28, 2007 and Zürich, Jun. 30-Jul. 1, 2008. Neuromuscul Disord. 2009; 19(7):502-506. doi:10.1016/j.nmd.2009.05.003
Ozawa E, Hagiwara Y, Yoshida M. Creatine kinase, cell membrane and Duchenne muscular dystrophy. Mol Cell Biochem. 1999; 190(1-2):143-151.
Pratt S J P, Xu S, Mullins R J, Lovering R M. Temporal changes in magnetic resonance imaging in the mdx mouse. BMC Res Notes. 2013; 6:262. Published 2013 Jul. 9. doi:10.1186/1756-0500-6-262
Preisig D F, Kulic L, Kruger M, et al. High-speed video gait analysis reveals early and characteristic locomotor phenotypes in mouse models of neurodegenerative movement disorders. Behav Brain Res. 2016; 311:340-353. doi:10.1016/j.bbr.2016.04.044
Rodrigues M, Echigoya Y, Fukada S I, Yokota T. Current Translational Research and Murine Models For Duchenne Muscular Dystrophy. J Neuromuscul Dis. 2016; 3(1):29-48. doi:10.3233/JND-150113 Ropars J, Gravot F, Ben Salem D, Rousseau F, Brochard S, Pons C. Muscle MRI:
A biomarker of disease severity in Duchenne muscular dystrophy? A systematic review. Neurology. 2020; 94(3):117-133. doi:10.1212/WNL.0000000000008811
Sadoulet-Puccio H M, Rajala M, Kunkel L M. Dystrobrevin and dystrophin: an interaction through coiled-coil motifs. Proc Natl Acad Sci USA. 1997; 94(23):12413-12418.doi:10.1073/pnas.94.23.12413
Sancar F, Touroutine D, Gao S, et al. The dystrophin-associated protein complex maintains muscle excitability by regulating Ca(2+)-dependent K(+) (BK) channel localization. J Biol Chem. 2011; 286(38):33501-33510. doi:10.1074/jbc.M111.227678
Scallan C D, Jiang H, Liu T, et al. Human immunoglobulin inhibits liver transduction by AAV vectors at low AAV2 neutralizing titers in SCID mice. Blood. 2006; 107(5):1810-1817. doi:10.1182/blood-2005-08-3229
Sicinski P, Geng Y, Ryder-Cook A S, Barnard E A, Darlison M G, Barnard P J. The molecular basis of muscular dystrophy in the mdx mouse: a point mutation. Science. 1989; 244(4912):1578-1580. doi:10.1126/science.2662404
Smith J P, Hicks P S, Ortiz L R, Martinez M J, Mandler R N. Quantitative measurement of muscle strength in the mouse. J Neurosci Methods. 1995; 62(1-2):15-19. doi:10.1016/0165-0270(95)00049-6
Turk R, Sterrenburg E, de Meijer E J, van Ommen G J, den Dunnen J T, 't Hoen P A. Muscle regeneration in dystrophin-deficient mdx mice studied by gene expression profiling. BMC Genomics. 2005; 6:98. Published 2005 Jul. 13. doi:10.1186/1471-2164-6-98
Uaesoontrachoon K, Srinivassane S, Warford J, et al. Orthogonal analysis of dystrophin protein and mRNA as a surrogate outcome for drug development. Biomark Med. 2019; 13(14):1209-1225. doi:10.2217/bmm-2019-0242
van Westering T L E, Lomonosova Y, Coenen-Stass A M L, et al. Uniform sarcolemmal dystrophin expression is required to prevent extracellular microRNA release and improve dystrophic pathology. J Cachexia Sarcopenia Muscle. 2020; 11(2):578-593. doi:10.1002/jcsm.12506
Vandeputte C, Taymans J M, Casteels C, et al. Automated quantitative gait analysis in animal models of movement disorders. BMC Neurosci. 2010; 11:92. Published 2010 Aug. 9. doi:10.1186/1471-2202-11-92
Wang B, Li J, Xiao X. Adeno-associated virus vector carrying human minidystrophin genes effectively ameliorates muscular dystrophy in mdx mouse model. Proc Natl Acad Sci USA. 2000; 97(25):13714-13719. doi:10.1073/pnas.240335297
Wokke B H, Van Den Bergen J C, Hooijmans M T, Verschuuren J J, Niks E H, Kan H E. T2 relaxation times are increased in Skeletal muscle of DMD but not BMD patients. Muscle Nerve. 2016; 53(1):38-43. doi:10.1002/mus.24679
Ropars J, Gravot F, Ben Salem D, Rousseau F, Brochard S, Pons C. Muscle MRI: A biomarker of disease severity in Duchenne muscular dystrophy? A systematic review. Neurology. 2020; 94(3):117-133. doi:10.1212/WNL.0000000000008811
Sadoulet-Puccio H M, Rajala M, Kunkel L M. Dystrobrevin and dystrophin: an interaction through coiled-coil motifs. Proc Natl Acad Sci USA. 1997; 94(23):12413-12418. doi:10.1073/pnas.94.23.12413
Sancar F, Touroutine D, Gao S, et al. The dystrophin-associated protein complex maintains muscle excitability by regulating Ca(2+)-dependent K(+) (BK) channel localization. J Biol Chem. 2011; 286(38):33501-33510. doi:10.1074/jbc.M111.227678
Scallan C D, Jiang H, Liu T, et al. Human immunoglobulin inhibits liver transduction by AAV vectors at low AAV2 neutralizing titers in SCID mice. Blood. 2006; 107(5):1810-1817. doi:10.1182/blood-2005-08-3229
Sicinski P, Geng Y, Ryder-Cook A S, Barnard E A, Darlison M G, Barnard P J. The molecular basis of muscular dystrophy in the mdx mouse: a point mutation. Science. 1989; 244(4912):1578-1580. doi:10.1126/science.2662404
Smith J P, Hicks P S, Ortiz L R, Martinez M J, Mandler R N. Quantitative measurement of muscle strength in the mouse. J Neurosci Methods. 1995; 62(1-2):15-19. doi:10.1016/0165-0270(95)00049-6
Turk R, Sterrenburg E, de Meijer E J, van Ommen G J, den Dunnen J T, 't Hoen P A. Muscle regeneration in dystrophin-deficient mdx mice studied by gene expression profiling. BMC Genomics. 2005; 6:98. Published 2005 Jul. 13. doi:10.1186/1471-2164-6-98
Uaesoontrachoon K, Srinivassane S, Warford J, et al. Orthogonal analysis of dystrophin protein and mRNA as a surrogate outcome for drug development. Biomark Med. 2019; 13(14):1209-1225. doi:10.2217/bmm-2019-0242
van Westering T L E, Lomonosova Y, Coenen-Stass A M L, et al. Uniform sarcolemmal dystrophin expression is required to prevent extracellular microRNA release and improve dystrophic pathology. J Cachexia Sarcopenia Muscle. 2020; 11(2):578-593. doi:10.1002/jcsm.12506
Vandeputte C, Taymans J M, Casteels C, et al. Automated quantitative gait analysis in animal models of movement disorders. BMC Neurosci. 2010; 11:92. Published 2010 Aug. 9. doi:10.1186/1471-2202-11-92
Wang B, Li J, Xiao X. Adeno-associated virus vector carrying human minidystrophin genes effectively ameliorates muscular dystrophy in mdx mouse model. Proc Natl Acad Sci USA. 2000; 97(25):13714-13719. doi:10.1073/pnas.240335297
Wokke B H, Van Den Bergen J C, Hooijmans M T, Verschuuren J J, Niks E H, Kan H E. T2 relaxation times are increased in Skeletal muscle of DMD but not BMD patients. Muscle Nerve. 2016; 53(1):38-43. doi:10.1002/mus.24679

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

What is claimed is:

1. A method of treating a dystrophinopathy in a subject in need thereof, comprising administering to the subject a first therapeutic and a second therapeutic which is different from said first therapeutic,
wherein the first therapeutic is a microdystrophin pharmaceutical composition comprising a therapeutically effective amount of a recombinant adeno-associated vector (rAAV) particle and a pharmaceutically acceptable carrier,
wherein the rAAV particle comprises a transgene that encodes a microdystrophin protein consisting of dystrophin domains arranged from amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site.

2. The method of claim 2, wherein the second therapeutic is a mutation suppression therapy, an exon skipping therapy, a steroid therapy, an immunosuppressive/anti-inflammatory therapy, or a therapy that treats one or more symptoms of the dystrophinopathy.

3. The method of claim 2, wherein the second therapeutic is a mutation suppression therapy.

4. The method of claim 3, wherein the second therapeutic is ataluren or gentamycin.

5.-6. (canceled)

7. The method of claim 2, wherein the second therapeutic is an exon skipping therapy.

8. (canceled)

9. The method of claim 7, wherein the exon skipping therapy results in skipping of one or more of exons 2, 43, 44, 45, 50, 51, 52, 53, 55 of the human dystrophin gene to express a form of dystrophin protein.

10. The method of claim 9, wherein the exon skipping therapy is:
a. casimersen SRP-5045 or DS-5141B and results in skipping exon 45,
b. SRP-5050 and results in skipping exon 50,
c. eteplirsen or SRP-5051 and results in skipping exon 51,
d. golodirsen, SRP-5053 or viltolarsen and results in skipping exon 53,
e. SRP-5052 and results in skipping exon 52,
f. SSRP-5044 or NS-089/NCNP-02 and results in skipping exon 44, and/or
g. scAAV9.U7.ACCA and results in skipping exon 2.

11.-31. (canceled)

32. The method of claim 2, wherein the second therapeutic is a steroid therapy.

33. The method of claim 32, wherein the steroid therapy is prednisone, deflazacort, Vamorolone, or Spironolactone, or a combination thereof.

34. The method of claim 2, wherein the second therapeutic is an immunosuppressive or anti-inflammatory therapy.

35. The method of claim 34, wherein the second therapeutic is edasalonexent, canakinumab, pamrevlumab, or imlifidase.

36.-40. (canceled)

41. The method of claim 2, wherein the second therapeutic is spironolactone, follistatin, SERCA2a, EDG-5506, tamoxifen, or Givinostat.

42.-64. (canceled)

65. The method of claim 1, wherein the therapeutically effective amount of the rAAV particle is administered intravenously at dose of 5×10¹³ to 1×10¹⁵ genome copies/kg.

66. The method of claim 1, wherein the CT comprises or consists of a) the proximal 194 amino acids of the C-terminus of dystrophin or b) at least the proximal portion of the C-terminus corresponding to human dystrophin amino acid residues 3361-3554 of SEQ ID NO:95 (UniProtKB-P11532) or c) at least the proximal portion of the C-terminus encoded by exons 70 to 74 and the first 36 amino acids of the amino acid sequence encoded by the nucleotide sequence of exon 75.

67. The method of claim 1, wherein the microdystrophin protein has the amino acid sequence of SEQ ID NO:1.

68. The method of claim 67, wherein the microdystrophin protein is encoded by the nucleic acid sequence of SEQ ID NO:20.

69.-71. (canceled)

72. The method of claim 1, wherein a transcription regulatory element that promotes expression in muscle is operably linked to the nucleic acid sequence that encodes the microdystrophin protein.

73.-74. (canceled)

75. The method of claim 72, wherein the transcription regulatory element comprises SPc5-12 or a transcriptionally active portion thereof.

76.-82. (canceled)

83. The method of claim 1, wherein the rAAV is an AAV8 serotype.

84. The method of claim 1, wherein the dystrophinopathy is Duchenne muscular dystrophy (DMD), Becker muscular dystrophy (BMD), or X-linked dilated cardiomyopathy.

85.-86. (canceled)
